(19) United States
(12) Patent Application Publication
(10) Pub. No.: US 2026/0337173 A1
(43) Pub. Date: Oct. 29, 2026

(54) ANTHRACENE COMPOUND AND ORGANIC ELECTROLUMINESCENT DEVICE

(71) Applicant: GUANGDONG AGLAIA OPTOELECTRONIC MATERIALS CO., LTD, Guangdong (CN)

(72) Inventors: Jiahui TAN, Guangdong (CN); Shaofu CHEN, Guangdong (CN); Lei DAI, Guangdong (CN); Lifei CAI, Guangdong (CN)

(73) Assignee: GUANGDONG AGLAIA OPTOELECTRONIC MATERIALS CO., LTD, Guangdong (CN)

(21) Appl. No.: 19/465,884

(22) Filed: Jan. 30, 2026

(30) Foreign Application Priority Data
Dec. 19, 2024 (CN) 2024118749278

(51) Int. Cl. C07D 307/91; H10K 85/60

(52) U.S. Cl. CPC C07D 307/91; H10K 85/615; H10K 85/6574

(57) ABSTRACT

The present disclosure relates to an anthracene compound and an organic electroluminescent device. The compound has a structure of formula (1), where ring A is selected from formula (2) or formula (3). The compound can be used in an OLED as a blue light host material, and has the advantages of low driving voltage, high luminous efficiency, long lifetime and the like, and has the potential of being applied to the AMOLED industry.

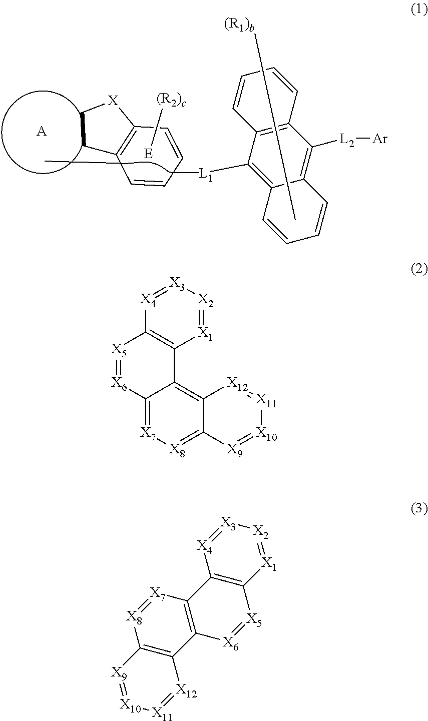

### CROSS-REFERENCE TO RELATED APPLICATION

This application claims priority to Chinese Patent Application No. 202411874927.8, filed Dec. 19, 2024, the disclosure of which is hereby incorporated by reference in its entirety.

### TECHNICAL FIELD

The present disclosure belongs to the technical field of organic electroluminescence, and particularly relates to an anthracene compound and an organic electroluminescent device.

### BACKGROUND

Currently, organic light-emitting devices (OLEDs), as a new-generation of display technology, have gained more and more attention in display and lighting technologies, and have very broad application prospects. However, compared with market application requirements, OLEDs still need further enhancement and improvement in in terms of luminous efficiency, driving voltage, service life, and the like.

Generally, the basic structure of an OLED is a thin film of organic functional materials with various functions sandwiched between metal electrodes, just like a sandwich structure. Driven by current, holes and electrons are injected from a cathode and an anode, respectively. After migrating a certain distance, the holes and electrons are recombined in a light-emitting layer and released in the form of light or heat to achieve luminescence of the OLED. In phosphorescent OLEDs, device properties are determined not only by the triplet emitter used but are also significantly influenced by other types of materials such as host materials. Host materials play a crucial role in reducing device driving voltage, improving luminous efficiency, and extending service life. However, the host materials disclosed in existing technology still leave room for optimization in terms of the above-mentioned performances.

Therefore, it is necessary to continue to develop new types of host materials to further improve the performance of organic electroluminescent devices.

### SUMMARY

The present disclosure aims to solve at least one of the technical problems in the existing technology. To this end, the present disclosure provides an anthracene compound, which can be used as a host material for an OLED, and is capable of significantly reducing the driving voltage of the device, improving luminous efficiency, and extending the service life of the device. The present disclosure further provides an organic electroluminescent device comprising the compound.

Specifically, an embodiment of a first aspect of the present disclosure relates to an anthracene compound having a structure of formula (1):

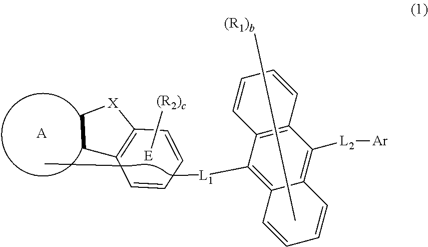

where ring A is selected from the following formula (2) or formula (3);

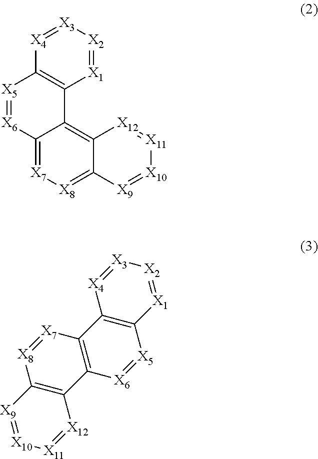

where X₁-X₁₂ are each independently selected from CR₀ or N; and two adjacent sites in X₁-X₄, X₅-X₆, X₇-X₈, X₉-X₁₂ are fused to a 5-membered ring containing X in formula (1);
R₀, R₁ and R₂ are each independently selected from hydrogen, deuterium, halogen, cyano, nitro, C₁-C₄₀ alkyl, C₁-C₄₀ heteroalkyl, C₂-C₄₀ alkenyl, C₂-C₄₀ alkynyl, C₃-C₄₀ cycloalkyl, C₃-C₄₀ heterocycloalkyl, C₆-C₆₀ aryl, C₃-C₆₀ heteroaryl, C₁-C₄₀ alkoxy, C₆-C₆₀ aryloxy, C₃-C₄₀ alkylsilyl, C₆-C₆₀ arylsilyl, C₁-C₄₀ alkylboranyl, C₆-C₆₀ arylboranyl, C₆-C₆₀ arylphosphino, or C₆-C₆₀ arylamino; and adjacent R₀ can be connected into a fused ring;
b is an integer of 0-8; if b≥2, each R₁ is the same or different;
c is an integer of 0-4; if c≥2, each R₂ is the same or different;
X is selected from O or S;
L₁ and L₂ are each independently selected from single bond, substituted or unsubstituted C₆-C₆₀ arylene, or substituted or unsubstituted C₃-C₆₀ heteroarylene;
Ar is selected from substituted or unsubstituted C₆-C₃₆ aryl, or substituted or unsubstituted C₂-C₃₆ heteroaryl;
each of substitutions in L₁, L₂ and Ar refers to being independently substituted with at least one of deuterium, halogen, cyano, isocyano, phosphino, C₁-C₆ alkyl, C₃-C₁₆ cycloalkyl, C₁-C₆ alkyl-substituted amino, C₁-C₆ hydrocarbyl-substituted or unsubstituted C₆-C₃₀ aryl, or C₁-C₆ hydrocarbyl-substituted or unsubstituted C₃-C₃₀ heteroaryl, wherein the number of substitutions ranges from monosubstitution to the maximum number of substitutions;
where a heteroatom in the heteroarylene, heteroaryl, heteroalkyl, or heterocycloalkyl is at least one selected from the group consisting of O, S, N, Se, Si, and Ge.

The present disclosure has the following beneficial effects:

The anthracene compound described in the present disclosure can be used in host materials for OLEDs, has the advantages of low driving voltage, high luminous efficiency, long lifetime and the like, and is particularly suitable for blue light-emitting devices. In addition, the compound has high optical and electrical stability, has the potential of being applied to active-matrix organic light emitting diode (AMOLED) industry.

### BRIEF DESCRIPTION OF THE DRAWINGS

FIG. 1 is a ¹H NMR spectrum of Compound 7 of an example of the present disclosure;

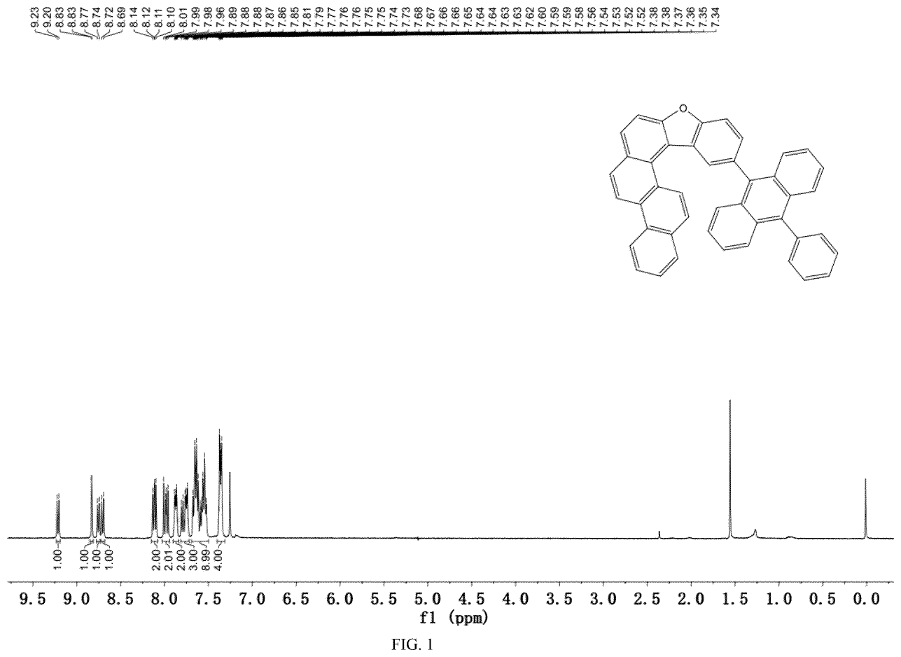

FIG. 2 is a schematic structural diagram of an organic electroluminescent device of an example of the present disclosure.

### DETAILED DESCRIPTION

In order to make the technical schemes described in the present disclosure more clearly apparent to those skilled in the art, the following examples are now listed for illustration. It should be noted that the following examples do not limit the scope of protection claimed by the present disclosure.

In a first aspect of the present disclosure, an embodiment provides an anthracene compound having a structure shown in formula (1).

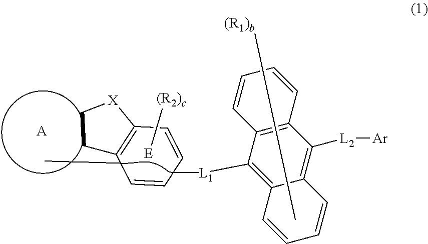

where ring A is selected from the following formula (2) or formula (3);

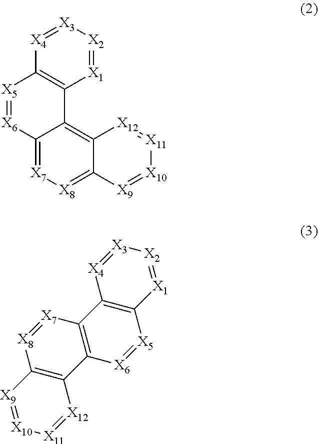

where X₁-X₁₂ are each independently selected from CR₀ or N; and two adjacent sites in X₁-X₄, X₅-X₆, X₇-X₈, X₉-X₁₂ are fused to a 5-membered ring containing X in formula (1);
R₀, R₁ and R₂ are each independently selected from hydrogen, deuterium, halogen, cyano, nitro, C₁-C₄₀ alkyl, C₁-C₄₀ heteroalkyl, C₂-C₄₀ alkenyl, C₂-C₄₀ alkynyl, C₃-C₄₀ cycloalkyl, C₃-C₄₀ heterocycloalkyl, C₆-C₆₀ aryl, C₃-C₆₀ heteroaryl, C₁-C₄₀ alkoxy, C₆-C₆₀ aryloxy, C₃-C₄₀ alkylsilyl, C₆-C₆₀ arylsilyl, C₁-C₄₀ alkylboranyl, C₆-C₆₀ arylboranyl, C₆-C₆₀ arylphosphino, or C₆-C₆₀ arylamino; and adjacent R₀ can be connected into a fused ring;
b is an integer of 0-8; if b≥2, each R₁ is the same or different;
c is an integer of 0-4; if c≥2, each R₂ is the same or different;
X is selected from O or S;
L₁ and L₂ are each independently selected from single bond, substituted or unsubstituted C₆-C₆₀ arylene, or substituted or unsubstituted C₃-C₆₀ heteroarylene;
Ar is selected from substituted or unsubstituted C₆-C₃₆ aryl, or substituted or unsubstituted C₂-C₃₆ heteroaryl;
each of substitutions in L₁, L₂ and Ar refers to being independently substituted with at least one of deuterium, halogen, cyano, isocyano, phosphino, C₁-C₆ alkyl, C₃-C₁₆ cycloalkyl, C₁-C₆ alkyl-substituted amino, C₁-C₆ hydrocarbyl-substituted or unsubstituted C₆-C₃₀ aryl, or C₁-C₆ hydrocarbyl-substituted or unsubstituted C₃-C₃₀ heteroaryl, wherein the number of substitutions ranges from monosubstitution to the maximum number of substitutions;
where a heteroatom in the heteroarylene, heteroaryl, heteroalkyl, or heterocycloalkyl is at least one selected from the group consisting of O, S, N, Se, Si, and Ge.

It can be understood that in formula (1), L₁ can be connected to ring A or ring E.

“Adjacent R₀” refers to R₀ located on adjacent carbon atoms, and it can be further understood that “adjacent R₀ can be connected into a fused ring” means that R₀ located on adjacent carbon atoms can be connected to form a fused ring. For example, when two adjacent R₀ are both phenyl groups, they can be connected to form a naphthalene ring.

In some embodiments, X₁-X₁₂ are all selected from CR₀.

In some embodiments, X₁-X₁₂ contains at least one N.

In some embodiments, the anthracene compound has a structure as shown in the following formula (4):

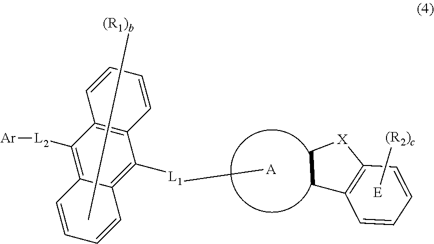

where the connection structure of ring A and L₁ moiety is:

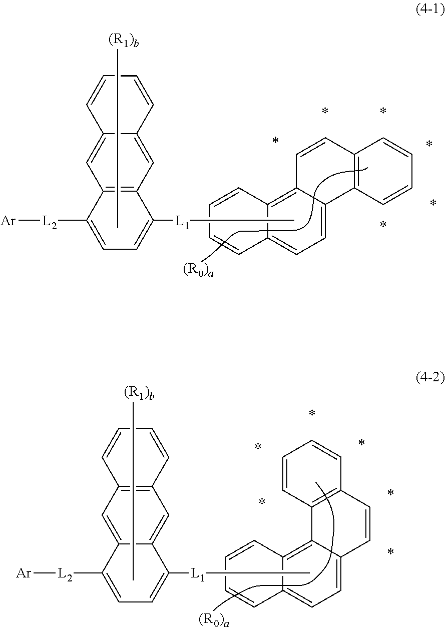

where * represents a site fused to a 5-membered ring containing X in formula (1);
a is an integer of 0-9; if a≥2, each R₀ is the same or different, and adjacent R₀ can be connected into a fused ring.

In some embodiments, the anthracene compound has a structure as shown in the following formula (6):

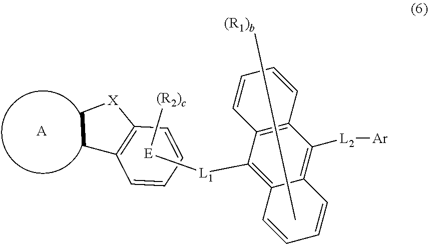

where c is an integer of 0-3; if c is 2 or 3, each R₂ is the same or different;
ring A is one of the structures shown in the following formulas (A-1) to (A-8):

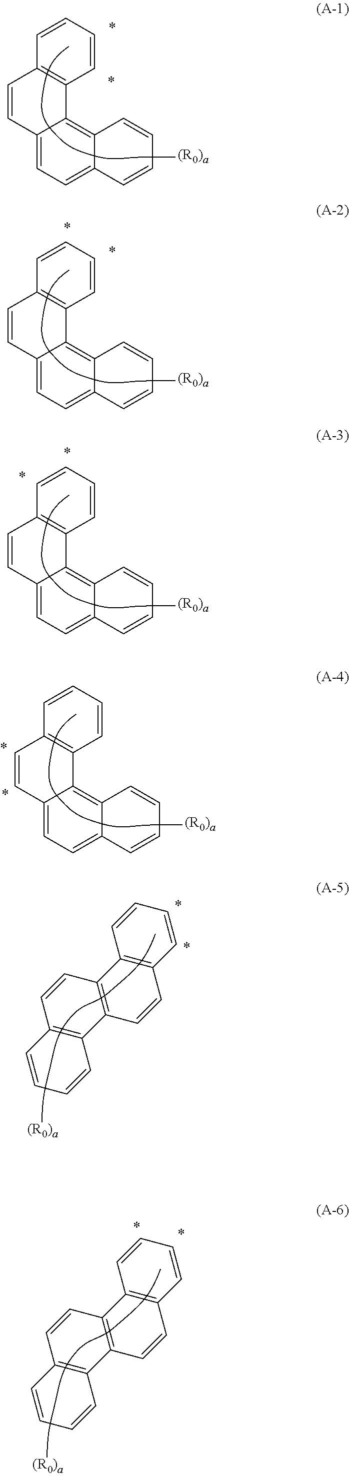

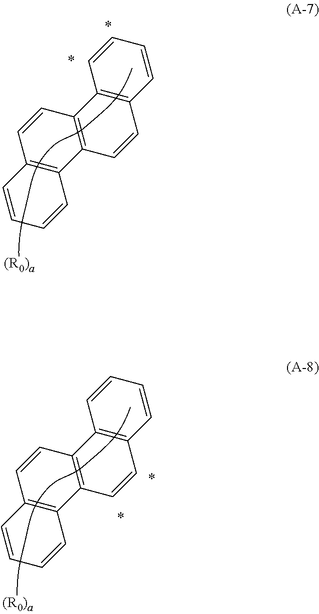

where * represents a site fused to a 5-membered ring containing X in formula (1);
a is an integer of ≥0-10; if a≥2, each R₀ is the same or different, and adjacent R₀ can be connected into a fused ring.

In some embodiments, ring A is one of the structures shown in the following formulas (A-9) to (A-16):

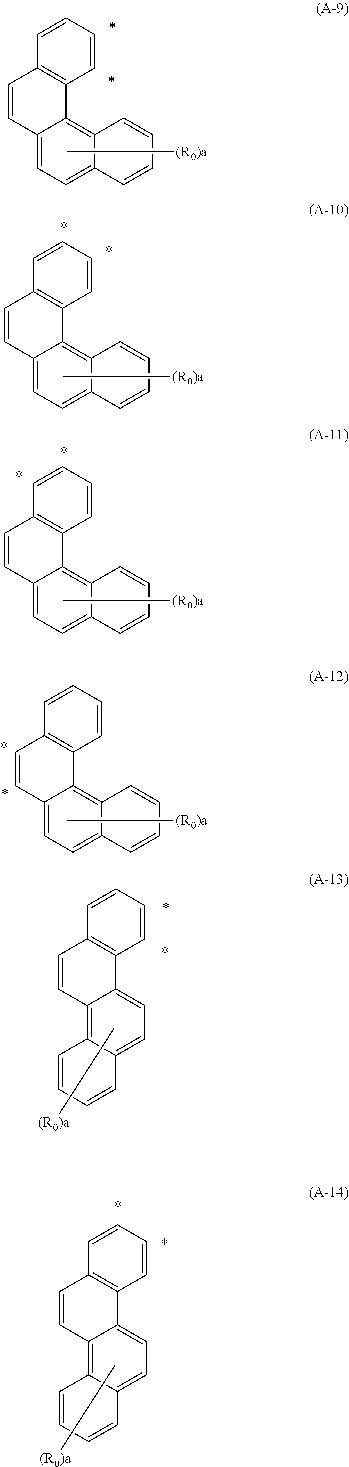

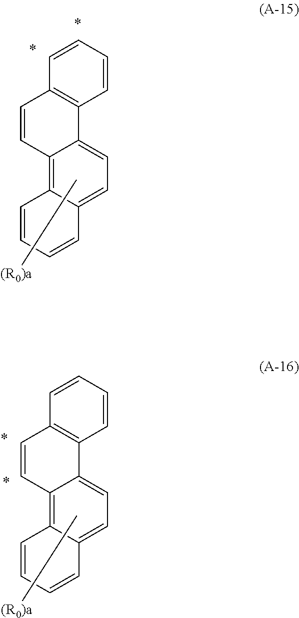

a is an integer of 0-6; if a≥2, each R₀ is the same or different, and adjacent R₀ can be connected into a fused ring.

In some embodiments, the anthracene compound has the following structures:

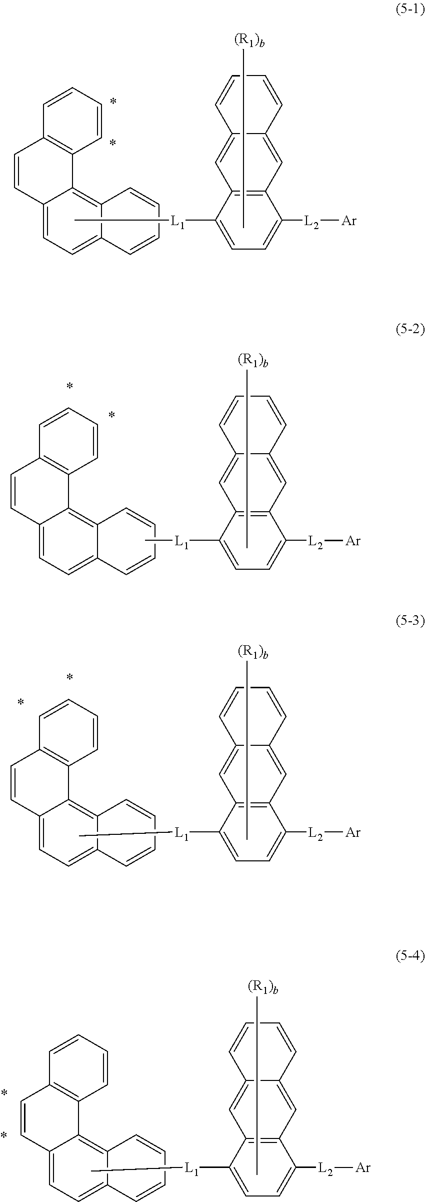

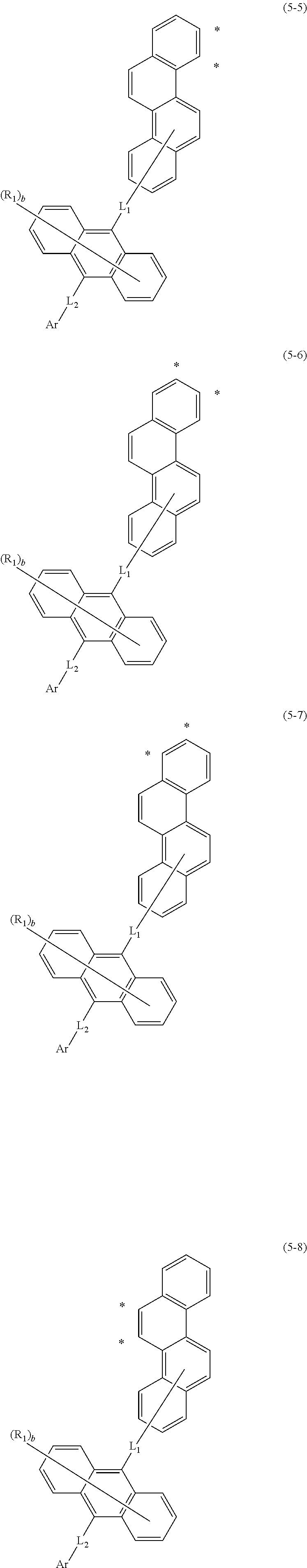

where * represents a site fused to a 5-membered ring containing X in formula (1).

In some embodiments, R₀ is selected from hydrogen, deuterium, halogen, cyano, nitro, C₁-C₄₀ alkyl, C₁-C₄₀ heteroalkyl, C₂-C₃₀ alkenyl, C₂-C₃₀ alkynyl, C₃-C₄₀ cycloalkyl, C₃-C₄₀ heterocycloalkyl, C₆-C₆₀ aryl, C₃-C₆₀ heteroaryl, C₁-C₃₀ alkoxy, C₆-C₃₀ aryloxy, C₃-C₃₀ alkylsilyl, C₆-C₃₀ arylsilyl, C₁-C₃₀ alkylboranyl, C₆-C₃₀ arylboranyl, C₆-C₃₀ arylphosphino, or C₆-C₃₀ arylamino; and adjacent R₀ can be connected into a fused ring.

In some embodiments, R₀ is selected from hydrogen, deuterium, halogen, cyano, nitro, C₁-C₄₀ alkyl, C₁-C₄₀ heteroalkyl, C₂-C₂₀ alkenyl, C₂-C₂₀ alkynyl, C₃-C₄₀ cycloalkyl, C₃-C₄₀ heterocycloalkyl, C₆-C₆₀ aryl, C₃-C₆₀ heteroaryl, C₁-C₂₀ alkoxy, C₆-C₂₀ aryloxy, C₃-C₂₀ alkylsilyl, C₆-C₂₀ arylsilyl, C₁-C₂₀ alkylboranyl, C₆-C₂₀ arylboranyl, C₆-C₂₀ arylphosphino, or C₆-C₂₀ arylamino; and adjacent R₀ can be connected into a fused ring.

In some embodiments, R₀ is selected from hydrogen, deuterium, halogen, cyano, nitro, C₁-C₄₀ alkyl, C₁-C₄₀ heteroalkyl, C₂-C₁₀ alkenyl, C₂-C₁₀ alkynyl, C₃-C₄₀ cycloalkyl, C₃-C₄₀ heterocycloalkyl, C₆-C₆₀ aryl, or C₃-C₆₀ heteroaryl; and adjacent R₀ can be connected into a fused ring.

In some embodiments, R₀, R₁ and R₂ are each independently selected from hydrogen, deuterium, halogen, cyano, C₁-C₄₀ alkyl, C₁-C₄₀ heteroalkyl, C₃-C₄₀ cycloalkyl, C₃-C₄₀ heterocycloalkyl, C₆-C₆₀ aryl, or C₃-C₆₀ heteroaryl; and adjacent R₀ can be connected into a fused ring.

In some embodiments, R₀, R₁ and R₂ are each independently selected from hydrogen, deuterium, halogen, cyano, C₁-C₃₀ alkyl, C₁-C₃₀ heteroalkyl, C₃-C₃₀ cycloalkyl, C₃-C₃₀ heterocycloalkyl, C₆-C₃₀ aryl, or C₃-C₃₀ heteroaryl; and adjacent R₀ can be connected into a fused ring.

In some embodiments, R₀, R₁ and R₂ are each independently selected from hydrogen, deuterium, halogen, cyano, C₁-C₂₀ alkyl, C₁-C₂₀ heteroalkyl, C₃-C₂₀ cycloalkyl, C₃-C₂₀ heterocycloalkyl, C₆-C₂₀ aryl, or C₃-C₂₀ heteroaryl.

In some embodiments, R₀, R₁ and R₂ are each independently selected from hydrogen, deuterium, halogen, cyano, C₁-C₁₂ alkyl, C₁-C₁₂ heteroalkyl, C₃-C₁₈ cycloalkyl, C₃-C₁₈ heterocycloalkyl, C₆-C₁₂ aryl, or C₃-C₁₂ heteroaryl.

In some embodiments, R₀, R₁ and R₂ are each independently selected from hydrogen, deuterium, halogen, cyano, C₁-C₁₀ alkyl, C₁-C₁₀ heteroalkyl, C₃-C₁₀ cycloalkyl, C₃-C₁₀ heterocycloalkyl, C₆-C₁₂ aryl, or C₃-C₁₂ heteroaryl.

In some embodiments, L₁ and L₂ are each independently selected from single bond, substituted or unsubstituted C₆-C₃₀ arylene, or substituted or unsubstituted C₃-C₃₀ heteroarylene.

In some embodiments, L₁ and L₂ are each independently selected from single bond, substituted or unsubstituted C₆-C₂₄ arylene, or substituted or unsubstituted C₃-C₂₀ heteroarylene.

In some embodiments, L₁ and L₂ are each independently selected from single bond, substituted or unsubstituted C₆-C₂₀ arylene, or substituted or unsubstituted C₃-C₂₀ heteroarylene.

In some embodiments, L₁ and L₂ are each independently selected from single bond, substituted or unsubstituted C₆-C₁₄ arylene, or substituted or unsubstituted C₃-C₁₂ heteroarylene.

In some embodiments, each of substitutions in L₁, L₂ and Ar refers to being independently substituted with at least one of deuterium, halogen, cyano, isocyano, phosphino, C₁-C₆ alkyl, C₃-C₁₆ cycloalkyl, C₁-C₆ alkyl-substituted amino, C₁-C₆ hydrocarbyl-substituted or unsubstituted C₆-C₁₈ aryl, or C₁-C₆ hydrocarbyl-substituted or unsubstituted C₃-C₁₈ heteroaryl, wherein the number of substitutions ranges from monosubstitution to the maximum number of substitutions. As an example, where C₃-C₁₆ cycloalkyl can specifically be C₃-C₁₂ cycloalkyl or C₃-C₁₀ cycloalkyl.

In some embodiments, each of substitutions in L₁, L₂ and Ar refers to being independently substituted with at least one of deuterium, halogen, cyano, isocyano, phosphino, C₁-C₆ alkyl, C₃-C₁₆ cycloalkyl, C₁-C₆ alkyl-substituted amino, C₁-C₆ hydrocarbyl-substituted or unsubstituted C₆-C₁₂ aryl, or C₁-C₆ hydrocarbyl-substituted or unsubstituted C₃-C₁₂ heteroaryl, wherein the number of substitutions ranges from monosubstitution to the maximum number of substitutions.

In some embodiments, each of substitutions in L₁, L₂ and Ar refers to being independently substituted with at least one of deuterium, halogen, cyano, isocyano, C₁-C₆ alkyl, C₃-C₁₆ cycloalkyl, C₁-C₆ hydrocarbyl-substituted or unsubstituted C₆-C₁₂ aryl, or C₁-C₆ hydrocarbyl-substituted or unsubstituted C₃-C₁₂ heteroaryl, wherein the number of substitutions ranges from monosubstitution to the maximum number of substitutions.

In some embodiments, Ar is selected from substituted or unsubstituted C₆-C₃₀ aryl, or substituted or unsubstituted C₂-C₃₀ heteroaryl.

In some embodiments, Ar is selected from substituted or unsubstituted C₆-C₂₅ aryl or substituted or unsubstituted C₃-C₂₅ heteroaryl.

In some embodiments, Ar is selected from substituted or unsubstituted phenyl, substituted or unsubstituted biphenyl, substituted or unsubstituted naphthyl, substituted or unsubstituted anthryl, substituted or unsubstituted dibenzofuranyl, substituted or unsubstituted dibenzothienyl, substituted or unsubstituted 9,9-dimethylfluorenyl, substituted or unsubstituted 9,9-diphenylfluorenyl, substituted or unsubstituted spirobifluorenyl, substituted or unsubstituted phenanthryl, substituted or unsubstituted pyrenyl, substituted or unsubstituted chrysenyl, substituted or unsubstituted carbazolyl, substituted or unsubstituted pyridyl, substituted or unsubstituted pyrimidinyl, substituted or unsubstituted fluoranthenyl, substituted or unsubstituted benzoxazolyl, substituted or unsubstituted benzimidazolyl, substituted or unsubstituted benzotriazolyl, substituted or unsubstituted benzophenanthryl, substituted or unsubstituted benzocyclohexyl, substituted or unsubstituted phenanthrooxazolyl, oxaspirofluorenyl, or substituted or unsubstituted benzocarbazolyl, or a combination of at least two thereof; the substitutions are substitutions with at least one of deuterium, halogen, cyano, isocyano, C₁-C₆ alkyl, or C₃-C₁₆ cycloalkyl, wherein the number of substitutions ranges from monosubstitution to the maximum number of substitutions. “Combination of at least two thereof” means that at least two groups as defined above are combined to form Ar, for example, phenyl and naphthyl are combined to form Ar. It should be understood that the groups are combined in such a manner as to satisfy the carbon number range of Ar defined above.

In some embodiments, the anthracene compound satisfies at least one of the following conditions:
(1) X₁-X₁₂ are all selected from CR₀, and R₀ is selected from hydrogen or deuterium;
(2) X is selected from O;
(3) R₁ is selected from hydrogen, deuterium, or substituted or unsubstituted phenyl;
(4) R₂ is selected from hydrogen or deuterium;
(5) L₁ and L₂ are each independently selected from single bond, substituted or unsubstituted phenylene, substituted or unsubstituted biphenylene, substituted or unsubstituted naphthylene, substituted or unsubstituted phenanthrylene, substituted or unsubstituted anthrylene, a divalent group of dibenzofuran, or a divalent group of dibenzothiophene;
(6) Ar is selected from the group consisting of substituted or unsubstituted phenyl, substituted or unsubstituted biphenyl, substituted or unsubstituted naphthyl, substituted or unsubstituted phenanthryl, substituted or unsubstituted anthryl, dibenzofuranyl, and dibenzothienyl;
where the substitutions are each independently substitutions with deuterium or fluorine, wherein the number of substitutions ranges from monosubstitution to the maximum number of substitutions.

In some embodiments, the compound is one of the following structural formulas, or a structure in which hydrogen in the following structural formulas is partially or completely substituted by deuterium, fluorine.

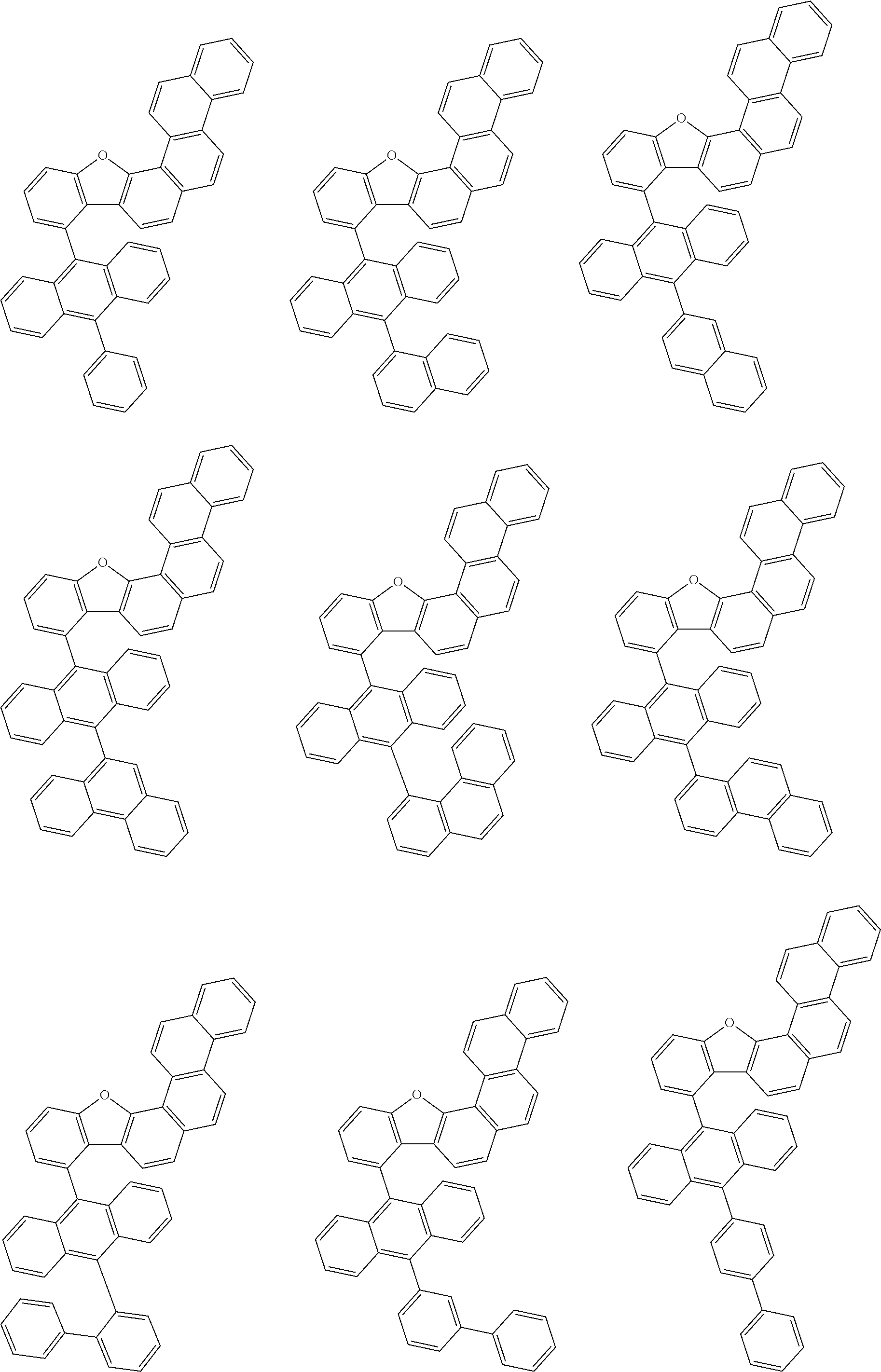

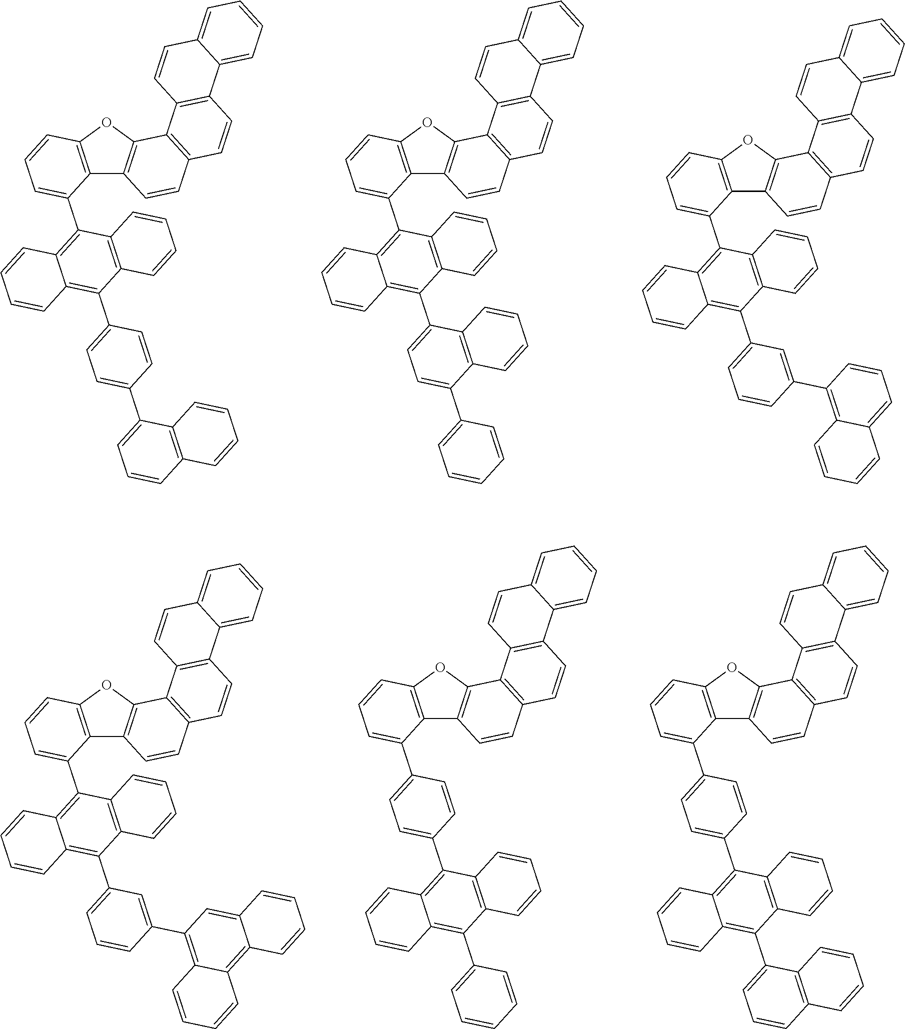

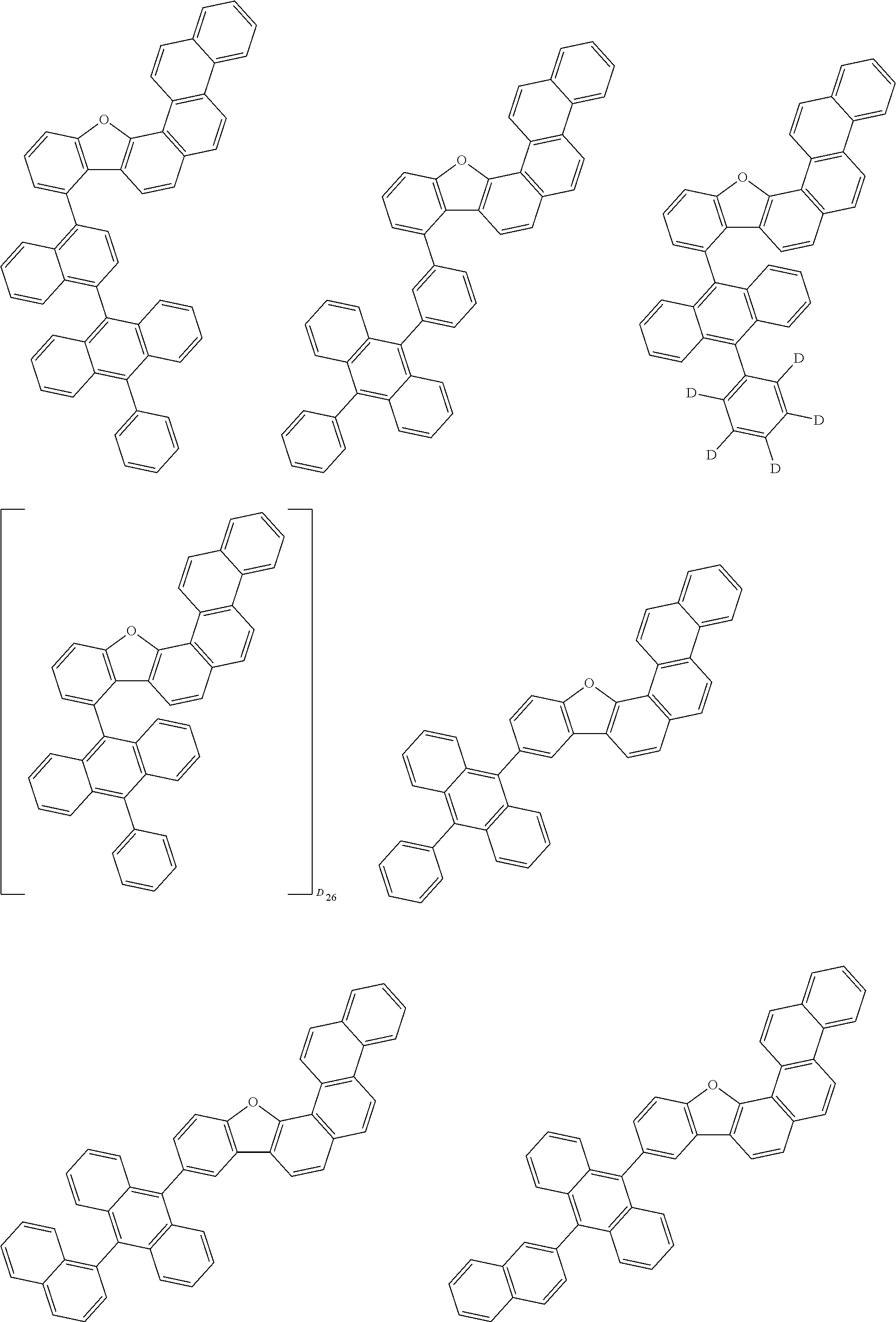

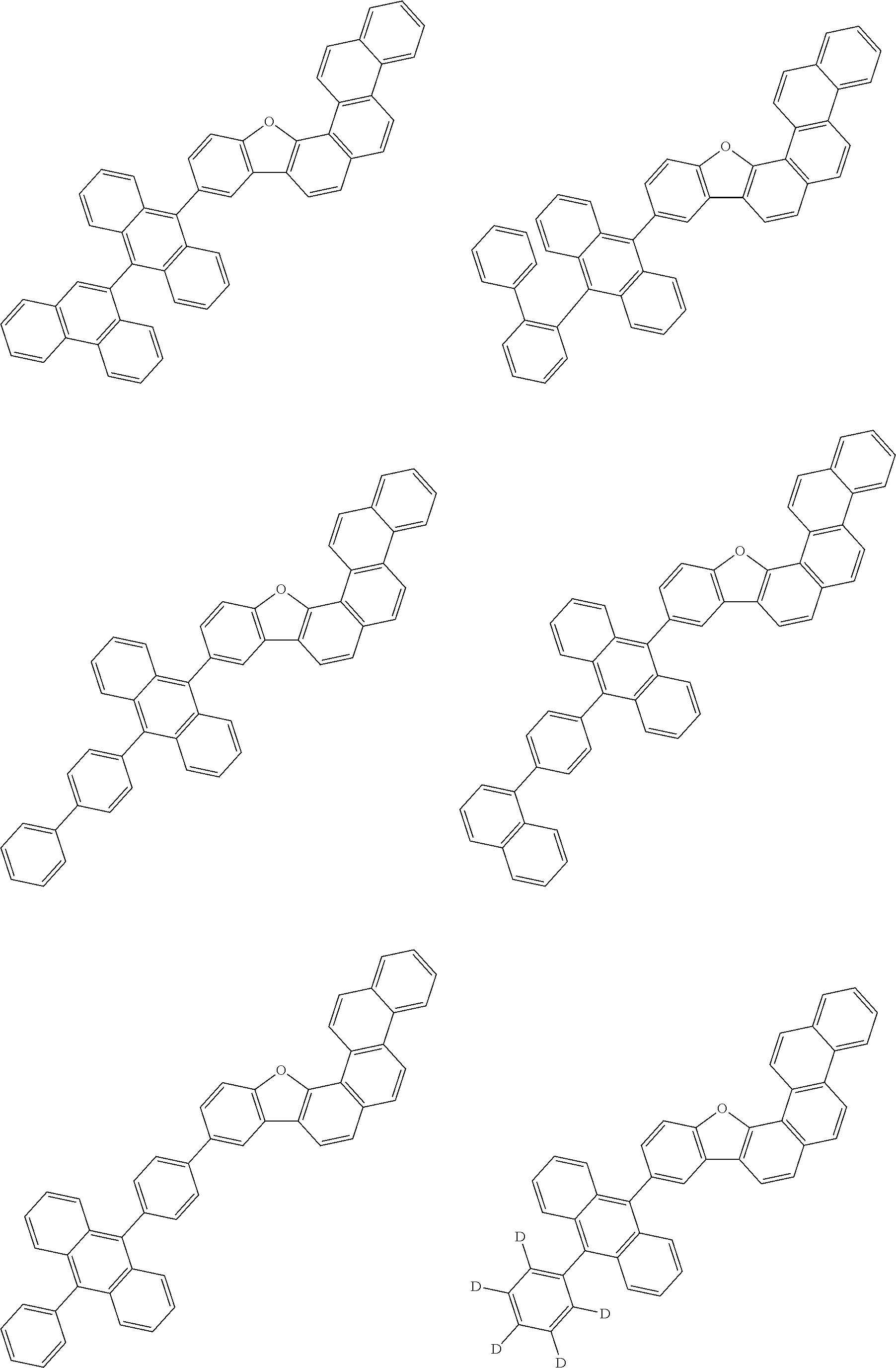

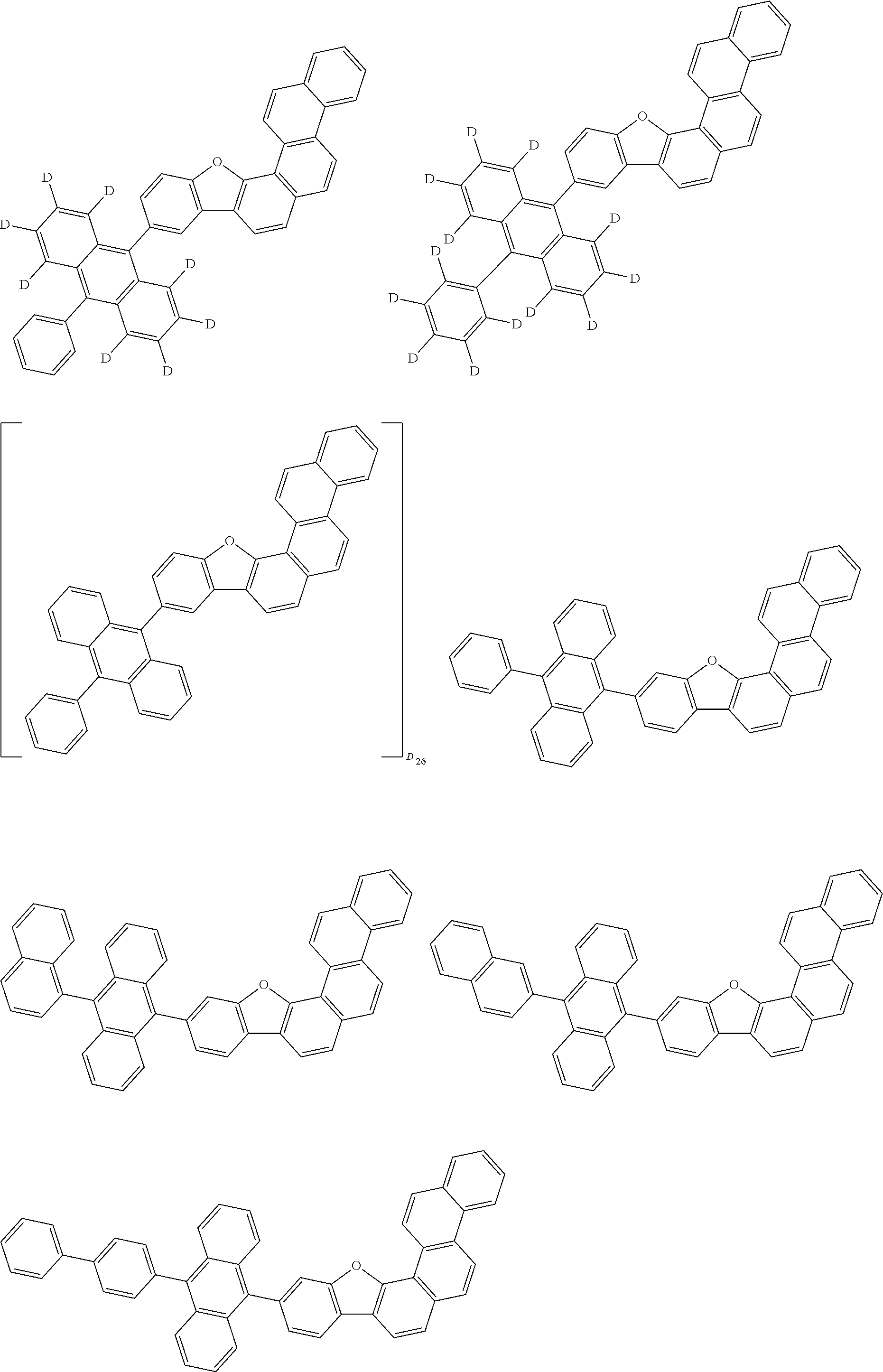

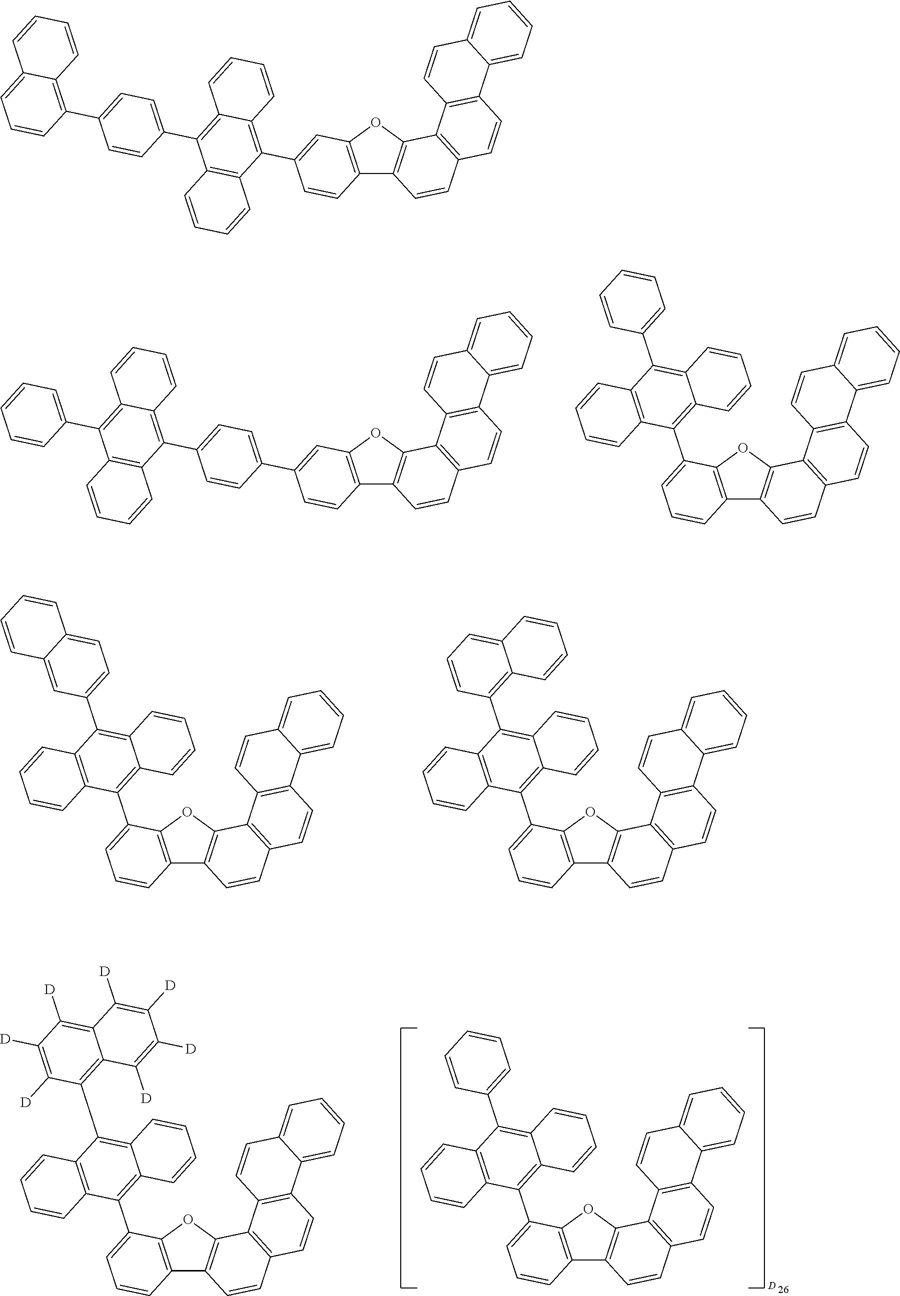

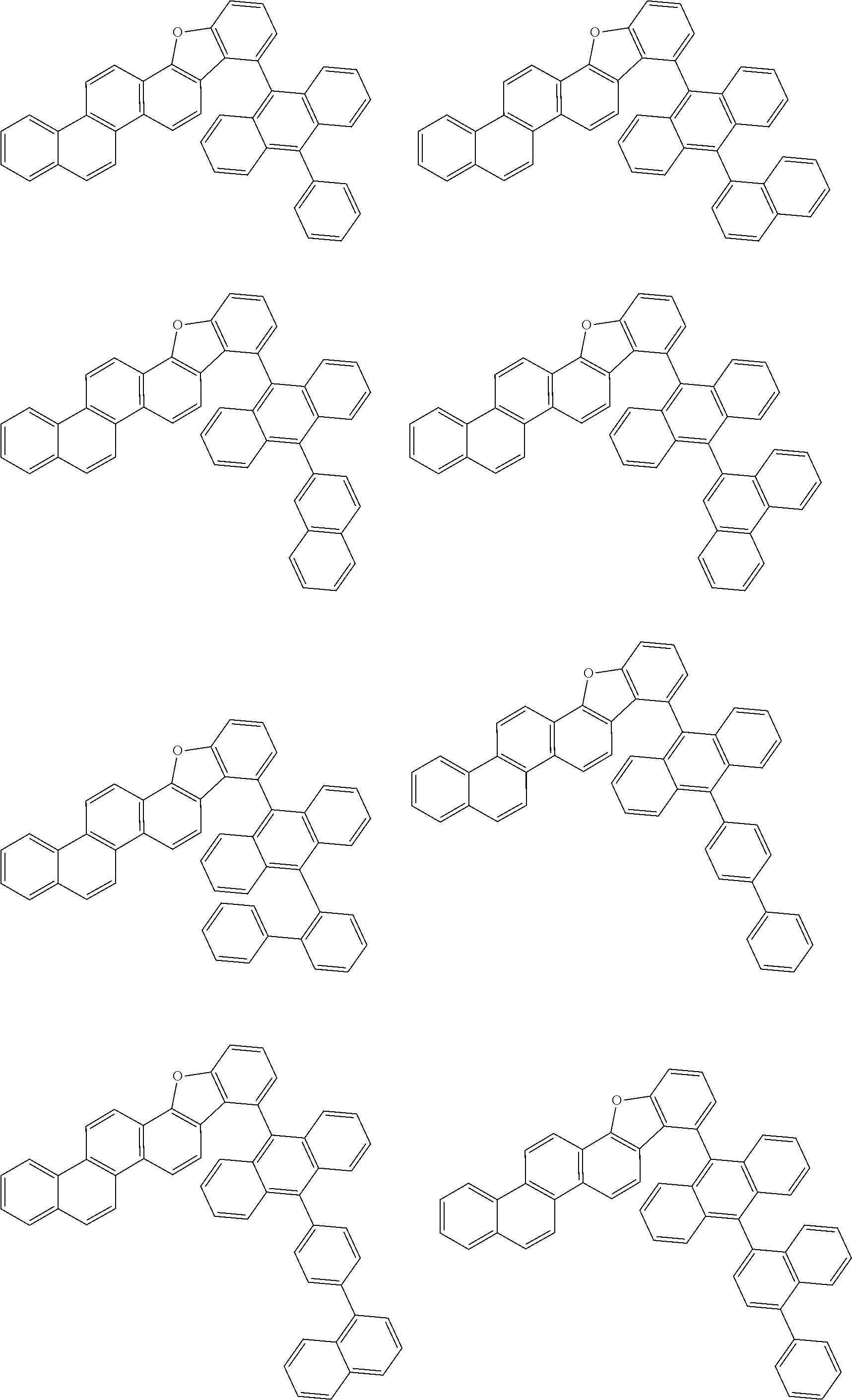

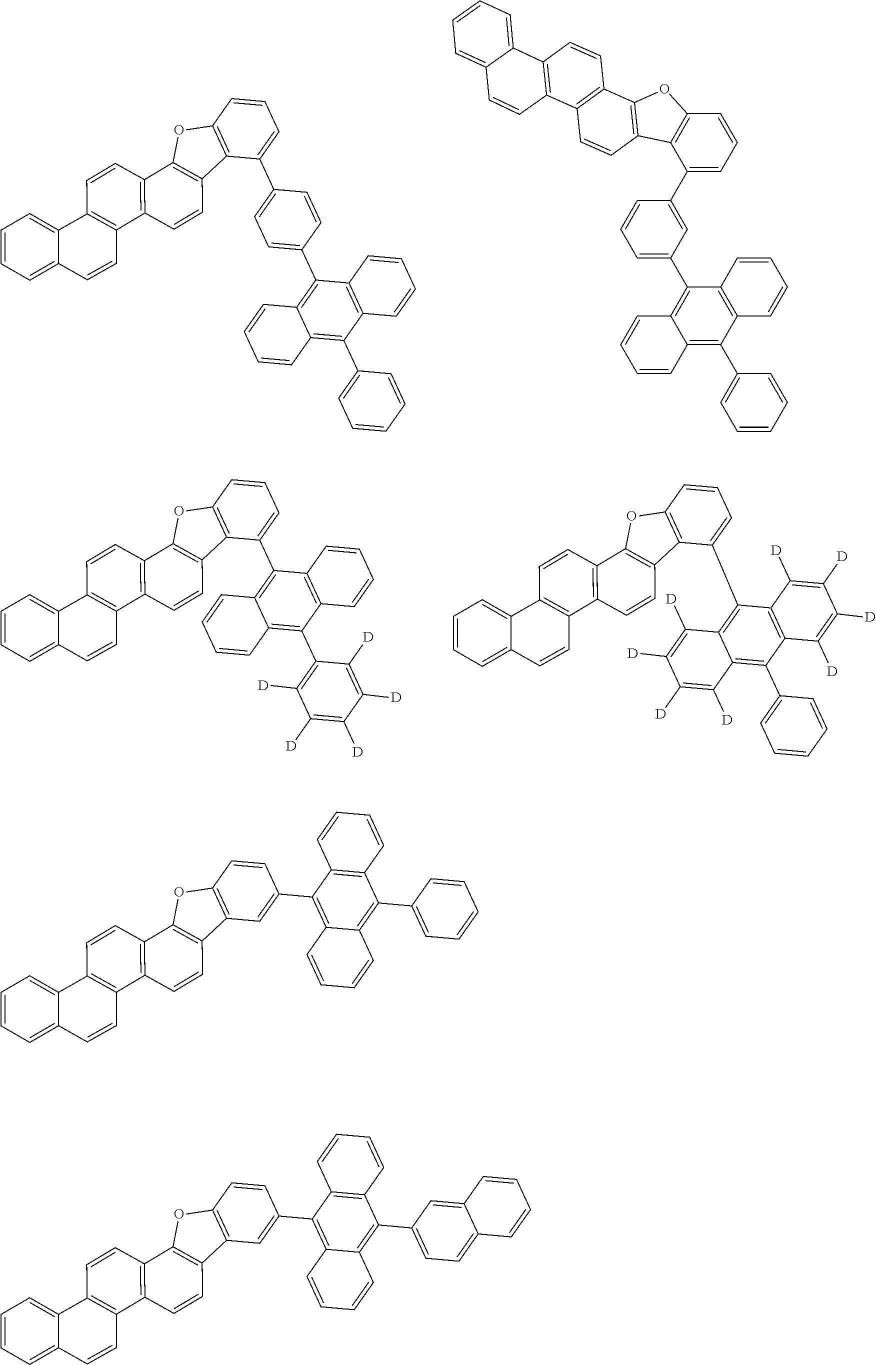

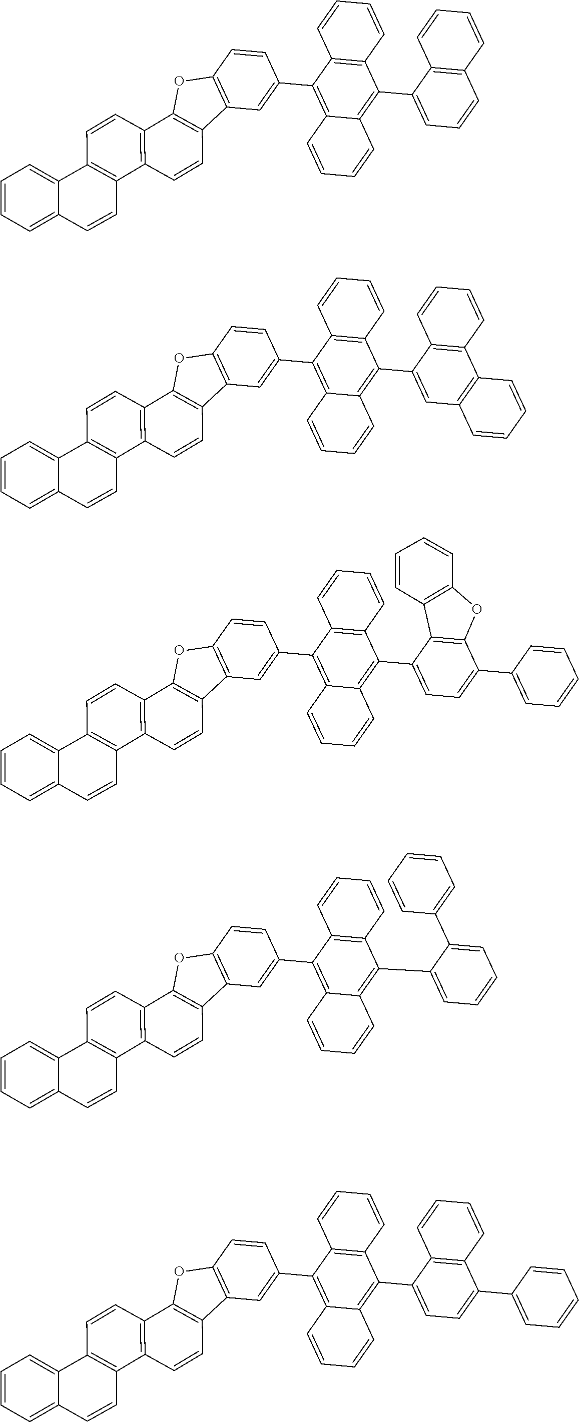 [SMILES: c1ccc2c(-c3c4ccccc4c(-c4ccc5oc6c(ccc7c8ccc9ccccc9c8ccc76)c5c4)c4ccccc34)cccc2c1 ; c1ccc2c(c1)cc(-c1c3ccccc3c(-c3ccc4oc5c(ccc6c7ccc8ccccc8c7ccc65)c4c3)c3ccccc13)c1ccccc12 ; c1ccc(-c2ccc(-c3c4ccccc4c(-c4ccc5oc6c(ccc7c8ccc9ccccc9c8ccc76)c5c4)c4ccccc34)c3c2oc2ccccc23)cc1 ; c1ccc(-c2ccccc2-c2c3ccccc3c(-c3ccc4oc5c(ccc6c7ccc8ccccc8c7ccc65)c4c3)c3ccccc23)cc1 ; c1ccc(-c2ccc(-c3c4ccccc4c(-c4ccc5oc6c(ccc7c8ccc9ccccc9c8ccc76)c5c4)c4ccccc34)c3ccccc23)cc1]

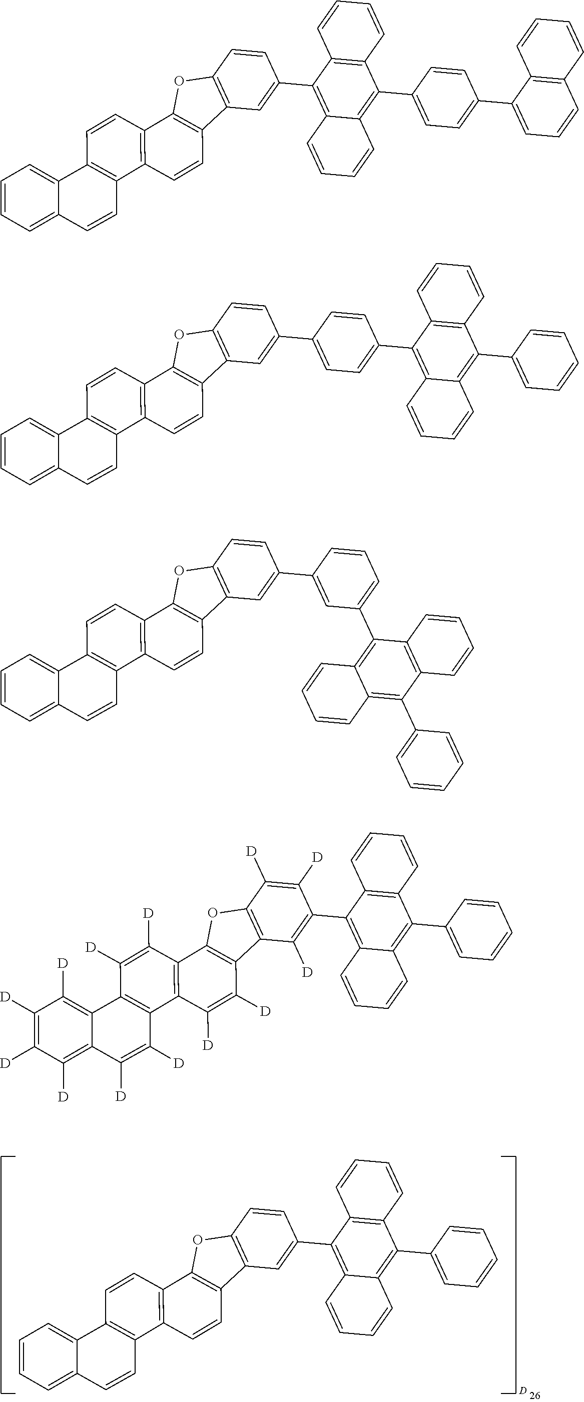

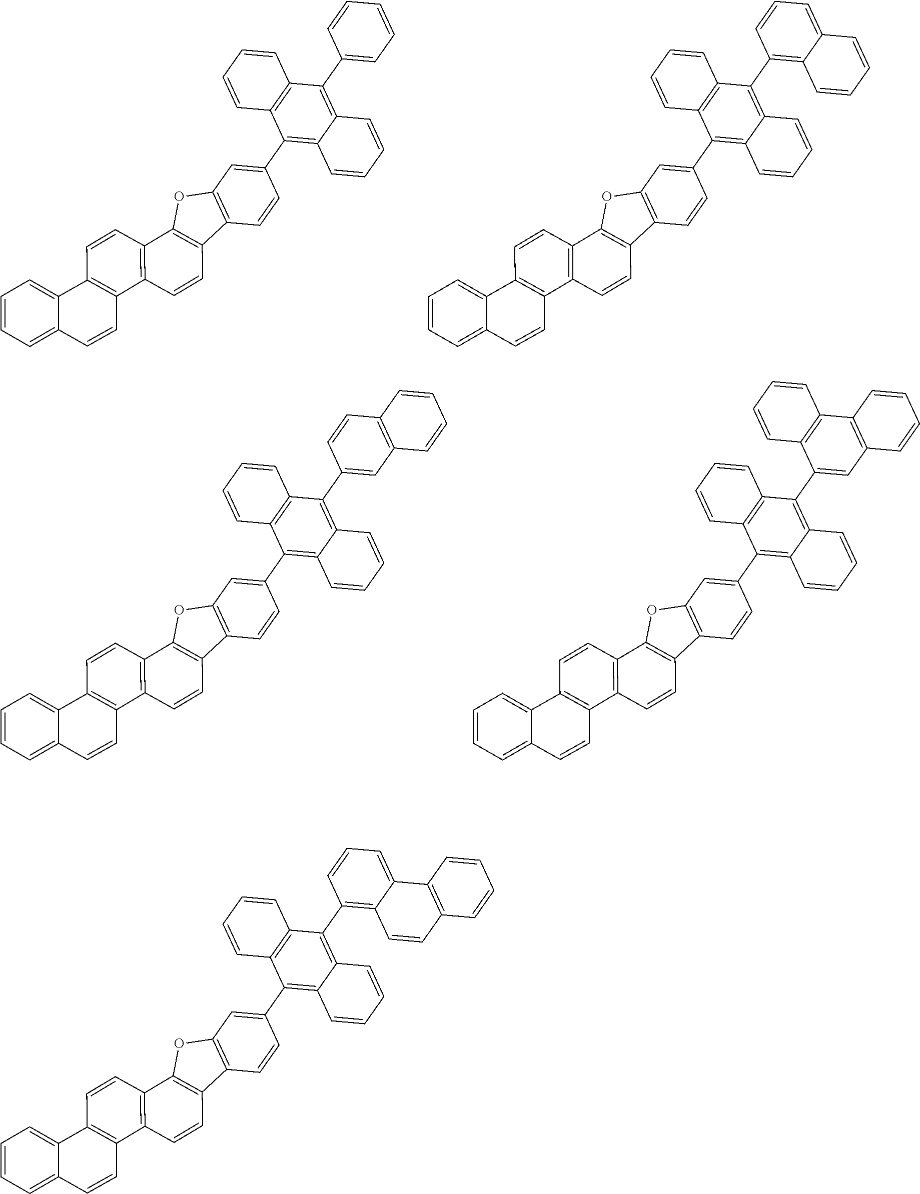 [SMILES: c1ccc(-c2c3ccccc3c(-c3ccc4c(c3)oc3c4ccc4c5ccc6ccccc6c5ccc43)c3ccccc23)cc1 ; c1ccc2c(-c3c4ccccc4c(-c4ccc5c(c4)oc4c5ccc5c6ccc7ccccc7c6ccc54)c4ccccc34)cccc2c1 ; c1ccc2cc(-c3c4ccccc4c(-c4ccc5c(c4)oc4c5ccc5c6ccc7ccccc7c6ccc54)c4ccccc34)ccc2c1 ; c1ccc2c(c1)cc(-c1c3ccccc3c(-c3ccc4c(c3)oc3c4ccc4c5ccc6ccccc6c5ccc43)c3ccccc13)c1ccccc12 ; c1ccc2c(c1)ccc1c(-c3c4ccccc4c(-c4ccc5c(c4)oc4c5ccc5c6ccc7ccccc7c6ccc54)c4ccccc34)cccc12]

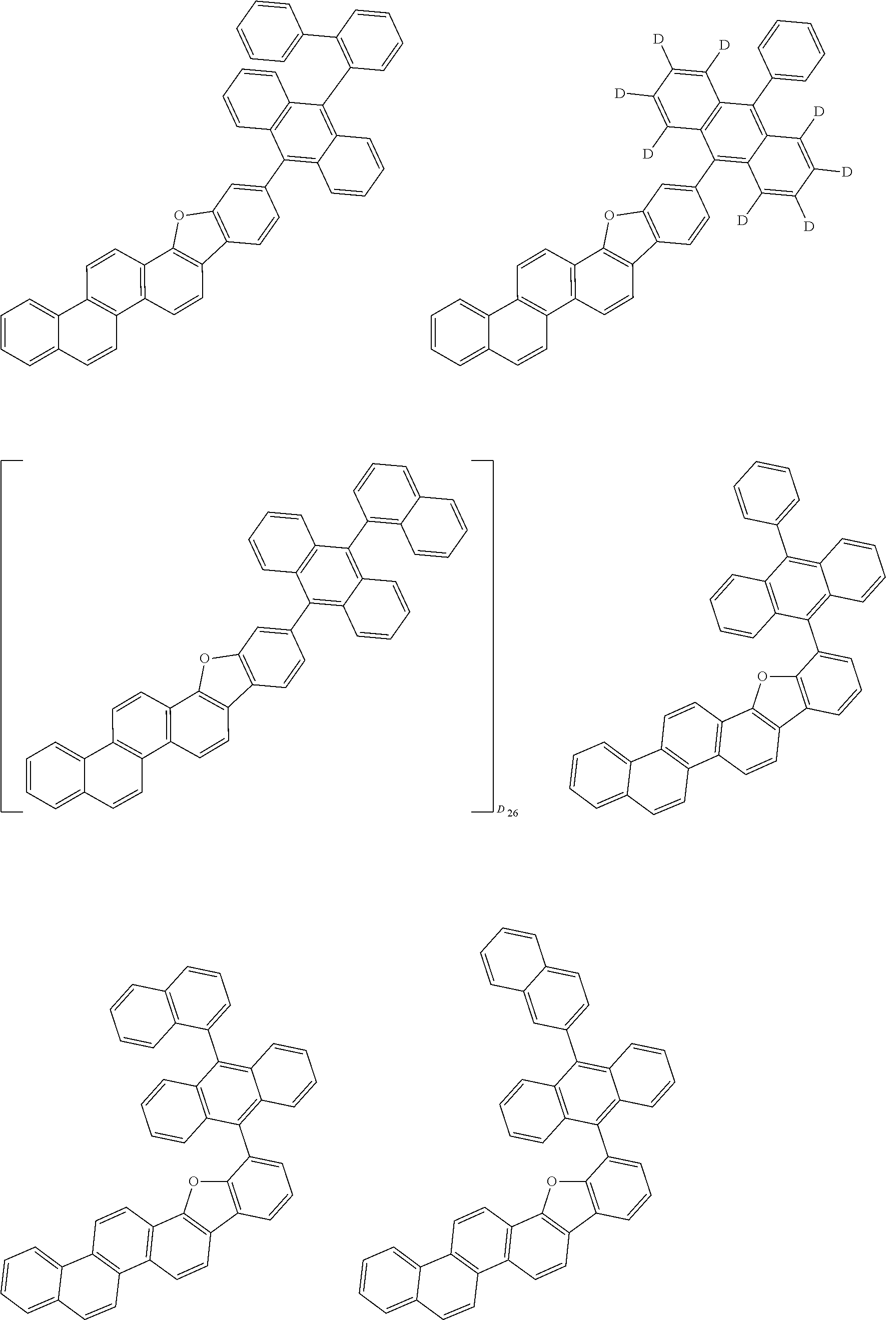

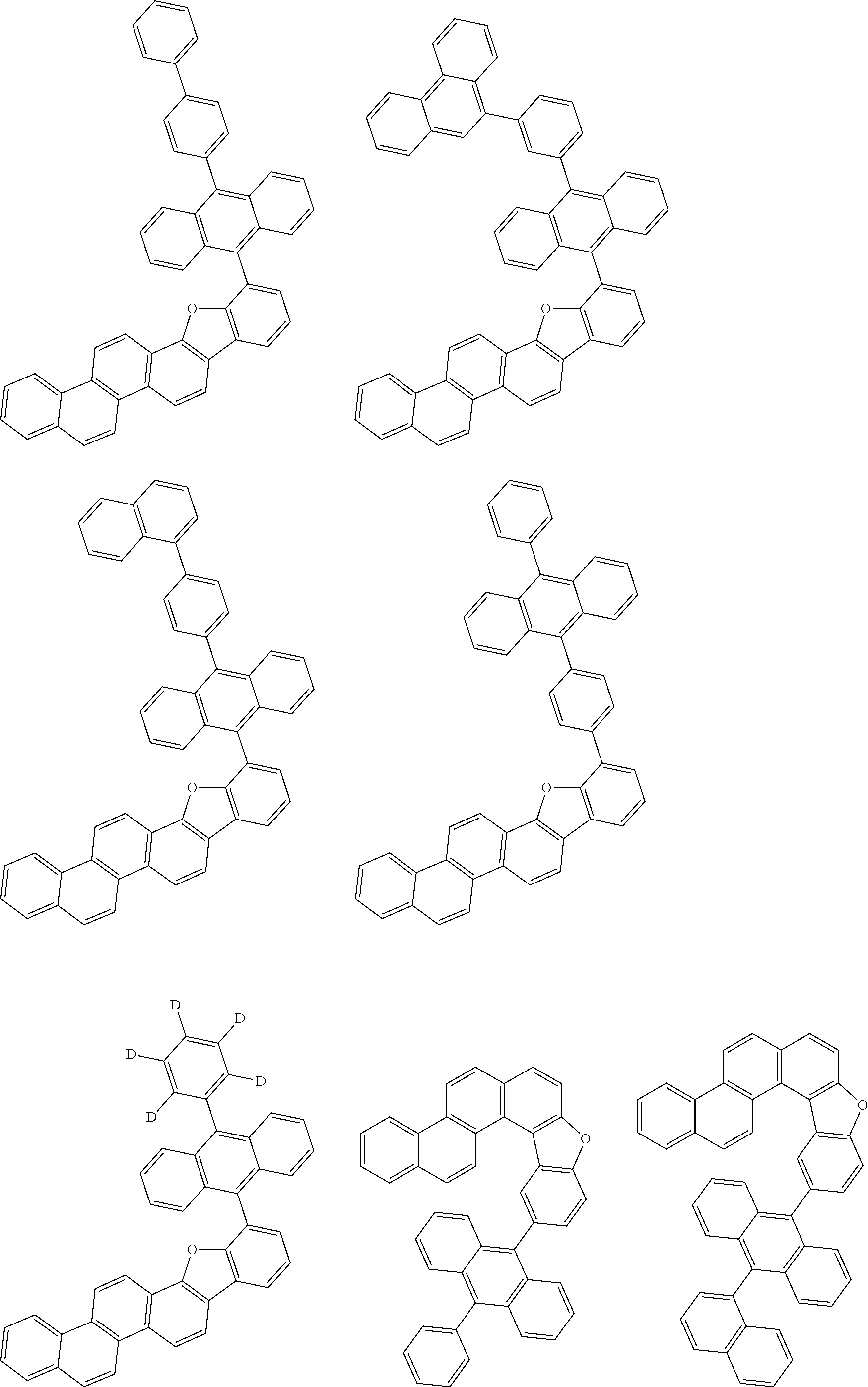

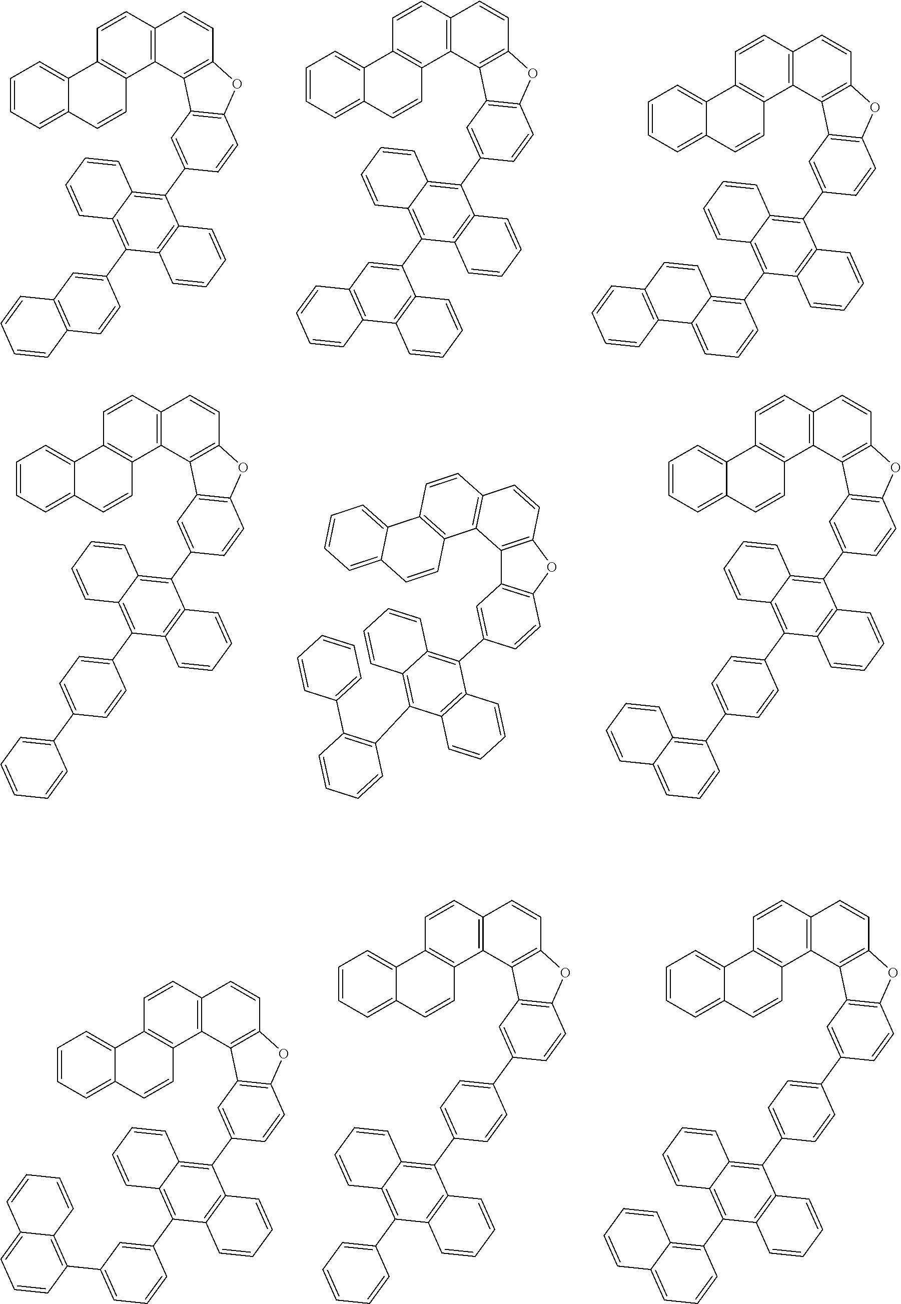

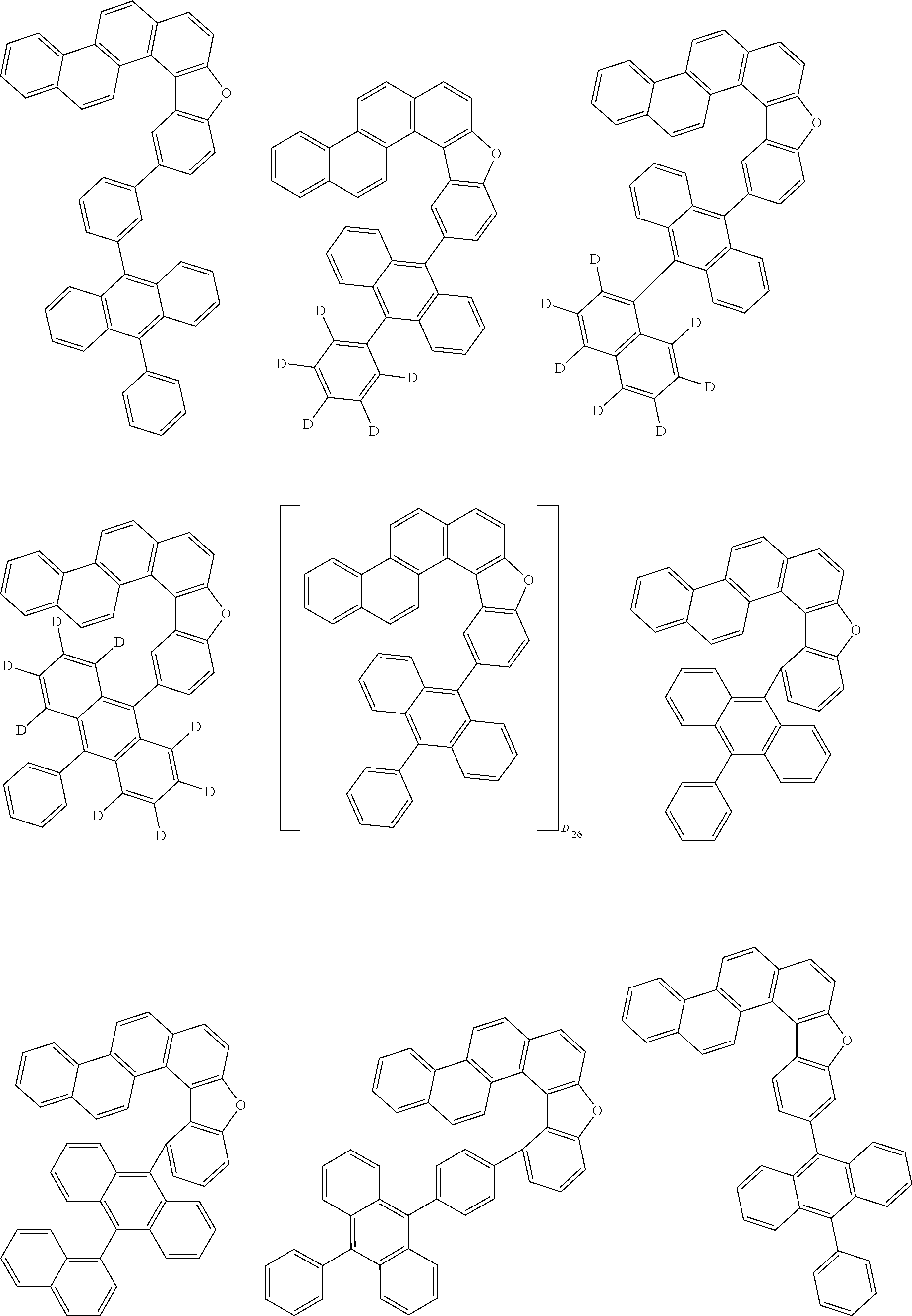

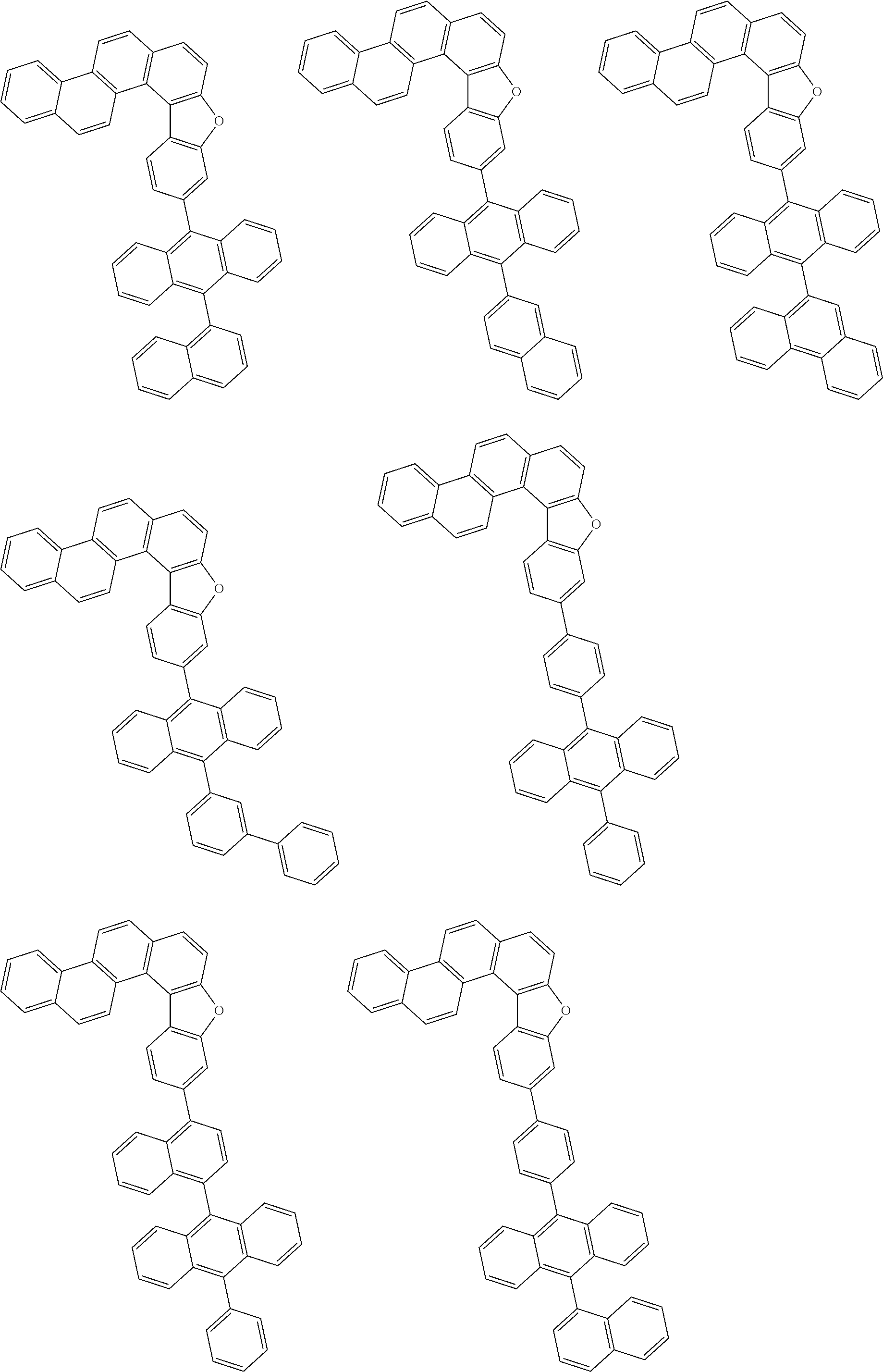

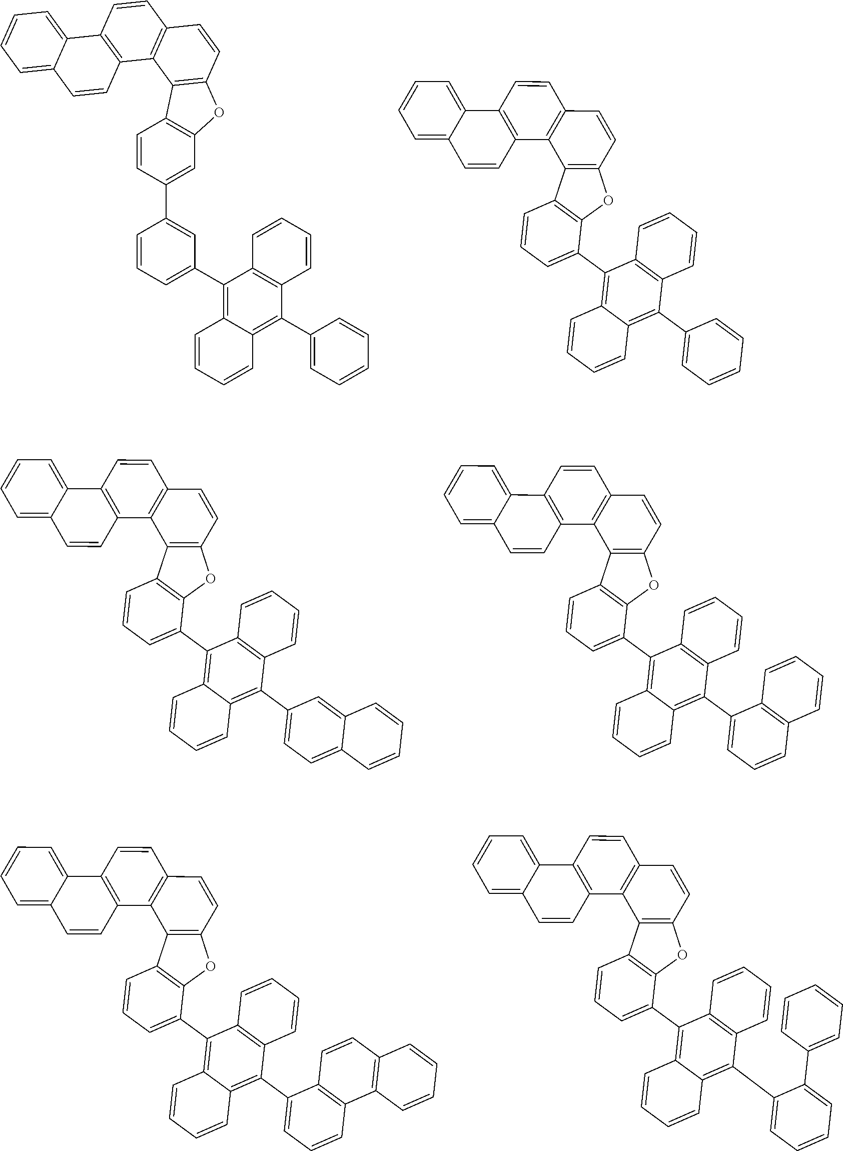 [SMILES: c1ccc(-c2c3ccccc3c(-c3cccc(-c4ccc5c(c4)oc4ccc6ccc7c8ccccc8ccc7c6c45)c3)c3ccccc23)cc1 ; c1ccc(-c2c3ccccc3c(-c3cccc4c3oc3ccc5ccc6c7ccccc7ccc6c5c34)c3ccccc23)cc1 ; c1ccc2cc(-c3c4ccccc4c(-c4cccc5c4oc4ccc6ccc7c8ccccc8ccc7c6c45)c4ccccc34)ccc2c1 ; c1ccc2c(-c3c4ccccc4c(-c4cccc5c4oc4ccc6ccc7c8ccccc8ccc7c6c45)c4ccccc34)cccc2c1 ; c1ccc2c(c1)ccc1c(-c3c4ccccc4c(-c4cccc5c4oc4ccc6ccc7c8ccccc8ccc7c6c45)c4ccccc34)cccc12 ; c1ccc(-c2ccccc2-c2c3ccccc3c(-c3cccc4c3oc3ccc5ccc6c7ccccc7ccc6c5c34)c3ccccc23)cc1]

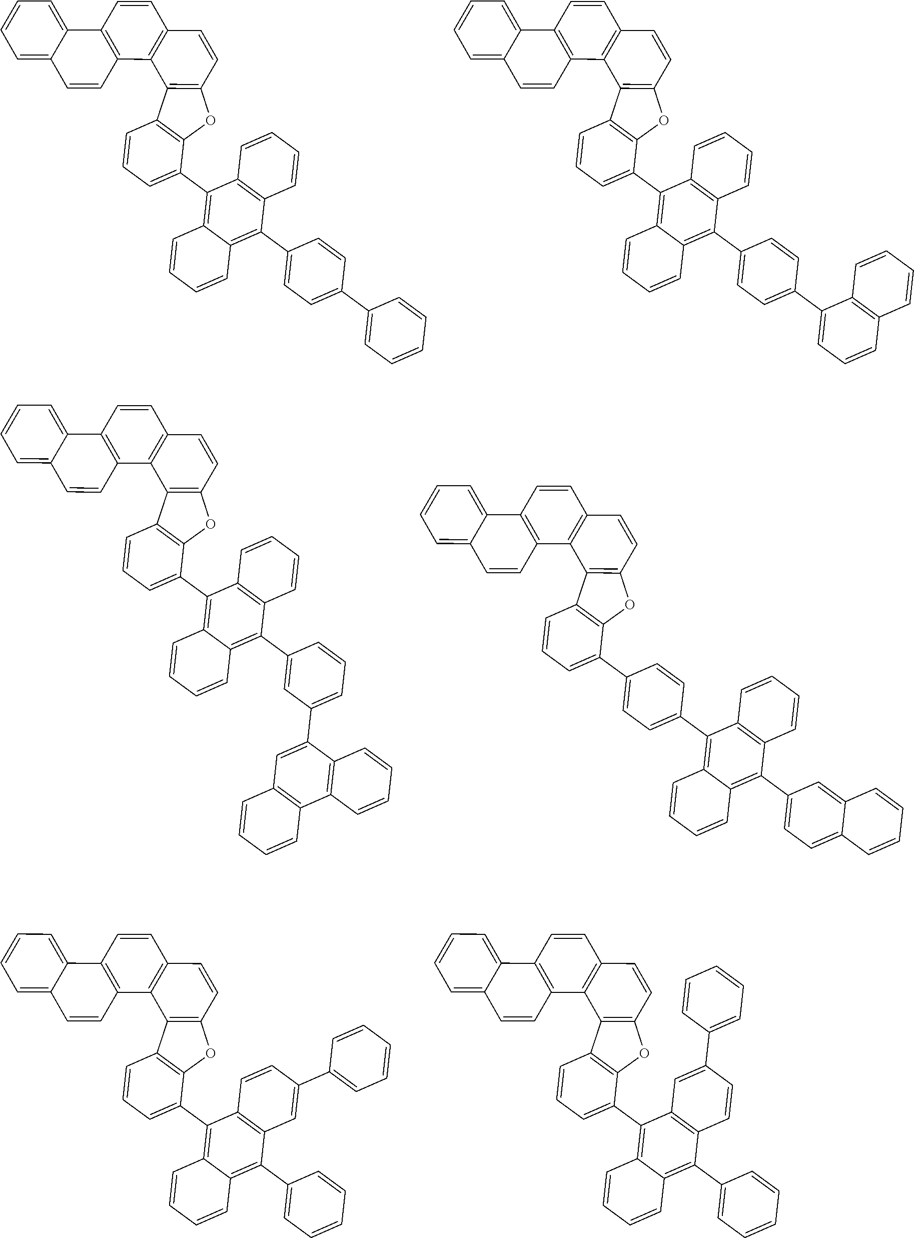

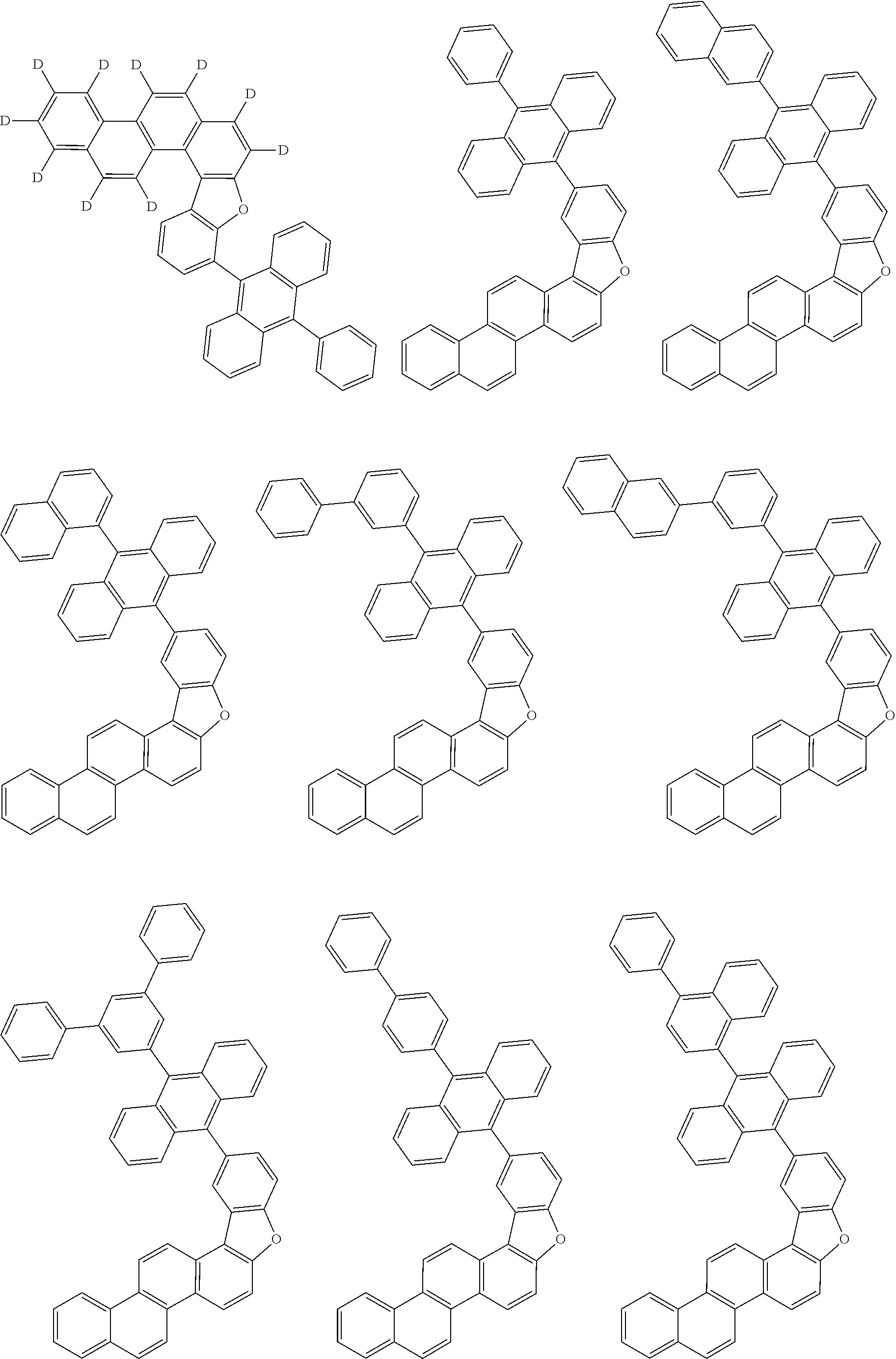

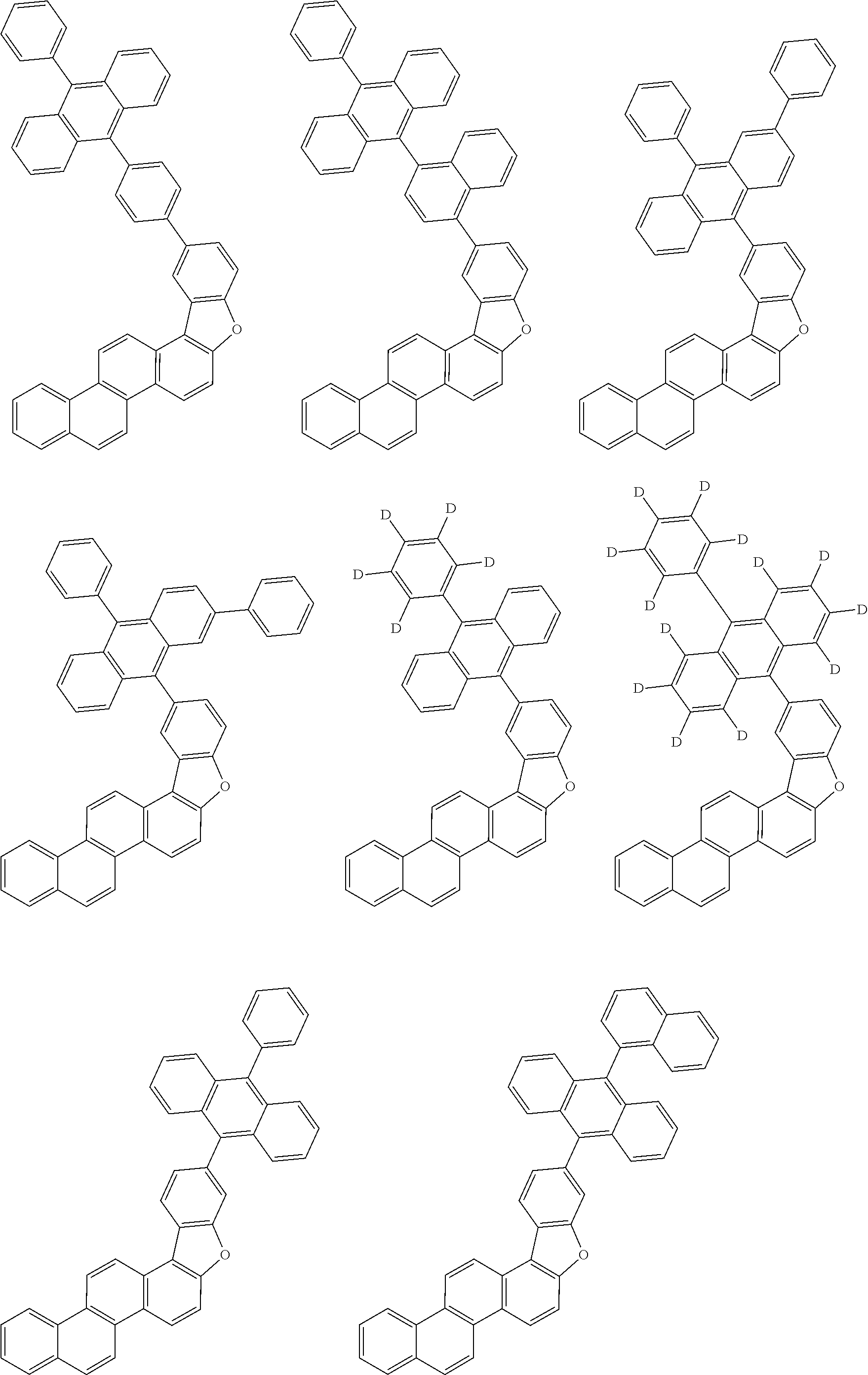

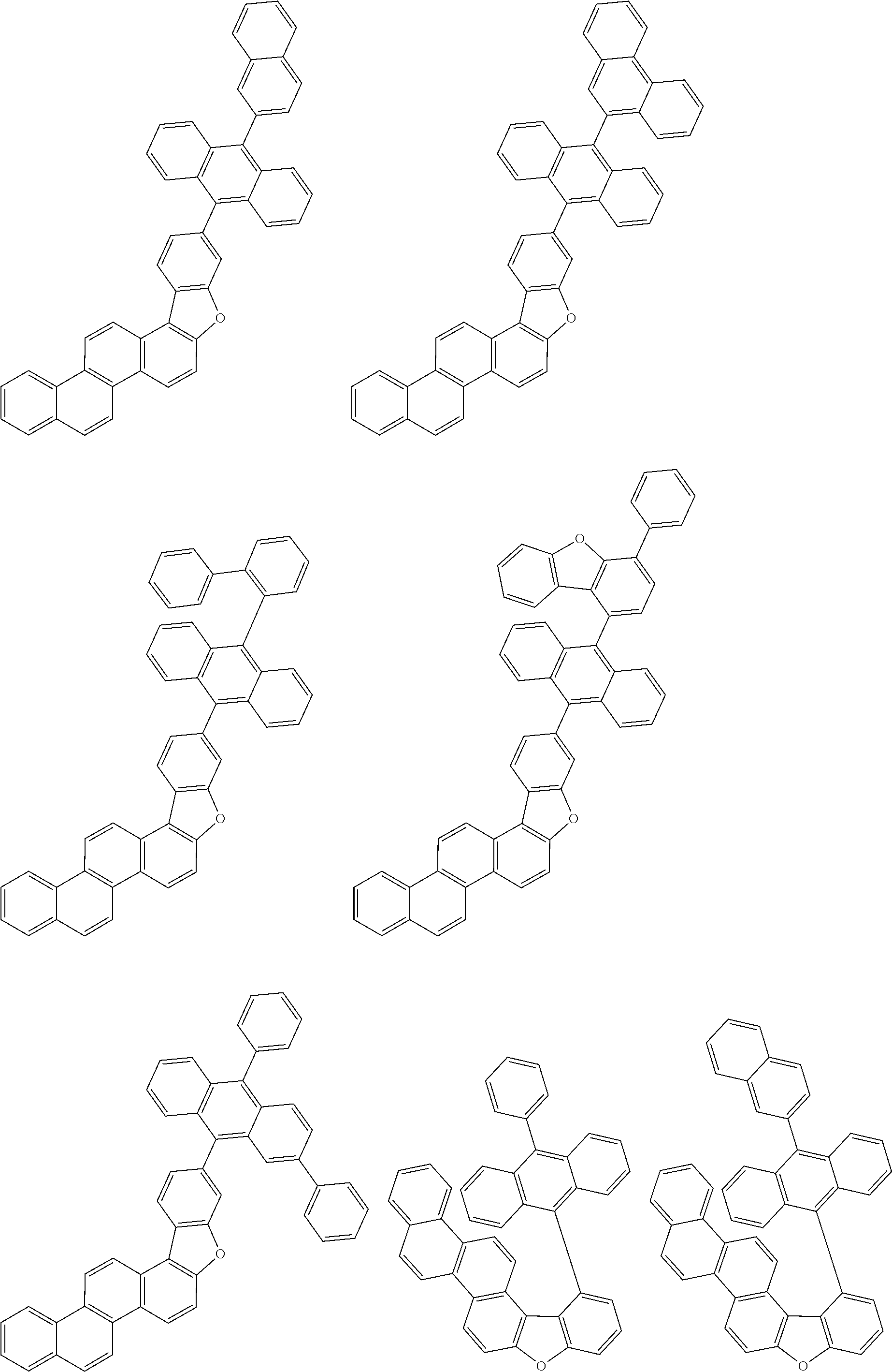

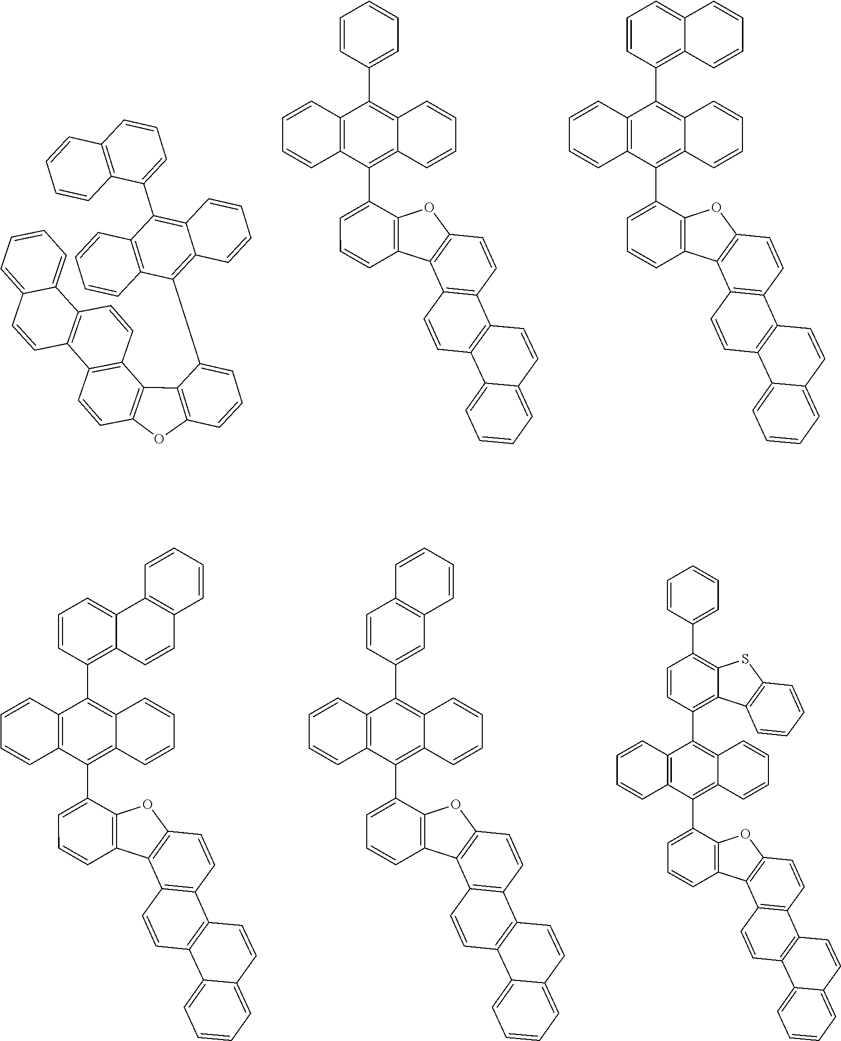 [SMILES: c1ccc2c(-c3c4ccccc4c(-c4cccc5oc6ccc7c8ccc9ccccc9c8ccc7c6c45)c4ccccc34)cccc2c1 ; c1ccc(-c2c3ccccc3c(-c3cccc4c3oc3ccc5c6ccc7ccccc7c6ccc5c34)c3ccccc23)cc1 ; c1ccc2c(-c3c4ccccc4c(-c4cccc5c4oc4ccc6c7ccc8ccccc8c7ccc6c45)c4ccccc34)cccc2c1 ; c1ccc2c(c1)ccc1c(-c3c4ccccc4c(-c4cccc5c4oc4ccc6c7ccc8ccccc8c7ccc6c45)c4ccccc34)cccc12 ; c1ccc2cc(-c3c4ccccc4c(-c4cccc5c4oc4ccc6c7ccc8ccccc8c7ccc6c45)c4ccccc34)ccc2c1 ; c1ccc(-c2ccc(-c3c4ccccc4c(-c4cccc5c4oc4ccc6c7ccc8ccccc8c7ccc6c45)c4ccccc34)c3c2sc2ccccc23)cc1]

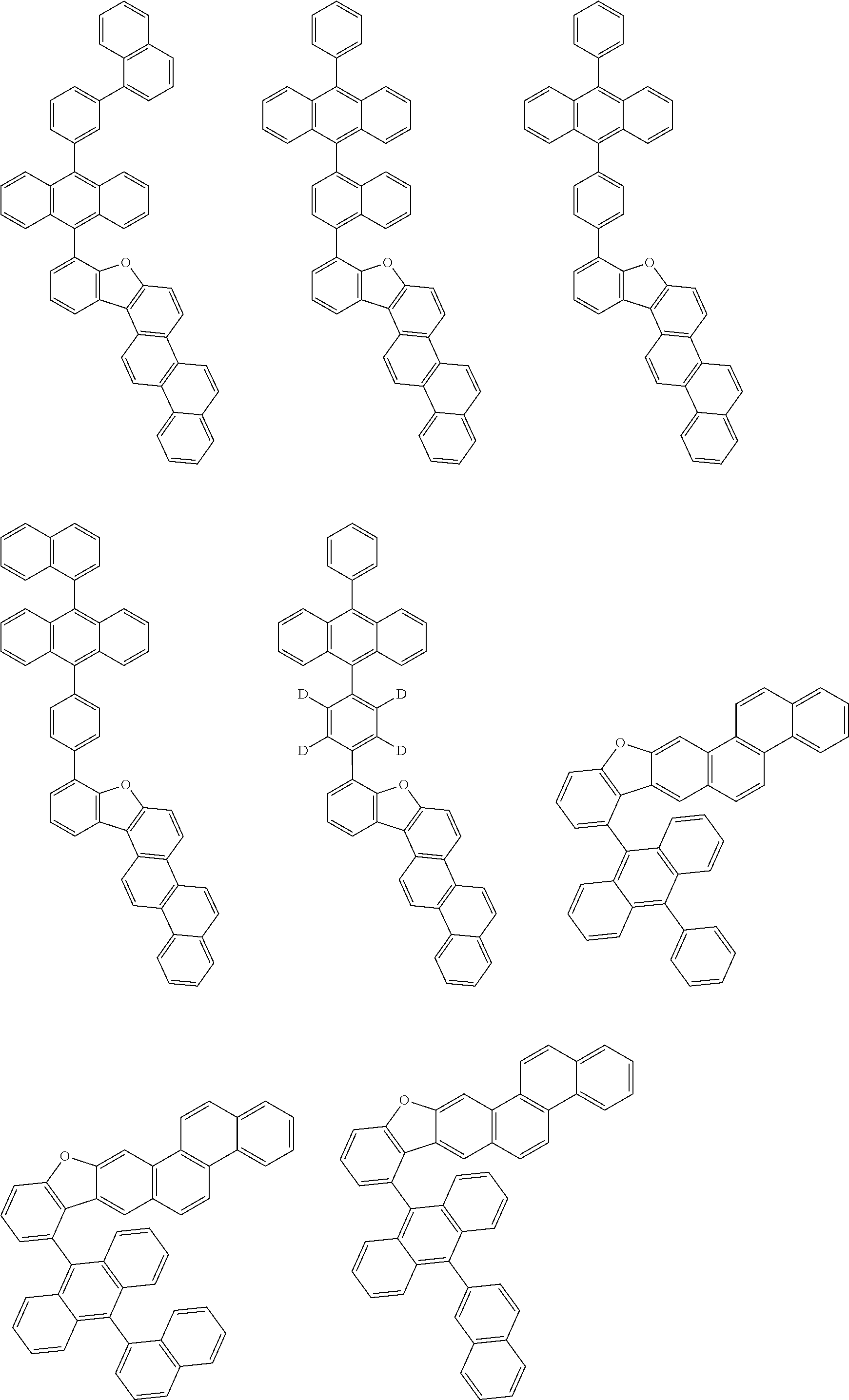

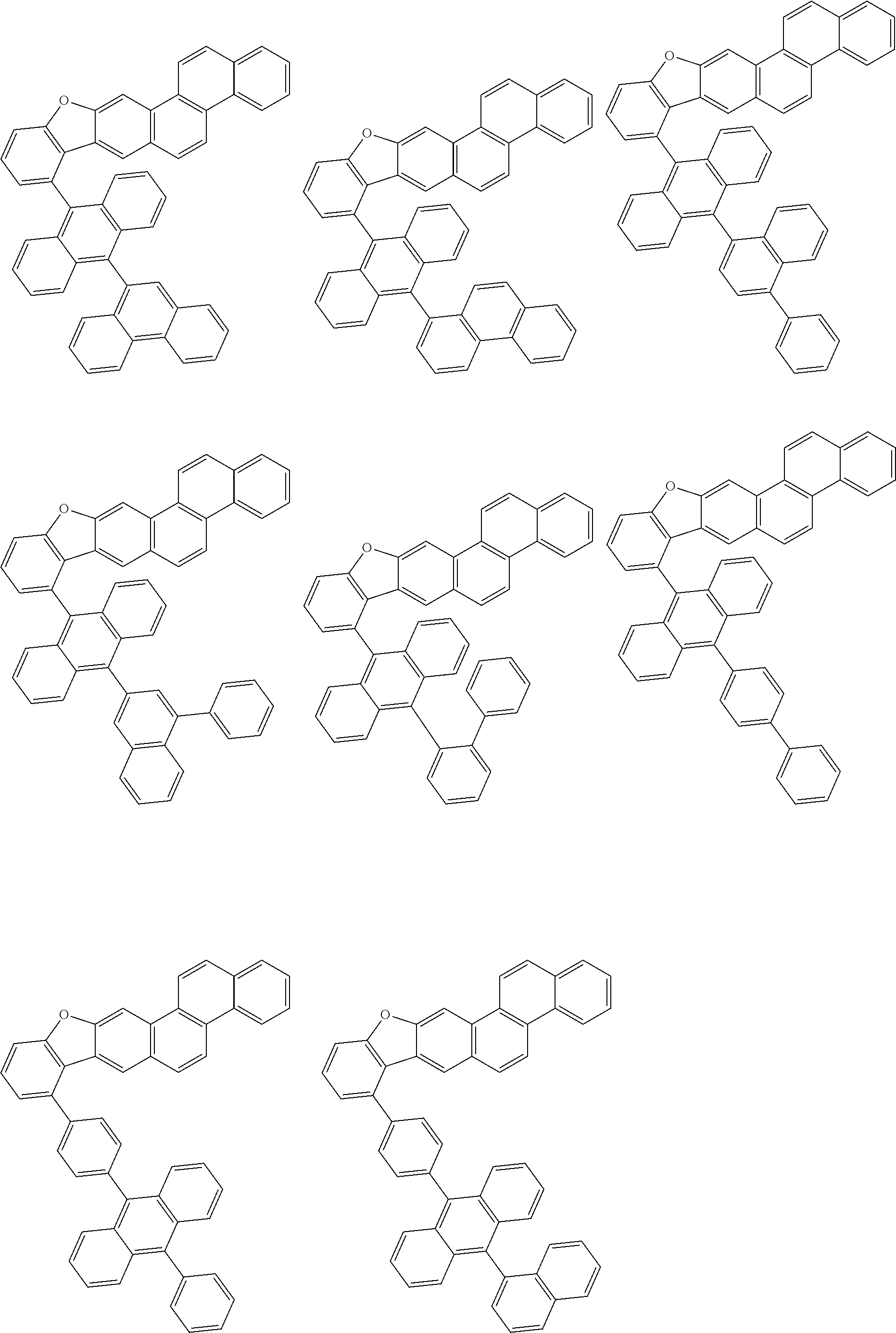

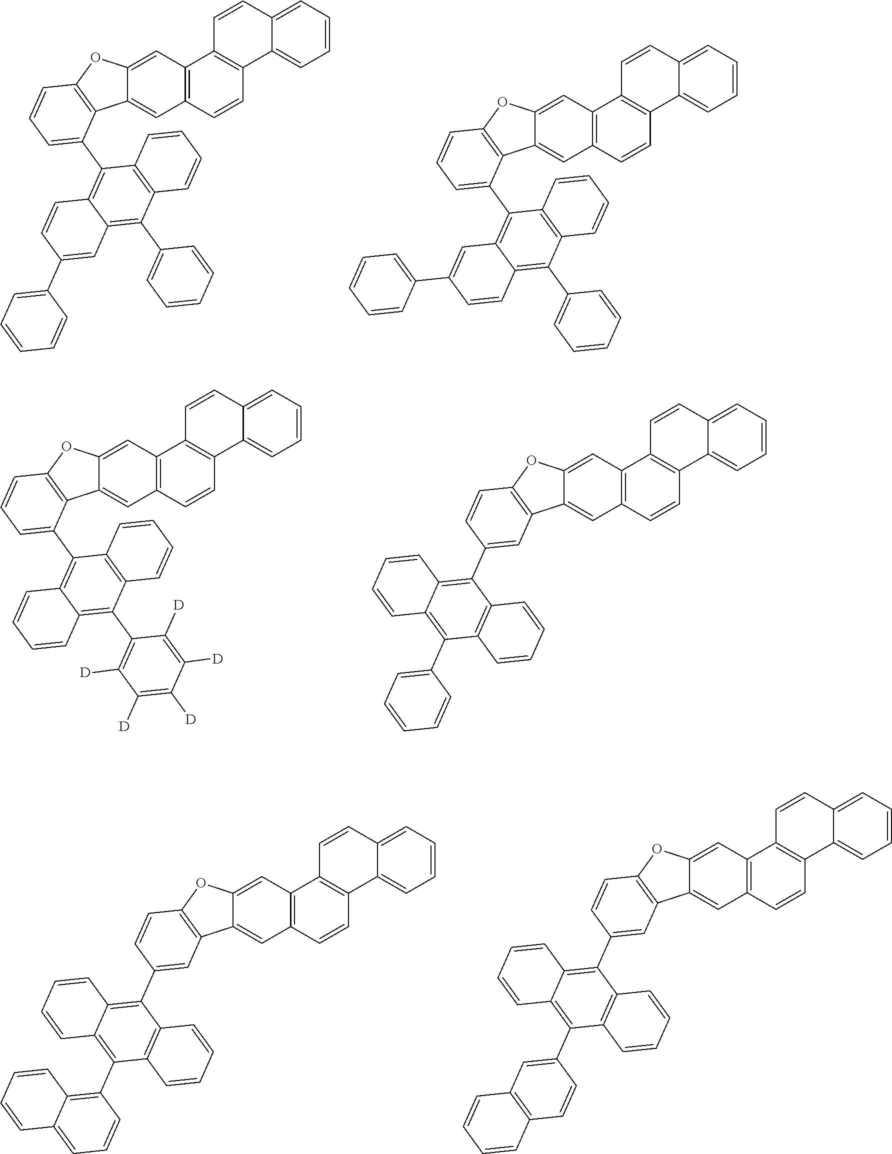

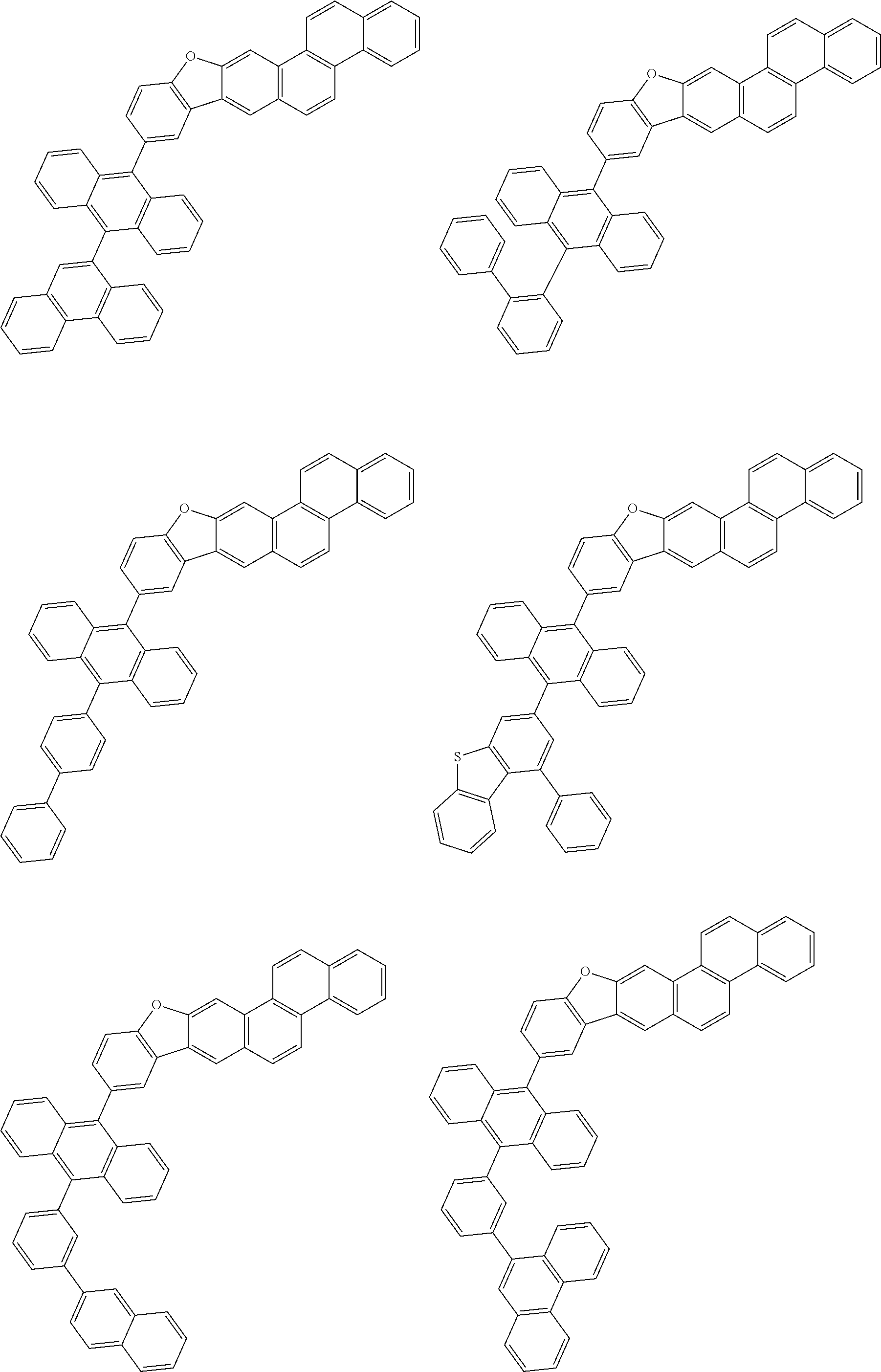

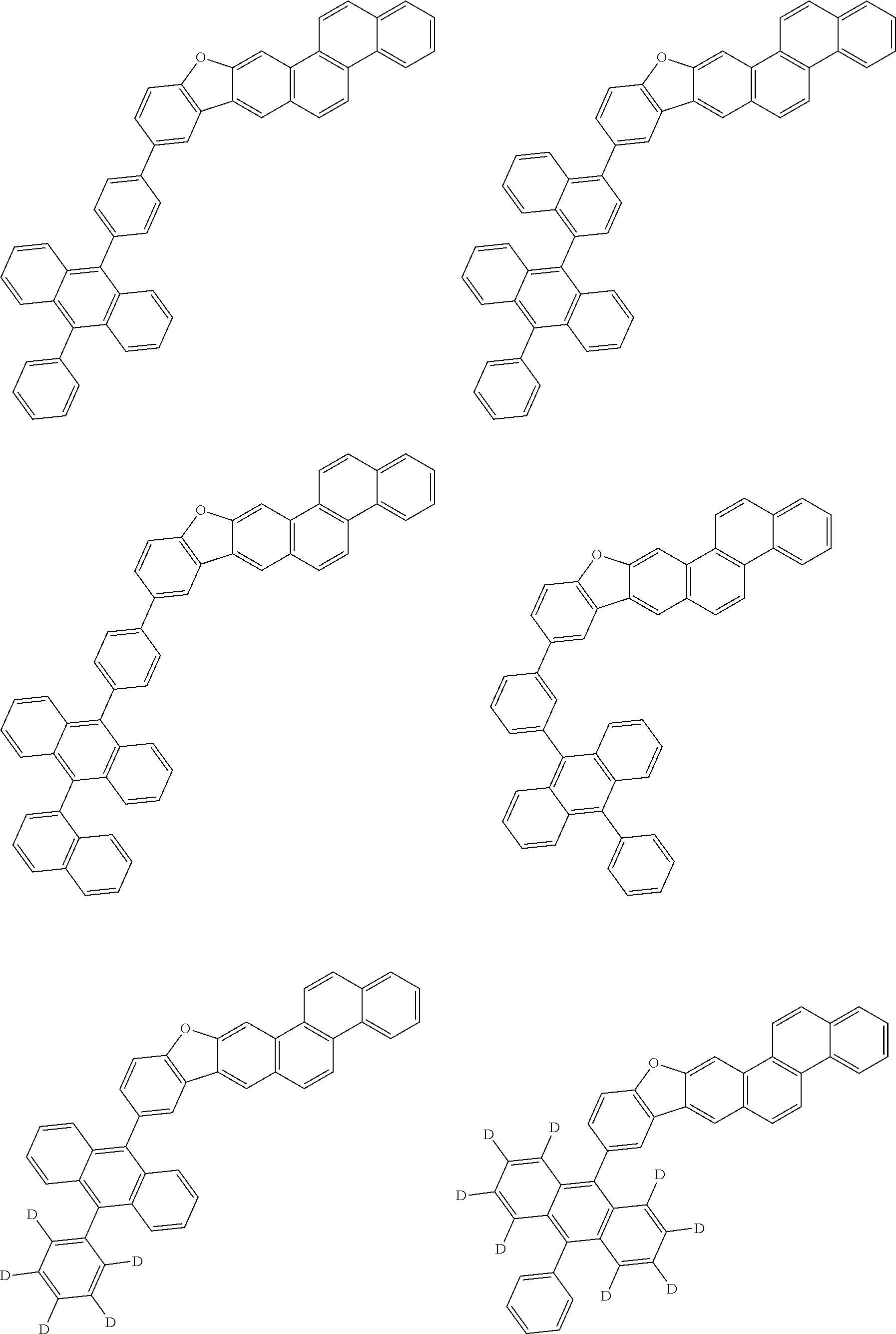

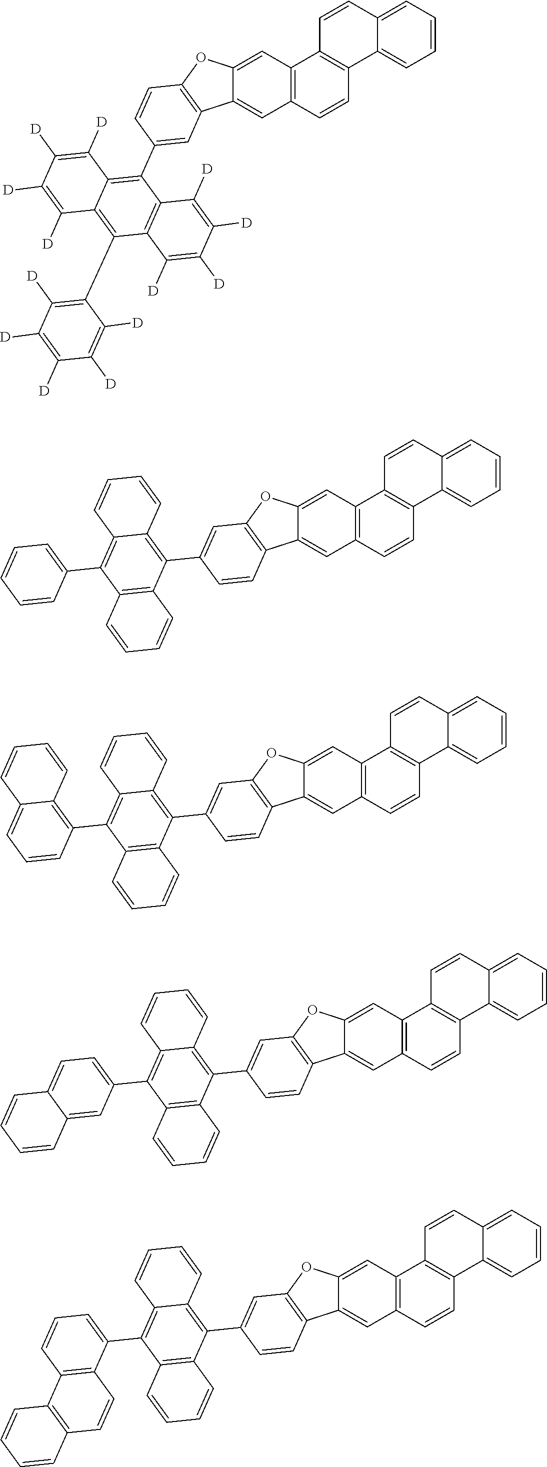 [SMILES: [2H]c1c([2H])c([2H])c(-c2c3c([2H])c([2H])c([2H])c([2H])c3c(-c3ccc4oc5cc6c(ccc7c8ccccc8ccc67)cc5c4c3)c3c([2H])c([2H])c([2H])c([2H])c23)c([2H])c1[2H] ; c1ccc(-c2c3ccccc3c(-c3ccc4c(c3)oc3cc5c(ccc6c7ccccc7ccc56)cc34)c3ccccc23)cc1 ; c1ccc2c(-c3c4ccccc4c(-c4ccc5c(c4)oc4cc6c(ccc7c8ccccc8ccc67)cc45)c4ccccc34)cccc2c1 ; c1ccc2cc(-c3c4ccccc4c(-c4ccc5c(c4)oc4cc6c(ccc7c8ccccc8ccc67)cc45)c4ccccc34)ccc2c1 ; c1ccc2c(c1)ccc1c(-c3c4ccccc4c(-c4ccc5c(c4)oc4cc6c(ccc7c8ccccc8ccc67)cc45)c4ccccc34)cccc12]

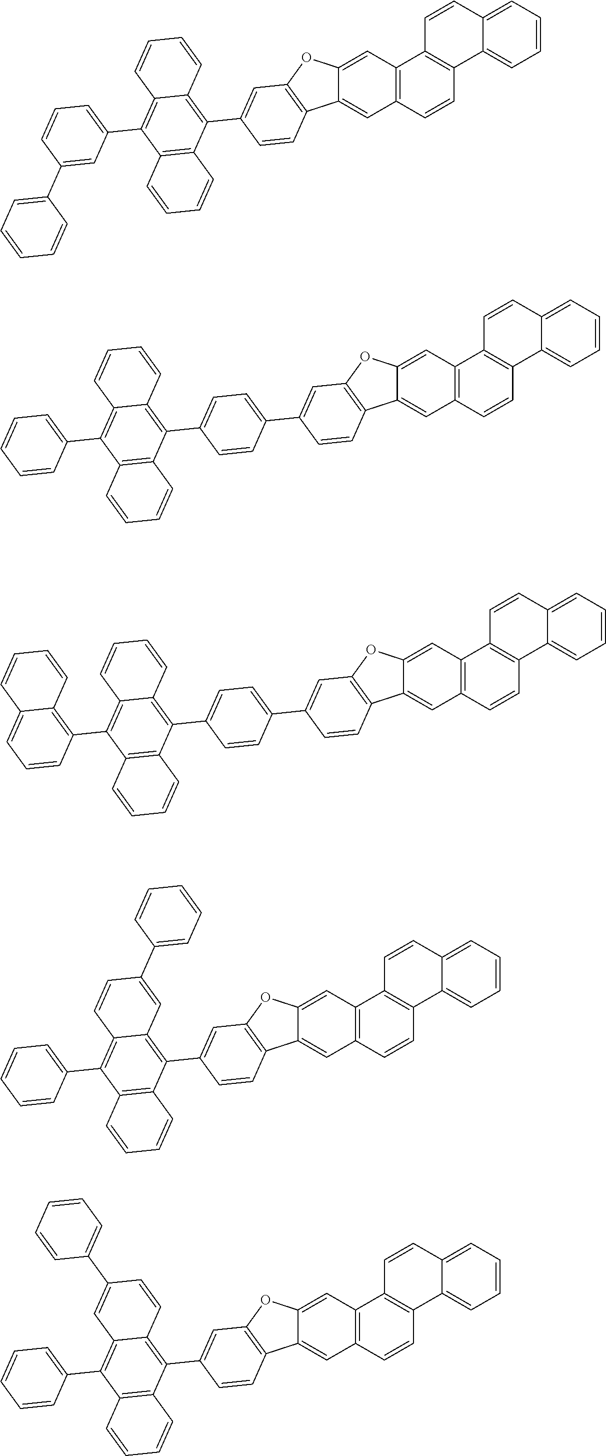 [SMILES: c1ccc(-c2cccc(-c3c4ccccc4c(-c4ccc5c(c4)oc4cc6c(ccc7c8ccccc8ccc67)cc45)c4ccccc34)c2)cc1 ; c1ccc(-c2c3ccccc3c(-c3ccc(-c4ccc5c(c4)oc4cc6c(ccc7c8ccccc8ccc67)cc45)cc3)c3ccccc23)cc1 ; c1ccc2c(-c3c4ccccc4c(-c4ccc(-c5ccc6c(c5)oc5cc7c(ccc8c9ccccc9ccc78)cc56)cc4)c4ccccc34)cccc2c1 ; c1ccc(-c2ccc3c(-c4ccccc4)c4ccccc4c(-c4ccc5c(c4)oc4cc6c(ccc7c8ccccc8ccc67)cc45)c3c2)cc1 ; c1ccc(-c2ccc3c(-c4ccc5c(c4)oc4cc6c(ccc7c8ccccc8ccc67)cc45)c4ccccc4c(-c4ccccc4)c3c2)cc1]

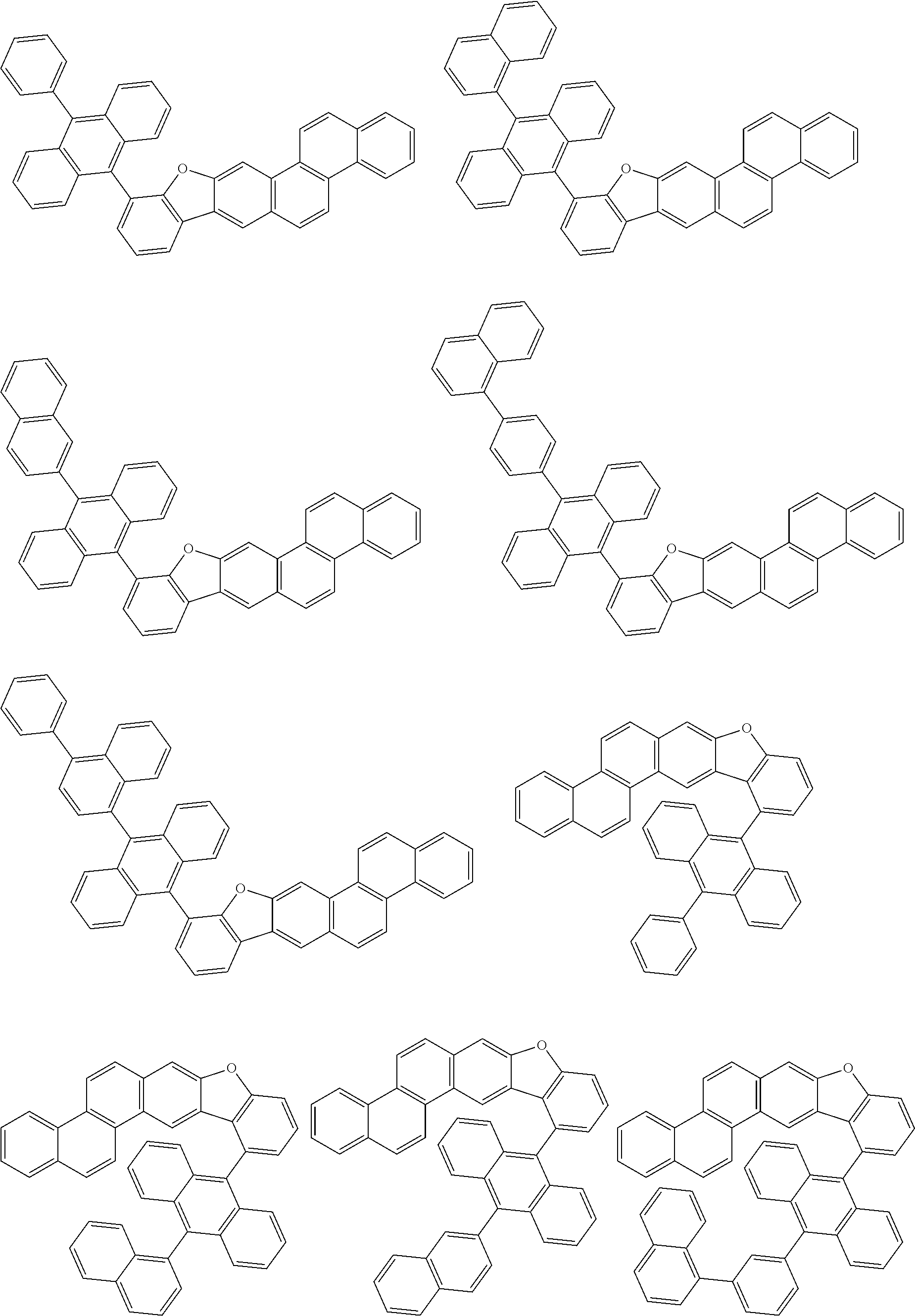

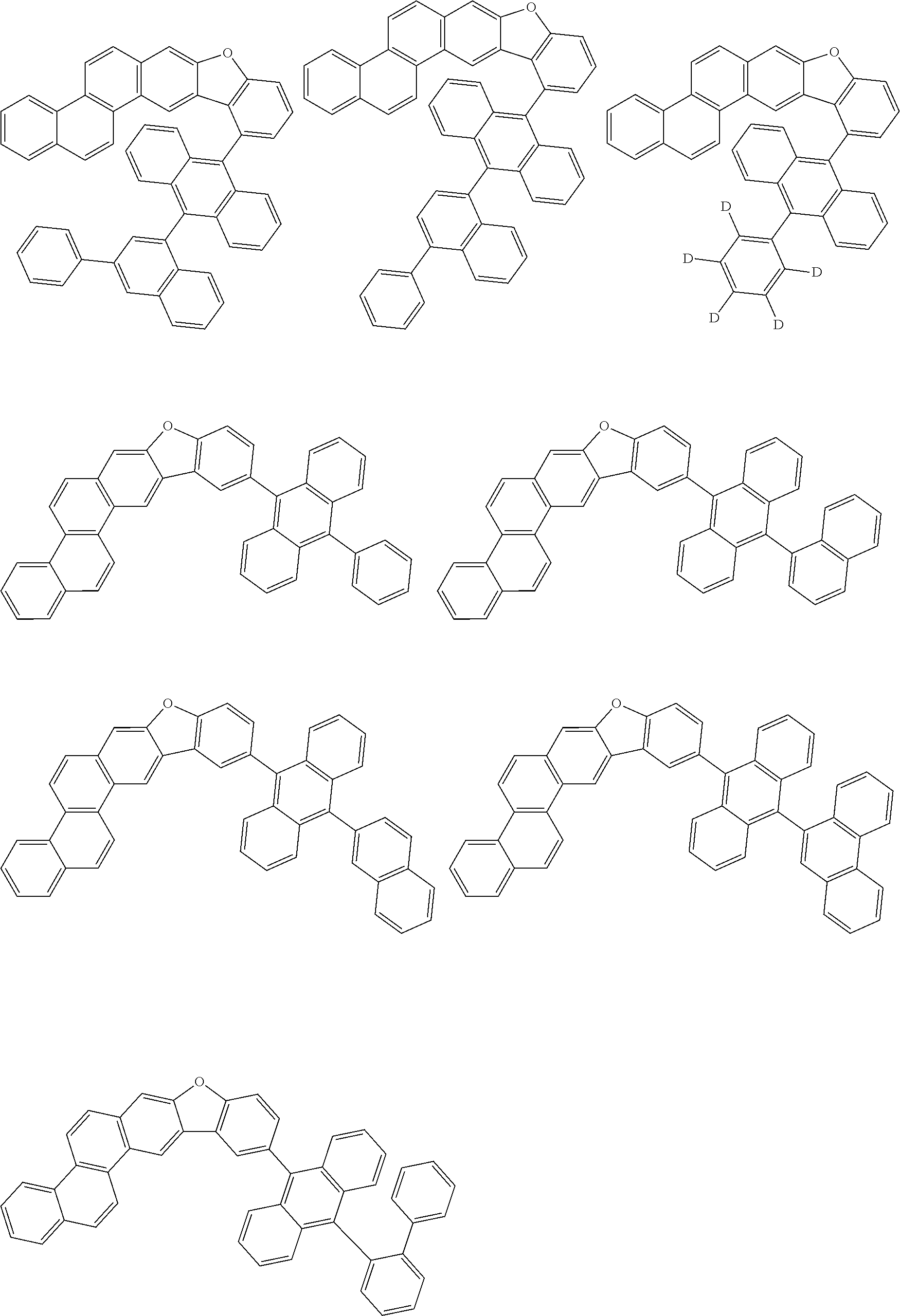

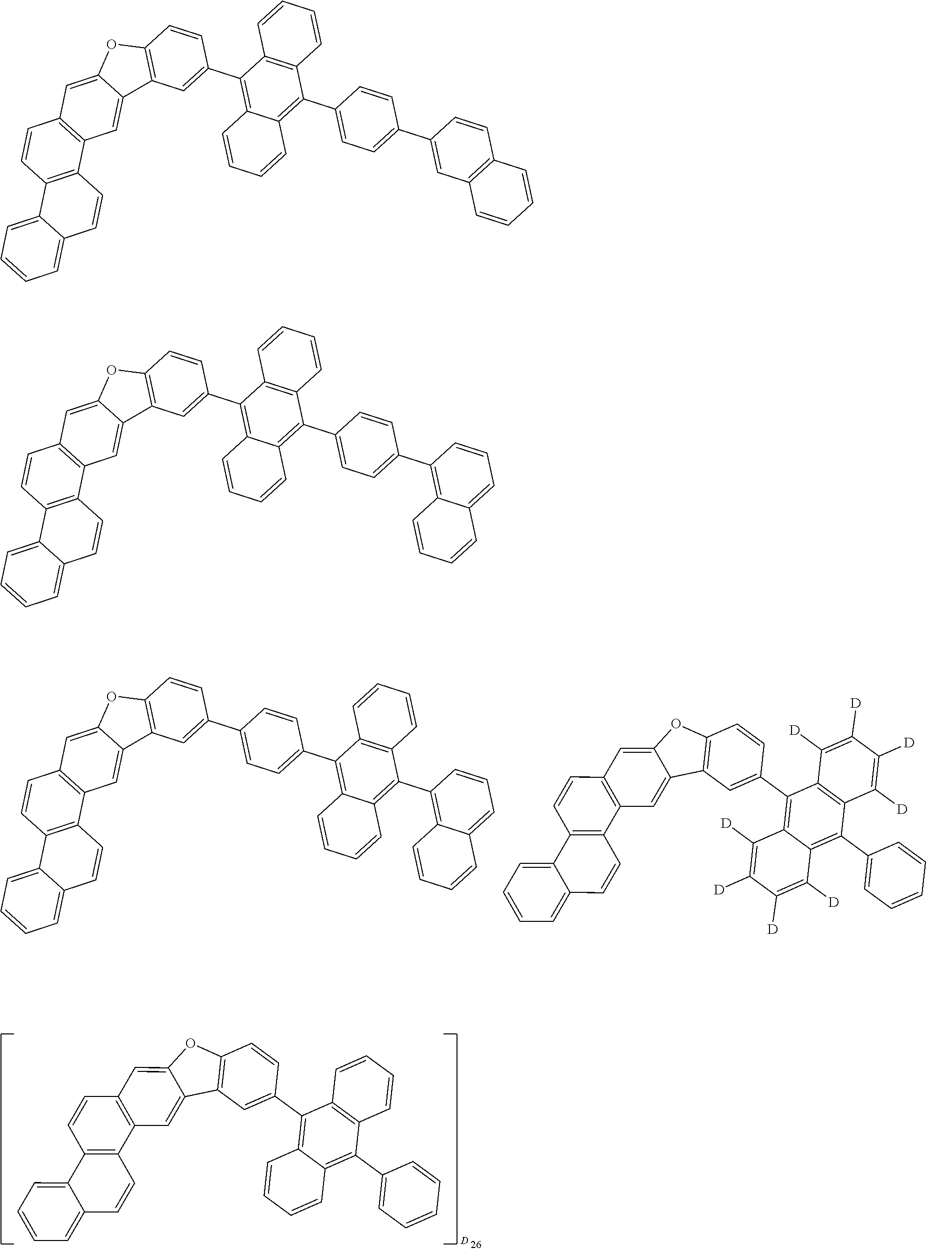

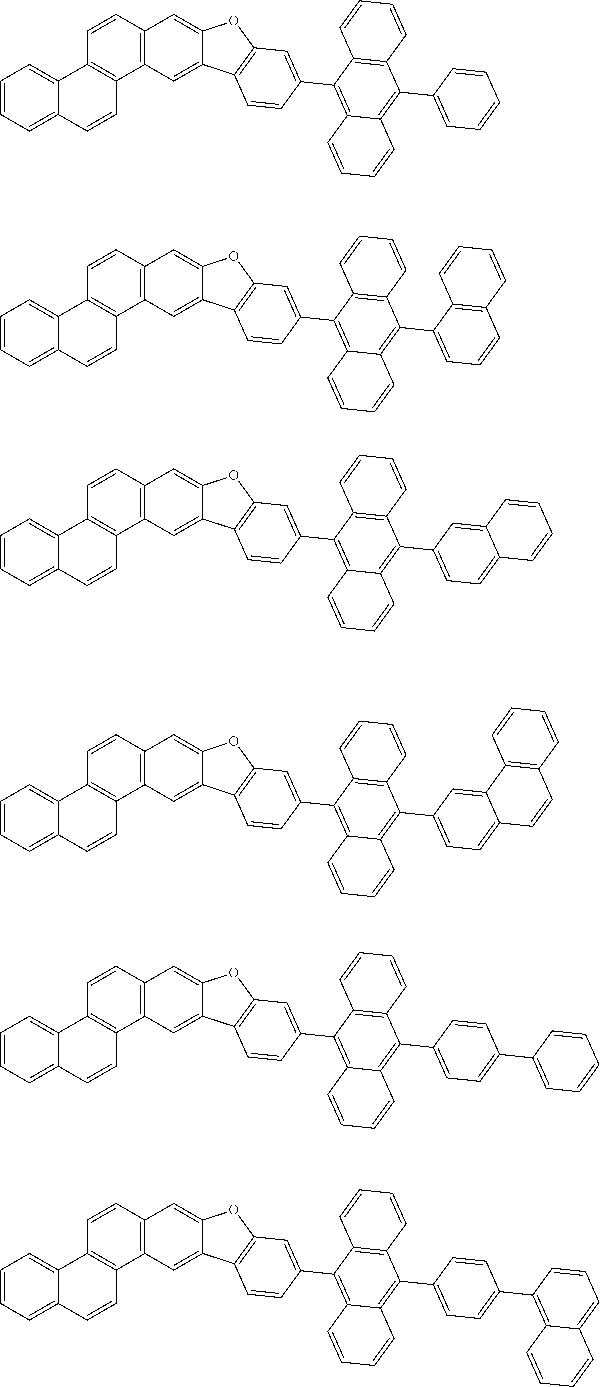

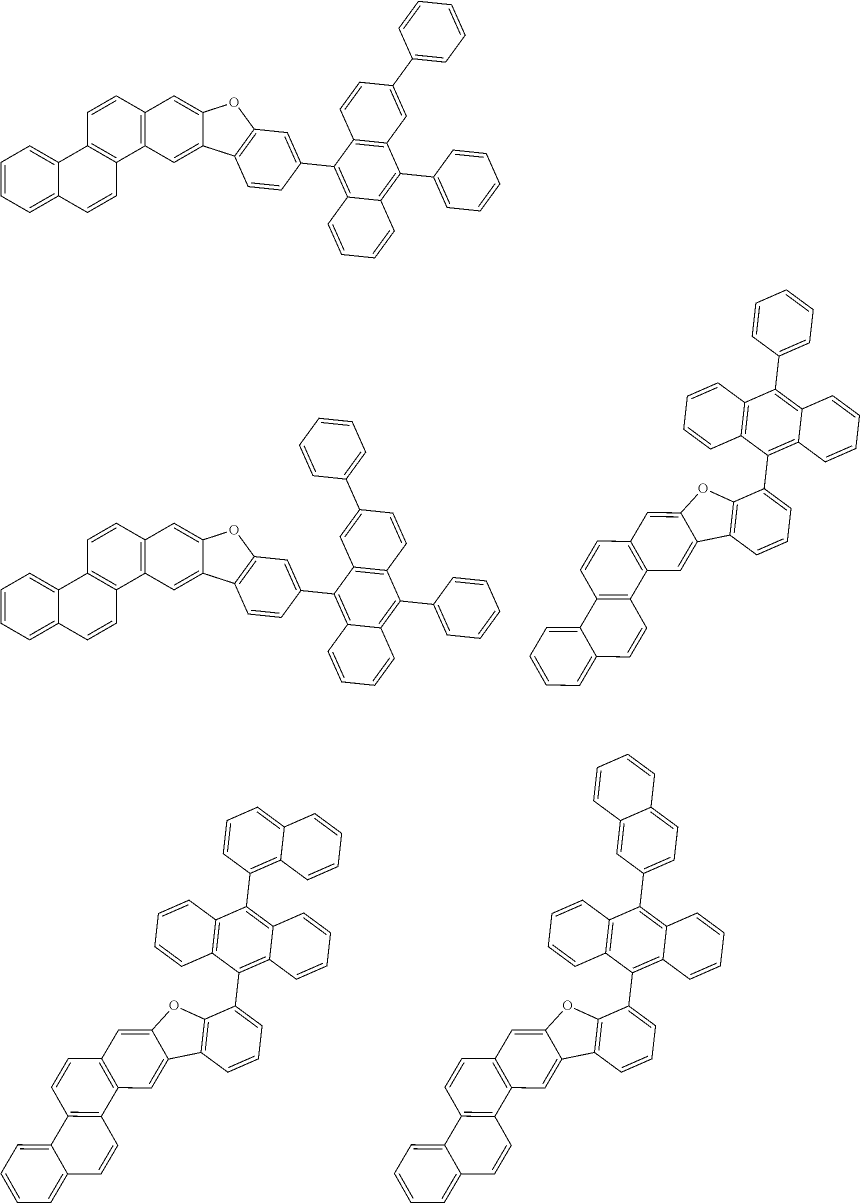 [SMILES: c1ccc(-c2ccc3c(-c4ccc5c(c4)oc4cc6ccc7c8ccccc8ccc7c6cc45)c4ccccc4c(-c4ccccc4)c3c2)cc1 ; c1ccc(-c2ccc3c(-c4ccccc4)c4ccccc4c(-c4ccc5c(c4)oc4cc6ccc7c8ccccc8ccc7c6cc45)c3c2)cc1 ; c1ccc(-c2c3ccccc3c(-c3cccc4c3oc3cc5ccc6c7ccccc7ccc6c5cc34)c3ccccc23)cc1 ; c1ccc2c(-c3c4ccccc4c(-c4cccc5c4oc4cc6ccc7c8ccccc8ccc7c6cc45)c4ccccc34)cccc2c1 ; c1ccc2cc(-c3c4ccccc4c(-c4cccc5c4oc4cc6ccc7c8ccccc8ccc7c6cc45)c4ccccc34)ccc2c1]

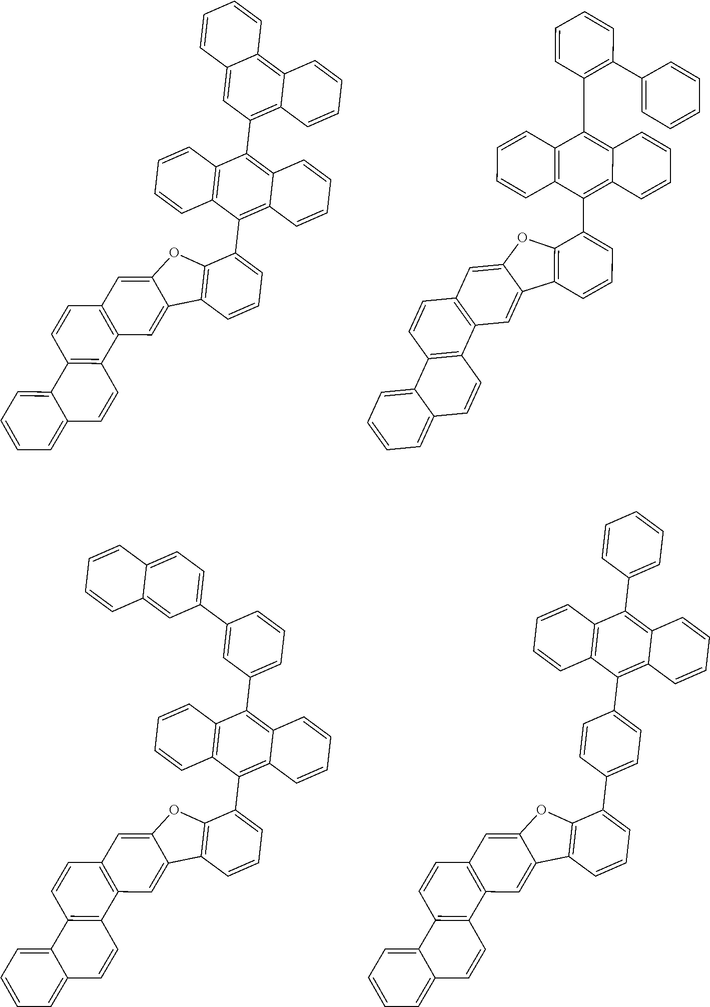 [SMILES: c1ccc2c(c1)cc(-c1c3ccccc3c(-c3cccc4c3oc3cc5ccc6c7ccccc7ccc6c5cc34)c3ccccc13)c1ccccc12 ; c1ccc(-c2ccccc2-c2c3ccccc3c(-c3cccc4c3oc3cc5ccc6c7ccccc7ccc6c5cc34)c3ccccc23)cc1 ; c1cc(-c2ccc3ccccc3c2)cc(-c2c3ccccc3c(-c3cccc4c3oc3cc5ccc6c7ccccc7ccc6c5cc34)c3ccccc23)c1 ; c1ccc(-c2c3ccccc3c(-c3ccc(-c4cccc5c4oc4cc6ccc7c8ccccc8ccc7c6cc45)cc3)c3ccccc23)cc1]

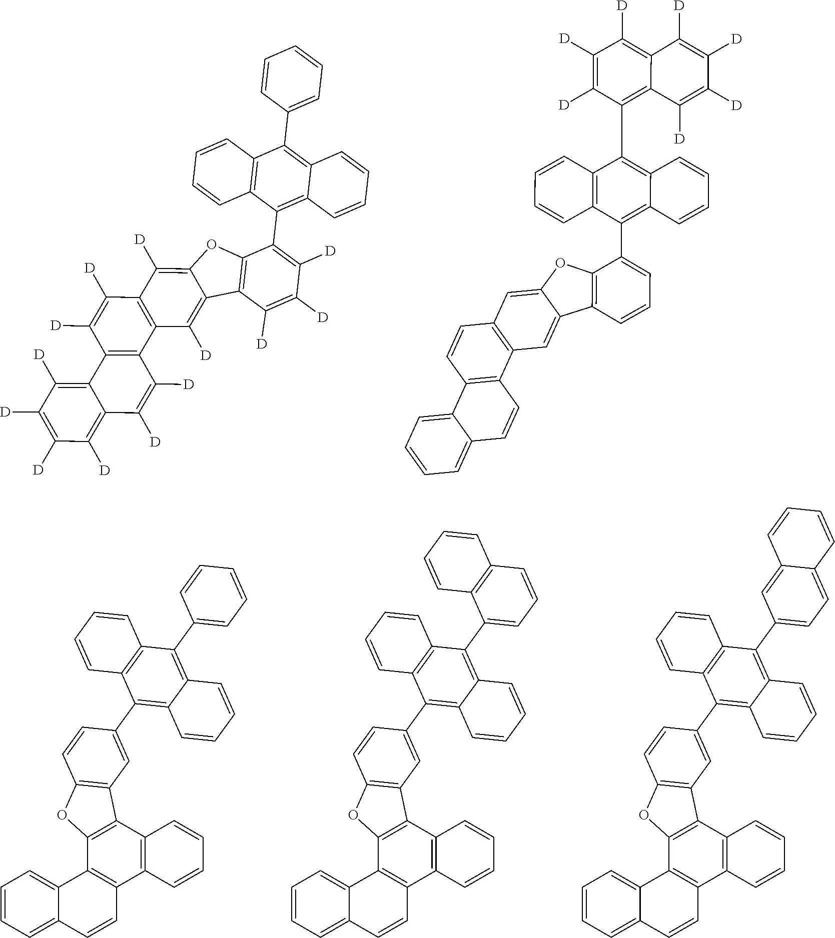 [SMILES: [2H]c1c([2H])c([2H])c2c(oc3c([2H])c4c([2H])c([2H])c5c6c([2H])c([2H])c([2H])c([2H])c6c([2H])c([2H])c5c4c([2H])c32)c1-c1c2ccccc2c(-c2ccccc2)c2ccccc12 ; [2H]c1c([2H])c([2H])c2c(-c3c4ccccc4c(-c4cccc5c4oc4cc6ccc7c8ccccc8ccc7c6cc45)c4ccccc34)c([2H])c([2H])c([2H])c2c1[2H] ; c1ccc(-c2c3ccccc3c(-c3ccc4oc5c6c7ccccc7ccc6c6ccccc6c5c4c3)c3ccccc23)cc1 ; c1ccc2c(-c3c4ccccc4c(-c4ccc5oc6c7c8ccccc8ccc7c7ccccc7c6c5c4)c4ccccc34)cccc2c1 ; c1ccc2cc(-c3c4ccccc4c(-c4ccc5oc6c7c8ccccc8ccc7c7ccccc7c6c5c4)c4ccccc34)ccc2c1]

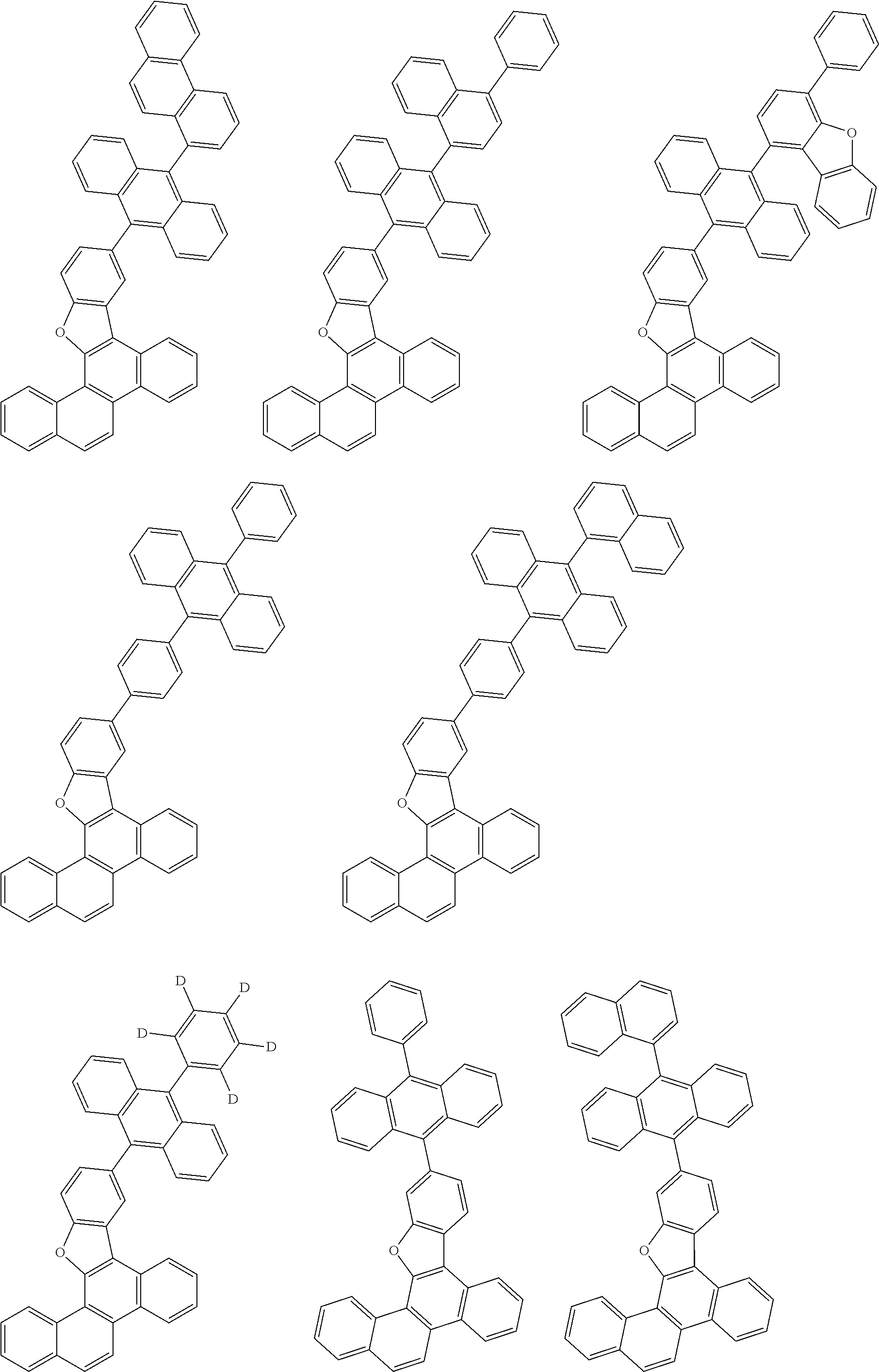

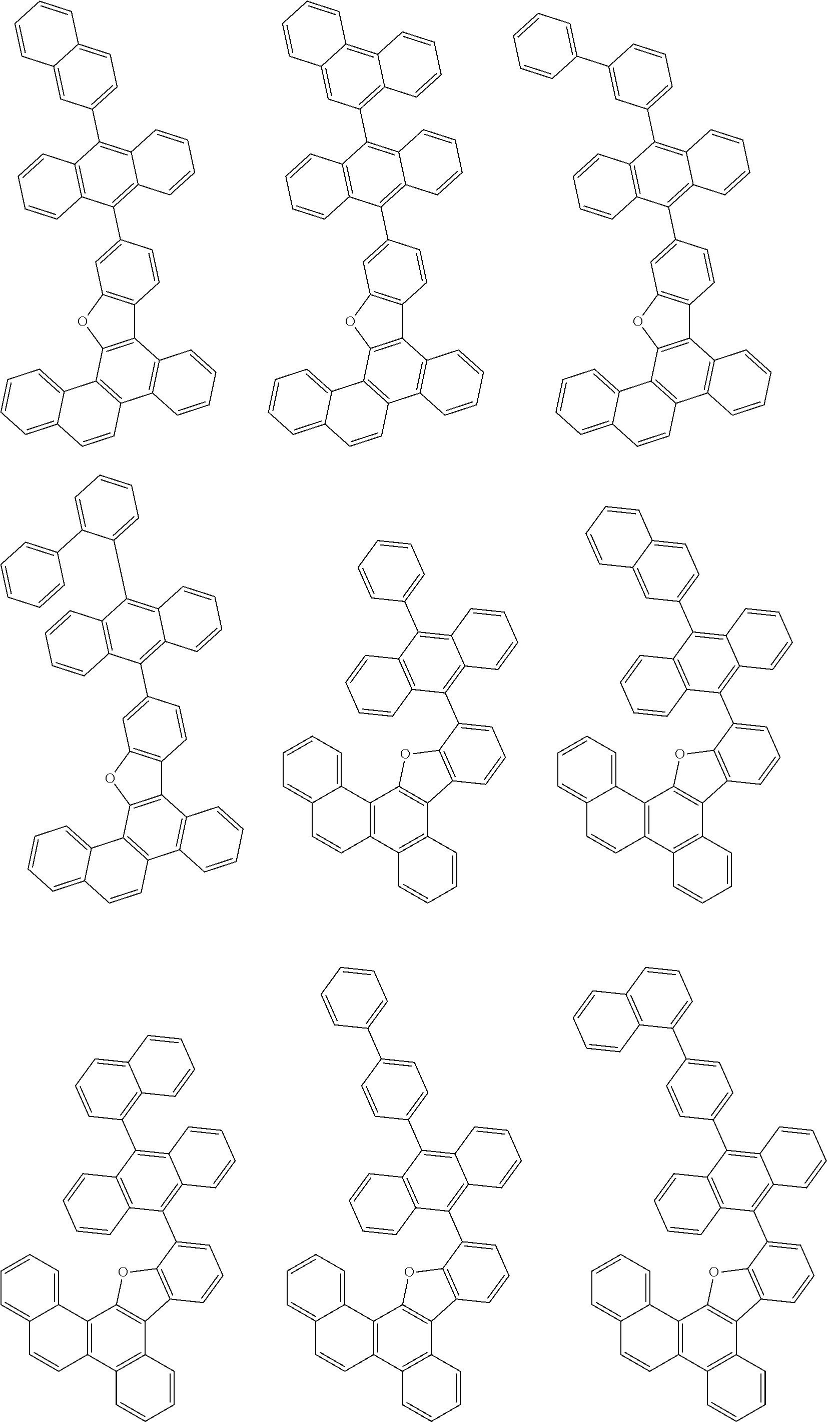

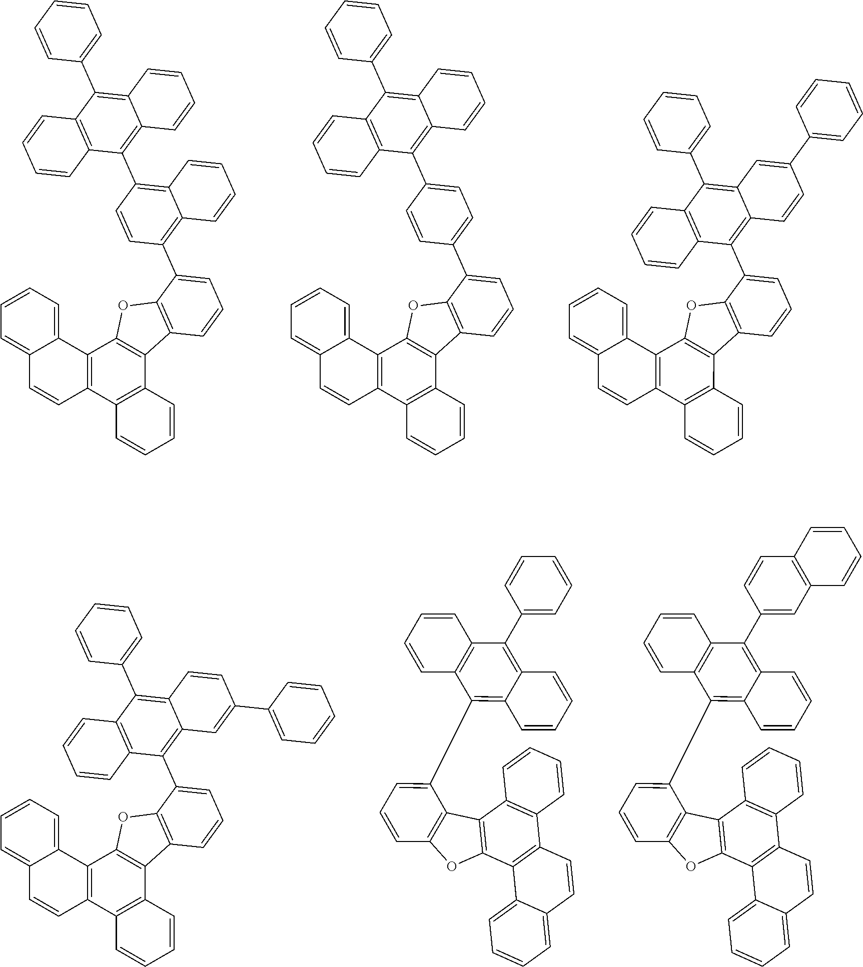 [SMILES: c1ccc(-c2c3ccccc3c(-c3ccc(-c4cccc5c4oc4c6c7ccccc7ccc6c6ccccc6c54)c4ccccc34)c3ccccc23)cc1 ; c1ccc(-c2c3ccccc3c(-c3ccc(-c4cccc5c4oc4c6c7ccccc7ccc6c6ccccc6c54)cc3)c3ccccc23)cc1 ; c1ccc(-c2ccc3c(-c4cccc5c4oc4c6c7ccccc7ccc6c6ccccc6c54)c4ccccc4c(-c4ccccc4)c3c2)cc1 ; c1ccc(-c2ccc3c(-c4ccccc4)c4ccccc4c(-c4cccc5c4oc4c6c7ccccc7ccc6c6ccccc6c54)c3c2)cc1 ; c1ccc(-c2c3ccccc3c(-c3cccc4oc5c6c7ccccc7ccc6c6ccccc6c5c34)c3ccccc23)cc1 ; c1ccc2cc(-c3c4ccccc4c(-c4cccc5oc6c7c8ccccc8ccc7c7ccccc7c6c45)c4ccccc34)ccc2c1]

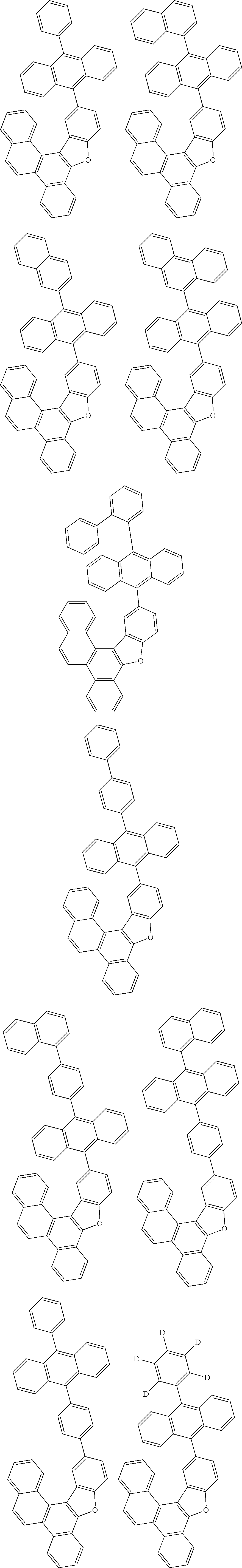

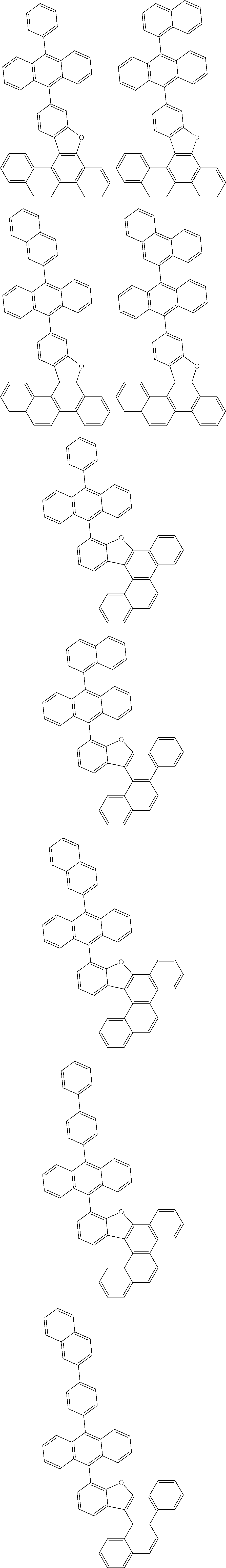

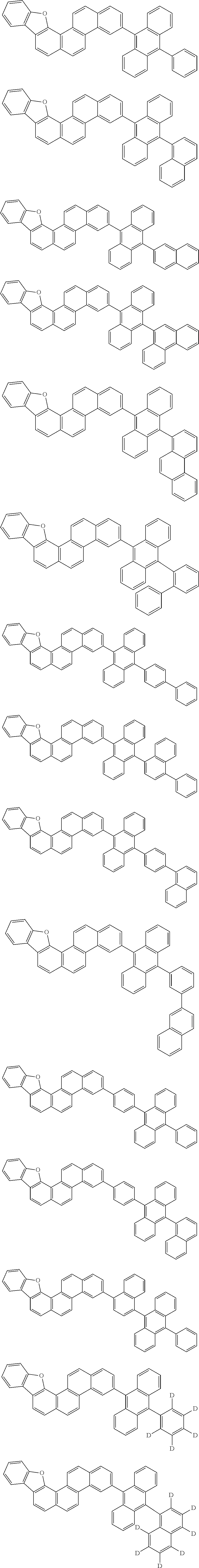

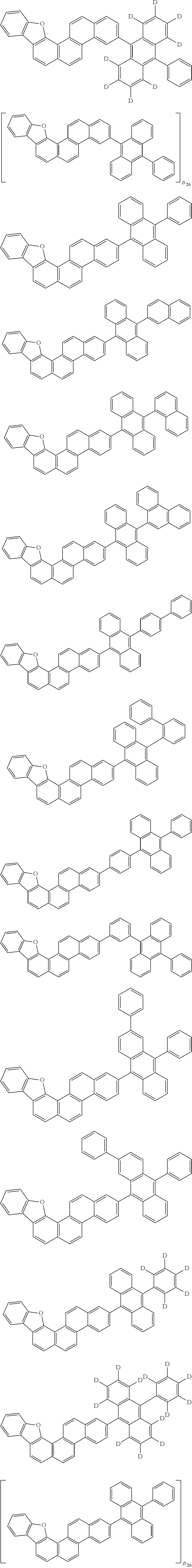

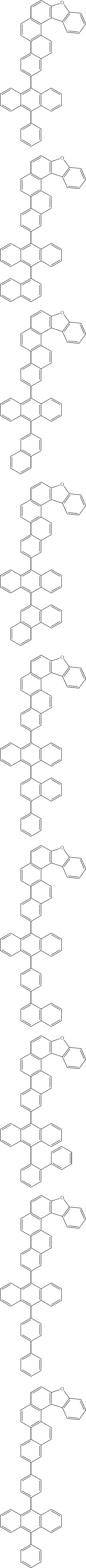

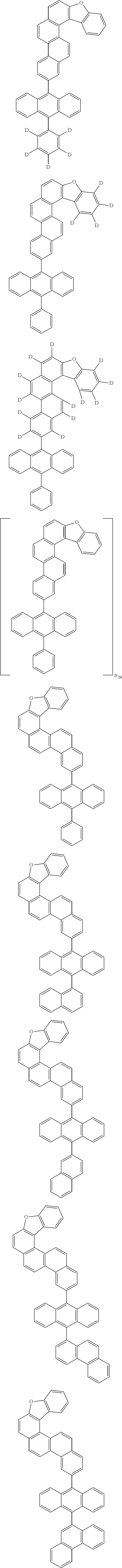

[chemical structure]

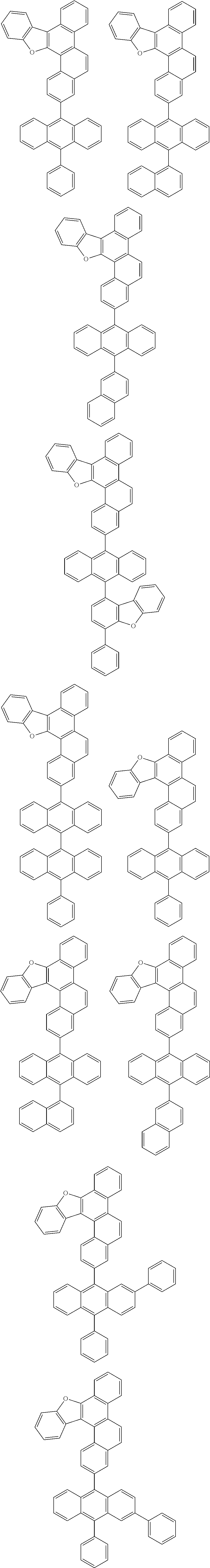

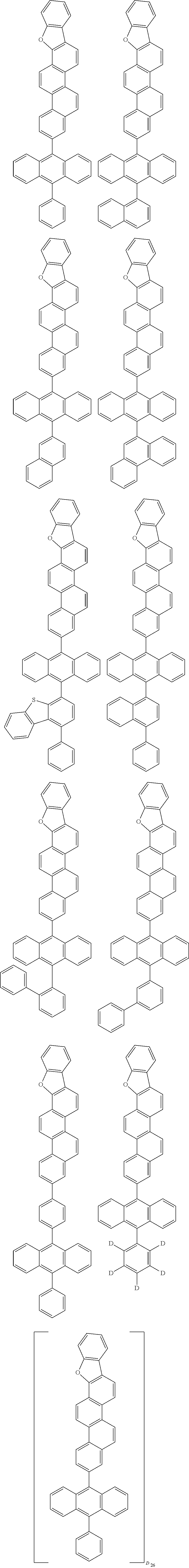

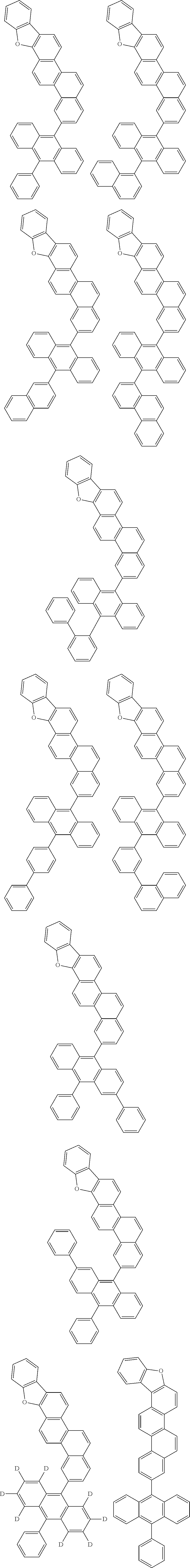

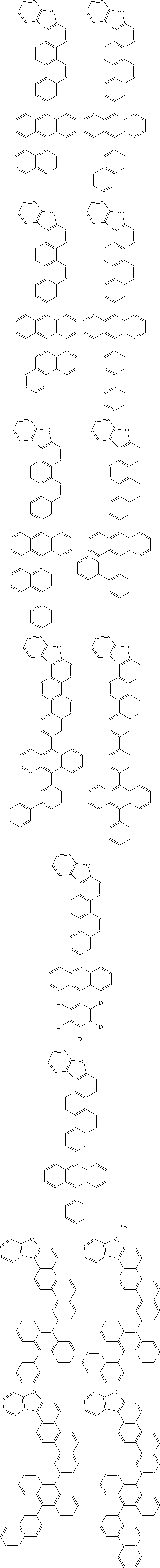

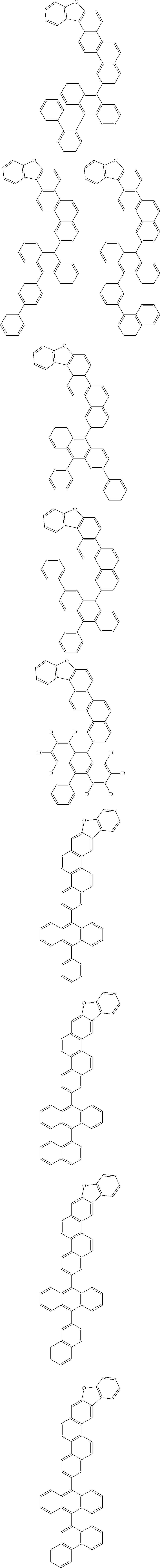

[chemical structure]

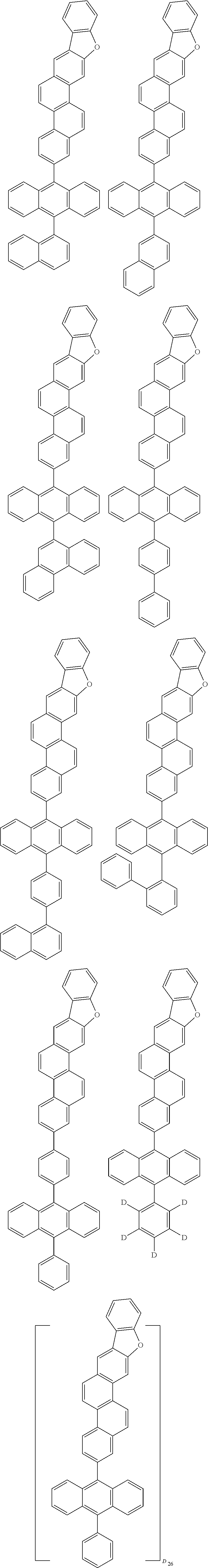

[chemical structure]

[chemical structure]

[chemical structure]

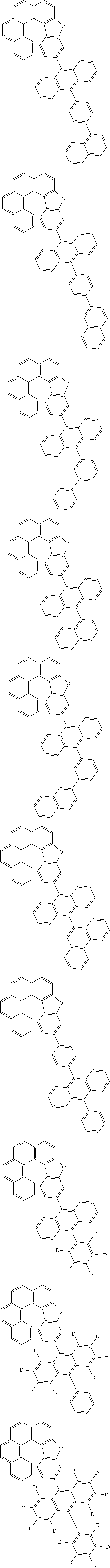

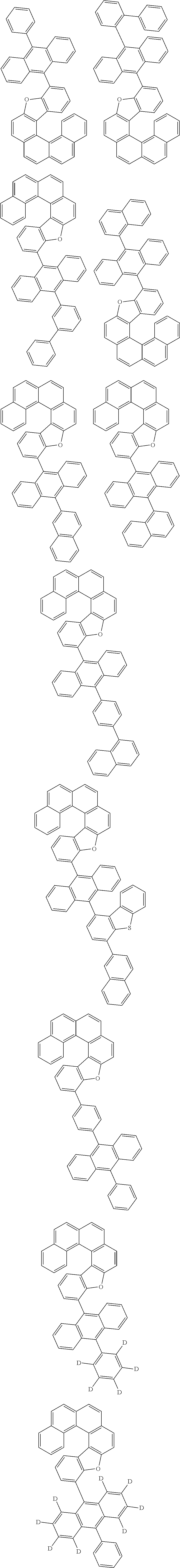

[chemical structure]

[chemical structure]

[chemical structure]

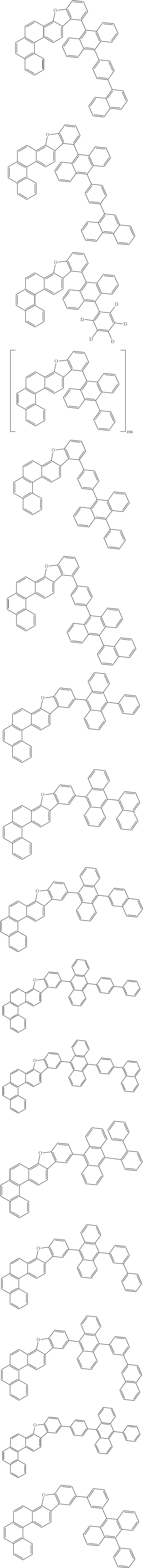

[chemical structure]

[chemical structure]

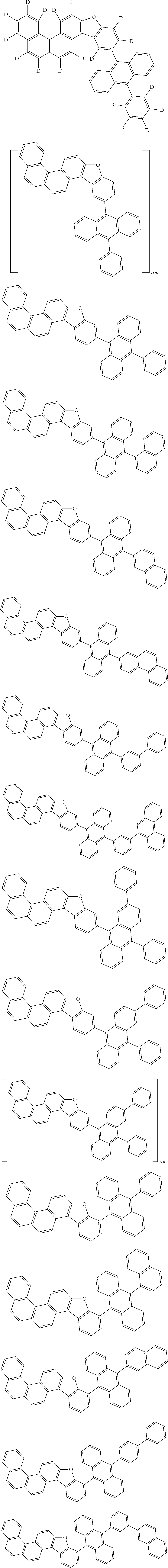

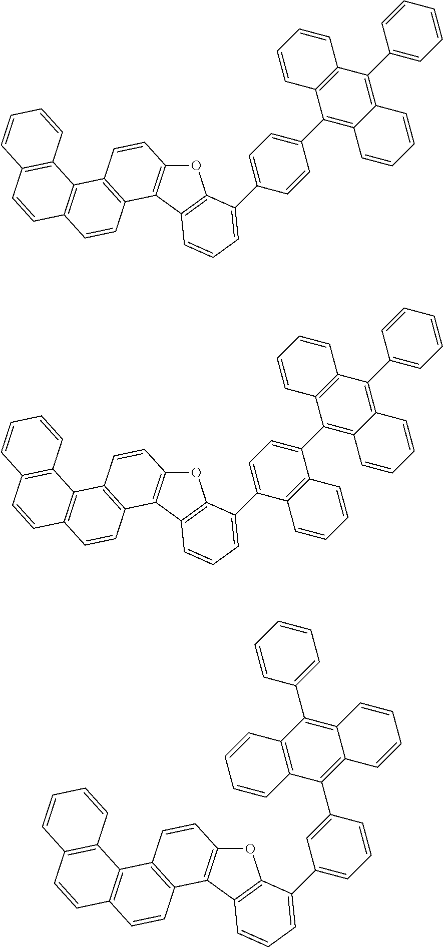 [SMILES: c1ccc(-c2c3ccccc3c(-c3ccc(-c4cccc5c4oc4ccc6c(ccc7ccc8ccccc8c76)c45)cc3)c3ccccc23)cc1 ; c1ccc(-c2c3ccccc3c(-c3ccc(-c4cccc5c4oc4ccc6c(ccc7ccc8ccccc8c76)c45)c4ccccc34)c3ccccc23)cc1 ; c1ccc(-c2c3ccccc3c(-c3cccc(-c4cccc5c4oc4ccc6c(ccc7ccc8ccccc8c76)c45)c3)c3ccccc23)cc1]

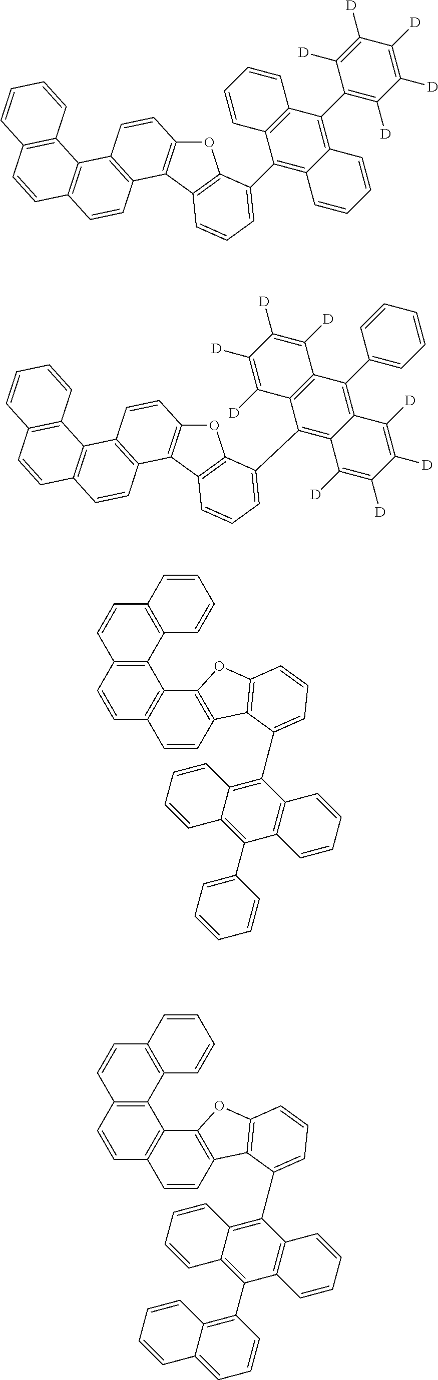 [SMILES: [2H]c1c([2H])c([2H])c(-c2c3ccccc3c(-c3cccc4c3oc3ccc5c(ccc6ccc7ccccc7c65)c34)c3ccccc23)c([2H])c1[2H] ; [2H]c1c([2H])c([2H])c2c(-c3cccc4c3oc3ccc5c(ccc6ccc7ccccc7c65)c34)c3c([2H])c([2H])c([2H])c([2H])c3c(-c3ccccc3)c2c1[2H] ; c1ccc(-c2c3ccccc3c(-c3cccc4oc5c(ccc6ccc7ccc8ccccc8c7c65)c34)c3ccccc23)cc1 ; c1ccc2c(-c3c4ccccc4c(-c4cccc5oc6c(ccc7ccc8ccc9ccccc9c8c76)c45)c4ccccc34)cccc2c1]

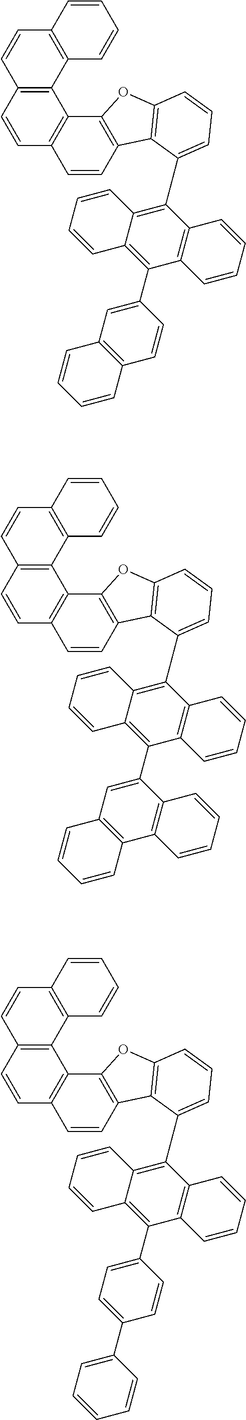 [SMILES: c1ccc2cc(-c3c4ccccc4c(-c4cccc5oc6c(ccc7ccc8ccc9ccccc9c8c76)c45)c4ccccc34)ccc2c1 ; c1ccc2c(c1)cc(-c1c3ccccc3c(-c3cccc4oc5c(ccc6ccc7ccc8ccccc8c7c65)c34)c3ccccc13)c1ccccc12 ; c1ccc(-c2ccc(-c3c4ccccc4c(-c4cccc5oc6c(ccc7ccc8ccc9ccccc9c8c76)c45)c4ccccc34)cc2)cc1]

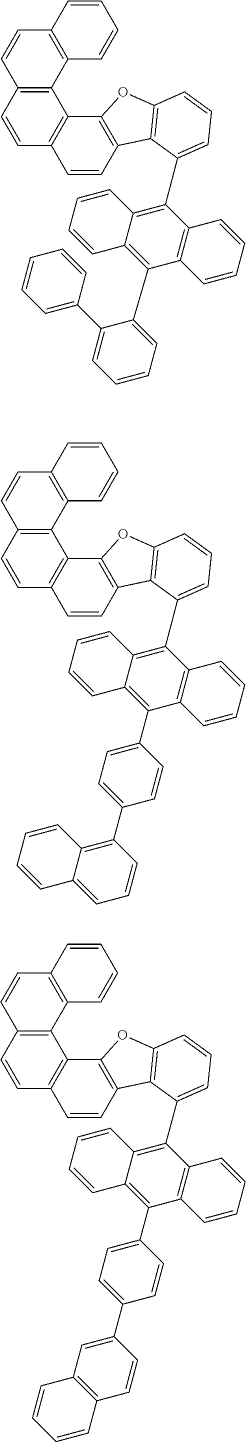

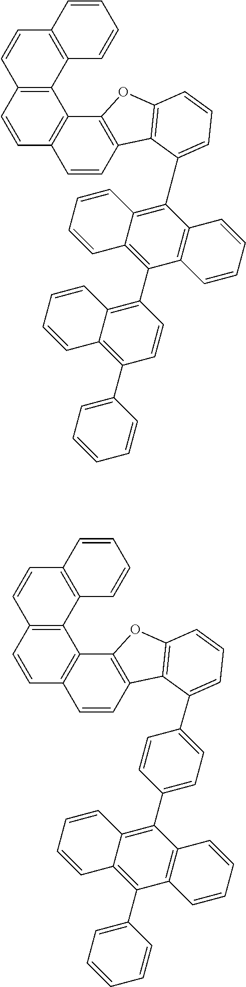 [SMILES: c1ccc(-c2ccc(-c3c4ccccc4c(-c4cccc5oc6c(ccc7ccc8ccc9ccccc9c8c76)c45)c4ccccc34)c3ccccc23)cc1 ; c1ccc(-c2c3ccccc3c(-c3ccc(-c4cccc5oc6c(ccc7ccc8ccc9ccccc9c8c76)c45)cc3)c3ccccc23)cc1]

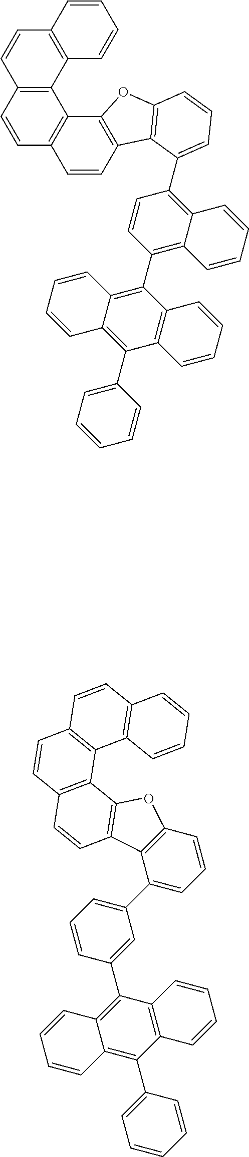 [SMILES: c1ccc(-c2c3ccccc3c(-c3ccc(-c4cccc5oc6c(ccc7ccc8ccc9ccccc9c8c76)c45)c4ccccc34)c3ccccc23)cc1 ; c1ccc(-c2c3ccccc3c(-c3cccc(-c4cccc5oc6c(ccc7ccc8ccc9ccccc9c8c76)c45)c3)c3ccccc23)cc1]

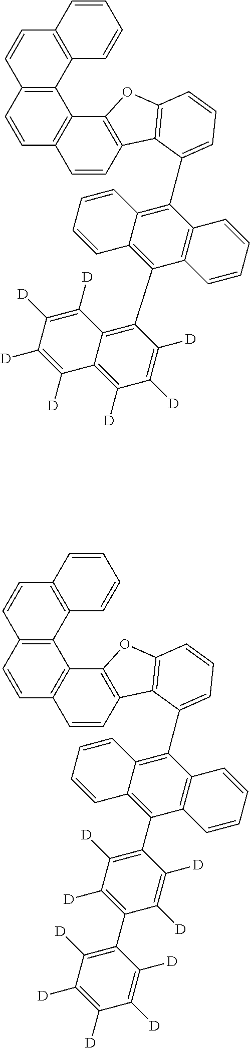 [SMILES: [2H]c1c([2H])c([2H])c2c(-c3c4ccccc4c(-c4cccc5oc6c(ccc7ccc8ccc9ccccc9c8c76)c45)c4ccccc34)c([2H])c([2H])c([2H])c2c1[2H] ; [2H]c1c([2H])c([2H])c(-c2c([2H])c([2H])c(-c3c4ccccc4c(-c4cccc5oc6c(ccc7ccc8ccc9ccccc9c8c76)c45)c4ccccc34)c([2H])c2[2H])c([2H])c1[2H]]

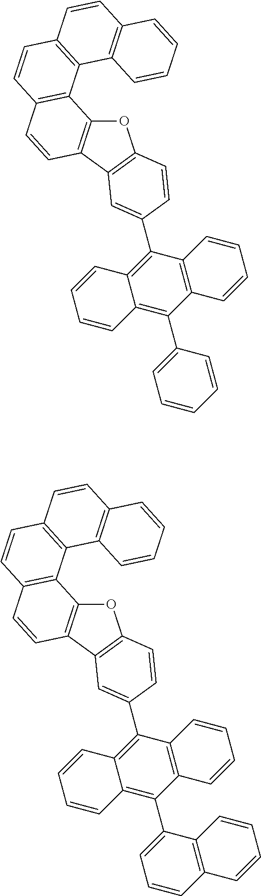 [SMILES: c1ccc(-c2c3ccccc3c(-c3ccc4oc5c(ccc6ccc7ccc8ccccc8c7c65)c4c3)c3ccccc23)cc1 ; c1ccc2c(-c3c4ccccc4c(-c4ccc5oc6c(ccc7ccc8ccc9ccccc9c8c76)c5c4)c4ccccc34)cccc2c1]

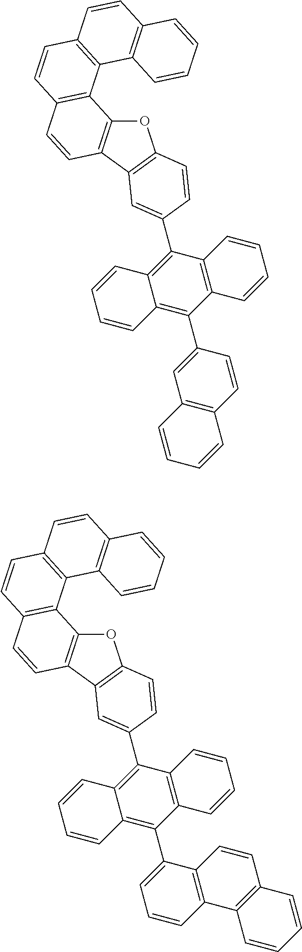 [SMILES: c1ccc2cc(-c3c4ccccc4c(-c4ccc5oc6c(ccc7ccc8ccc9ccccc9c8c76)c5c4)c4ccccc34)ccc2c1 ; c1ccc2c(c1)ccc1c(-c3c4ccccc4c(-c4ccc5oc6c(ccc7ccc8ccc9ccccc9c8c76)c5c4)c4ccccc34)cccc12]

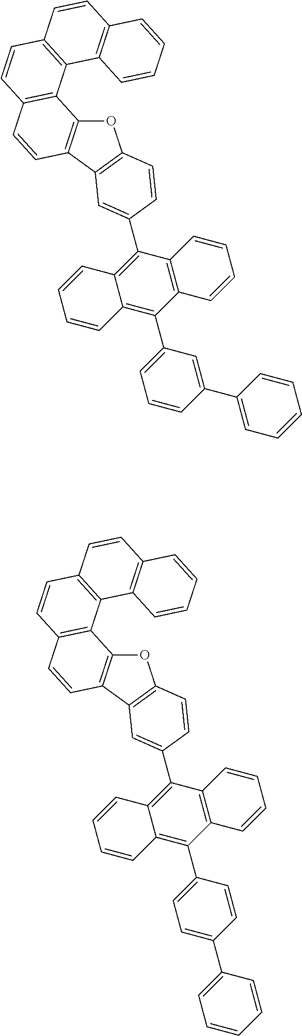 [SMILES: c1ccc(-c2cccc(-c3c4ccccc4c(-c4ccc5oc6c(ccc7ccc8ccc9ccccc9c8c76)c5c4)c4ccccc34)c2)cc1 ; c1ccc(-c2ccc(-c3c4ccccc4c(-c4ccc5oc6c(ccc7ccc8ccc9ccccc9c8c76)c5c4)c4ccccc34)cc2)cc1]

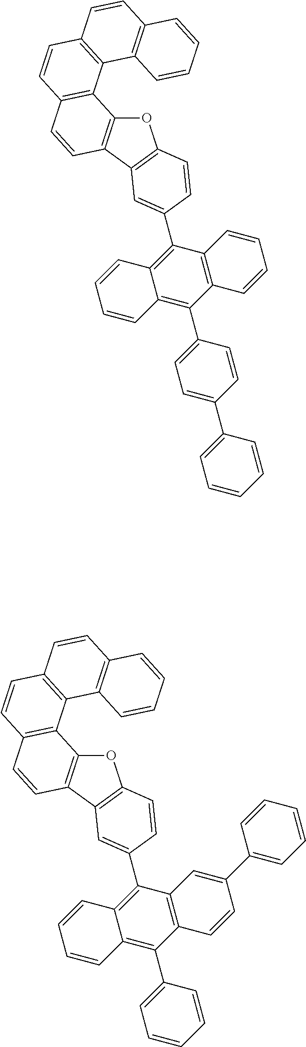 [SMILES: c1ccc(-c2ccc(-c3c4ccccc4c(-c4ccc5oc6c(ccc7ccc8ccc9ccccc9c8c76)c5c4)c4ccccc34)cc2)cc1 ; c1ccc(-c2ccc3c(-c4ccccc4)c4ccccc4c(-c4ccc5oc6c(ccc7ccc8ccc9ccccc9c8c76)c5c4)c3c2)cc1]

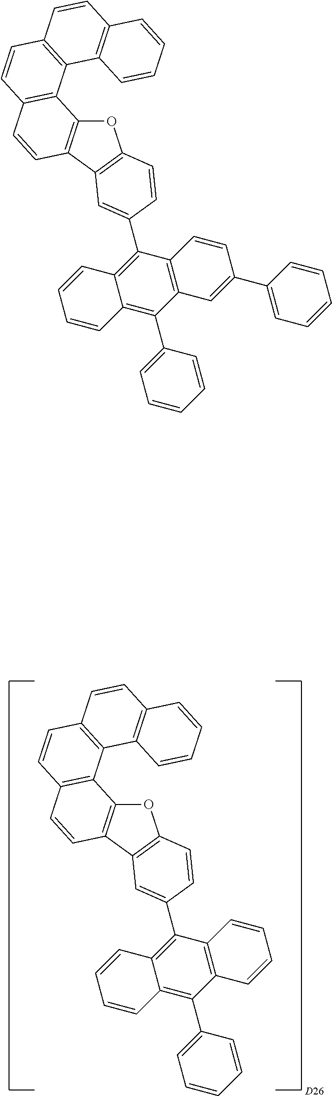

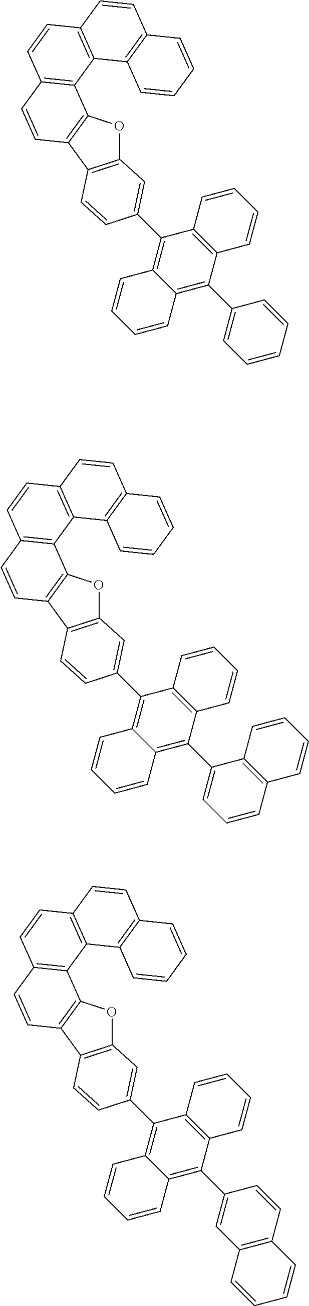 [SMILES: c1ccc(-c2c3ccccc3c(-c3ccc4c(c3)oc3c4ccc4ccc5ccc6ccccc6c5c43)c3ccccc23)cc1 ; c1ccc2c(-c3c4ccccc4c(-c4ccc5c(c4)oc4c5ccc5ccc6ccc7ccccc7c6c54)c4ccccc34)cccc2c1 ; c1ccc2cc(-c3c4ccccc4c(-c4ccc5c(c4)oc4c5ccc5ccc6ccc7ccccc7c6c54)c4ccccc34)ccc2c1]

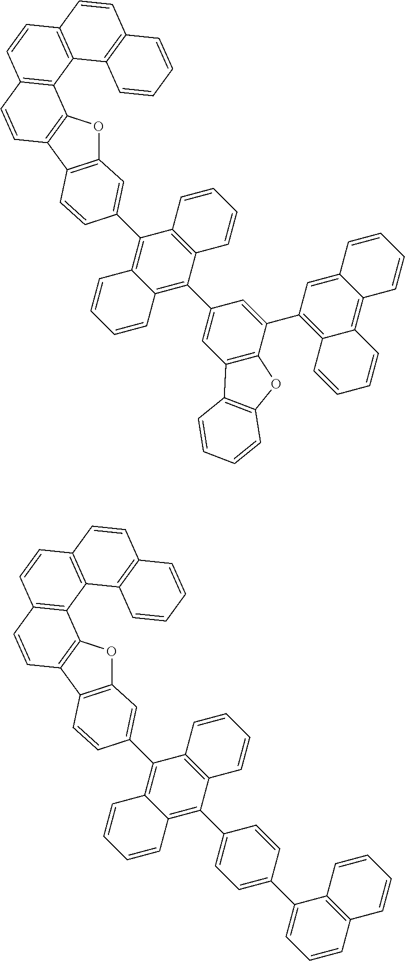 [SMILES: c1ccc2c(c1)cc(-c1cc(-c3c4ccccc4c(-c4ccc5c(c4)oc4c5ccc5ccc6ccc7ccccc7c6c54)c4ccccc34)cc3c1oc1ccccc13)c1ccccc12 ; c1ccc2c(-c3ccc(-c4c5ccccc5c(-c5ccc6c(c5)oc5c6ccc6ccc7ccc8ccccc8c7c65)c5ccccc45)cc3)cccc2c1]

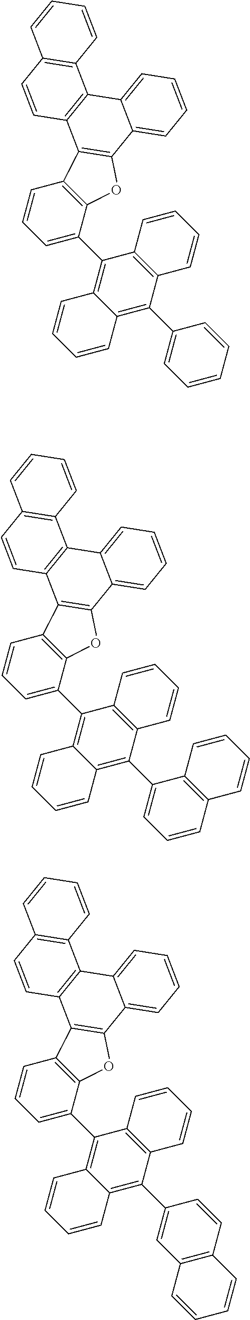 [SMILES: c1ccc(-c2c3ccccc3c(-c3cccc4c3oc3c5ccccc5c5c6ccccc6ccc5c43)c3ccccc23)cc1 ; c1ccc2c(-c3c4ccccc4c(-c4cccc5c4oc4c6ccccc6c6c7ccccc7ccc6c54)c4ccccc34)cccc2c1 ; c1ccc2cc(-c3c4ccccc4c(-c4cccc5c4oc4c6ccccc6c6c7ccccc7ccc6c54)c4ccccc34)ccc2c1]

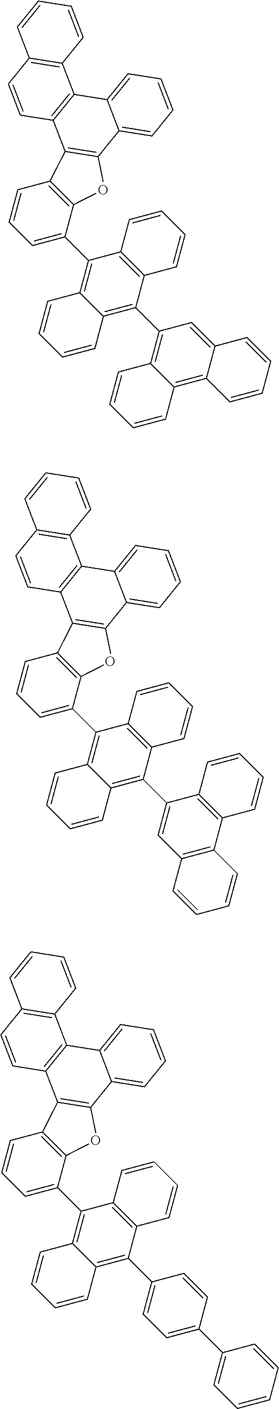 [SMILES: c1ccc2c(c1)cc(-c1c3ccccc3c(-c3cccc4c3oc3c5ccccc5c5c6ccccc6ccc5c43)c3ccccc13)c1ccccc12 ; c1ccc2c(c1)cc(-c1c3ccccc3c(-c3cccc4c3oc3c5ccccc5c5c6ccccc6ccc5c43)c3ccccc13)c1ccccc12 ; c1ccc(-c2ccc(-c3c4ccccc4c(-c4cccc5c4oc4c6ccccc6c6c7ccccc7ccc6c54)c4ccccc34)cc2)cc1]

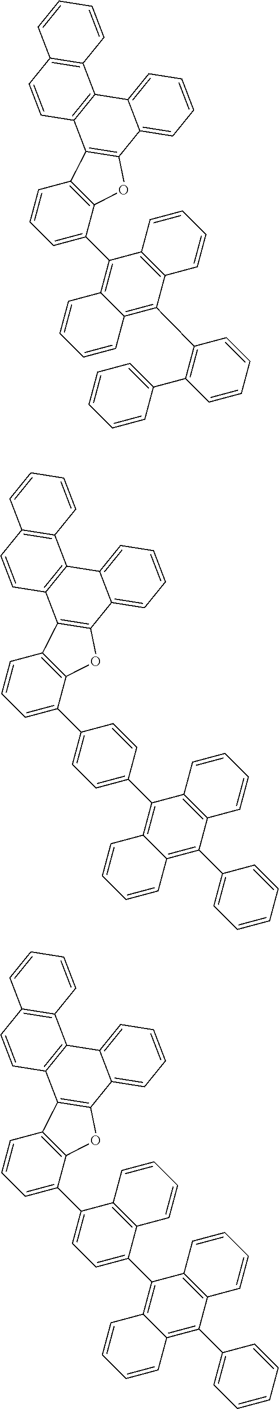 [SMILES: c1ccc(-c2ccccc2-c2c3ccccc3c(-c3cccc4c3oc3c5ccccc5c5c6ccccc6ccc5c43)c3ccccc23)cc1 ; c1ccc(-c2c3ccccc3c(-c3ccc(-c4cccc5c4oc4c6ccccc6c6c7ccccc7ccc6c54)cc3)c3ccccc23)cc1 ; c1ccc(-c2c3ccccc3c(-c3ccc(-c4cccc5c4oc4c6ccccc6c6c7ccccc7ccc6c54)c4ccccc34)c3ccccc23)cc1]

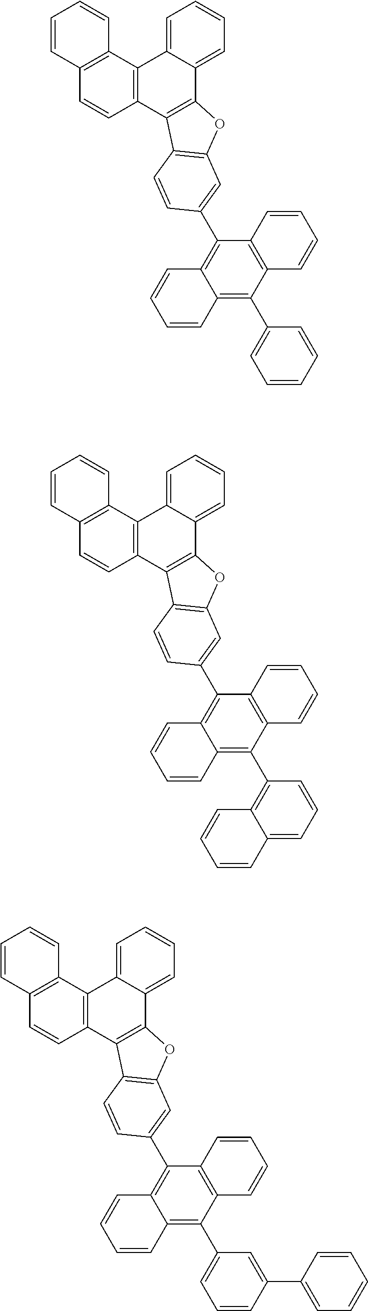 [SMILES: c1ccc(-c2c3ccccc3c(-c3ccc4c(c3)oc3c5ccccc5c5c6ccccc6ccc5c43)c3ccccc23)cc1 ; c1ccc2c(-c3c4ccccc4c(-c4ccc5c(c4)oc4c6ccccc6c6c7ccccc7ccc6c54)c4ccccc34)cccc2c1 ; c1ccc(-c2cccc(-c3c4ccccc4c(-c4ccc5c(c4)oc4c6ccccc6c6c7ccccc7ccc6c54)c4ccccc34)c2)cc1]

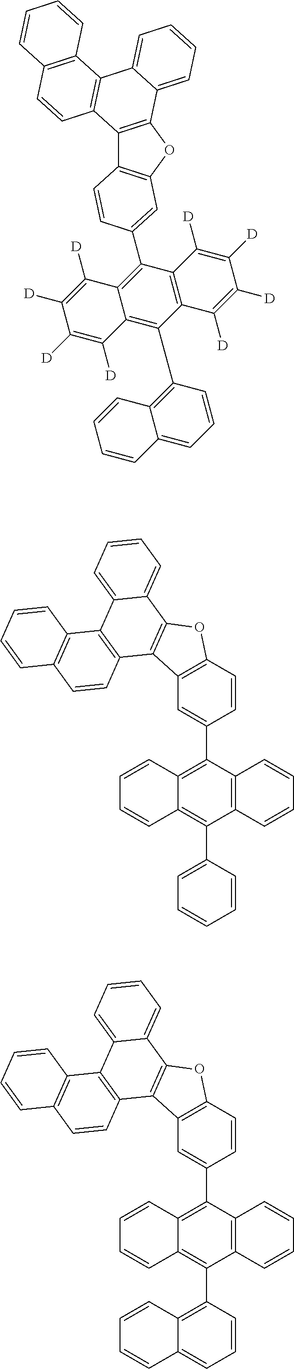 [SMILES: [2H]c1c([2H])c([2H])c2c(-c3cccc4ccccc34)c3c([2H])c([2H])c([2H])c([2H])c3c(-c3ccc4c(c3)oc3c5ccccc5c5c6ccccc6ccc5c43)c2c1[2H] ; c1ccc(-c2c3ccccc3c(-c3ccc4oc5c6ccccc6c6c7ccccc7ccc6c5c4c3)c3ccccc23)cc1 ; c1ccc2c(-c3c4ccccc4c(-c4ccc5oc6c7ccccc7c7c8ccccc8ccc7c6c5c4)c4ccccc34)cccc2c1]

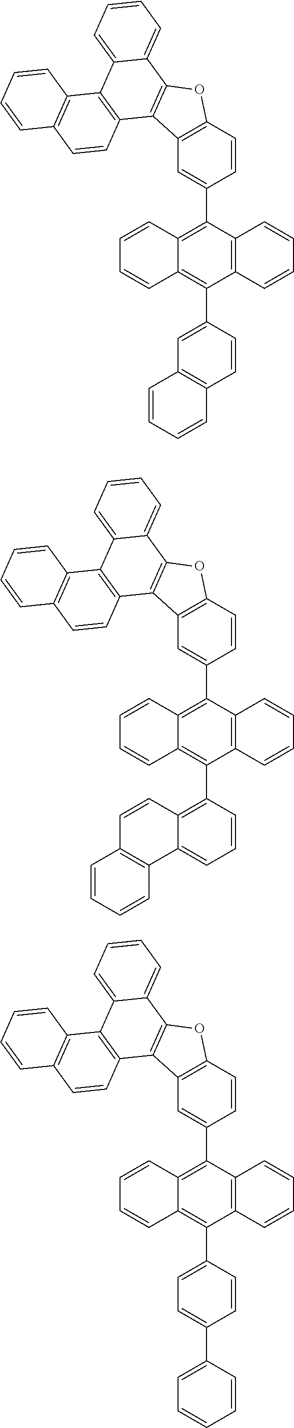

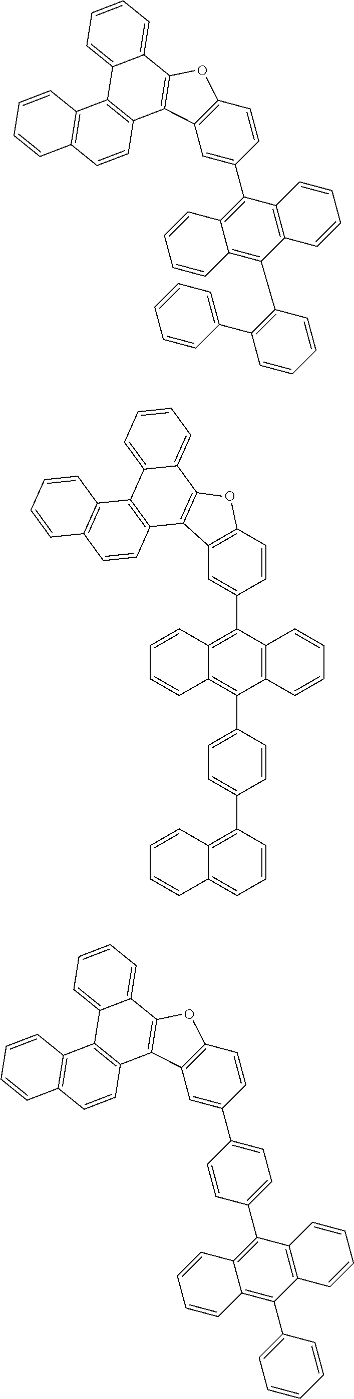 [SMILES: c1ccc(-c2ccccc2-c2c3ccccc3c(-c3ccc4oc5c6ccccc6c6c7ccccc7ccc6c5c4c3)c3ccccc23)cc1 ; c1ccc2c(-c3ccc(-c4c5ccccc5c(-c5ccc6oc7c8ccccc8c8c9ccccc9ccc8c7c6c5)c5ccccc45)cc3)cccc2c1 ; c1ccc(-c2c3ccccc3c(-c3ccc(-c4ccc5oc6c7ccccc7c7c8ccccc8ccc7c6c5c4)cc3)c3ccccc23)cc1]

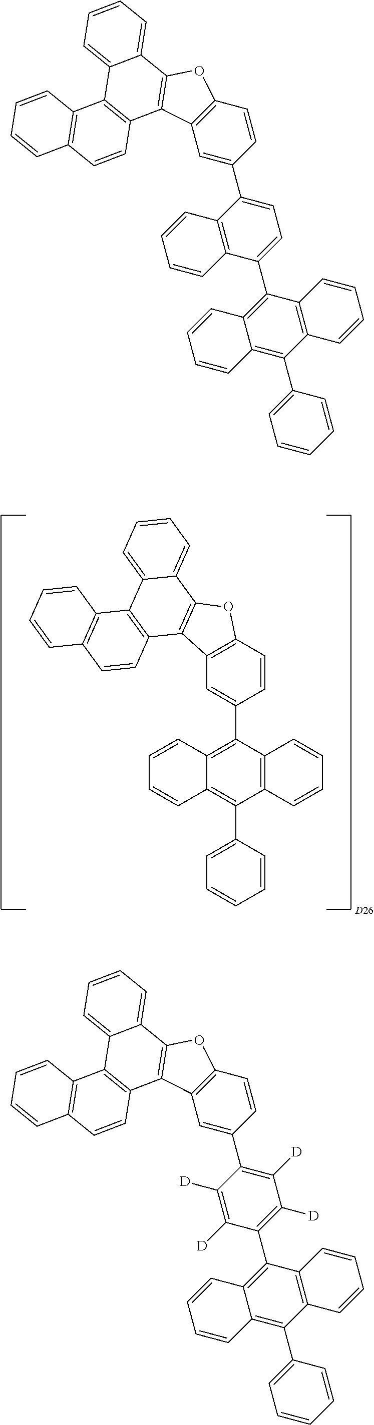

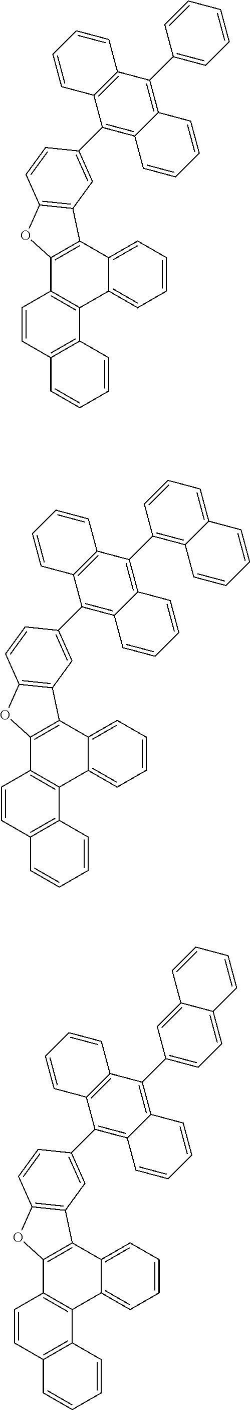 [SMILES: c1ccc(-c2c3ccccc3c(-c3ccc4oc5c6ccc7ccccc7c6c6ccccc6c5c4c3)c3ccccc23)cc1 ; c1ccc2c(-c3c4ccccc4c(-c4ccc5oc6c7ccc8ccccc8c7c7ccccc7c6c5c4)c4ccccc34)cccc2c1 ; c1ccc2cc(-c3c4ccccc4c(-c4ccc5oc6c7ccc8ccccc8c7c7ccccc7c6c5c4)c4ccccc34)ccc2c1]

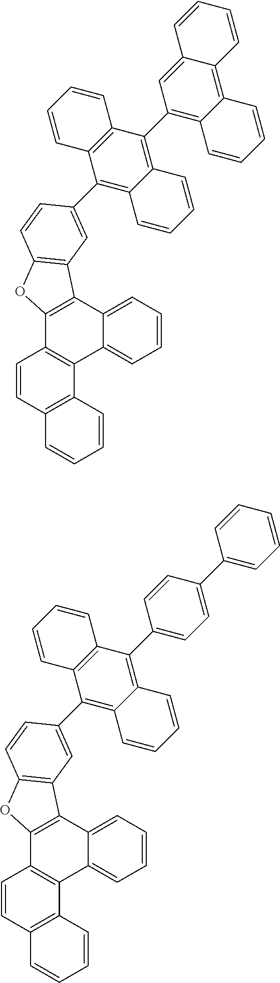 [SMILES: c1ccc2c(c1)cc(-c1c3ccccc3c(-c3ccc4oc5c6ccc7ccccc7c6c6ccccc6c5c4c3)c3ccccc13)c1ccccc12 ; c1ccc(-c2ccc(-c3c4ccccc4c(-c4ccc5oc6c7ccc8ccccc8c7c7ccccc7c6c5c4)c4ccccc34)cc2)cc1]

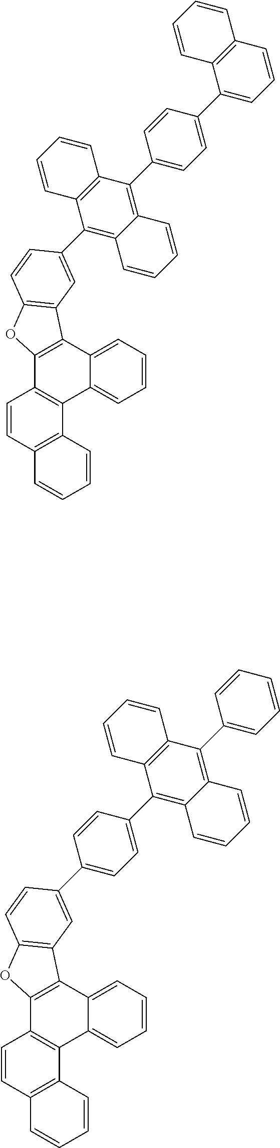 [SMILES: c1ccc2c(-c3ccc(-c4c5ccccc5c(-c5ccc6oc7c8ccc9ccccc9c8c8ccccc8c7c6c5)c5ccccc45)cc3)cccc2c1 ; c1ccc(-c2c3ccccc3c(-c3ccc(-c4ccc5oc6c7ccc8ccccc8c7c7ccccc7c6c5c4)cc3)c3ccccc23)cc1]

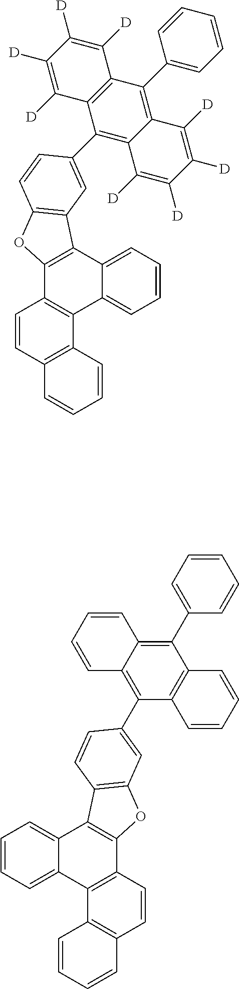 [SMILES: [2H]c1c([2H])c([2H])c2c(-c3ccc4oc5c6ccc7ccccc7c6c6ccccc6c5c4c3)c3c([2H])c([2H])c([2H])c([2H])c3c(-c3ccccc3)c2c1[2H] ; c1ccc(-c2c3ccccc3c(-c3ccc4c(c3)oc3c5ccc6ccccc6c5c5ccccc5c43)c3ccccc23)cc1]

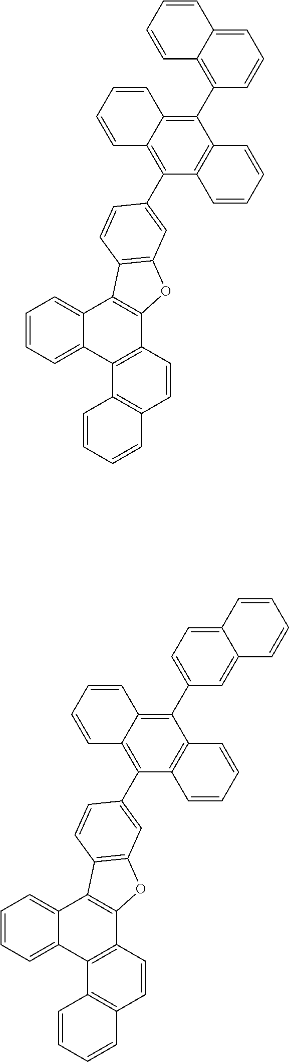 [SMILES: c1ccc2c(-c3c4ccccc4c(-c4ccc5c(c4)oc4c6ccc7ccccc7c6c6ccccc6c54)c4ccccc34)cccc2c1 ; c1ccc2cc(-c3c4ccccc4c(-c4ccc5c(c4)oc4c6ccc7ccccc7c6c6ccccc6c54)c4ccccc34)ccc2c1]

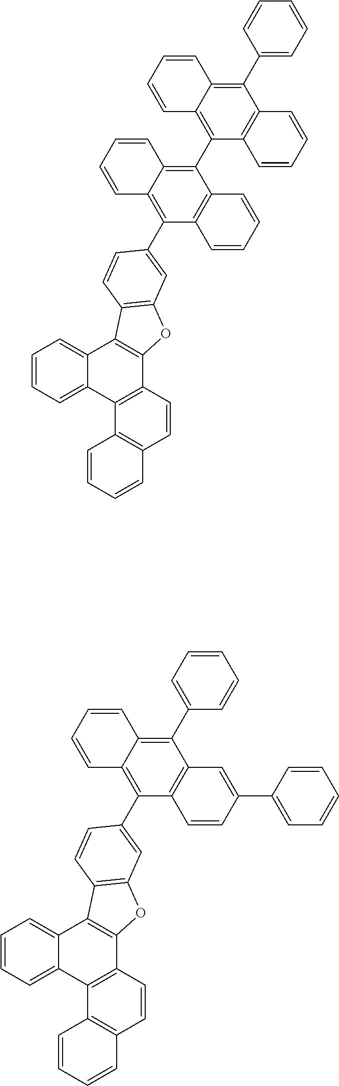 [SMILES: c1ccc(-c2c3ccccc3c(-c3c4ccccc4c(-c4ccc5c(c4)oc4c6ccc7ccccc7c6c6ccccc6c54)c4ccccc34)c3ccccc23)cc1 ; c1ccc(-c2ccc3c(-c4ccc5c(c4)oc4c6ccc7ccccc7c6c6ccccc6c54)c4ccccc4c(-c4ccccc4)c3c2)cc1]

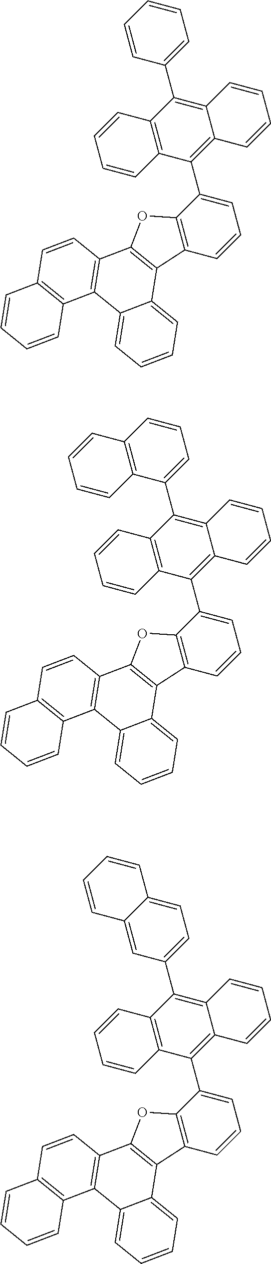 [SMILES: c1ccc(-c2c3ccccc3c(-c3cccc4c3oc3c5ccc6ccccc6c5c5ccccc5c43)c3ccccc23)cc1 ; c1ccc2c(-c3c4ccccc4c(-c4cccc5c4oc4c6ccc7ccccc7c6c6ccccc6c54)c4ccccc34)cccc2c1 ; c1ccc2cc(-c3c4ccccc4c(-c4cccc5c4oc4c6ccc7ccccc7c6c6ccccc6c54)c4ccccc34)ccc2c1]

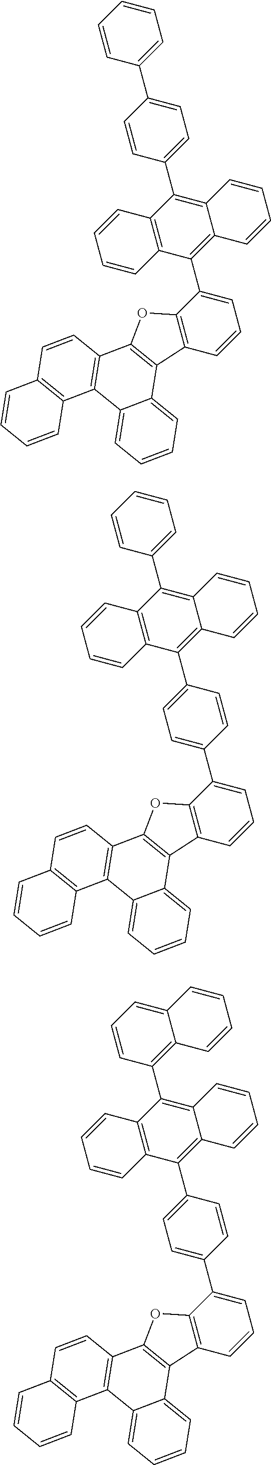

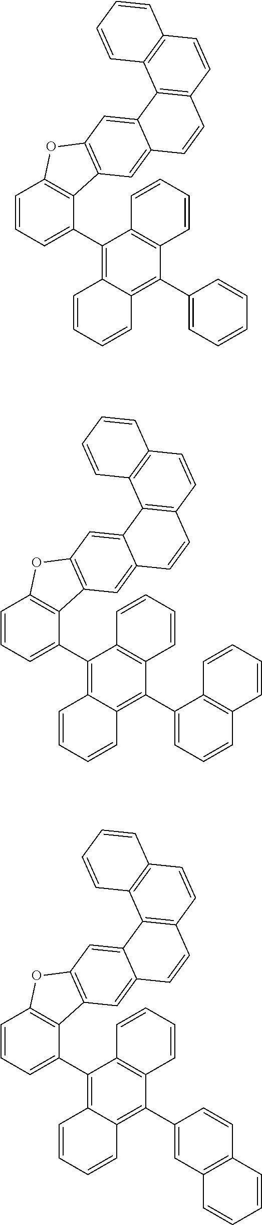 [SMILES: c1ccc(-c2c3ccccc3c(-c3cccc4oc5cc6c(ccc7ccc8ccccc8c76)cc5c34)c3ccccc23)cc1 ; c1ccc2c(-c3c4ccccc4c(-c4cccc5oc6cc7c(ccc8ccc9ccccc9c87)cc6c45)c4ccccc34)cccc2c1 ; c1ccc2cc(-c3c4ccccc4c(-c4cccc5oc6cc7c(ccc8ccc9ccccc9c87)cc6c45)c4ccccc34)ccc2c1]

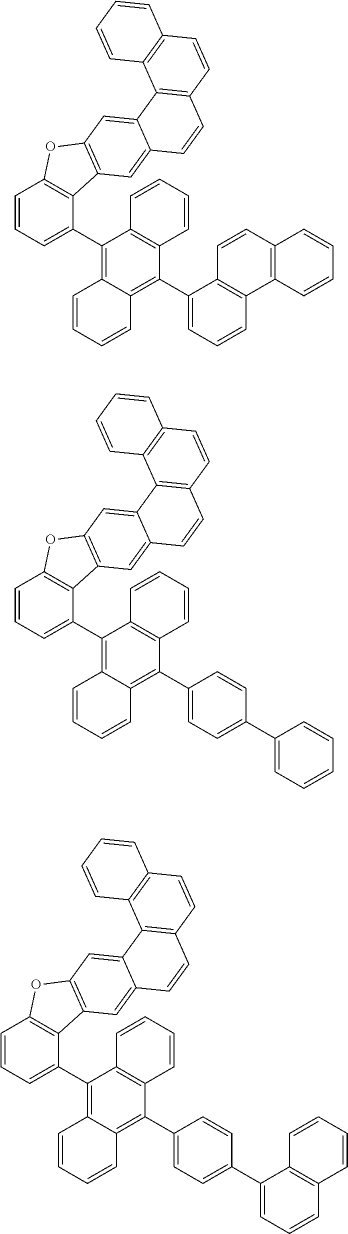 [SMILES: c1ccc2c(c1)ccc1c(-c3c4ccccc4c(-c4cccc5oc6cc7c(ccc8ccc9ccccc9c87)cc6c45)c4ccccc34)cccc12 ; c1ccc(-c2ccc(-c3c4ccccc4c(-c4cccc5oc6cc7c(ccc8ccc9ccccc9c87)cc6c45)c4ccccc34)cc2)cc1 ; c1ccc2c(-c3ccc(-c4c5ccccc5c(-c5cccc6oc7cc8c(ccc9ccc%10ccccc%10c98)cc7c56)c5ccccc45)cc3)cccc2c1]

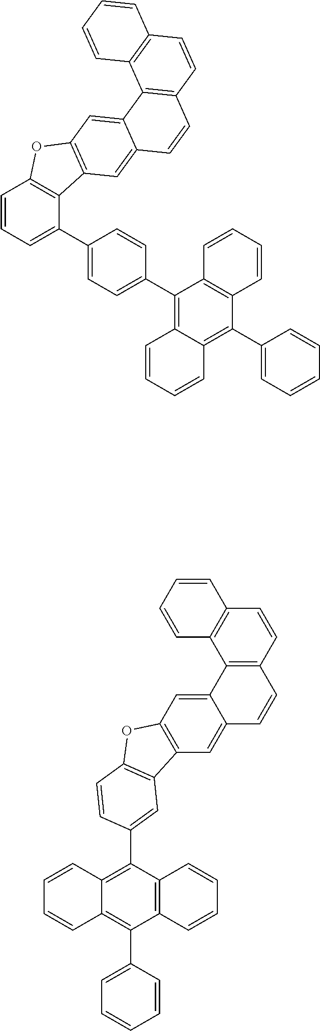 [SMILES: c1ccc(-c2c3ccccc3c(-c3ccc(-c4cccc5oc6cc7c(ccc8ccc9ccccc9c87)cc6c45)cc3)c3ccccc23)cc1 ; c1ccc(-c2c3ccccc3c(-c3ccc4oc5cc6c(ccc7ccc8ccccc8c76)cc5c4c3)c3ccccc23)cc1]

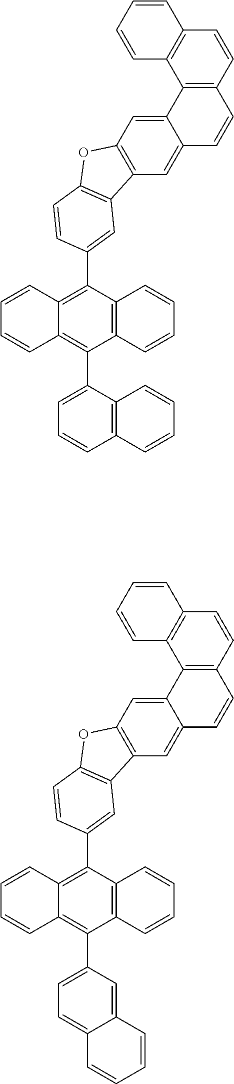 [SMILES: c1ccc2c(-c3c4ccccc4c(-c4ccc5oc6cc7c(ccc8ccc9ccccc9c87)cc6c5c4)c4ccccc34)cccc2c1 ; c1ccc2cc(-c3c4ccccc4c(-c4ccc5oc6cc7c(ccc8ccc9ccccc9c87)cc6c5c4)c4ccccc34)ccc2c1]

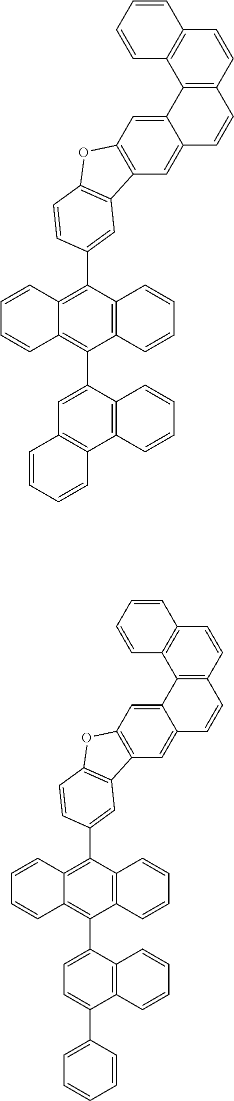 [SMILES: c1ccc2c(c1)cc(-c1c3ccccc3c(-c3ccc4oc5cc6c(ccc7ccc8ccccc8c76)cc5c4c3)c3ccccc13)c1ccccc12 ; c1ccc(-c2ccc(-c3c4ccccc4c(-c4ccc5oc6cc7c(ccc8ccc9ccccc9c87)cc6c5c4)c4ccccc34)c3ccccc23)cc1]

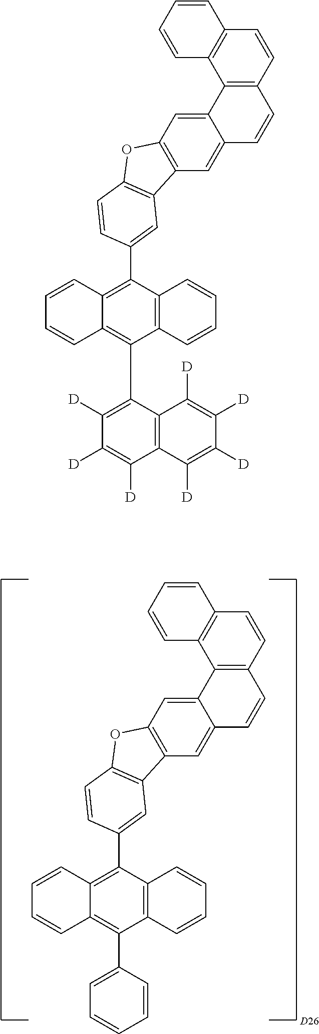

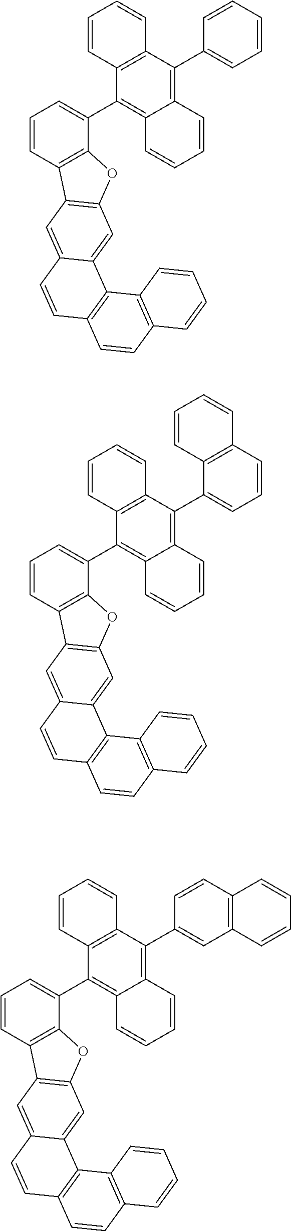 [SMILES: c1ccc(-c2c3ccccc3c(-c3cccc4c3oc3cc5c(ccc6ccc7ccccc7c65)cc34)c3ccccc23)cc1 ; c1ccc2c(-c3c4ccccc4c(-c4cccc5c4oc4cc6c(ccc7ccc8ccccc8c76)cc45)c4ccccc34)cccc2c1 ; c1ccc2cc(-c3c4ccccc4c(-c4cccc5c4oc4cc6c(ccc7ccc8ccccc8c76)cc45)c4ccccc34)ccc2c1]

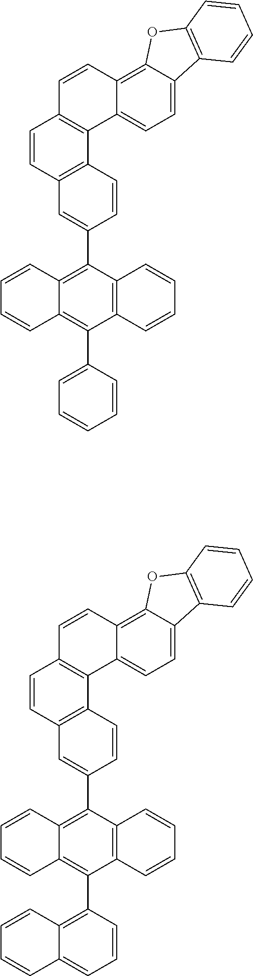 [SMILES: c1ccc(-c2c3ccccc3c(-c3ccc4c(ccc5ccc6c(ccc7c8ccccc8oc76)c54)c3)c3ccccc23)cc1 ; c1ccc2c(-c3c4ccccc4c(-c4ccc5c(ccc6ccc7c(ccc8c9ccccc9oc87)c65)c4)c4ccccc34)cccc2c1]

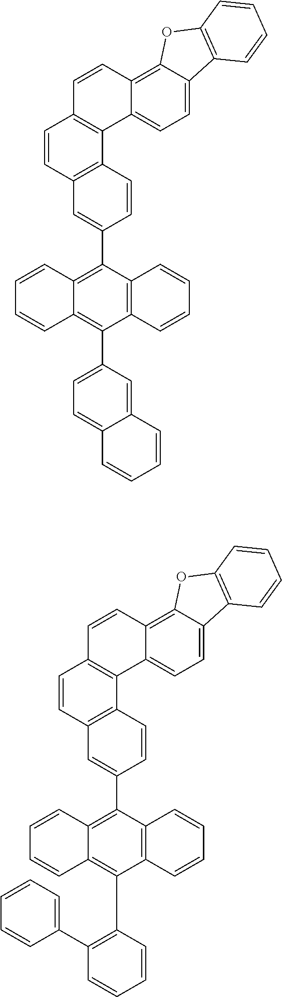 [SMILES: c1ccc2cc(-c3c4ccccc4c(-c4ccc5c(ccc6ccc7c(ccc8c9ccccc9oc87)c65)c4)c4ccccc34)ccc2c1 ; c1ccc(-c2ccccc2-c2c3ccccc3c(-c3ccc4c(ccc5ccc6c(ccc7c8ccccc8oc76)c54)c3)c3ccccc23)cc1]

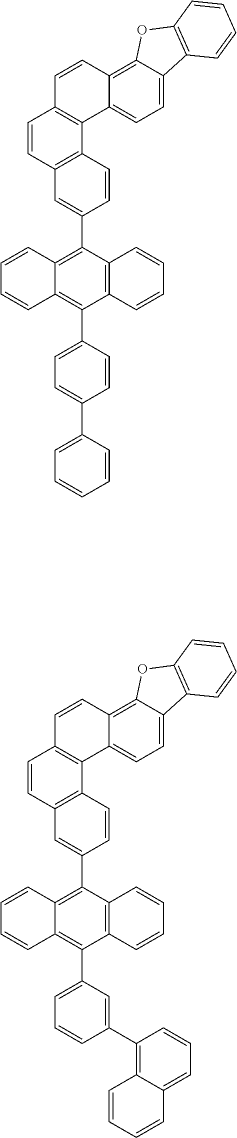 [SMILES: c1ccc(-c2ccc(-c3c4ccccc4c(-c4ccc5c(ccc6ccc7c(ccc8c9ccccc9oc87)c65)c4)c4ccccc34)cc2)cc1 ; c1cc(-c2cccc3ccccc23)cc(-c2c3ccccc3c(-c3ccc4c(ccc5ccc6c(ccc7c8ccccc8oc76)c54)c3)c3ccccc23)c1]

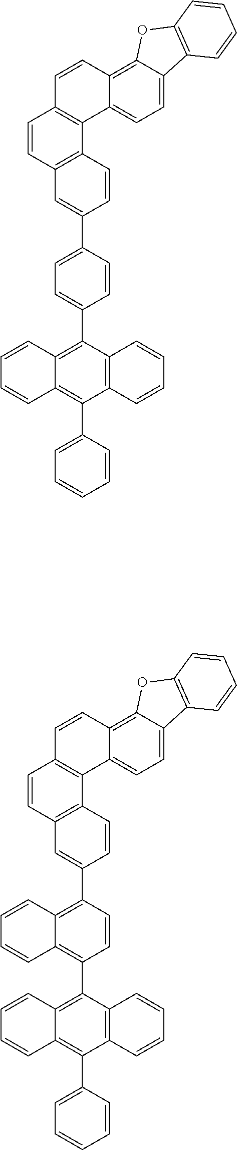 [SMILES: c1ccc(-c2c3ccccc3c(-c3ccc(-c4ccc5c(ccc6ccc7c(ccc8c9ccccc9oc87)c65)c4)cc3)c3ccccc23)cc1 ; c1ccc(-c2c3ccccc3c(-c3ccc(-c4ccc5c(ccc6ccc7c(ccc8c9ccccc9oc87)c65)c4)c4ccccc34)c3ccccc23)cc1]

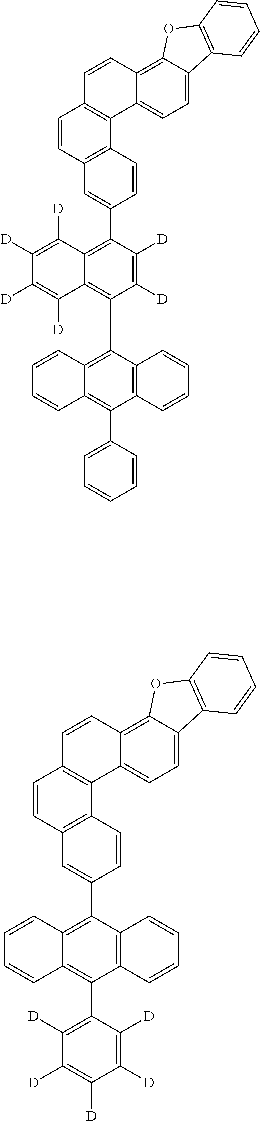 [SMILES: [2H]c1c([2H])c([2H])c2c(-c3c4ccccc4c(-c4ccccc4)c4ccccc34)c([2H])c([2H])c(-c3ccc4c(ccc5ccc6c(ccc7c8ccccc8oc76)c54)c3)c2c1[2H] ; [2H]c1c([2H])c([2H])c(-c2c3ccccc3c(-c3ccc4c(ccc5ccc6c(ccc7c8ccccc8oc76)c54)c3)c3ccccc23)c([2H])c1[2H]]

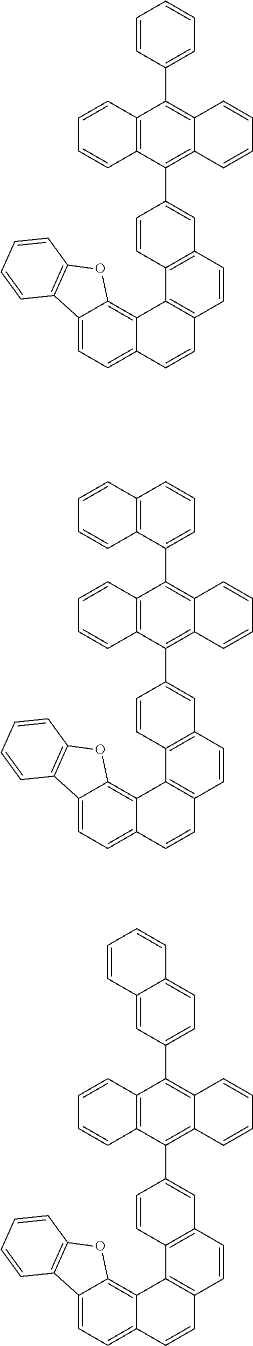 [SMILES: c1ccc(-c2c3ccccc3c(-c3ccc4c(ccc5ccc6ccc7c8ccccc8oc7c6c54)c3)c3ccccc23)cc1 ; c1ccc2c(-c3c4ccccc4c(-c4ccc5c(ccc6ccc7ccc8c9ccccc9oc8c7c65)c4)c4ccccc34)cccc2c1 ; c1ccc2cc(-c3c4ccccc4c(-c4ccc5c(ccc6ccc7ccc8c9ccccc9oc8c7c65)c4)c4ccccc34)ccc2c1]

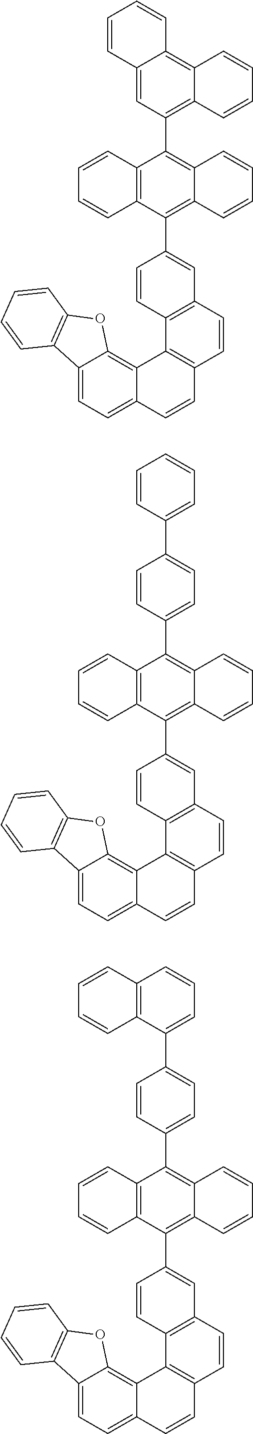

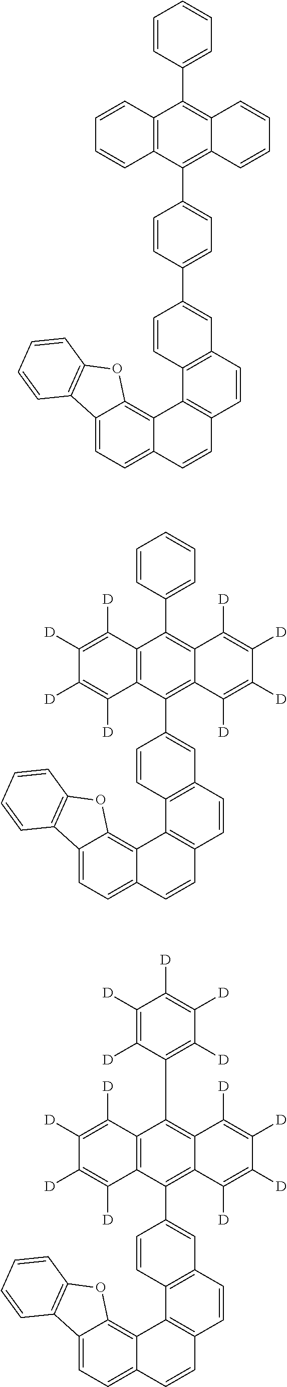 [SMILES: c1ccc(-c2c3ccccc3c(-c3ccc(-c4ccc5c(ccc6ccc7ccc8c9ccccc9oc8c7c65)c4)cc3)c3ccccc23)cc1 ; [2H]c1c([2H])c([2H])c2c(-c3ccc4c(ccc5ccc6ccc7c8ccccc8oc7c6c54)c3)c3c([2H])c([2H])c([2H])c([2H])c3c(-c3ccccc3)c2c1[2H] ; [2H]c1c([2H])c([2H])c(-c2c3c([2H])c([2H])c([2H])c([2H])c3c(-c3ccc4c(ccc5ccc6ccc7c8ccccc8oc7c6c54)c3)c3c([2H])c([2H])c([2H])c([2H])c23)c([2H])c1[2H]]

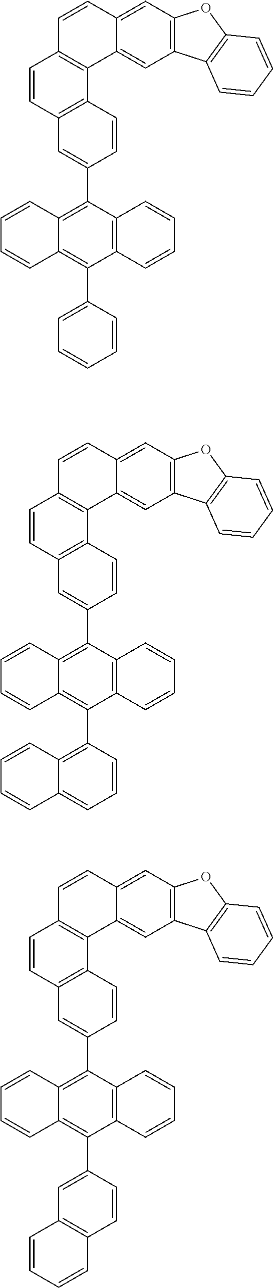 [SMILES: c1ccc(-c2c3ccccc3c(-c3ccc4c(ccc5ccc6cc7oc8ccccc8c7cc6c54)c3)c3ccccc23)cc1 ; c1ccc2c(-c3c4ccccc4c(-c4ccc5c(ccc6ccc7cc8oc9ccccc9c8cc7c65)c4)c4ccccc34)cccc2c1 ; c1ccc2cc(-c3c4ccccc4c(-c4ccc5c(ccc6ccc7cc8oc9ccccc9c8cc7c65)c4)c4ccccc34)ccc2c1]

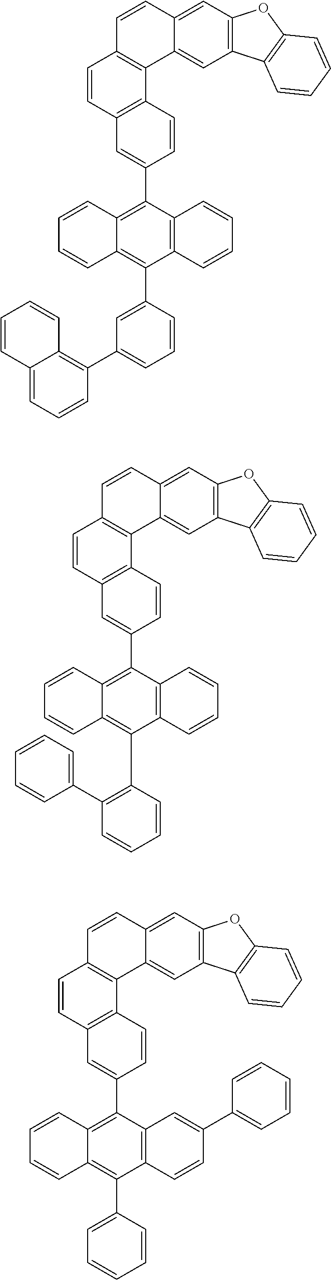 [SMILES: c1cc(-c2cccc3ccccc23)cc(-c2c3ccccc3c(-c3ccc4c(ccc5ccc6cc7oc8ccccc8c7cc6c54)c3)c3ccccc23)c1 ; c1ccc(-c2ccccc2-c2c3ccccc3c(-c3ccc4c(ccc5ccc6cc7oc8ccccc8c7cc6c54)c3)c3ccccc23)cc1 ; c1ccc(-c2ccc3c(-c4ccccc4)c4ccccc4c(-c4ccc5c(ccc6ccc7cc8oc9ccccc9c8cc7c65)c4)c3c2)cc1]

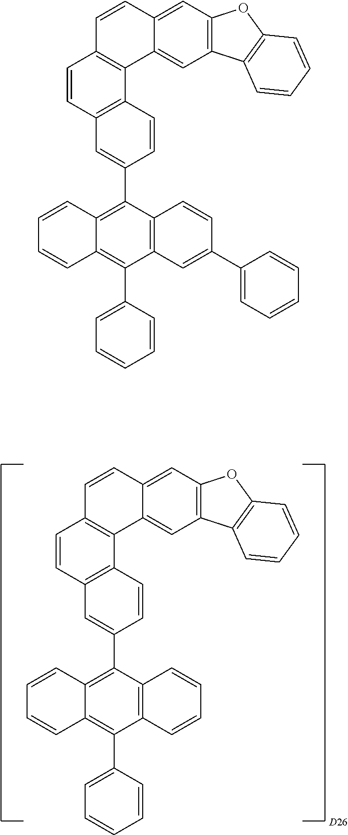

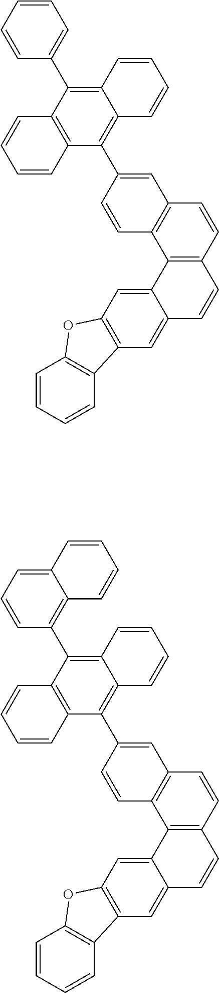 [SMILES: c1ccc(-c2c3ccccc3c(-c3ccc4c(ccc5ccc6cc7c(cc6c54)oc4ccccc47)c3)c3ccccc23)cc1 ; c1ccc2c(-c3c4ccccc4c(-c4ccc5c(ccc6ccc7cc8c(cc7c65)oc5ccccc58)c4)c4ccccc34)cccc2c1]

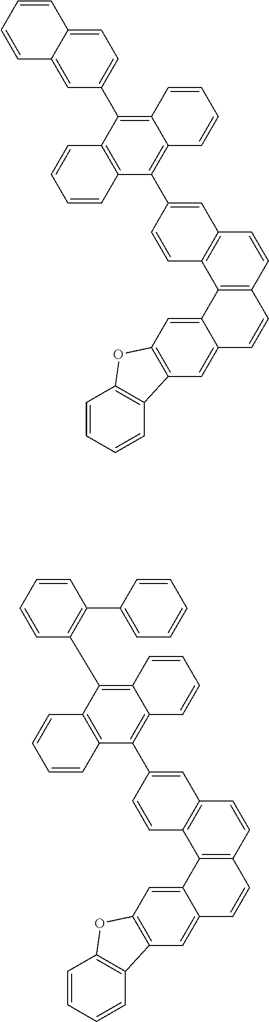 [SMILES: c1ccc2cc(-c3c4ccccc4c(-c4ccc5c(ccc6ccc7cc8c(cc7c65)oc5ccccc58)c4)c4ccccc34)ccc2c1 ; c1ccc(-c2ccccc2-c2c3ccccc3c(-c3ccc4c(ccc5ccc6cc7c(cc6c54)oc4ccccc47)c3)c3ccccc23)cc1]

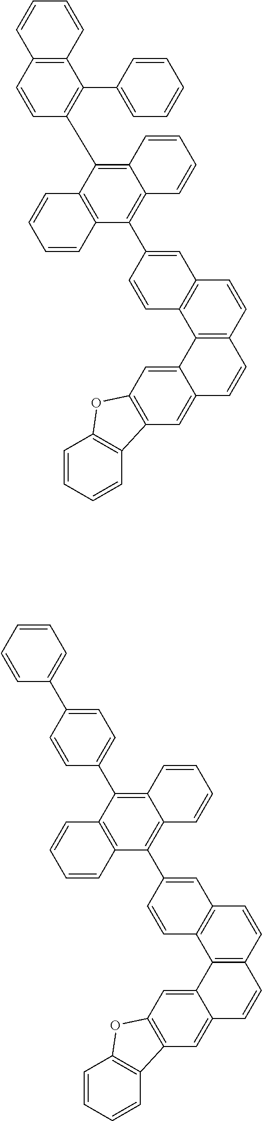 [SMILES: c1ccc(-c2c(-c3c4ccccc4c(-c4ccc5c(ccc6ccc7cc8c(cc7c65)oc5ccccc58)c4)c4ccccc34)ccc3ccccc23)cc1 ; c1ccc(-c2ccc(-c3c4ccccc4c(-c4ccc5c(ccc6ccc7cc8c(cc7c65)oc5ccccc58)c4)c4ccccc34)cc2)cc1]

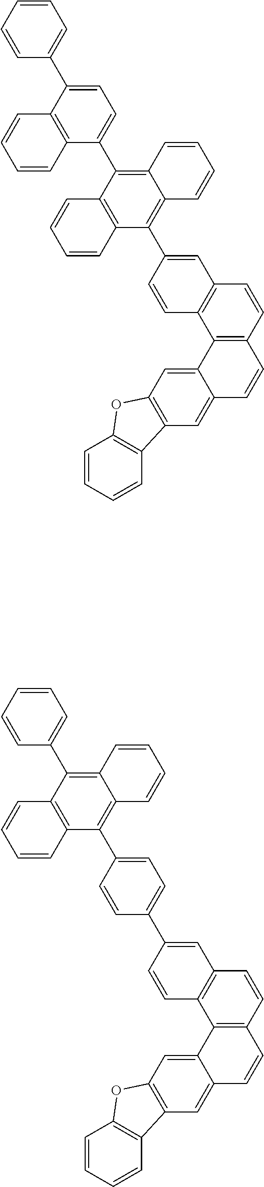 [SMILES: c1ccc(-c2ccc(-c3c4ccccc4c(-c4ccc5c(ccc6ccc7cc8c(cc7c65)oc5ccccc58)c4)c4ccccc34)c3ccccc23)cc1 ; c1ccc(-c2c3ccccc3c(-c3ccc(-c4ccc5c(ccc6ccc7cc8c(cc7c65)oc5ccccc58)c4)cc3)c3ccccc23)cc1]

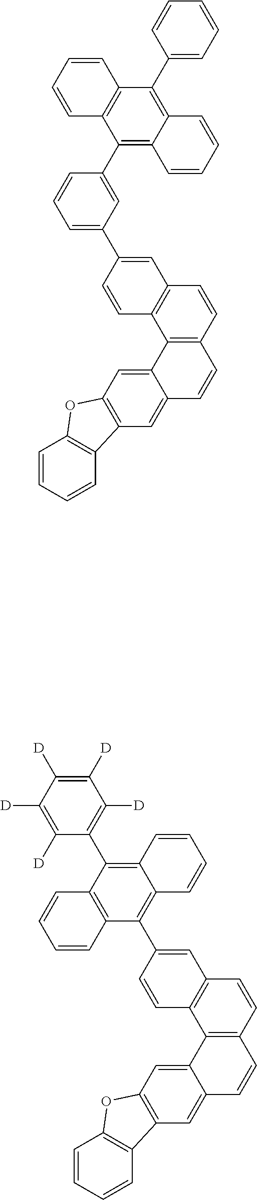 [SMILES: c1ccc(-c2c3ccccc3c(-c3cccc(-c4ccc5c(ccc6ccc7cc8c(cc7c65)oc5ccccc58)c4)c3)c3ccccc23)cc1 ; [2H]c1c([2H])c([2H])c(-c2c3ccccc3c(-c3ccc4c(ccc5ccc6cc7c(cc6c54)oc4ccccc47)c3)c3ccccc23)c([2H])c1[2H]]

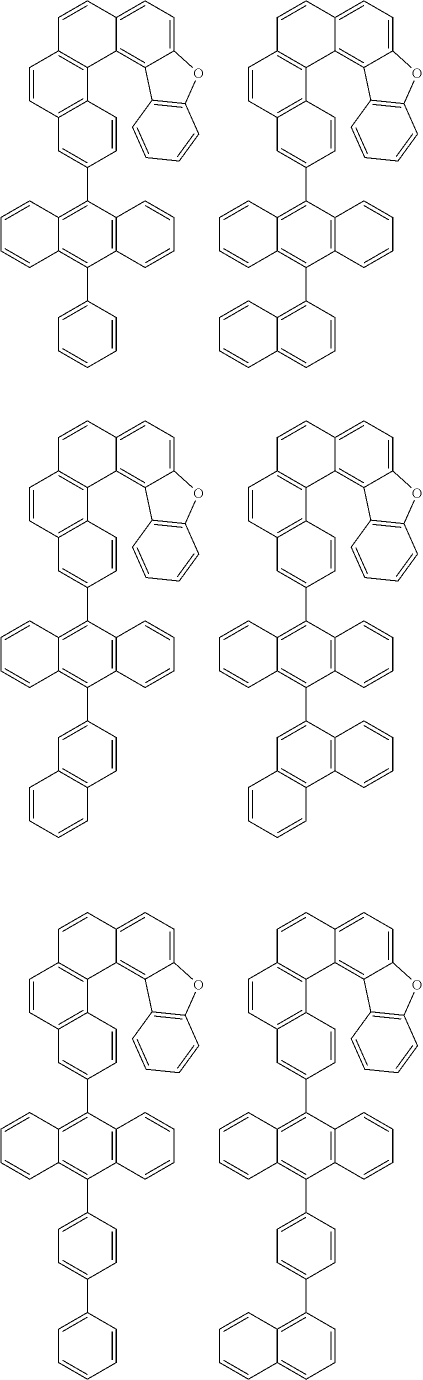

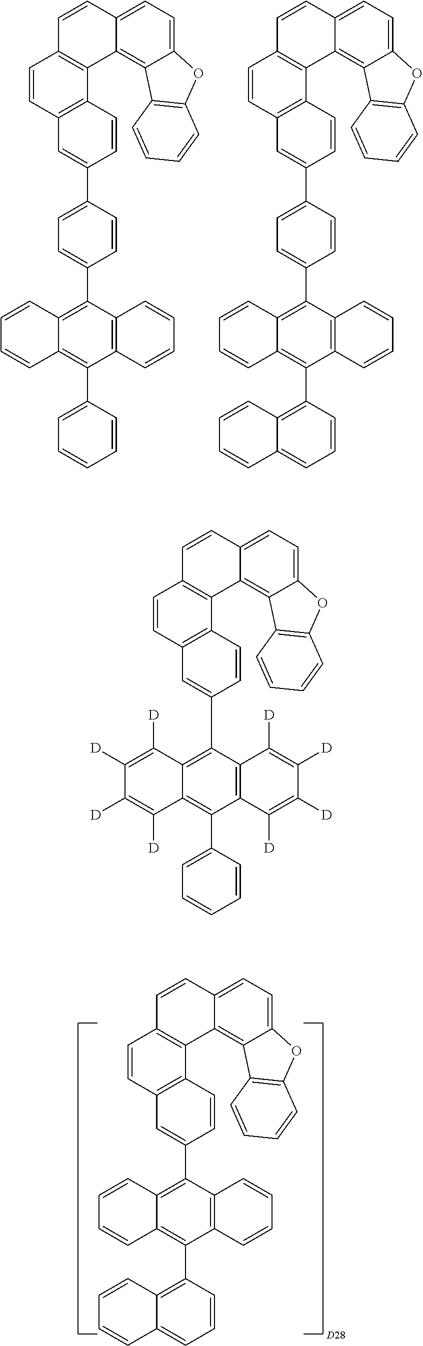

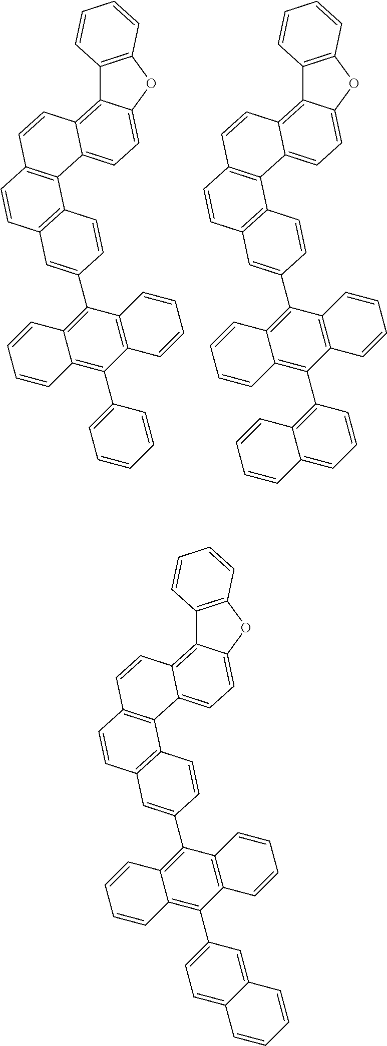

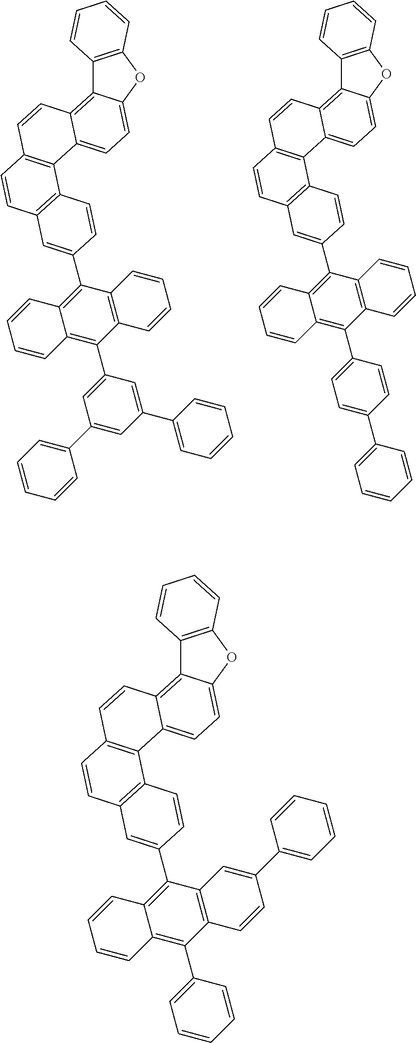 [SMILES: c1ccc(-c2cc(-c3ccccc3)cc(-c3c4ccccc4c(-c4ccc5c(ccc6ccc7c(ccc8oc9ccccc9c87)c65)c4)c4ccccc34)c2)cc1 ; c1ccc(-c2ccc(-c3c4ccccc4c(-c4ccc5c(ccc6ccc7c(ccc8oc9ccccc9c87)c65)c4)c4ccccc34)cc2)cc1 ; c1ccc(-c2ccc3c(-c4ccccc4)c4ccccc4c(-c4ccc5c(ccc6ccc7c(ccc8oc9ccccc9c87)c65)c4)c3c2)cc1]

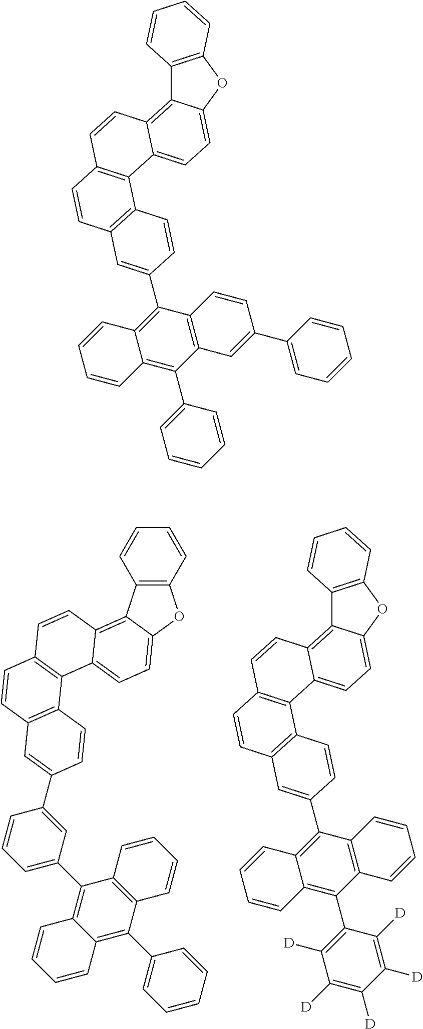 [SMILES: c1ccc(-c2ccc3c(-c4ccc5c(ccc6ccc7c(ccc8oc9ccccc9c87)c65)c4)c4ccccc4c(-c4ccccc4)c3c2)cc1 ; c1ccc(-c2c3ccccc3c(-c3cccc(-c4ccc5c(ccc6ccc7c(ccc8oc9ccccc9c87)c65)c4)c3)c3ccccc23)cc1 ; [2H]c1c([2H])c([2H])c(-c2c3ccccc3c(-c3ccc4c(ccc5ccc6c(ccc7oc8ccccc8c76)c54)c3)c3ccccc23)c([2H])c1[2H]]

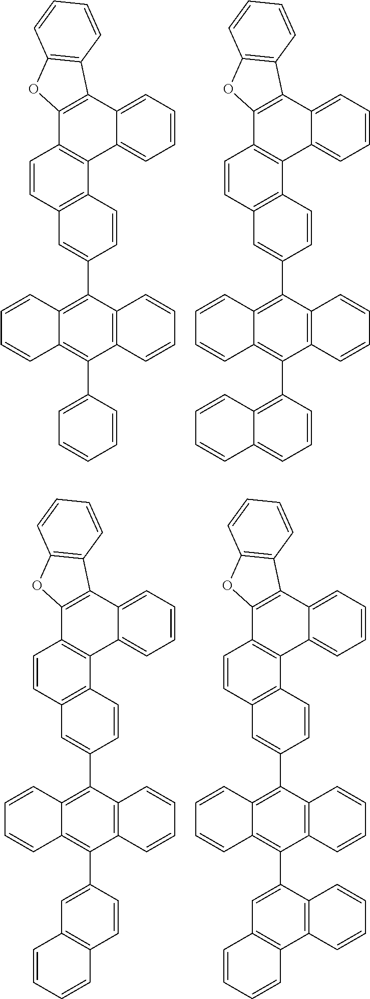

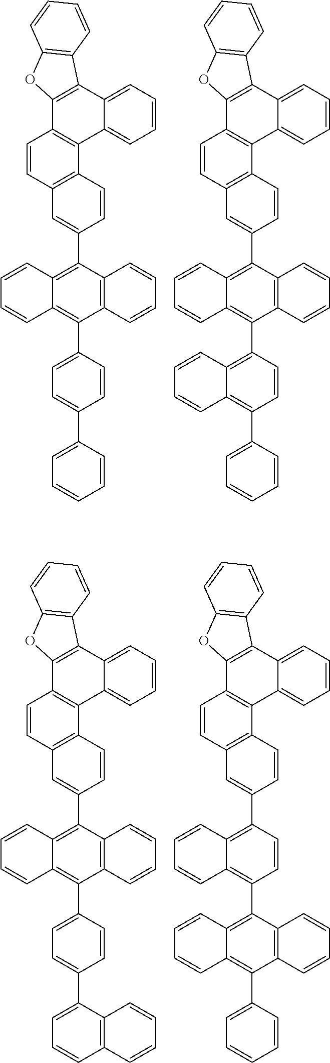

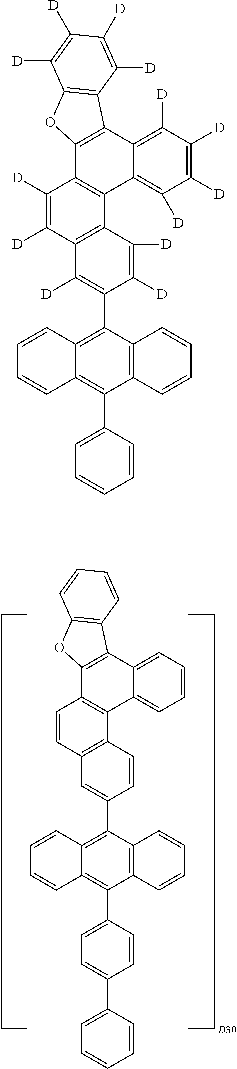

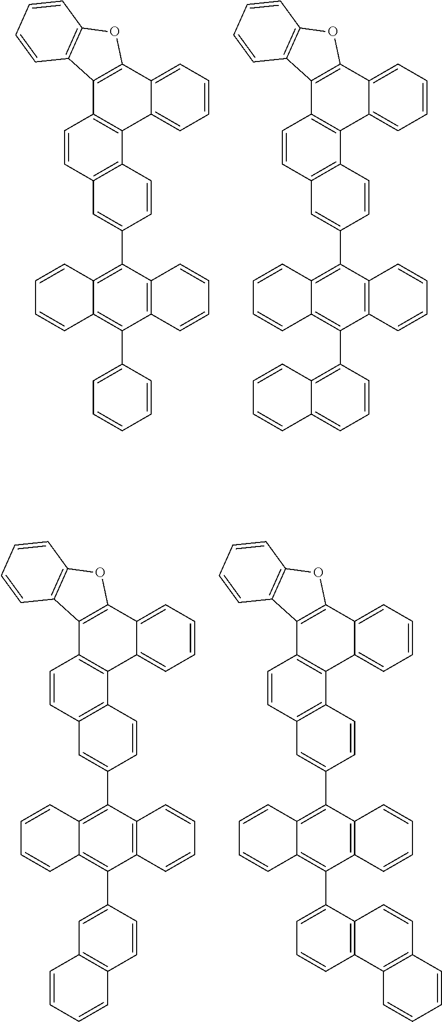

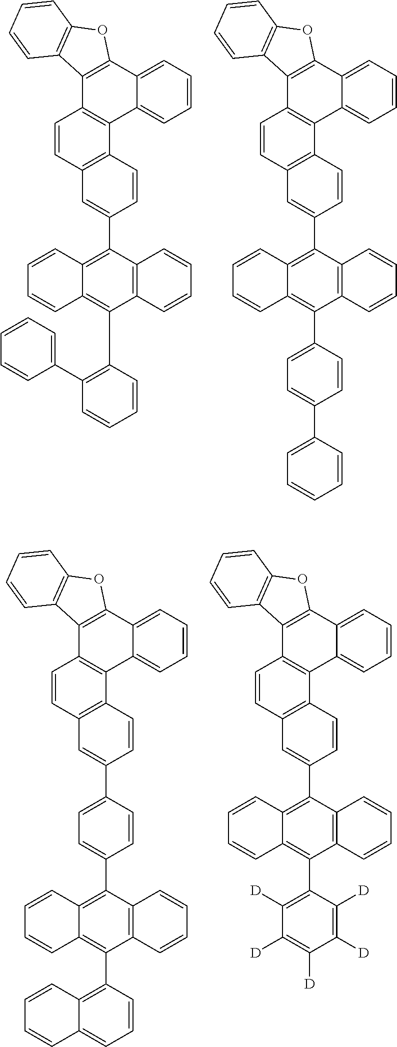

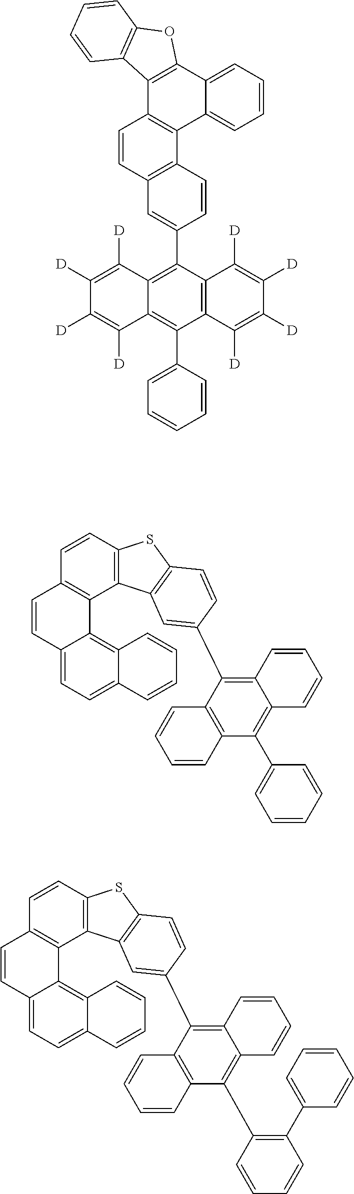 [SMILES: [2H]c1c([2H])c([2H])c2c(-c3ccc4c(ccc5c4c4ccccc4c4oc6ccccc6c54)c3)c3c([2H])c([2H])c([2H])c([2H])c3c(-c3ccccc3)c2c1[2H] ; c1ccc(-c2c3ccccc3c(-c3ccc4sc5ccc6ccc7ccc8ccccc8c7c6c5c4c3)c3ccccc23)cc1 ; c1ccc(-c2ccccc2-c2c3ccccc3c(-c3ccc4sc5ccc6ccc7ccc8ccccc8c7c6c5c4c3)c3ccccc23)cc1]

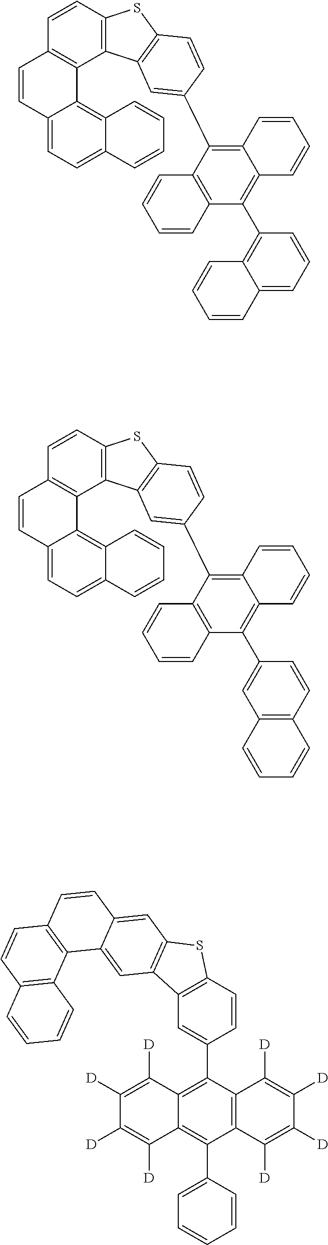 [SMILES: c1ccc2c(-c3c4ccccc4c(-c4ccc5sc6ccc7ccc8ccc9ccccc9c8c7c6c5c4)c4ccccc34)cccc2c1 ; c1ccc2cc(-c3c4ccccc4c(-c4ccc5sc6ccc7ccc8ccc9ccccc9c8c7c6c5c4)c4ccccc34)ccc2c1 ; [2H]c1c([2H])c([2H])c2c(-c3ccc4sc5cc6ccc7ccc8ccccc8c7c6cc5c4c3)c3c([2H])c([2H])c([2H])c([2H])c3c(-c3ccccc3)c2c1[2H]]

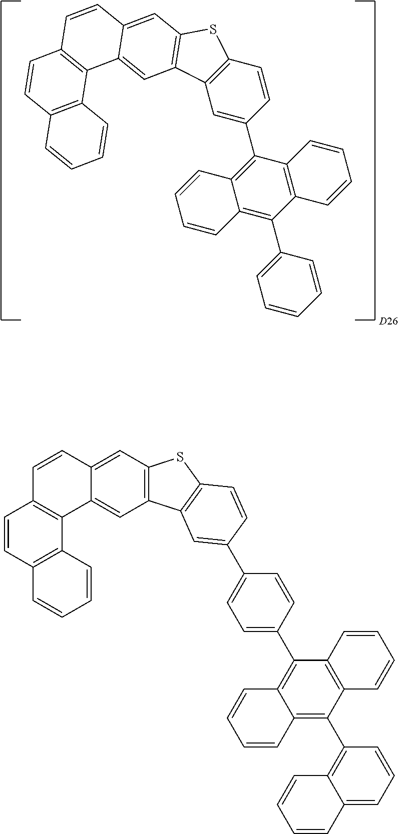

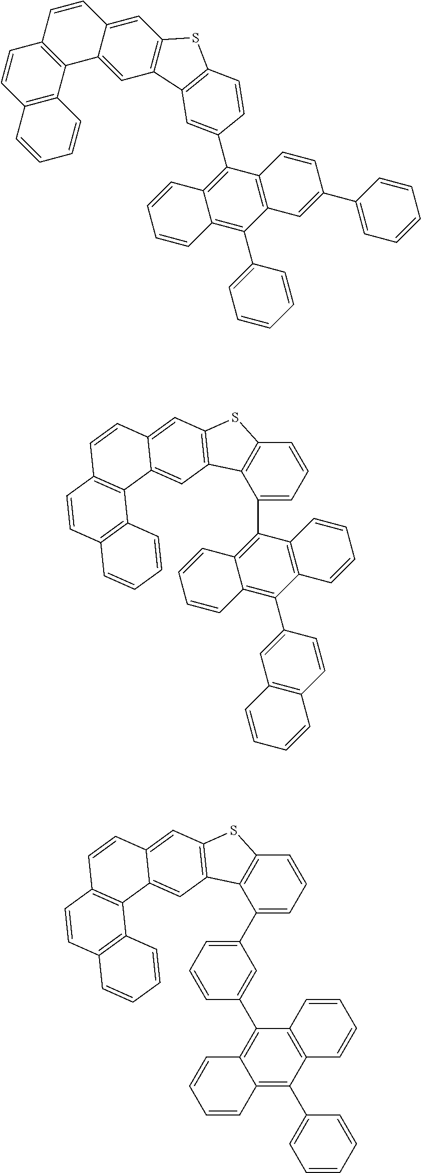 [SMILES: c1ccc(-c2ccc3c(-c4ccc5sc6cc7ccc8ccc9ccccc9c8c7cc6c5c4)c4ccccc4c(-c4ccccc4)c3c2)cc1 ; c1ccc2cc(-c3c4ccccc4c(-c4cccc5sc6cc7ccc8ccc9ccccc9c8c7cc6c45)c4ccccc34)ccc2c1 ; c1ccc(-c2c3ccccc3c(-c3cccc(-c4cccc5sc6cc7ccc8ccc9ccccc9c8c7cc6c45)c3)c3ccccc23)cc1]

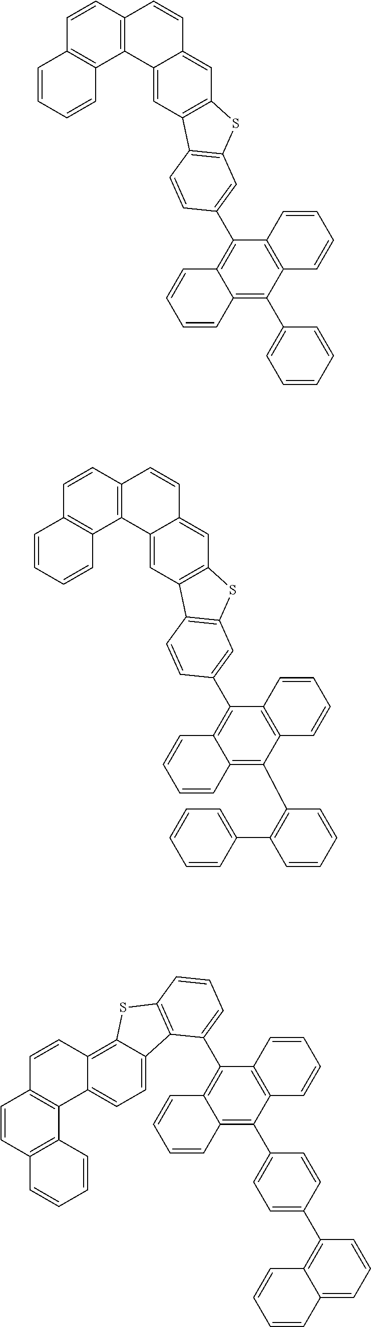 [SMILES: c1ccc(-c2c3ccccc3c(-c3ccc4c(c3)sc3cc5ccc6ccc7ccccc7c6c5cc34)c3ccccc23)cc1 ; c1ccc(-c2ccccc2-c2c3ccccc3c(-c3ccc4c(c3)sc3cc5ccc6ccc7ccccc7c6c5cc34)c3ccccc23)cc1 ; c1ccc2c(-c3ccc(-c4c5ccccc5c(-c5cccc6sc7c8ccc9ccc%10ccccc%10c9c8ccc7c56)c5ccccc45)cc3)cccc2c1]

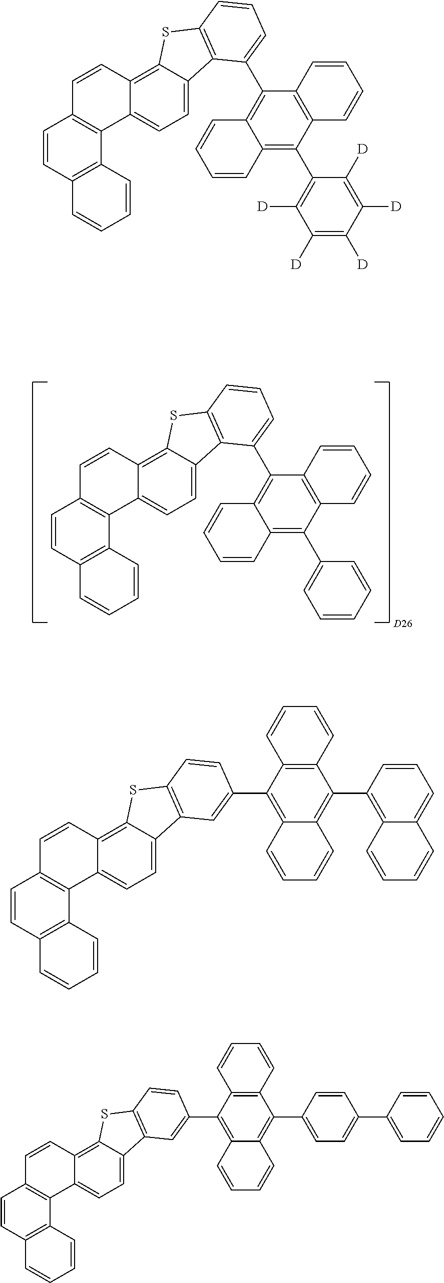

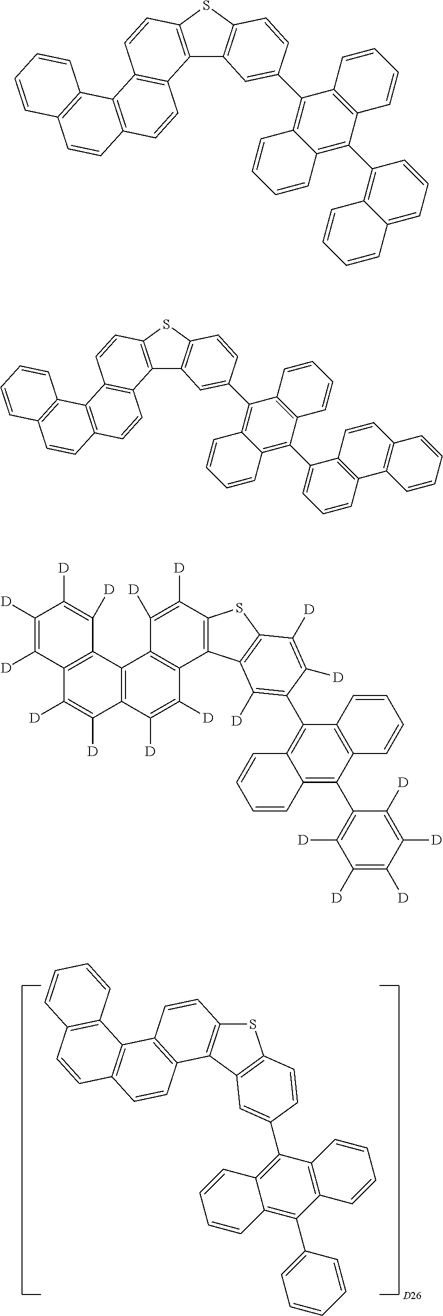

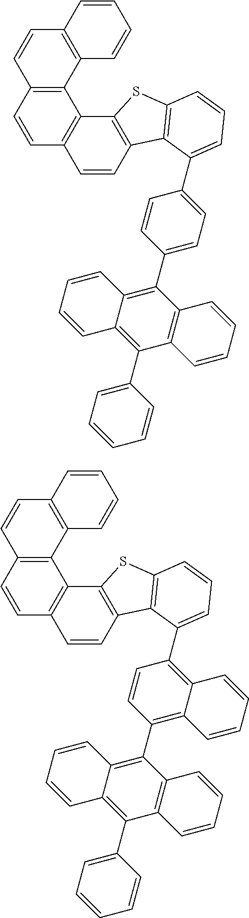

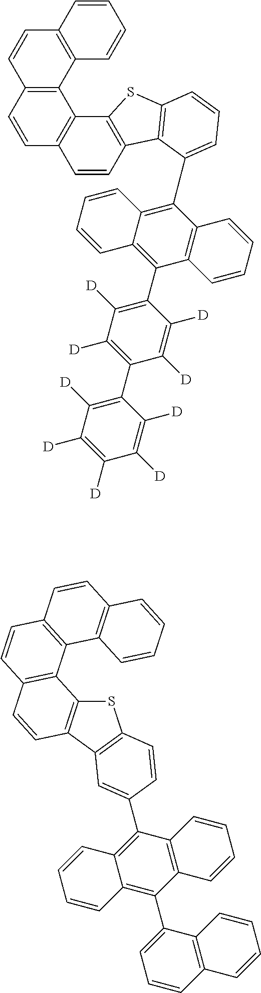 [SMILES: [2H]c1c([2H])c([2H])c(-c2c([2H])c([2H])c(-c3c4ccccc4c(-c4cccc5sc6c(ccc7ccc8ccc9ccccc9c8c76)c45)c4ccccc34)c([2H])c2[2H])c([2H])c1[2H] ; c1ccc2c(-c3c4ccccc4c(-c4ccc5sc6c(ccc7ccc8ccc9ccccc9c8c76)c5c4)c4ccccc34)cccc2c1]

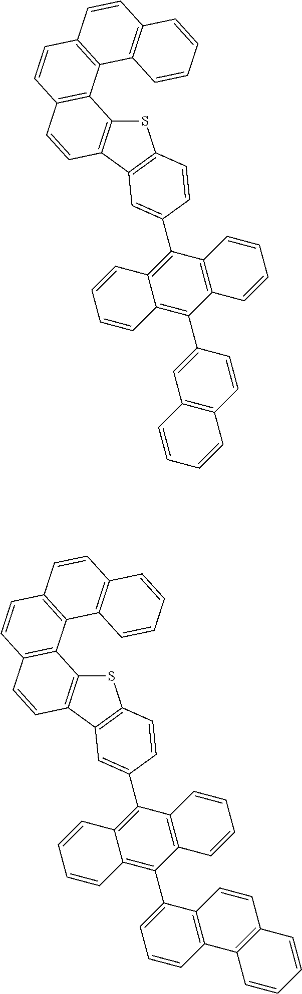 [SMILES: c1ccc2cc(-c3c4ccccc4c(-c4ccc5sc6c(ccc7ccc8ccc9ccccc9c8c76)c5c4)c4ccccc34)ccc2c1 ; c1ccc2c(c1)ccc1c(-c3c4ccccc4c(-c4ccc5sc6c(ccc7ccc8ccc9ccccc9c8c76)c5c4)c4ccccc34)cccc12]

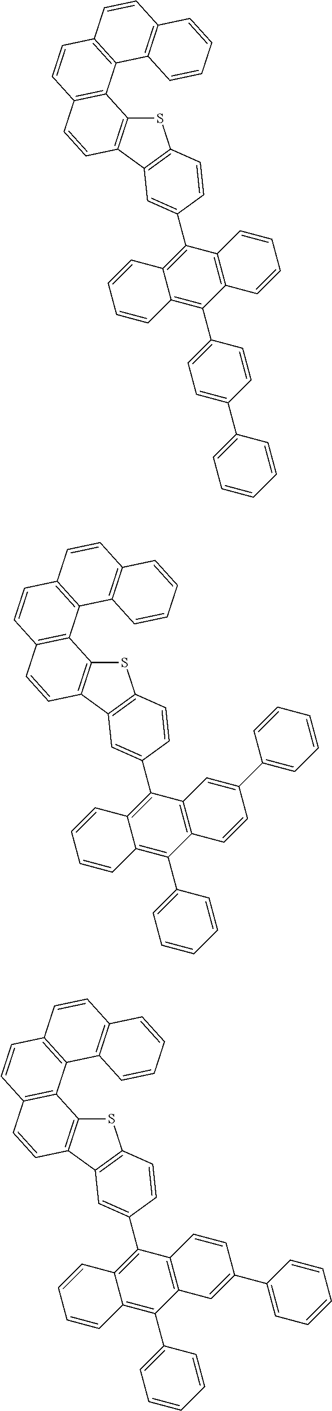 [SMILES: c1ccc(-c2ccc(-c3c4ccccc4c(-c4ccc5sc6c(ccc7ccc8ccc9ccccc9c8c76)c5c4)c4ccccc34)cc2)cc1 ; c1ccc(-c2ccc3c(-c4ccccc4)c4ccccc4c(-c4ccc5sc6c(ccc7ccc8ccc9ccccc9c8c76)c5c4)c3c2)cc1 ; c1ccc(-c2ccc3c(-c4ccc5sc6c(ccc7ccc8ccc9ccccc9c8c76)c5c4)c4ccccc4c(-c4ccccc4)c3c2)cc1]

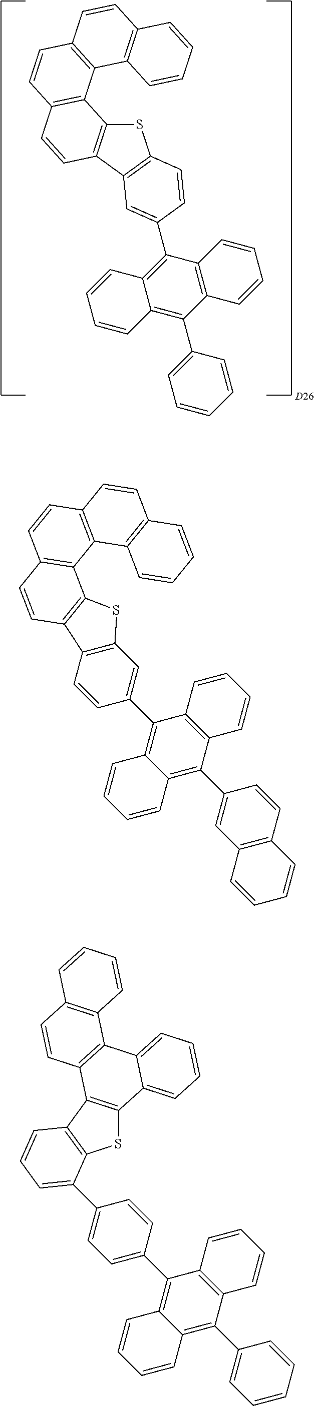

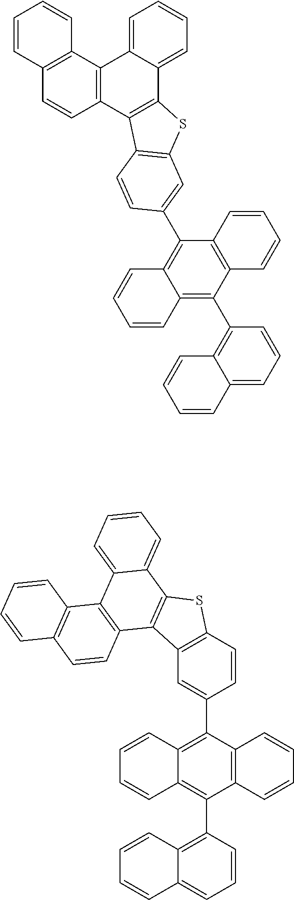 [SMILES: c1ccc2c(-c3c4ccccc4c(-c4ccc5c(c4)sc4c6ccccc6c6c7ccccc7ccc6c54)c4ccccc34)cccc2c1 ; c1ccc2c(-c3c4ccccc4c(-c4ccc5sc6c7ccccc7c7c8ccccc8ccc7c6c5c4)c4ccccc34)cccc2c1]

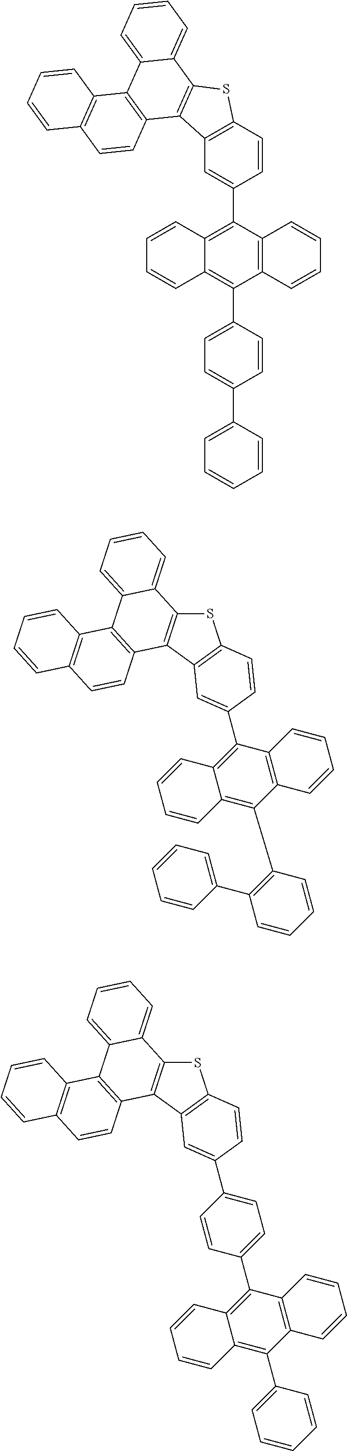 [SMILES: c1ccc(-c2ccc(-c3c4ccccc4c(-c4ccc5sc6c7ccccc7c7c8ccccc8ccc7c6c5c4)c4ccccc34)cc2)cc1 ; c1ccc(-c2ccccc2-c2c3ccccc3c(-c3ccc4sc5c6ccccc6c6c7ccccc7ccc6c5c4c3)c3ccccc23)cc1 ; c1ccc(-c2c3ccccc3c(-c3ccc(-c4ccc5sc6c7ccccc7c7c8ccccc8ccc7c6c5c4)cc3)c3ccccc23)cc1]

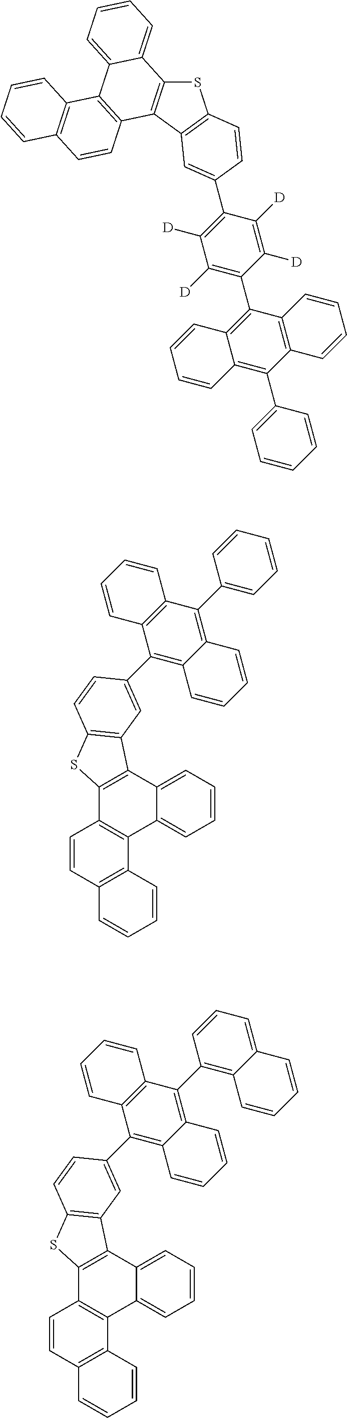 [SMILES: [2H]c1c([2H])c(-c2c3ccccc3c(-c3ccccc3)c3ccccc23)c([2H])c([2H])c1-c1ccc2sc3c4ccccc4c4c5ccccc5ccc4c3c2c1 ; c1ccc(-c2c3ccccc3c(-c3ccc4sc5c6ccc7ccccc7c6c6ccccc6c5c4c3)c3ccccc23)cc1 ; c1ccc2c(-c3c4ccccc4c(-c4ccc5sc6c7ccc8ccccc8c7c7ccccc7c6c5c4)c4ccccc34)cccc2c1]

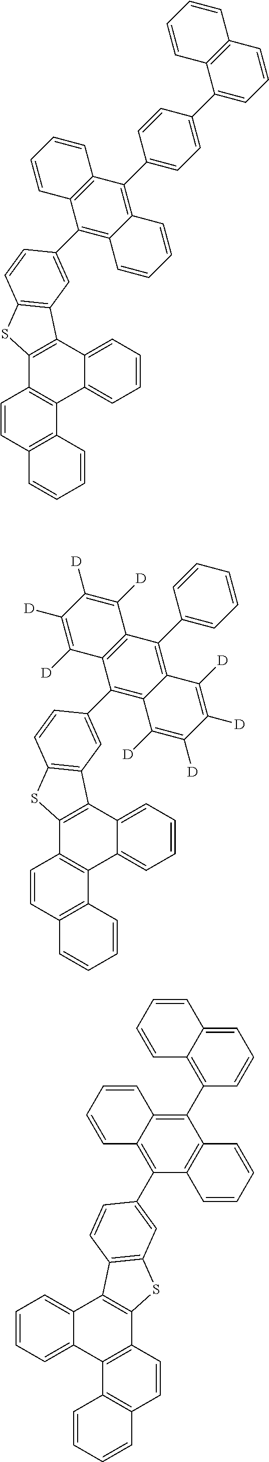 [SMILES: c1ccc2c(-c3ccc(-c4c5ccccc5c(-c5ccc6sc7c8ccc9ccccc9c8c8ccccc8c7c6c5)c5ccccc45)cc3)cccc2c1 ; [2H]c1c([2H])c([2H])c2c(-c3ccc4sc5c6ccc7ccccc7c6c6ccccc6c5c4c3)c3c([2H])c([2H])c([2H])c([2H])c3c(-c3ccccc3)c2c1[2H] ; c1ccc2c(-c3c4ccccc4c(-c4ccc5c(c4)sc4c6ccc7ccccc7c6c6ccccc6c54)c4ccccc34)cccc2c1]

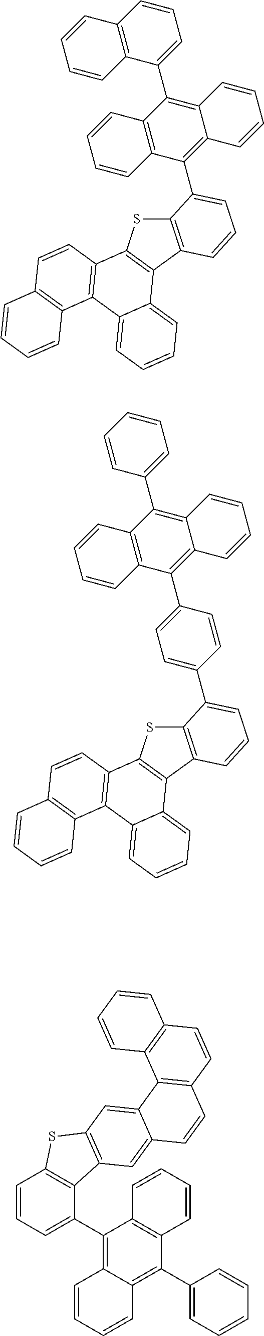

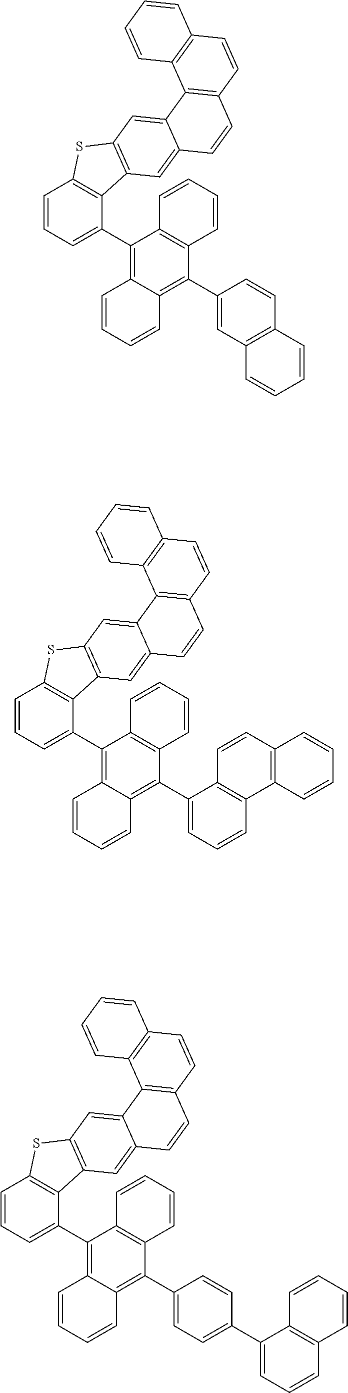 [SMILES: c1ccc2cc(-c3c4ccccc4c(-c4cccc5sc6cc7c(ccc8ccc9ccccc9c87)cc6c45)c4ccccc34)ccc2c1 ; c1ccc2c(c1)ccc1c(-c3c4ccccc4c(-c4cccc5sc6cc7c(ccc8ccc9ccccc9c87)cc6c45)c4ccccc34)cccc12 ; c1ccc2c(-c3ccc(-c4c5ccccc5c(-c5cccc6sc7cc8c(ccc9ccc%10ccccc%10c98)cc7c56)c5ccccc45)cc3)cccc2c1]

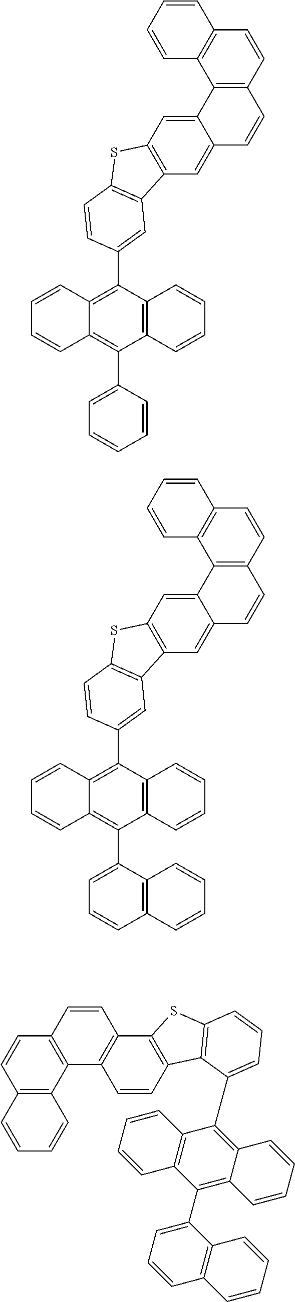 [SMILES: c1ccc(-c2c3ccccc3c(-c3ccc4sc5cc6c(ccc7ccc8ccccc8c76)cc5c4c3)c3ccccc23)cc1 ; c1ccc2c(-c3c4ccccc4c(-c4ccc5sc6cc7c(ccc8ccc9ccccc9c87)cc6c5c4)c4ccccc34)cccc2c1 ; c1ccc2c(-c3c4ccccc4c(-c4cccc5sc6c7ccc8ccc9ccccc9c8c7ccc6c45)c4ccccc34)cccc2c1]

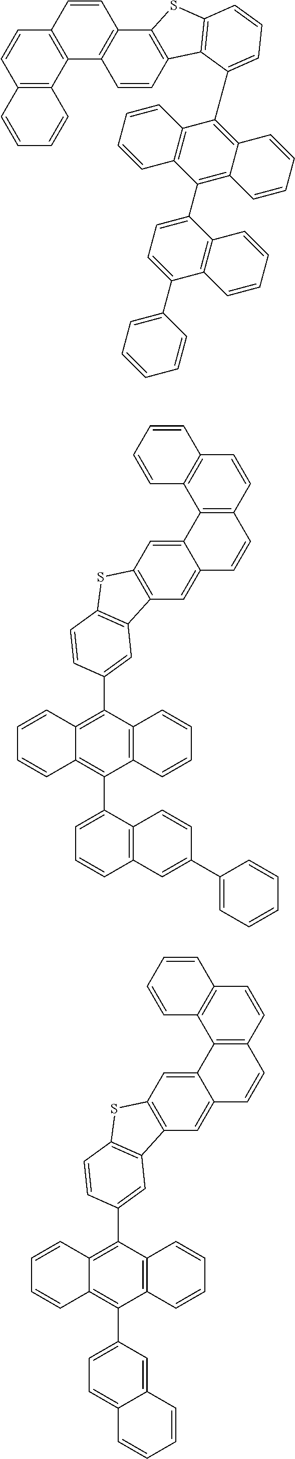 [SMILES: c1ccc(-c2ccc(-c3c4ccccc4c(-c4cccc5sc6c7ccc8ccc9ccccc9c8c7ccc6c45)c4ccccc34)c3ccccc23)cc1 ; c1ccc(-c2ccc3c(-c4c5ccccc5c(-c5ccc6sc7cc8c(ccc9ccc%10ccccc%10c98)cc7c6c5)c5ccccc45)cccc3c2)cc1 ; c1ccc2cc(-c3c4ccccc4c(-c4ccc5sc6cc7c(ccc8ccc9ccccc9c87)cc6c5c4)c4ccccc34)ccc2c1]

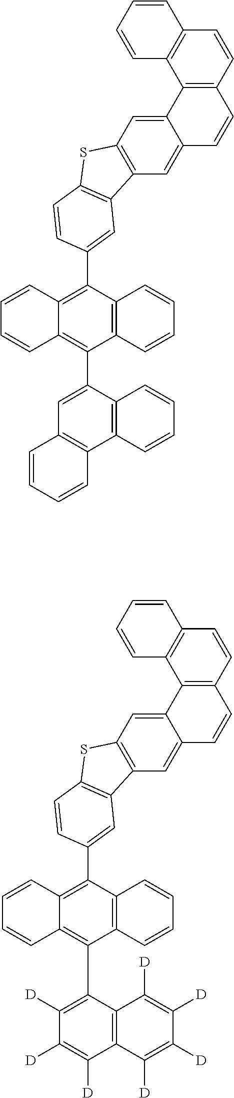 [SMILES: c1ccc2c(c1)cc(-c1c3ccccc3c(-c3ccc4sc5cc6c(ccc7ccc8ccccc8c76)cc5c4c3)c3ccccc13)c1ccccc12 ; [2H]c1c([2H])c([2H])c2c(-c3c4ccccc4c(-c4ccc5sc6cc7c(ccc8ccc9ccccc9c87)cc6c5c4)c4ccccc34)c([2H])c([2H])c([2H])c2c1[2H]]

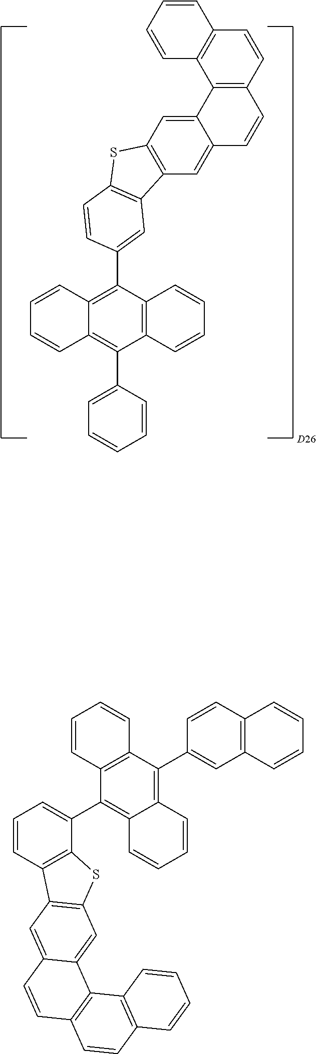

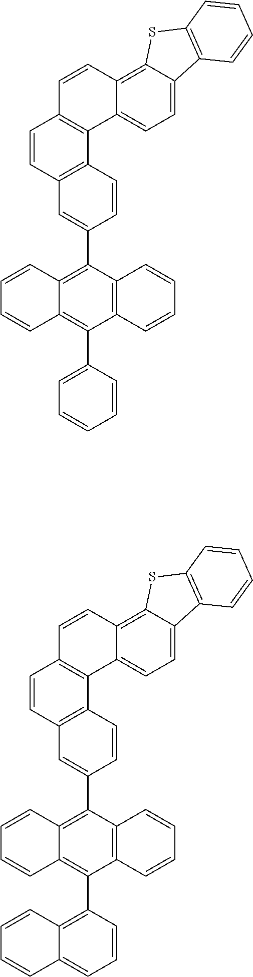 [SMILES: c1ccc(-c2c3ccccc3c(-c3ccc4c(ccc5ccc6c(ccc7c8ccccc8sc76)c54)c3)c3ccccc23)cc1 ; c1ccc2c(-c3c4ccccc4c(-c4ccc5c(ccc6ccc7c(ccc8c9ccccc9sc87)c65)c4)c4ccccc34)cccc2c1]

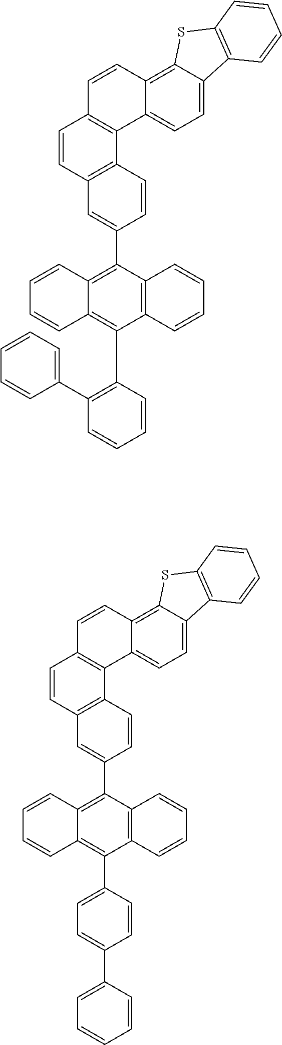 [SMILES: c1ccc(-c2ccccc2-c2c3ccccc3c(-c3ccc4c(ccc5ccc6c(ccc7c8ccccc8sc76)c54)c3)c3ccccc23)cc1 ; c1ccc(-c2ccc(-c3c4ccccc4c(-c4ccc5c(ccc6ccc7c(ccc8c9ccccc9sc87)c65)c4)c4ccccc34)cc2)cc1]

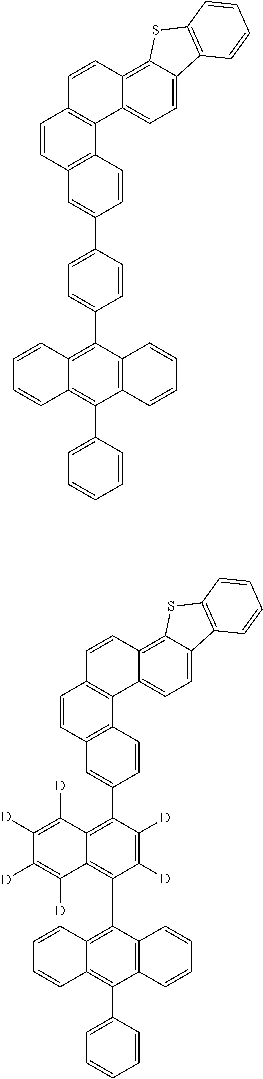 [SMILES: c1ccc(-c2c3ccccc3c(-c3ccc(-c4ccc5c(ccc6ccc7c(ccc8c9ccccc9sc87)c65)c4)cc3)c3ccccc23)cc1 ; [2H]c1c([2H])c([2H])c2c(-c3c4ccccc4c(-c4ccccc4)c4ccccc34)c([2H])c([2H])c(-c3ccc4c(ccc5ccc6c(ccc7c8ccccc8sc76)c54)c3)c2c1[2H]]

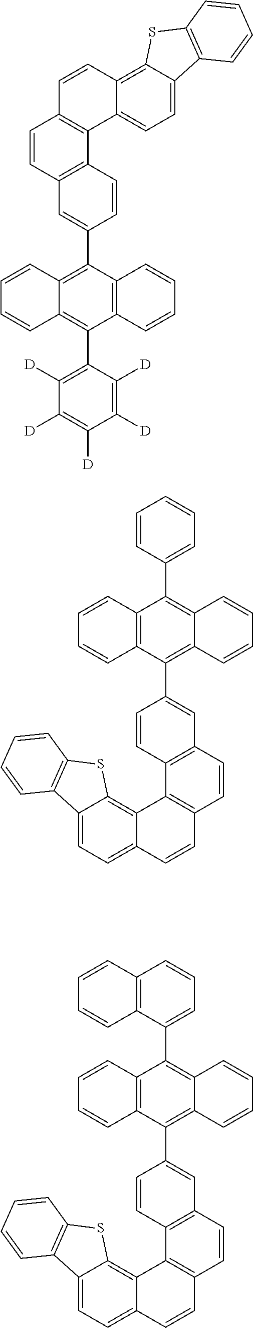 [SMILES: [2H]c1c([2H])c([2H])c(-c2c3ccccc3c(-c3ccc4c(ccc5ccc6c(ccc7c8ccccc8sc76)c54)c3)c3ccccc23)c([2H])c1[2H] ; c1ccc(-c2c3ccccc3c(-c3ccc4c(ccc5ccc6ccc7c8ccccc8sc7c6c54)c3)c3ccccc23)cc1 ; c1ccc2c(-c3c4ccccc4c(-c4ccc5c(ccc6ccc7ccc8c9ccccc9sc8c7c65)c4)c4ccccc34)cccc2c1]

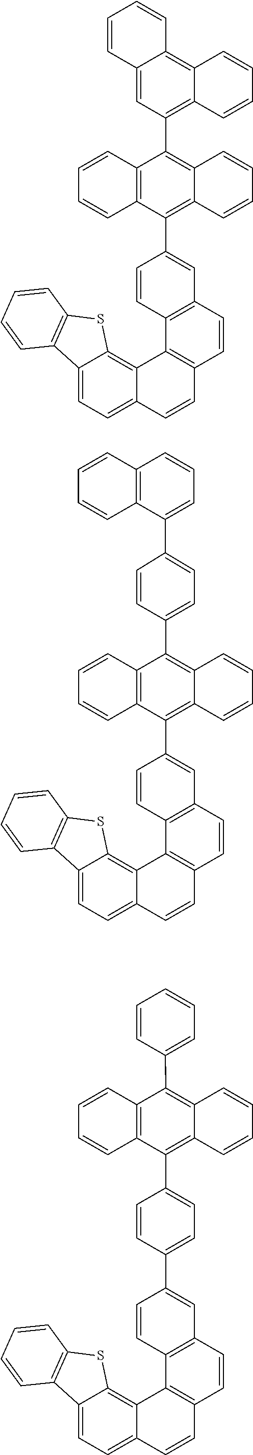

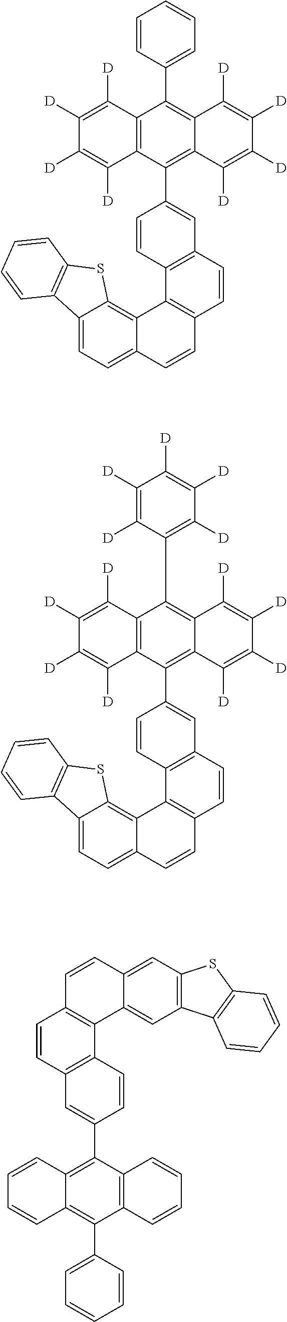 [SMILES: [2H]c1c([2H])c([2H])c2c(-c3ccc4c(ccc5ccc6ccc7c8ccccc8sc7c6c54)c3)c3c([2H])c([2H])c([2H])c([2H])c3c(-c3ccccc3)c2c1[2H] ; [2H]c1c([2H])c([2H])c(-c2c3c([2H])c([2H])c([2H])c([2H])c3c(-c3ccc4c(ccc5ccc6ccc7c8ccccc8sc7c6c54)c3)c3c([2H])c([2H])c([2H])c([2H])c23)c([2H])c1[2H] ; c1ccc(-c2c3ccccc3c(-c3ccc4c(ccc5ccc6cc7sc8ccccc8c7cc6c54)c3)c3ccccc23)cc1]

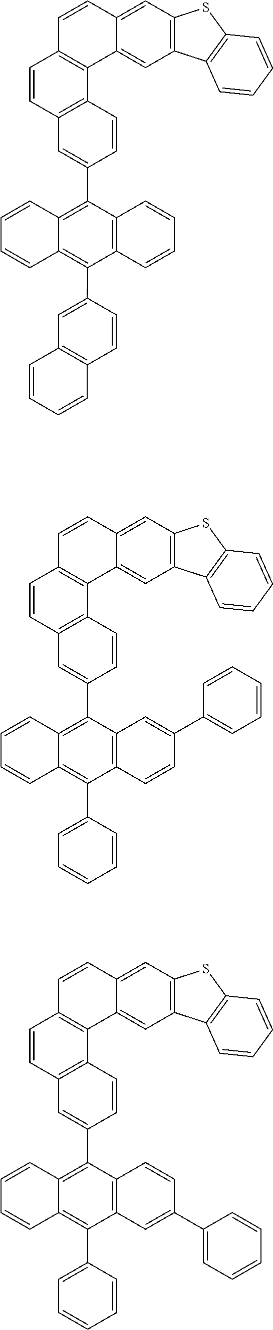 [SMILES: c1ccc2cc(-c3c4ccccc4c(-c4ccc5c(ccc6ccc7cc8sc9ccccc9c8cc7c65)c4)c4ccccc34)ccc2c1 ; c1ccc(-c2ccc3c(-c4ccccc4)c4ccccc4c(-c4ccc5c(ccc6ccc7cc8sc9ccccc9c8cc7c65)c4)c3c2)cc1 ; c1ccc(-c2ccc3c(-c4ccc5c(ccc6ccc7cc8sc9ccccc9c8cc7c65)c4)c4ccccc4c(-c4ccccc4)c3c2)cc1]

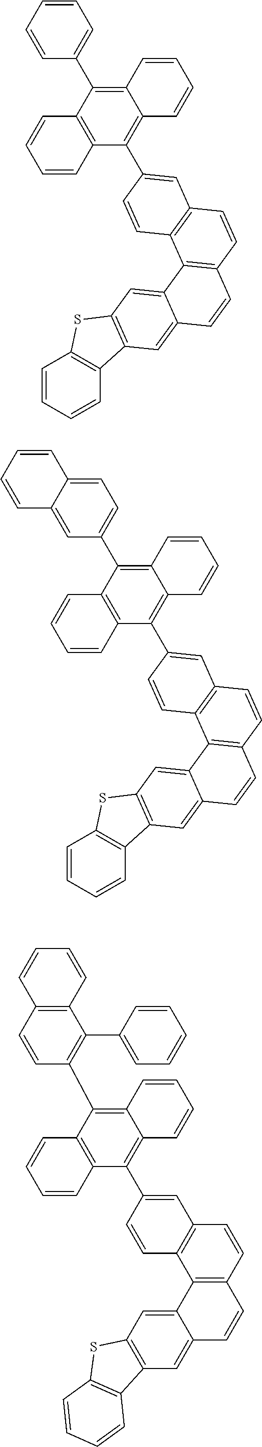

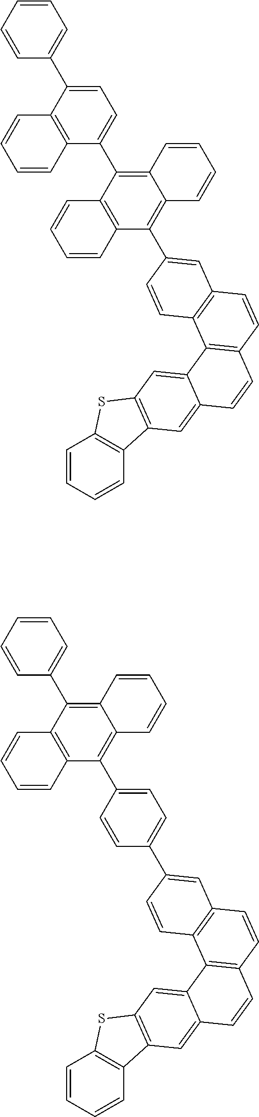 [SMILES: c1ccc(-c2ccc(-c3c4ccccc4c(-c4ccc5c(ccc6ccc7cc8c(cc7c65)sc5ccccc58)c4)c4ccccc34)c3ccccc23)cc1 ; c1ccc(-c2c3ccccc3c(-c3ccc(-c4ccc5c(ccc6ccc7cc8c(cc7c65)sc5ccccc58)c4)cc3)c3ccccc23)cc1]

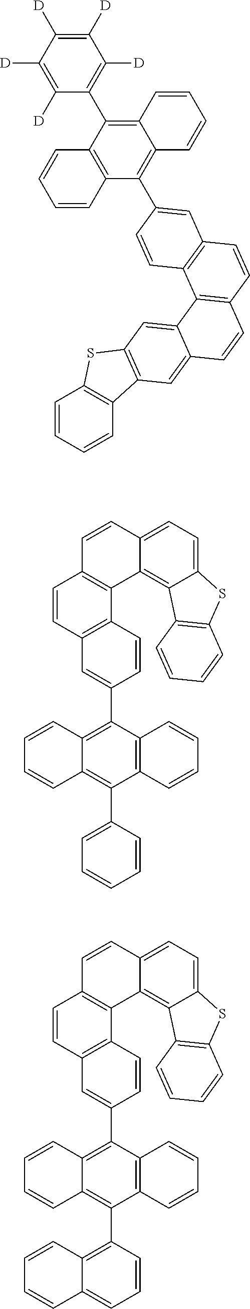 [SMILES: [2H]c1c([2H])c([2H])c(-c2c3ccccc3c(-c3ccc4c(ccc5ccc6cc7c(cc6c54)sc4ccccc47)c3)c3ccccc23)c([2H])c1[2H] ; c1ccc(-c2c3ccccc3c(-c3ccc4c(ccc5ccc6ccc7sc8ccccc8c7c6c54)c3)c3ccccc23)cc1 ; c1ccc2c(-c3c4ccccc4c(-c4ccc5c(ccc6ccc7ccc8sc9ccccc9c8c7c65)c4)c4ccccc34)cccc2c1]

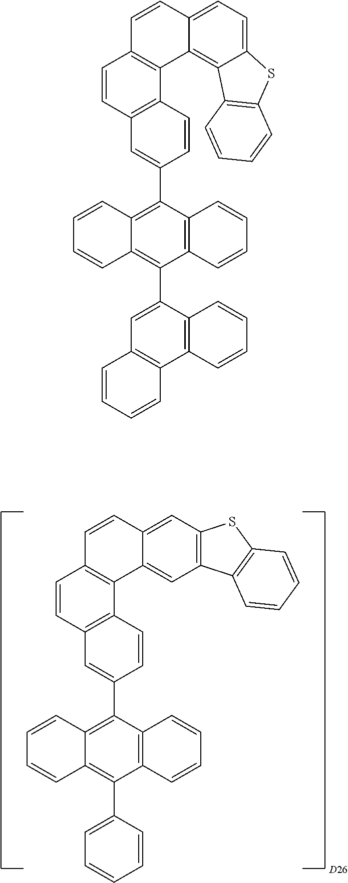

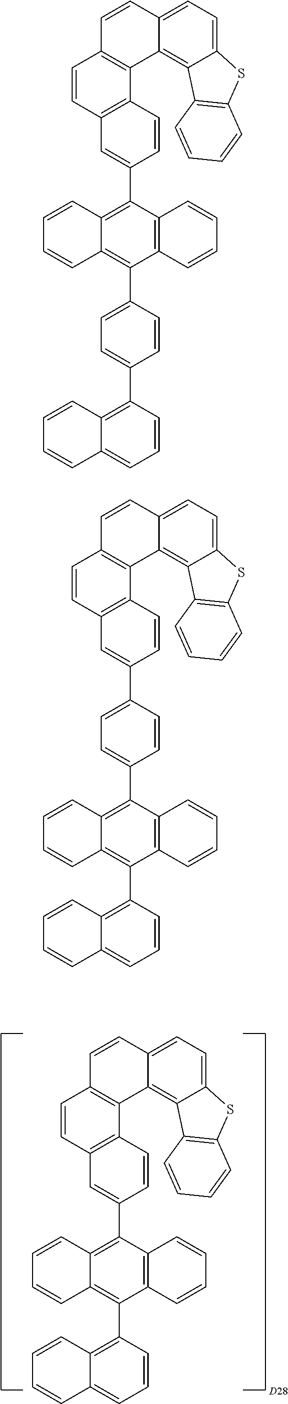

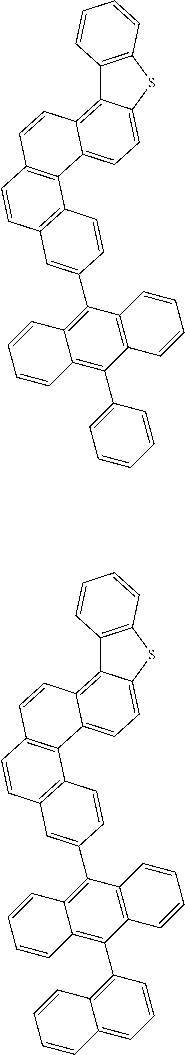 [SMILES: c1ccc(-c2c3ccccc3c(-c3ccc4c(ccc5ccc6c(ccc7sc8ccccc8c76)c54)c3)c3ccccc23)cc1 ; c1ccc2c(-c3c4ccccc4c(-c4ccc5c(ccc6ccc7c(ccc8sc9ccccc9c87)c65)c4)c4ccccc34)cccc2c1]

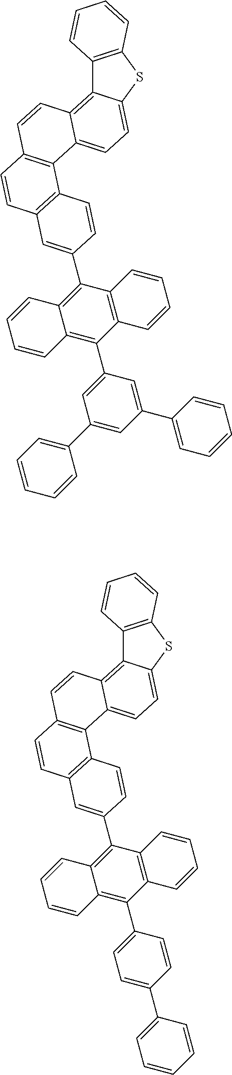 [SMILES: c1ccc(-c2cc(-c3ccccc3)cc(-c3c4ccccc4c(-c4ccc5c(ccc6ccc7c(ccc8sc9ccccc9c87)c65)c4)c4ccccc34)c2)cc1 ; c1ccc(-c2ccc(-c3c4ccccc4c(-c4ccc5c(ccc6ccc7c(ccc8sc9ccccc9c87)c65)c4)c4ccccc34)cc2)cc1]

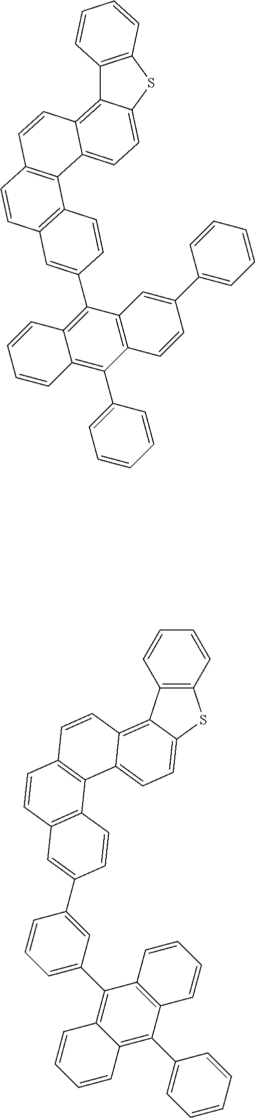 [SMILES: c1ccc(-c2ccc3c(-c4ccccc4)c4ccccc4c(-c4ccc5c(ccc6ccc7c(ccc8sc9ccccc9c87)c65)c4)c3c2)cc1 ; c1ccc(-c2c3ccccc3c(-c3cccc(-c4ccc5c(ccc6ccc7c(ccc8sc9ccccc9c87)c65)c4)c3)c3ccccc23)cc1]

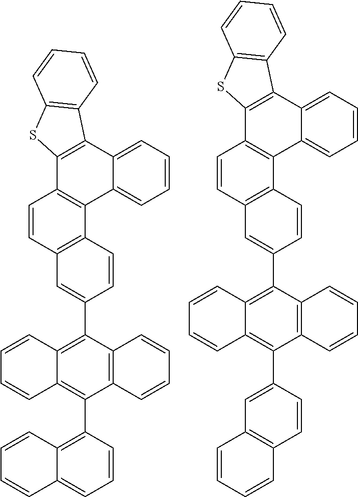

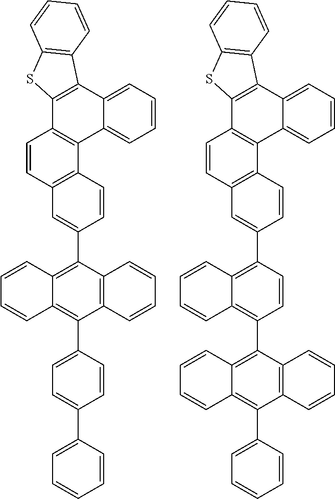

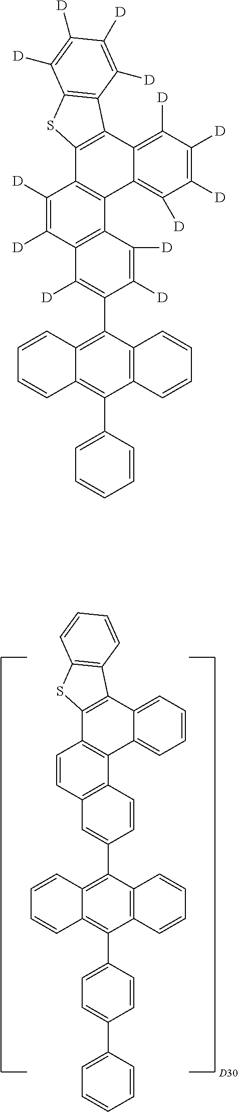

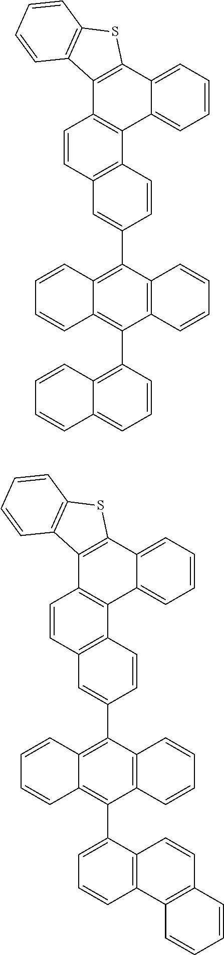 [SMILES: c1ccc2c(-c3c4ccccc4c(-c4ccc5c(ccc6c5c5ccccc5c5sc7ccccc7c65)c4)c4ccccc34)cccc2c1 ; c1ccc2c(c1)ccc1c(-c3c4ccccc4c(-c4ccc5c(ccc6c5c5ccccc5c5sc7ccccc7c65)c4)c4ccccc34)cccc12]

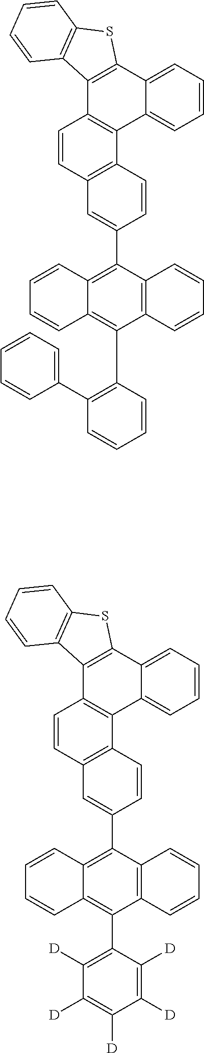 [SMILES: c1ccc(-c2ccccc2-c2c3ccccc3c(-c3ccc4c(ccc5c4c4ccccc4c4sc6ccccc6c54)c3)c3ccccc23)cc1 ; [2H]c1c([2H])c([2H])c(-c2c3ccccc3c(-c3ccc4c(ccc5c4c4ccccc4c4sc6ccccc6c54)c3)c3ccccc23)c([2H])c1[2H]]

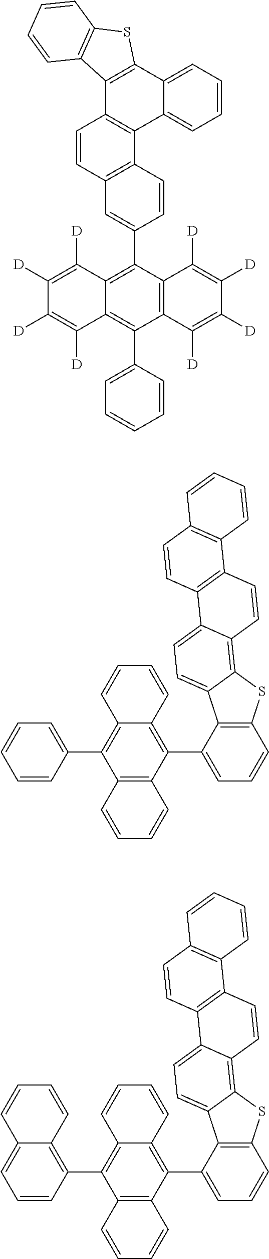 [SMILES: [2H]c1c([2H])c([2H])c2c(-c3ccc4c(ccc5c4c4ccccc4c4sc6ccccc6c54)c3)c3c([2H])c([2H])c([2H])c([2H])c3c(-c3ccccc3)c2c1[2H] ; c1ccc(-c2c3ccccc3c(-c3cccc4sc5c6ccc7c8ccccc8ccc7c6ccc5c34)c3ccccc23)cc1 ; c1ccc2c(-c3c4ccccc4c(-c4cccc5sc6c7ccc8c9ccccc9ccc8c7ccc6c45)c4ccccc34)cccc2c1]

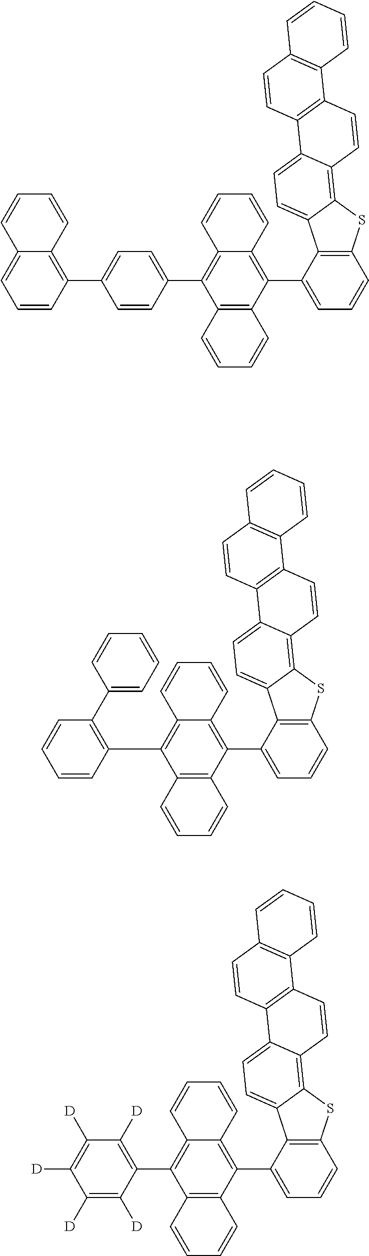 [SMILES: c1ccc2c(-c3ccc(-c4c5ccccc5c(-c5cccc6sc7c8ccc9c%10ccccc%10ccc9c8ccc7c56)c5ccccc45)cc3)cccc2c1 ; c1ccc(-c2ccccc2-c2c3ccccc3c(-c3cccc4sc5c6ccc7c8ccccc8ccc7c6ccc5c34)c3ccccc23)cc1 ; [2H]c1c([2H])c([2H])c(-c2c3ccccc3c(-c3cccc4sc5c6ccc7c8ccccc8ccc7c6ccc5c34)c3ccccc23)c([2H])c1[2H]]

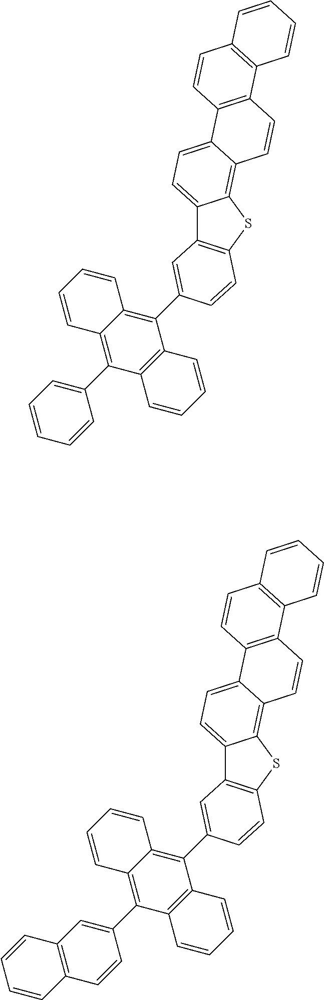 [SMILES: c1ccc(-c2c3ccccc3c(-c3ccc4sc5c(ccc6c7ccc8ccccc8c7ccc65)c4c3)c3ccccc23)cc1 ; c1ccc2cc(-c3c4ccccc4c(-c4ccc5sc6c(ccc7c8ccc9ccccc9c8ccc76)c5c4)c4ccccc34)ccc2c1]

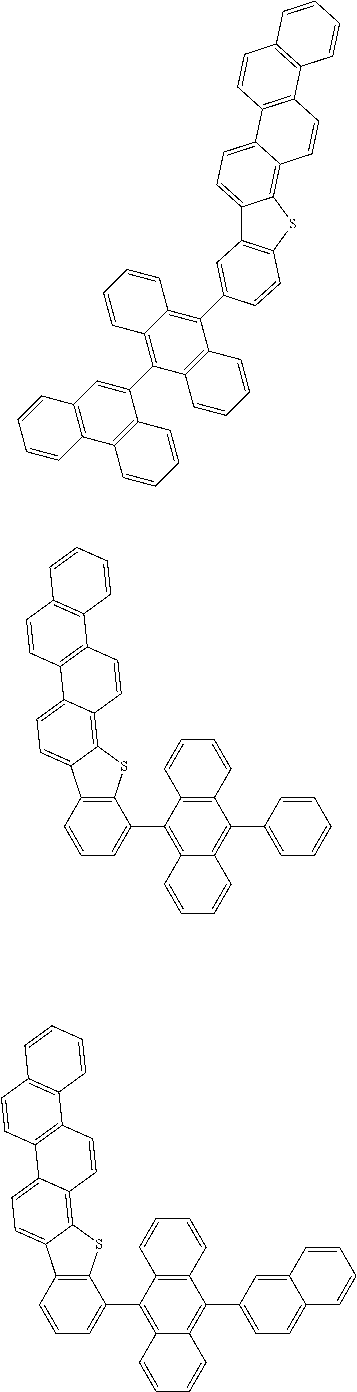 [SMILES: c1ccc2c(c1)cc(-c1c3ccccc3c(-c3ccc4sc5c(ccc6c7ccc8ccccc8c7ccc65)c4c3)c3ccccc13)c1ccccc12 ; c1ccc(-c2c3ccccc3c(-c3cccc4c3sc3c4ccc4c5ccc6ccccc6c5ccc43)c3ccccc23)cc1 ; c1ccc2cc(-c3c4ccccc4c(-c4cccc5c4sc4c5ccc5c6ccc7ccccc7c6ccc54)c4ccccc34)ccc2c1]

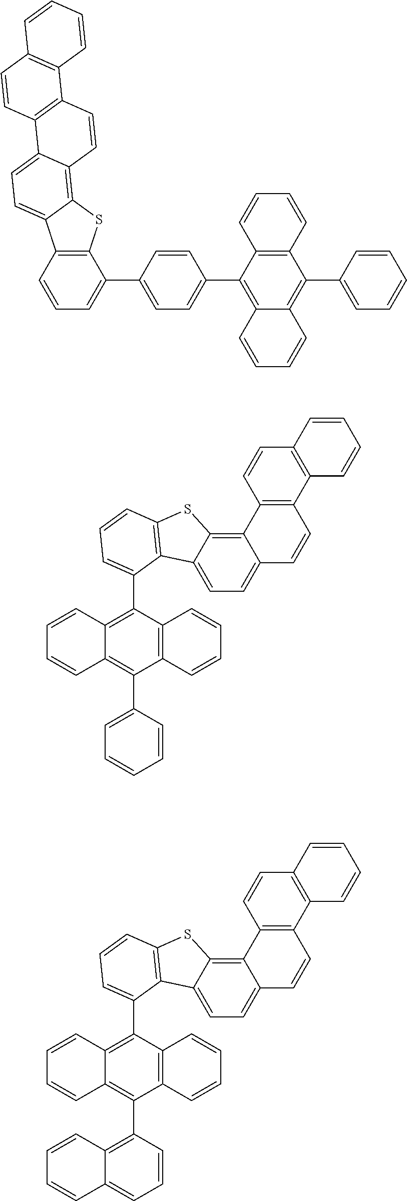 [SMILES: c1ccc(-c2c3ccccc3c(-c3ccc(-c4cccc5c4sc4c5ccc5c6ccc7ccccc7c6ccc54)cc3)c3ccccc23)cc1 ; c1ccc(-c2c3ccccc3c(-c3cccc4sc5c(ccc6ccc7c8ccccc8ccc7c65)c34)c3ccccc23)cc1 ; c1ccc2c(-c3c4ccccc4c(-c4cccc5sc6c(ccc7ccc8c9ccccc9ccc8c76)c45)c4ccccc34)cccc2c1]

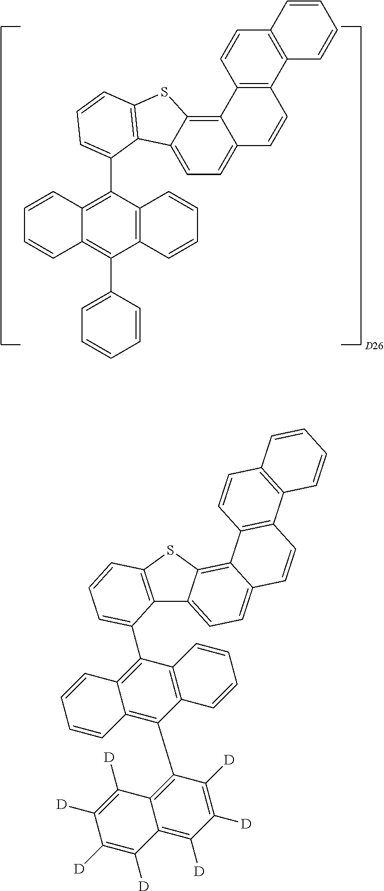

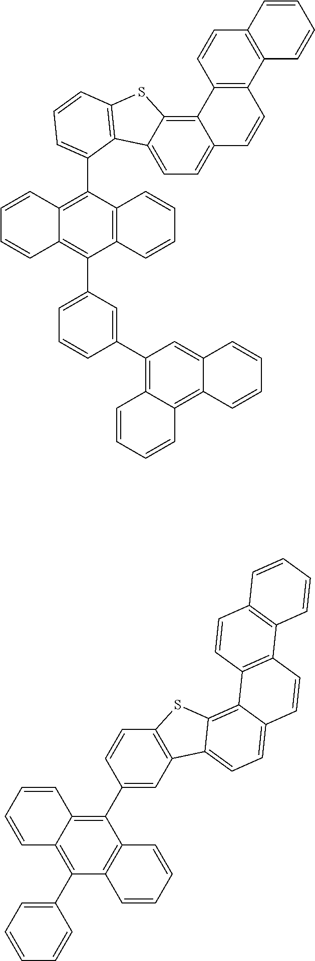 [SMILES: c1cc(-c2c3ccccc3c(-c3cccc4sc5c(ccc6ccc7c8ccccc8ccc7c65)c34)c3ccccc23)cc(-c2cc3ccccc3c3ccccc23)c1 ; c1ccc(-c2c3ccccc3c(-c3ccc4sc5c(ccc6ccc7c8ccccc8ccc7c65)c4c3)c3ccccc23)cc1]

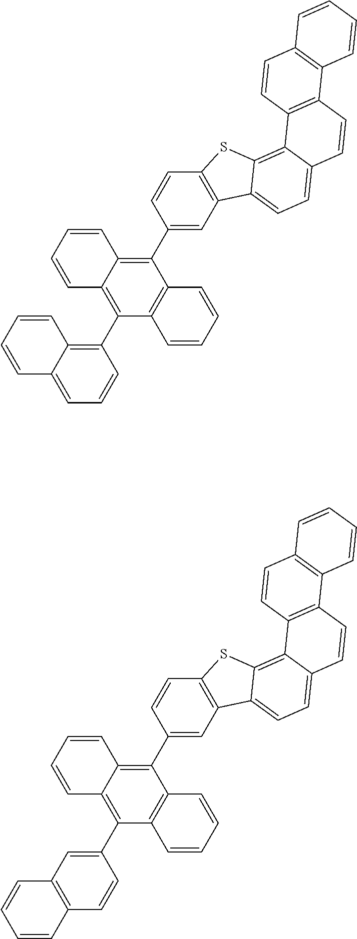 [SMILES: c1ccc2c(-c3c4ccccc4c(-c4ccc5sc6c(ccc7ccc8c9ccccc9ccc8c76)c5c4)c4ccccc34)cccc2c1 ; c1ccc2cc(-c3c4ccccc4c(-c4ccc5sc6c(ccc7ccc8c9ccccc9ccc8c76)c5c4)c4ccccc34)ccc2c1]

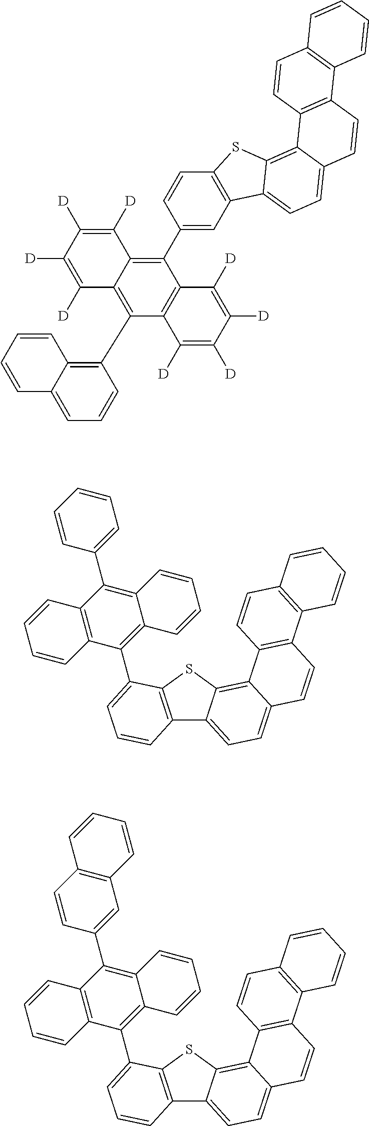 [SMILES: [2H]c1c([2H])c([2H])c2c(-c3cccc4ccccc34)c3c([2H])c([2H])c([2H])c([2H])c3c(-c3ccc4sc5c(ccc6ccc7c8ccccc8ccc7c65)c4c3)c2c1[2H] ; c1ccc(-c2c3ccccc3c(-c3cccc4c3sc3c4ccc4ccc5c6ccccc6ccc5c43)c3ccccc23)cc1 ; c1ccc2cc(-c3c4ccccc4c(-c4cccc5c4sc4c5ccc5ccc6c7ccccc7ccc6c54)c4ccccc34)ccc2c1]

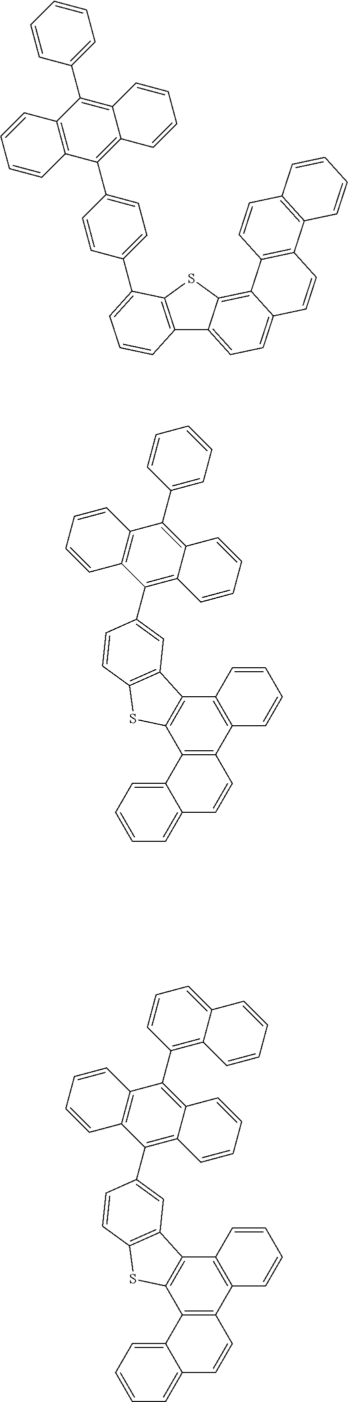 [SMILES: c1ccc(-c2c3ccccc3c(-c3ccc(-c4cccc5c4sc4c5ccc5ccc6c7ccccc7ccc6c54)cc3)c3ccccc23)cc1 ; c1ccc(-c2c3ccccc3c(-c3ccc4sc5c6c7ccccc7ccc6c6ccccc6c5c4c3)c3ccccc23)cc1 ; c1ccc2c(-c3c4ccccc4c(-c4ccc5sc6c7c8ccccc8ccc7c7ccccc7c6c5c4)c4ccccc34)cccc2c1]

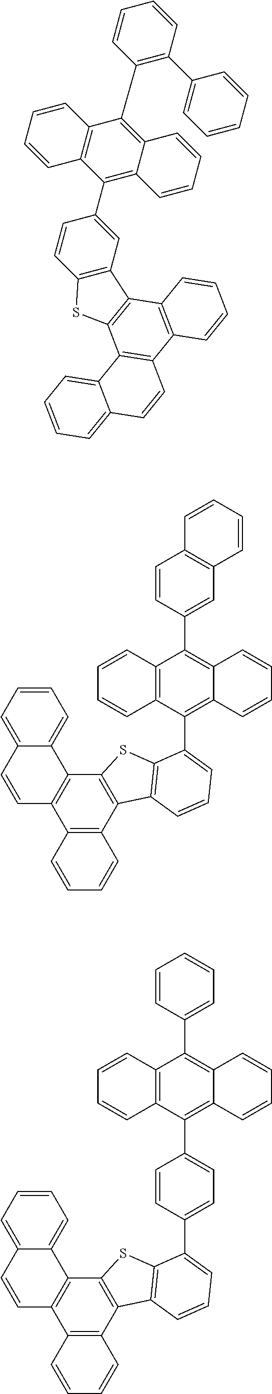 [SMILES: c1ccc(-c2ccccc2-c2c3ccccc3c(-c3ccc4sc5c6c7ccccc7ccc6c6ccccc6c5c4c3)c3ccccc23)cc1 ; c1ccc2cc(-c3c4ccccc4c(-c4cccc5c4sc4c6c7ccccc7ccc6c6ccccc6c54)c4ccccc34)ccc2c1 ; c1ccc(-c2c3ccccc3c(-c3ccc(-c4cccc5c4sc4c6c7ccccc7ccc6c6ccccc6c54)cc3)c3ccccc23)cc1]

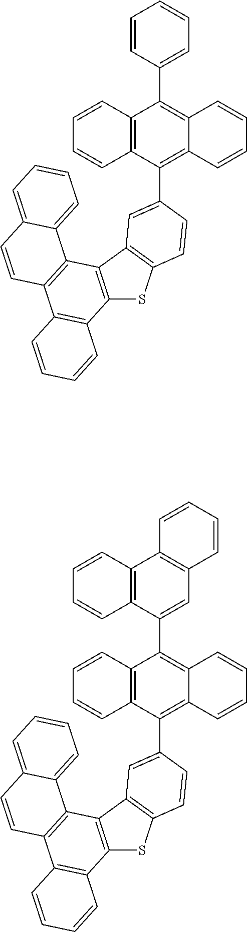 [SMILES: c1ccc(-c2c3ccccc3c(-c3ccc4sc5c6ccccc6c6ccc7ccccc7c6c5c4c3)c3ccccc23)cc1 ; c1ccc2c(c1)cc(-c1c3ccccc3c(-c3ccc4sc5c6ccccc6c6ccc7ccccc7c6c5c4c3)c3ccccc13)c1ccccc12]

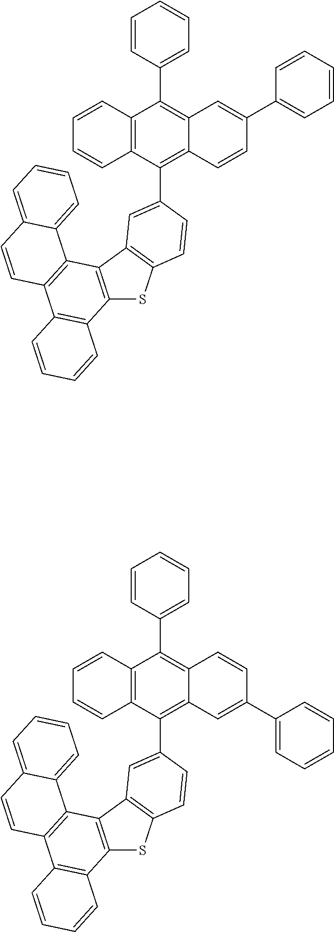 [SMILES: c1ccc(-c2ccc3c(-c4ccc5sc6c7ccccc7c7ccc8ccccc8c7c6c5c4)c4ccccc4c(-c4ccccc4)c3c2)cc1 ; c1ccc(-c2ccc3c(-c4ccccc4)c4ccccc4c(-c4ccc5sc6c7ccccc7c7ccc8ccccc8c7c6c5c4)c3c2)cc1]

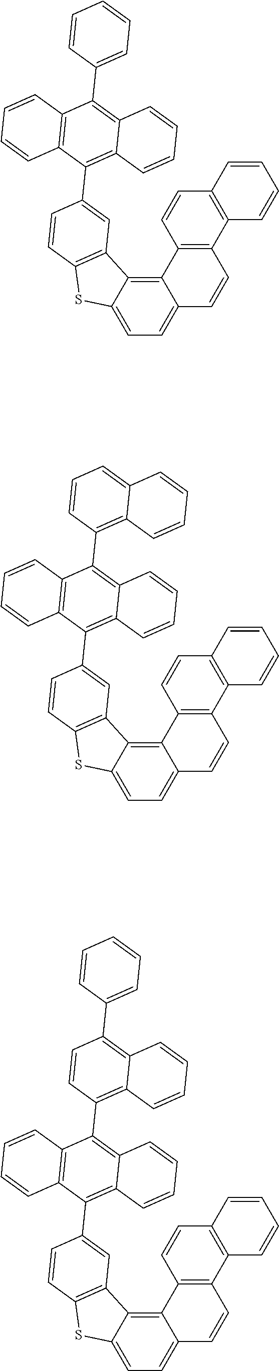 [SMILES: c1ccc(-c2c3ccccc3c(-c3ccc4sc5ccc6ccc7c8ccccc8ccc7c6c5c4c3)c3ccccc23)cc1 ; c1ccc2c(-c3c4ccccc4c(-c4ccc5sc6ccc7ccc8c9ccccc9ccc8c7c6c5c4)c4ccccc34)cccc2c1 ; c1ccc(-c2ccc(-c3c4ccccc4c(-c4ccc5sc6ccc7ccc8c9ccccc9ccc8c7c6c5c4)c4ccccc34)c3ccccc23)cc1]

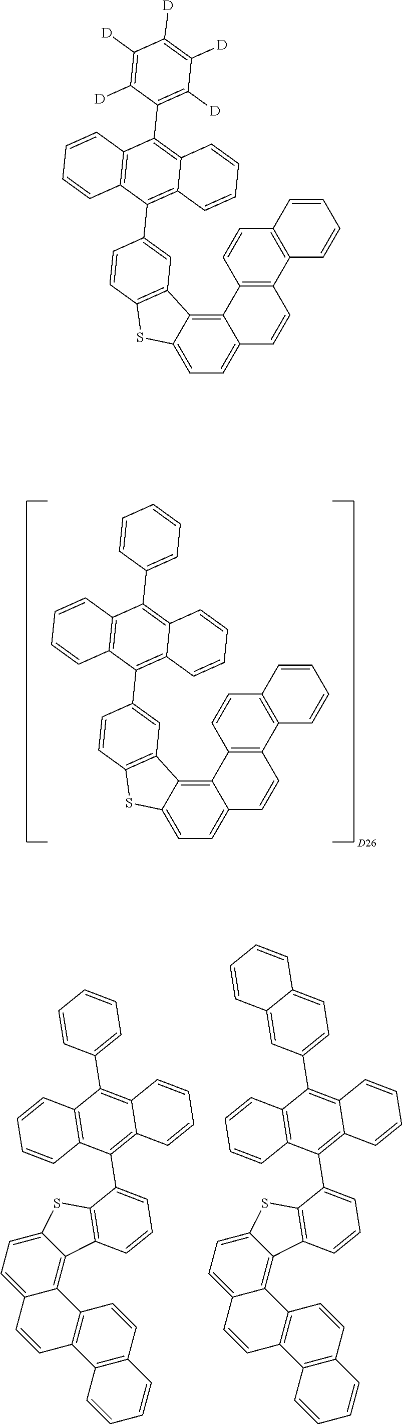

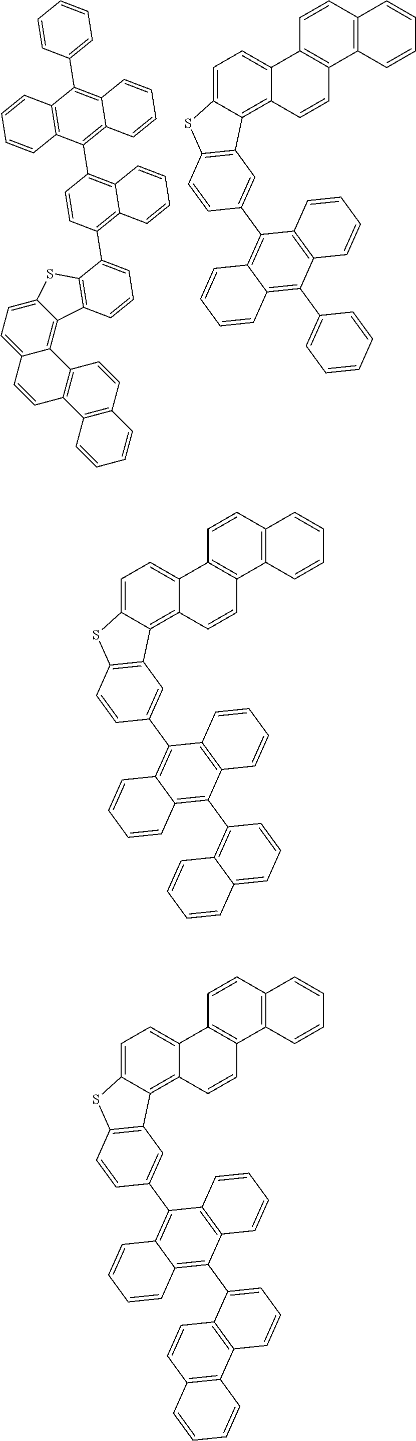

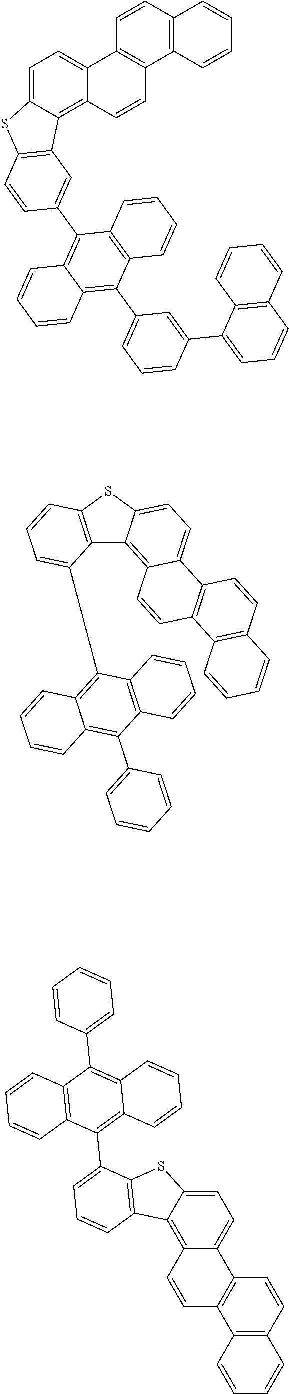 [SMILES: c1cc(-c2cccc3ccccc23)cc(-c2c3ccccc3c(-c3ccc4sc5ccc6c7ccc8ccccc8c7ccc6c5c4c3)c3ccccc23)c1 ; c1ccc(-c2c3ccccc3c(-c3cccc4sc5ccc6c7ccc8ccccc8c7ccc6c5c34)c3ccccc23)cc1 ; c1ccc(-c2c3ccccc3c(-c3cccc4c3sc3ccc5c6ccc7ccccc7c6ccc5c34)c3ccccc23)cc1]

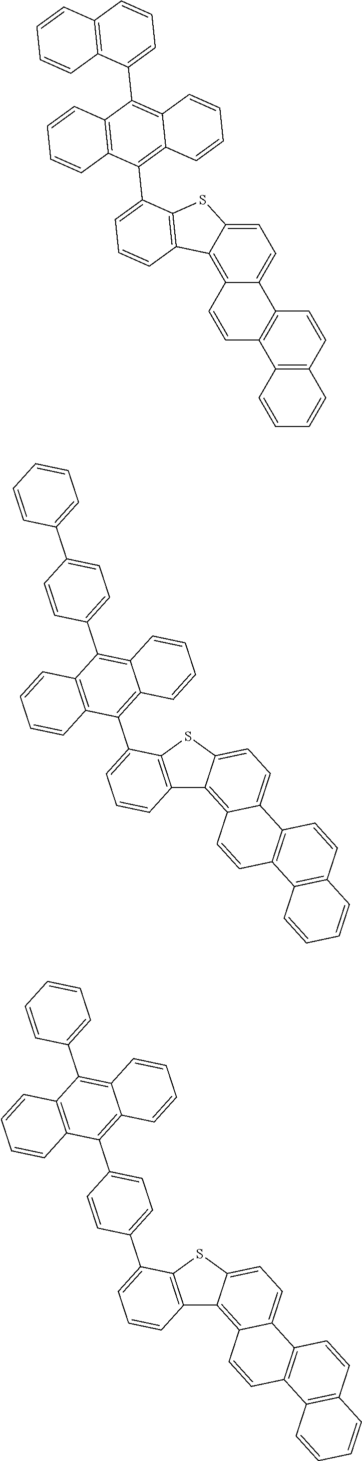 [SMILES: c1ccc2c(-c3c4ccccc4c(-c4cccc5c4sc4ccc6c7ccc8ccccc8c7ccc6c45)c4ccccc34)cccc2c1 ; c1ccc(-c2ccc(-c3c4ccccc4c(-c4cccc5c4sc4ccc6c7ccc8ccccc8c7ccc6c45)c4ccccc34)cc2)cc1 ; c1ccc(-c2c3ccccc3c(-c3ccc(-c4cccc5c4sc4ccc6c7ccc8ccccc8c7ccc6c45)cc3)c3ccccc23)cc1]

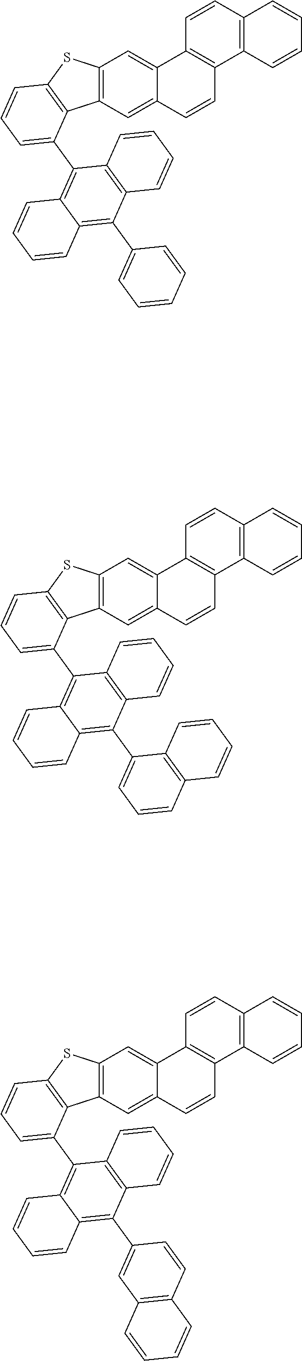 [SMILES: c1ccc(-c2c3ccccc3c(-c3cccc4sc5cc6c(ccc7c8ccccc8ccc67)cc5c34)c3ccccc23)cc1 ; c1ccc2c(-c3c4ccccc4c(-c4cccc5sc6cc7c(ccc8c9ccccc9ccc78)cc6c45)c4ccccc34)cccc2c1 ; c1ccc2cc(-c3c4ccccc4c(-c4cccc5sc6cc7c(ccc8c9ccccc9ccc78)cc6c45)c4ccccc34)ccc2c1]

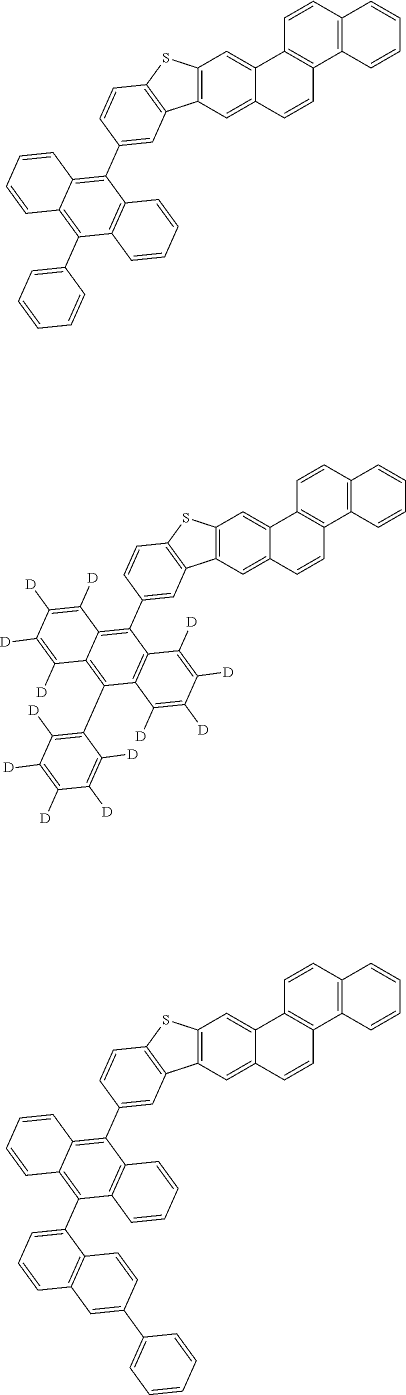 [SMILES: c1ccc(-c2c3ccccc3c(-c3ccc4sc5cc6c(ccc7c8ccccc8ccc67)cc5c4c3)c3ccccc23)cc1 ; [2H]c1c([2H])c([2H])c(-c2c3c([2H])c([2H])c([2H])c([2H])c3c(-c3ccc4sc5cc6c(ccc7c8ccccc8ccc67)cc5c4c3)c3c([2H])c([2H])c([2H])c([2H])c23)c([2H])c1[2H] ; c1ccc(-c2ccc3c(-c4c5ccccc5c(-c5ccc6sc7cc8c(ccc9c%10ccccc%10ccc89)cc7c6c5)c5ccccc45)cccc3c2)cc1]

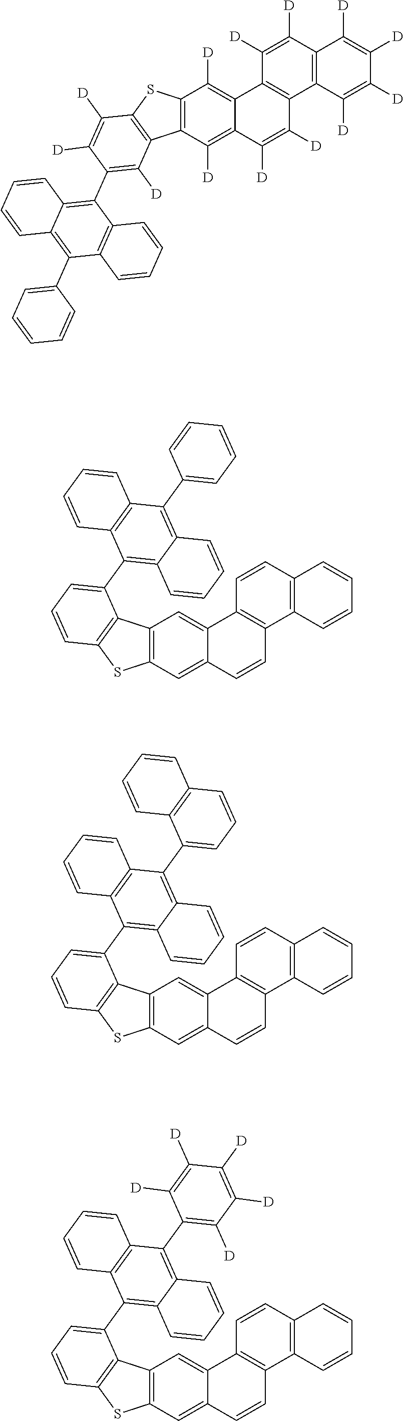 [SMILES: [2H]c1c(-c2c3ccccc3c(-c3ccccc3)c3ccccc23)c([2H])c2c(sc3c([2H])c4c(c([2H])c([2H])c5c6c([2H])c([2H])c([2H])c([2H])c6c([2H])c([2H])c45)c([2H])c32)c1[2H] ; c1ccc(-c2c3ccccc3c(-c3cccc4sc5cc6ccc7c8ccccc8ccc7c6cc5c34)c3ccccc23)cc1 ; c1ccc2c(-c3c4ccccc4c(-c4cccc5sc6cc7ccc8c9ccccc9ccc8c7cc6c45)c4ccccc34)cccc2c1 ; [2H]c1c([2H])c([2H])c(-c2c3ccccc3c(-c3cccc4sc5cc6ccc7c8ccccc8ccc7c6cc5c34)c3ccccc23)c([2H])c1[2H]]

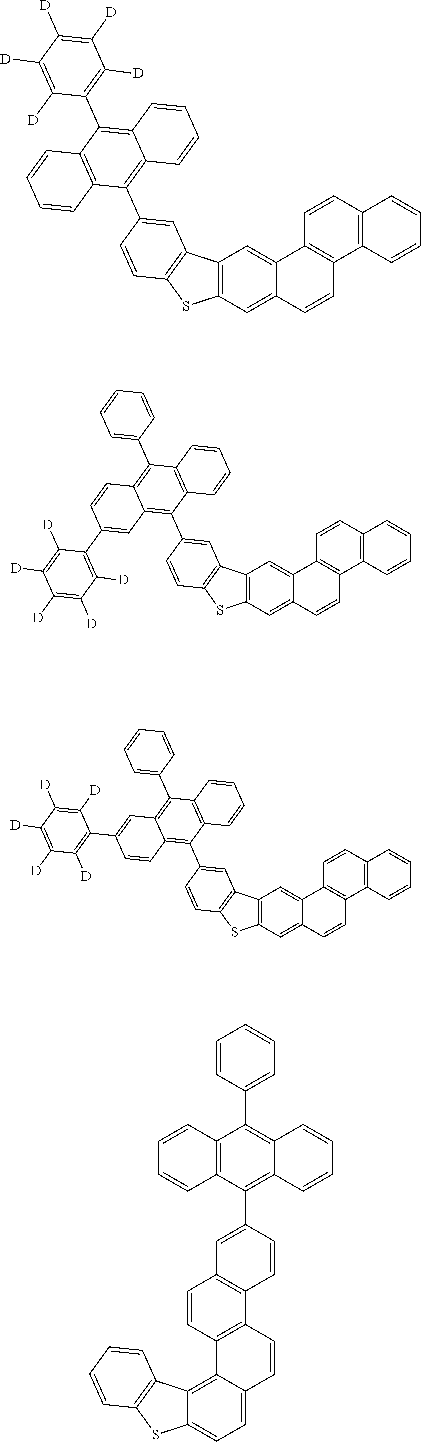 [SMILES: [2H]c1c([2H])c([2H])c(-c2c3ccccc3c(-c3ccc4sc5cc6ccc7c8ccccc8ccc7c6cc5c4c3)c3ccccc23)c([2H])c1[2H] ; [2H]c1c([2H])c([2H])c(-c2ccc3c(-c4ccccc4)c4ccccc4c(-c4ccc5sc6cc7ccc8c9ccccc9ccc8c7cc6c5c4)c3c2)c([2H])c1[2H] ; [2H]c1c([2H])c([2H])c(-c2ccc3c(-c4ccc5sc6cc7ccc8c9ccccc9ccc8c7cc6c5c4)c4ccccc4c(-c4ccccc4)c3c2)c([2H])c1[2H] ; c1ccc(-c2c3ccccc3c(-c3ccc4c(ccc5c4ccc4ccc6sc7ccccc7c6c45)c3)c3ccccc23)cc1]

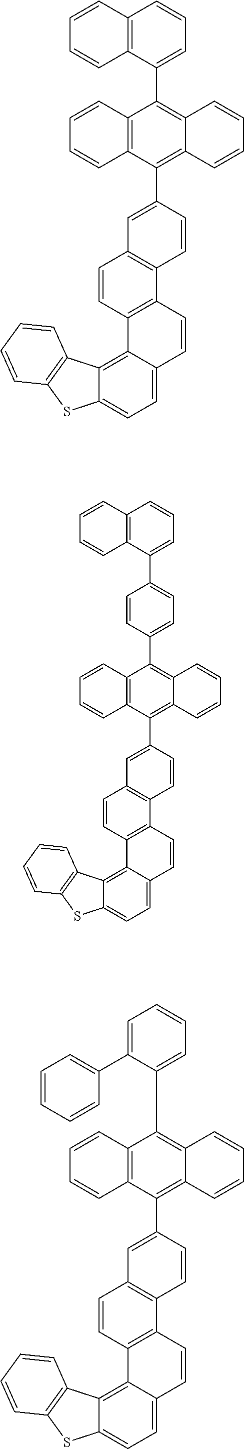 [SMILES: c1ccc2c(-c3c4ccccc4c(-c4ccc5c(ccc6c5ccc5ccc7sc8ccccc8c7c56)c4)c4ccccc34)cccc2c1 ; c1ccc2c(-c3ccc(-c4c5ccccc5c(-c5ccc6c(ccc7c6ccc6ccc8sc9ccccc9c8c67)c5)c5ccccc45)cc3)cccc2c1 ; c1ccc(-c2ccccc2-c2c3ccccc3c(-c3ccc4c(ccc5c4ccc4ccc6sc7ccccc7c6c45)c3)c3ccccc23)cc1]

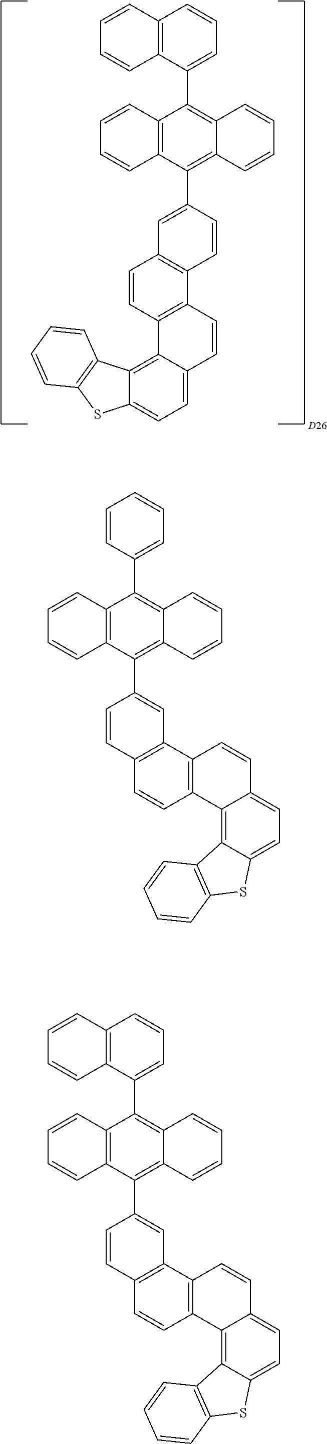

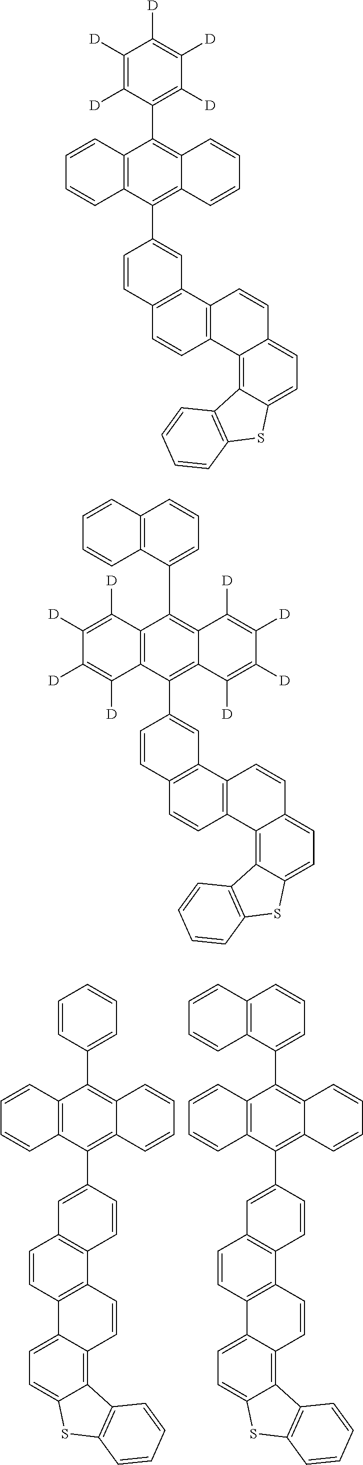

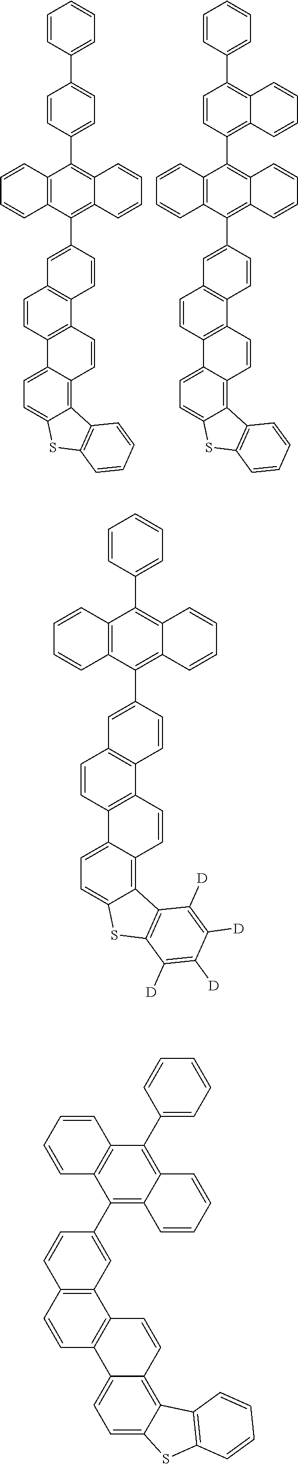

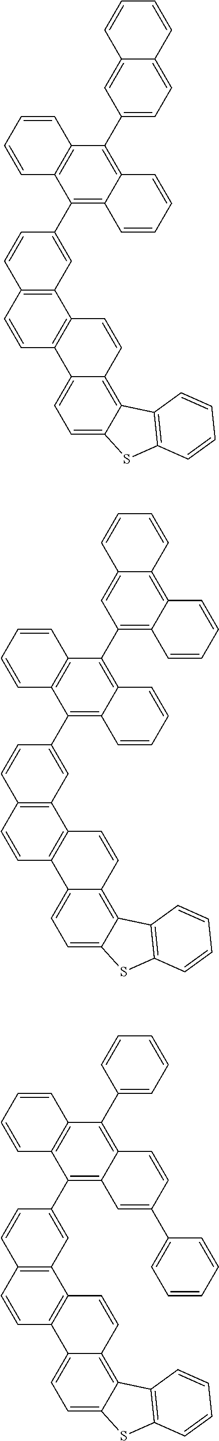 [SMILES: c1ccc2cc(-c3c4ccccc4c(-c4ccc5ccc6c(ccc7c6ccc6sc8ccccc8c67)c5c4)c4ccccc34)ccc2c1 ; c1ccc2c(c1)cc(-c1c3ccccc3c(-c3ccc4ccc5c(ccc6c5ccc5sc7ccccc7c56)c4c3)c3ccccc13)c1ccccc12 ; c1ccc(-c2ccc3c(-c4ccccc4)c4ccccc4c(-c4ccc5ccc6c(ccc7c6ccc6sc8ccccc8c67)c5c4)c3c2)cc1]

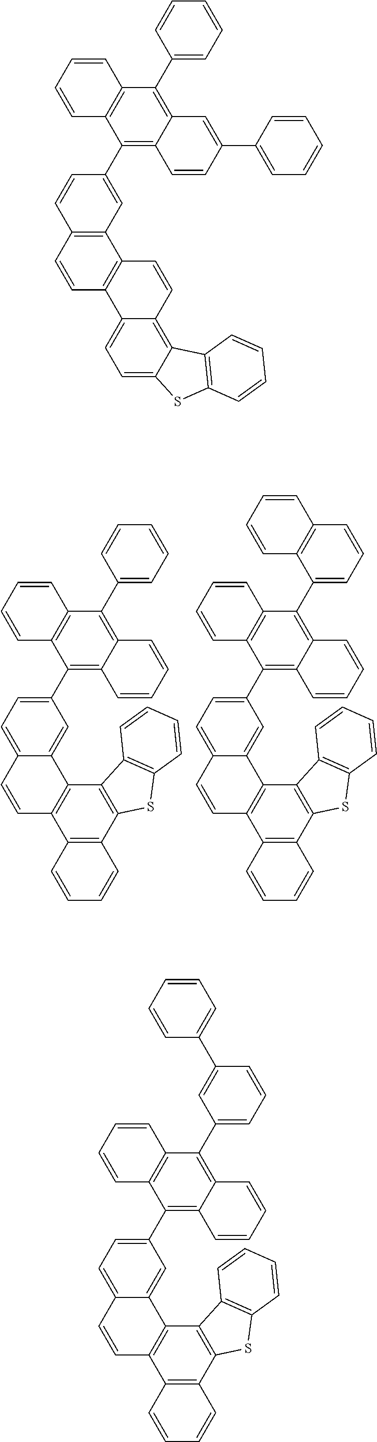

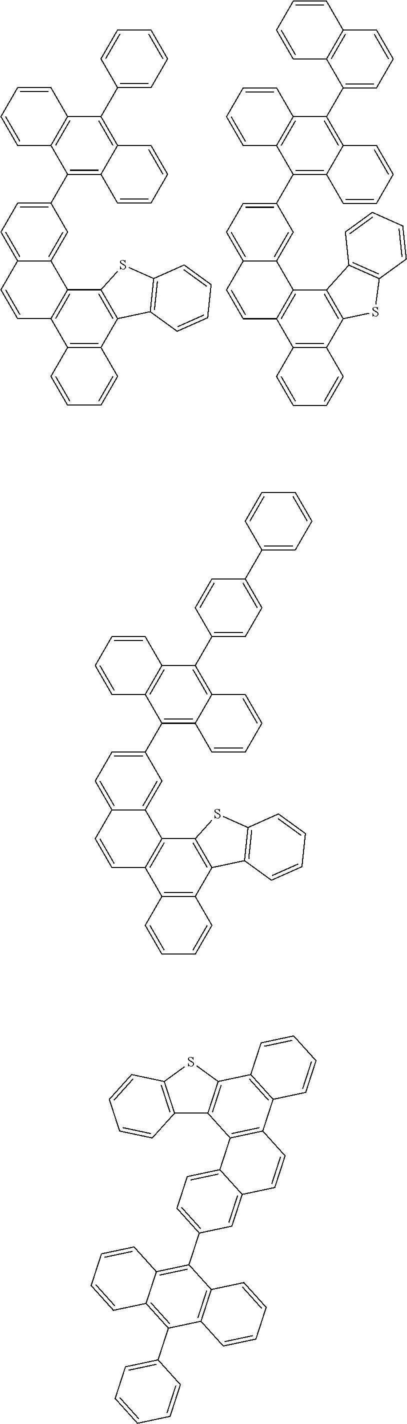

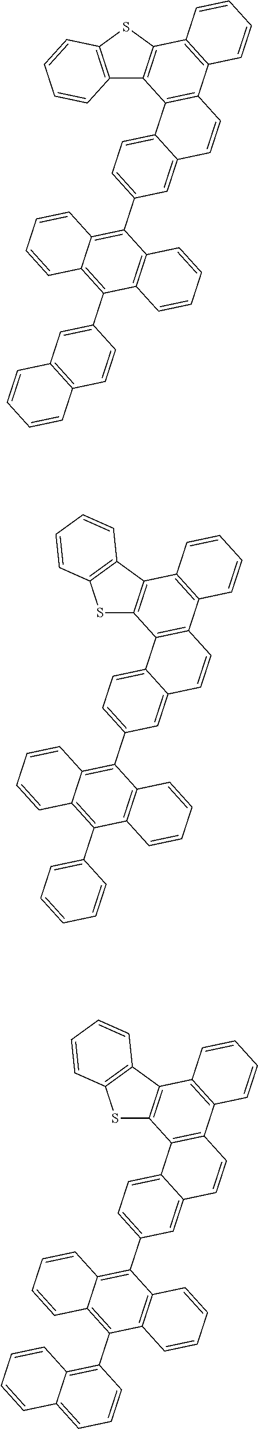 [SMILES: c1ccc2cc(-c3c4ccccc4c(-c4ccc5c(ccc6c7ccccc7c7sc8ccccc8c7c56)c4)c4ccccc34)ccc2c1 ; c1ccc(-c2c3ccccc3c(-c3ccc4c(ccc5c6ccccc6c6c7ccccc7sc6c45)c3)c3ccccc23)cc1 ; c1ccc2c(-c3c4ccccc4c(-c4ccc5c(ccc6c7ccccc7c7c8ccccc8sc7c56)c4)c4ccccc34)cccc2c1]

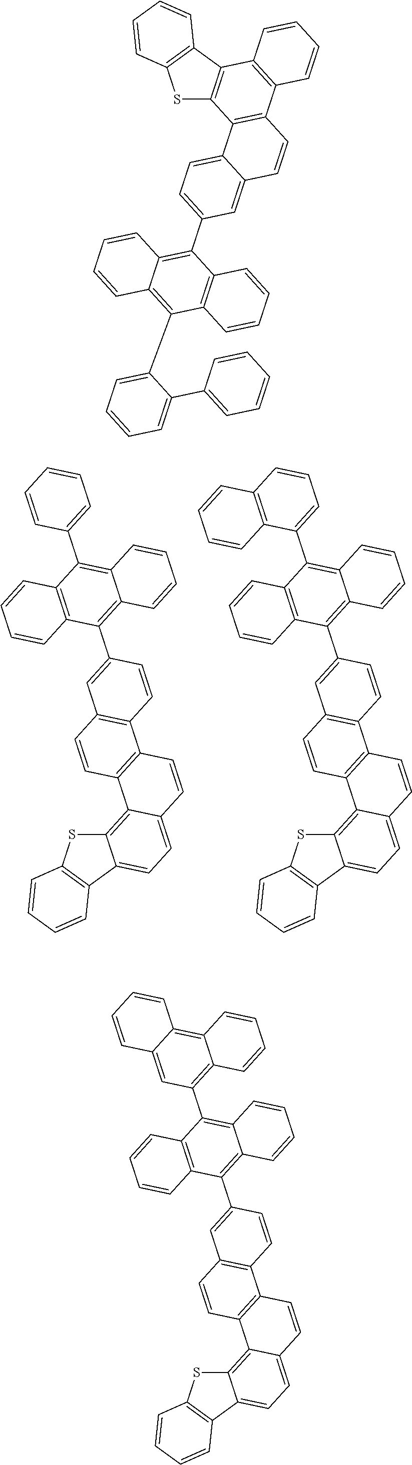

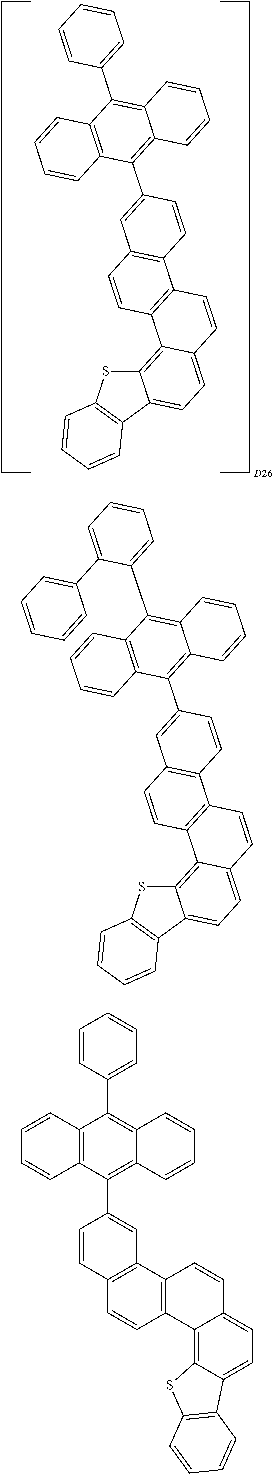

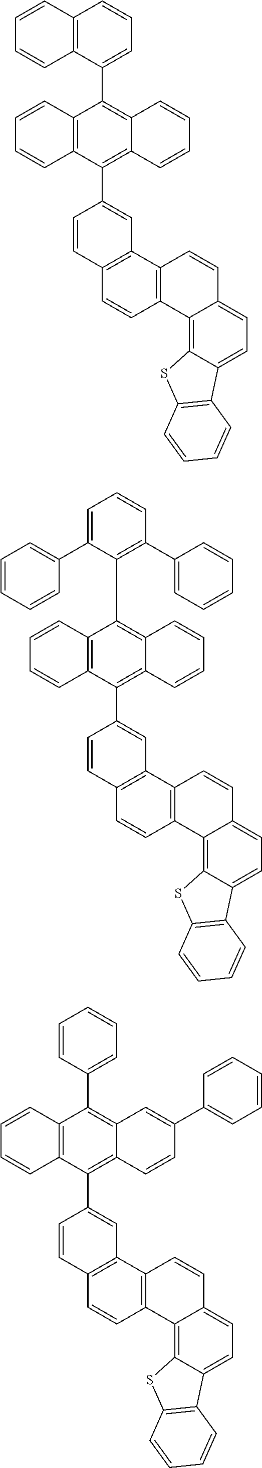 [SMILES: c1ccc2c(-c3c4ccccc4c(-c4ccc5ccc6c(ccc7ccc8c9ccccc9sc8c76)c5c4)c4ccccc34)cccc2c1 ; c1ccc(-c2cccc(-c3ccccc3)c2-c2c3ccccc3c(-c3ccc4ccc5c(ccc6ccc7c8ccccc8sc7c65)c4c3)c3ccccc23)cc1 ; c1ccc(-c2ccc3c(-c4ccc5ccc6c(ccc7ccc8c9ccccc9sc8c76)c5c4)c4ccccc4c(-c4ccccc4)c3c2)cc1]

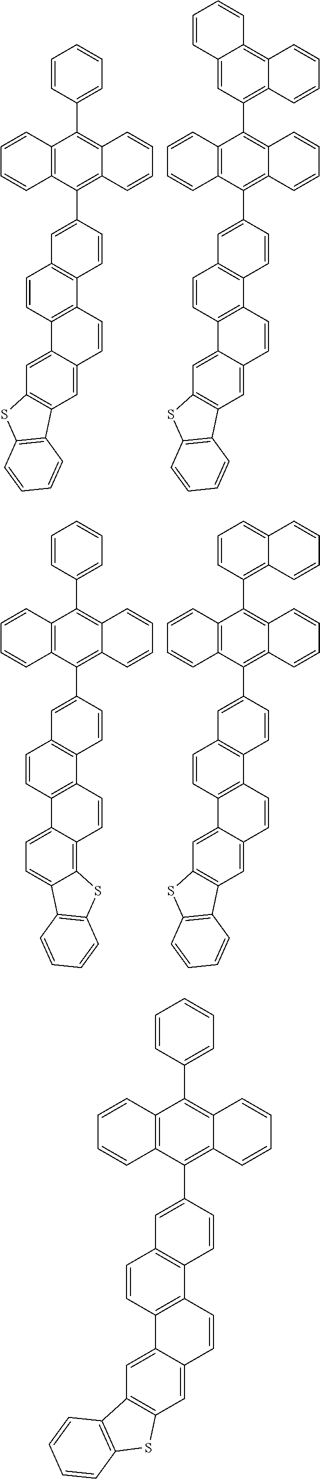

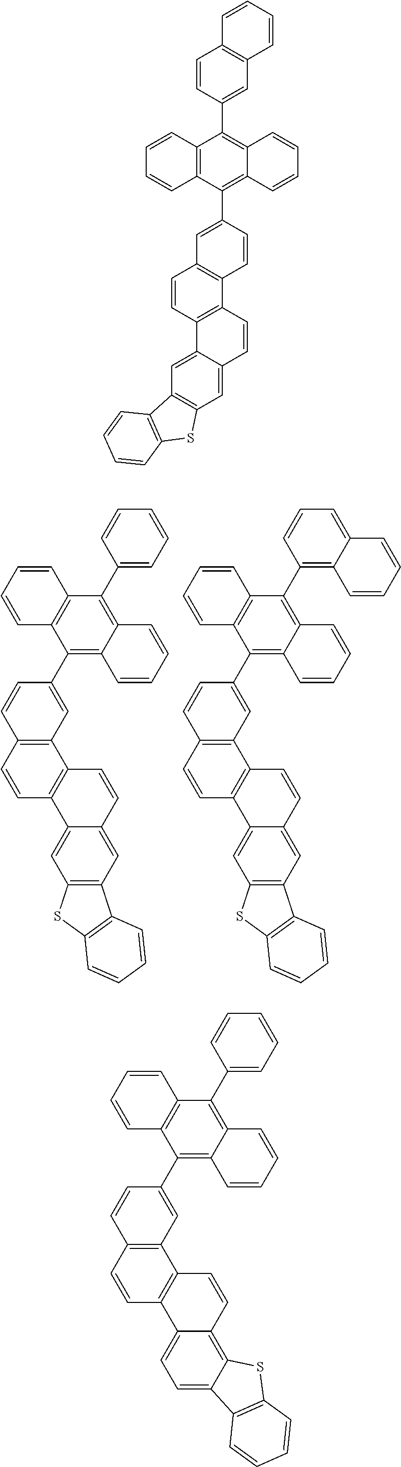

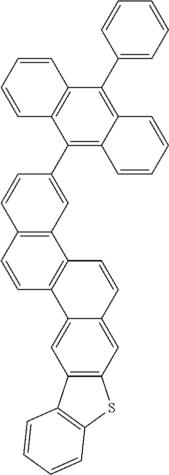 [SMILES: c1ccc(-c2c3ccccc3c(-c3ccc4ccc5c6cc7c(cc6ccc5c4c3)sc3ccccc37)c3ccccc23)cc1]

where the subscript “D” followed by a number indicates the number of deuterium substitutions; for example, D26 indicates that the structural formula contains 26 deuterium atoms.

In a second aspect of the present disclosure, an embodiment provides an organic electroluminescent device comprising the anthracene compound described above.

In some embodiments, the organic electroluminescent device comprises an anode and a cathode, where a light-emitting layer is disposed between the anode and the cathode, and the light-emitting layer includes the anthracene compound described above. The anthracene compound can be used alone or doped for preparing a light-emitting layer.

In some embodiments, the light-emitting layer comprises a dopant material and a host material comprising at least one of the anthracene compounds described above. A mass ratio of the host material in the light-emitting layer can be 90%-99%, or 95%-99%; and a mass ratio of the dopant material can be 1%-10%, or 1%-5%.

In some embodiments, the light-emitting layer is a blue light-emitting layer, and the dopant material is a blue light-emitting material.

In a third aspect of the present disclosure, an embodiment provides use of the anthracene compound described above in the semiconductor field.

In some embodiments, the anthracene compound described above is used to prepare a semiconductor device.

In some embodiments, the semiconductor device includes an optoelectronic device, such as a luminaire, a display (including an in-vehicle display, a computer display, or a television display, where the display can be an AMOLED display), or a sensor, or the like.

Compared with the existing technology, the present disclosure has the following beneficial effects:

The anthracene compound can be used to prepare a host material for an OLED, and is capable of reducing the driving voltage of the device, improving luminous efficiency, and prolonging the service life. In addition, the compound has high optical and electrical stability, and has the potential of being applied to the AMOLED industry.

### Definitions

Unless otherwise specified, the symbols in all general formulas and their sub-general formulas involved in the present disclosure (e.g., group code Ar, etc.) have the same interpretation scope in all embodiments of the present disclosure. The definition scopes of different symbols can be freely combined, and these combinations fall within the protection scope of the present disclosure.

The term “halogen” refers to one or more of fluorine, chlorine, bromine, or iodine, typically including fluorine, chlorine, or bromine.

The term “alkyl” refers to a linear or branched saturated hydrocarbon group, and “cycloalkyl” refers to a non-aromatic carbon-based ring composed of at least 3 carbon atoms, which can include monocyclic, polycyclic, and spirane structures.

The term “heteroalkyl” refers to an alkyl group as defined hereinbefore in which at least one carbon atom is replaced by a non-carbon atom, but excludes the case where a carbon atom as a connection site is replaced by a non-carbon atom (e.g., alkoxy, alkylsilyl). “Heterocycloalkyl” refers to a group formed on the basis of a cycloalkyl group in which at least one carbon atom is replaced by a non-carbon atom. Where, the non-carbon atom can be selected from at least one of O, S, N, Se, Si, or Ge.

The term “aryl” refers to an aromatic hydrocarbon group derived from the removal of one hydrogen atom on the basis of an aromatic ring compound, which can be a monocyclic aryl group or a polycyclic aryl group. At least one ring in a polycyclic aryl group is an aromatic ring system.

Multiple rings in a polycyclic aryl group can be connected to each other via single bond or can be fused to each other. As specific examples of the aryl group, for example, phenyl, naphthyl, anthryl, phenanthryl, tetracenyl, pyrenyl, chrysenyl, benzo[c]phenanthryl, benzo[g]chrysenyl, fluorenyl, benzofluorenyl, dibenzofluorenyl, biphenyl, terphenyl, quaterphenyl, fluoranthenyl.

The term “heteroaryl” refers to a monovalent group of a heteroaromatic system in which at least one carbon atom is replaced by a non-carbon atom on the basis of an aryl group, where the non-carbon atom can be selected from O, S, N, Se, Si, or Ge, but excludes the case where the aryl group takes a non-carbon atom as the connection site (e.g., aryloxy, arylsilyl, arylamino). As specific examples of heteroaryl groups, for example, pyrrolyl, pyrazinyl, pyridyl, pyrimidinyl, triazinyl, indolyl, isoindolyl, imidazolyl, furanyl, benzofuranyl, isobenzofuranyl, dibenzofuranyl, dibenzothienyl, azadibenzofuranyl, azadibenzothienyl, diazadibenzofuranyl, diazadibenzothienyl, quinolinyl, isoquinolinyl, quinoxalinyl, carbazolyl, phenanthridinyl, acridinyl, phenanthrolinyl, phenazinyl, phenothiazinyl, phenoxazinyl, oxazolinyl, oxadiazolyl, furazanyl, thienyl, benzothienyl, dihydroacridinyl, azacarbazolyl, diazacarbazolyl, quinazolinyl.

The terms “arylene” and “heteroarylene” correspond to divalent groups with the same structure as aryl, heteroaryl groups, respectively.

The term “hydrocarbyl” refers to a group consisting only of carbon and hydrogen atoms, including saturated hydrocarbyl (e.g., alkyl, cycloalkyl), unsaturated non-aromatic hydrocarbyl (e.g., alkenyl, alkynyl), and aromatic hydrocarbyl (e.g., phenyl).

In the term “alkylsilyl” or “arylsilyl”, the number of alkyl or aryl groups can be 1 to 3. “Alkylboranyl” includes monoalkylboranyl and dialkylboranyl, “arylboranyl” includes monoarylboranyl and diarylboranyl, and “arylphosphino” includes monoarylphosphino and diarylphosphino. In alkylsilyl, arylsilyl, alkylboranyl, arylboranyl, arylphosphino, the defined carbon number refers to the total carbon number of the corresponding group.

The term “adjacent” means that two groups or atoms are directly bonded, or that two groups or atoms are directly adjacent substituents to each other (i.e., two substitution sites are directly connected, rather than indirectly connected through other atoms or groups).

The “carbon number a-b” in the expression “substituted or unsubstituted X group with carbon number a-b” represents the carbon number in the case where X group is unsubstituted, excluding the carbon number of the substituent when X group is substituted.

“Substituted” in “substituted or unsubstituted” refers to the replacement of one or more hydrogen atoms by other atoms or functional groups (i.e., substituents), and unless otherwise limited by definition, also includes the replacement of one or more hydrogen atoms by a group formed by connecting of two or more of the above substituents.

The following examples are merely described to facilitate the understanding of the technical invention, and should not be considered as specific limitations of the present disclosure.

All starting materials, solvents and the like involved in the synthesis of compounds in the present disclosure are all purchased from Alfa, Acros, and other suppliers known to persons skilled in the art.

### Synthesis of Compounds 1 to 23

The structures of compounds 1 to 23 are as follows:

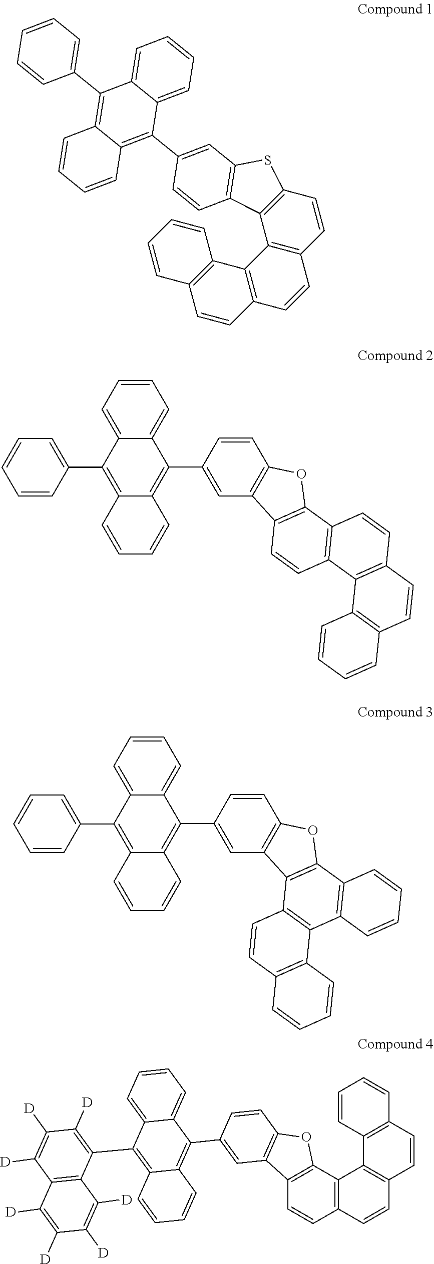 [SMILES: c1ccc(-c2c3ccccc3c(-c3ccc4c(c3)sc3ccc5ccc6ccc7ccccc7c6c5c34)c3ccccc23)cc1 ; c1ccc(-c2c3ccccc3c(-c3ccc4oc5c(ccc6c5ccc5ccc7ccccc7c56)c4c3)c3ccccc23)cc1 ; c1ccc(-c2c3ccccc3c(-c3ccc4oc5c6ccccc6c6c7ccccc7ccc6c5c4c3)c3ccccc23)cc1 ; [2H]c1c([2H])c([2H])c2c(-c3c4ccccc4c(-c4ccc5oc6c(ccc7ccc8ccc9ccccc9c8c76)c5c4)c4ccccc34)c([2H])c([2H])c([2H])c2c1[2H]]

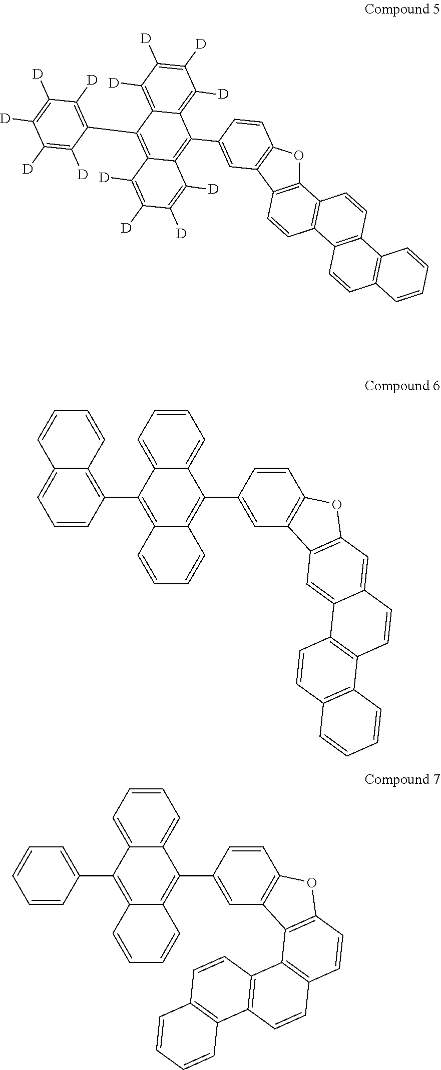 [SMILES: [2H]c1c([2H])c([2H])c(-c2c3c([2H])c([2H])c([2H])c([2H])c3c(-c3ccc4oc5c(ccc6c7ccc8ccccc8c7ccc65)c4c3)c3c([2H])c([2H])c([2H])c([2H])c23)c([2H])c1[2H] ; c1ccc2c(-c3c4ccccc4c(-c4ccc5oc6cc7ccc8c9ccccc9ccc8c7cc6c5c4)c4ccccc34)cccc2c1 ; c1ccc(-c2c3ccccc3c(-c3ccc4oc5ccc6ccc7c8ccccc8ccc7c6c5c4c3)c3ccccc23)cc1]

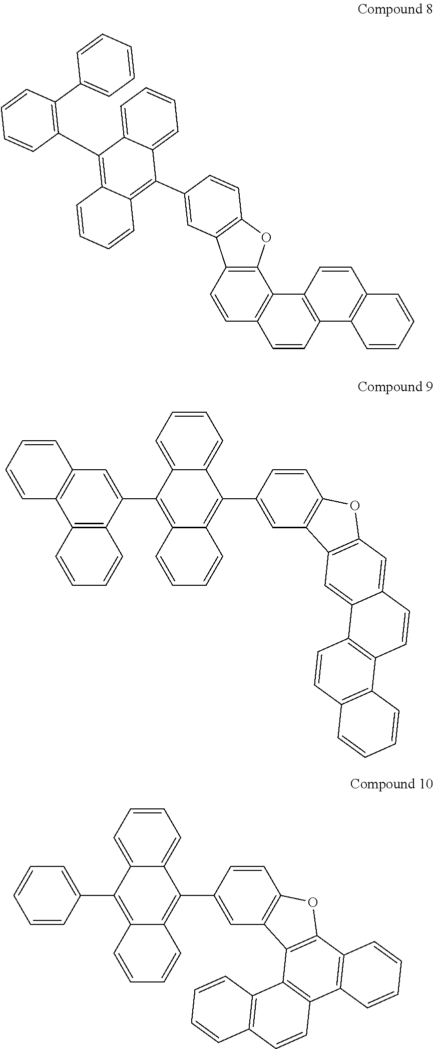 [SMILES: c1ccc(-c2ccccc2-c2c3ccccc3c(-c3ccc4oc5c(ccc6ccc7c8ccccc8ccc7c65)c4c3)c3ccccc23)cc1 ; c1ccc2c(c1)cc(-c1c3ccccc3c(-c3ccc4oc5cc6ccc7c8ccccc8ccc7c6cc5c4c3)c3ccccc13)c1ccccc12 ; c1ccc(-c2c3ccccc3c(-c3ccc4oc5c6ccccc6c6ccc7ccccc7c6c5c4c3)c3ccccc23)cc1]

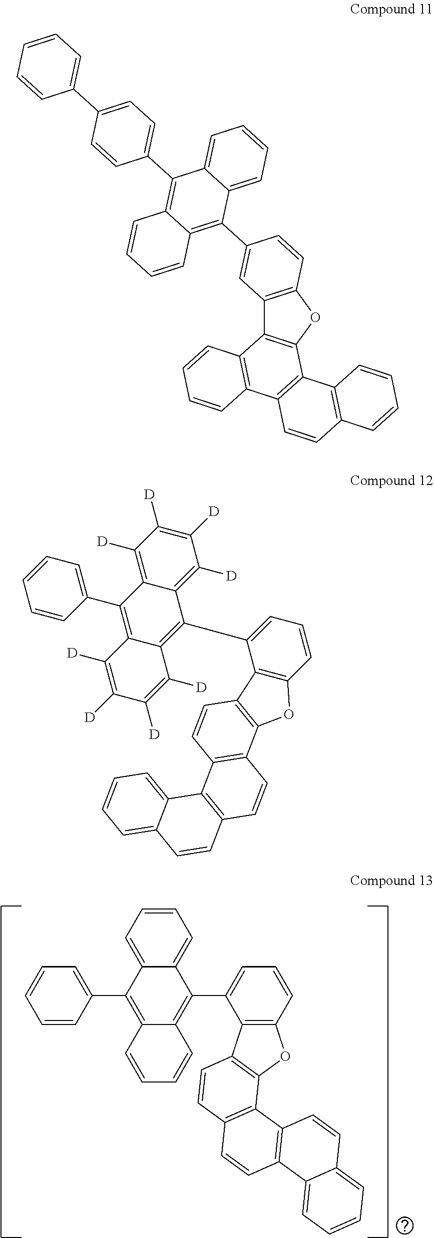

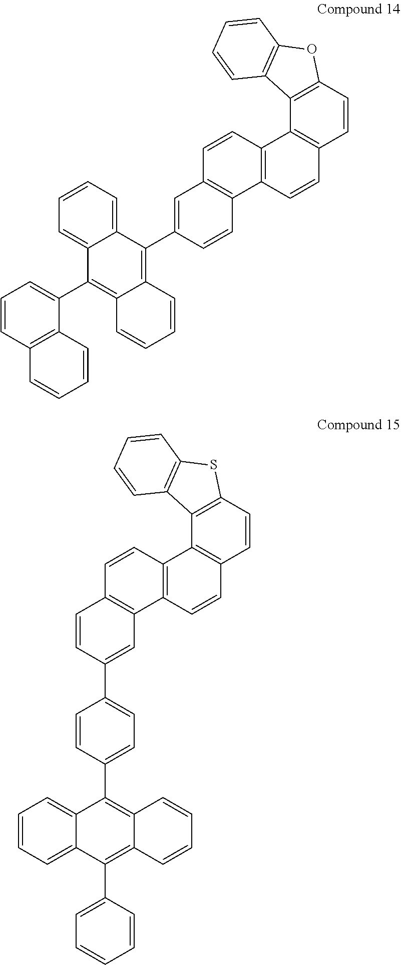 [SMILES: c1ccc2c(-c3c4ccccc4c(-c4ccc5c(ccc6c5ccc5ccc7oc8ccccc8c7c56)c4)c4ccccc34)cccc2c1 ; c1ccc(-c2c3ccccc3c(-c3ccc(-c4ccc5ccc6c(ccc7ccc8sc9ccccc9c8c76)c5c4)cc3)c3ccccc23)cc1]

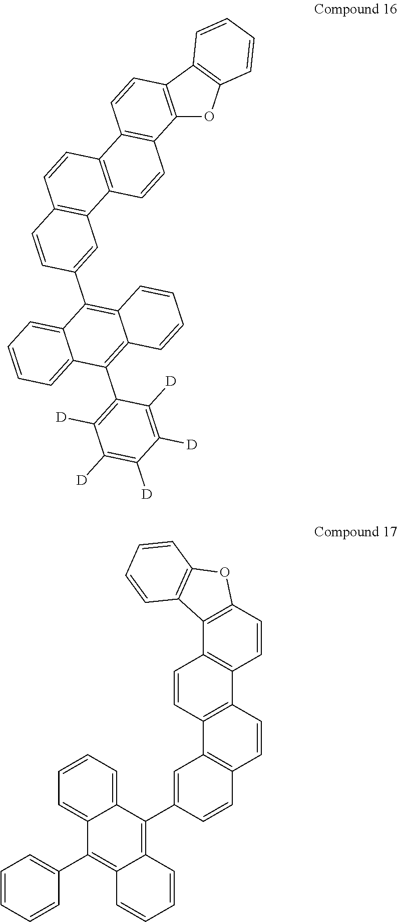 [SMILES: [2H]c1c([2H])c([2H])c(-c2c3ccccc3c(-c3ccc4ccc5c(ccc6c5ccc5c7ccccc7oc56)c4c3)c3ccccc23)c([2H])c1[2H] ; c1ccc(-c2c3ccccc3c(-c3ccc4ccc5c(ccc6c5ccc5oc7ccccc7c56)c4c3)c3ccccc23)cc1]

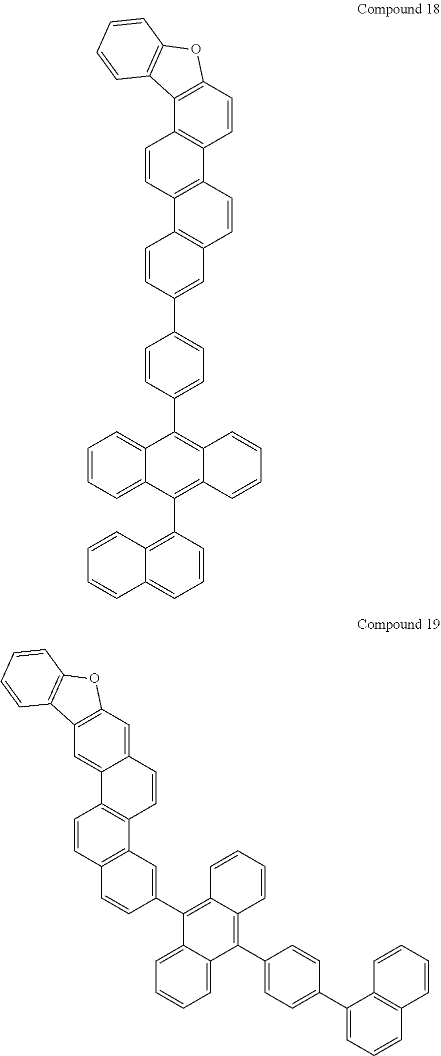 [SMILES: c1ccc2c(-c3c4ccccc4c(-c4ccc(-c5ccc6c(ccc7c6ccc6c7ccc7oc8ccccc8c76)c5)cc4)c4ccccc34)cccc2c1 ; c1ccc2c(-c3ccc(-c4c5ccccc5c(-c5ccc6ccc7c8cc9c(cc8ccc7c6c5)oc5ccccc59)c5ccccc45)cc3)cccc2c1]

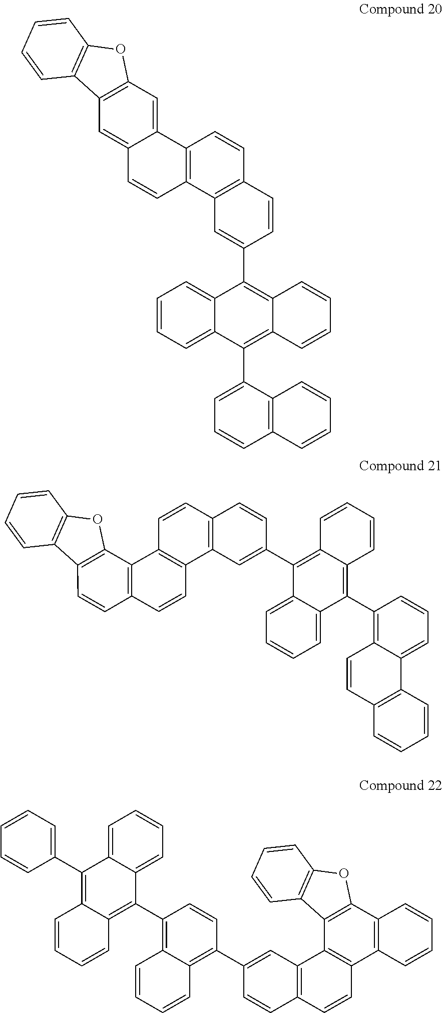 [SMILES: c1ccc2c(-c3c4ccccc4c(-c4ccc5ccc6c7cc8oc9ccccc9c8cc7ccc6c5c4)c4ccccc34)cccc2c1 ; c1ccc2c(c1)ccc1c(-c3c4ccccc4c(-c4ccc5ccc6c(ccc7ccc8c9ccccc9oc8c76)c5c4)c4ccccc34)cccc12 ; c1ccc(-c2c3ccccc3c(-c3ccc(-c4ccc5ccc6c7ccccc7c7oc8ccccc8c7c6c5c4)c4ccccc34)c3ccccc23)cc1]

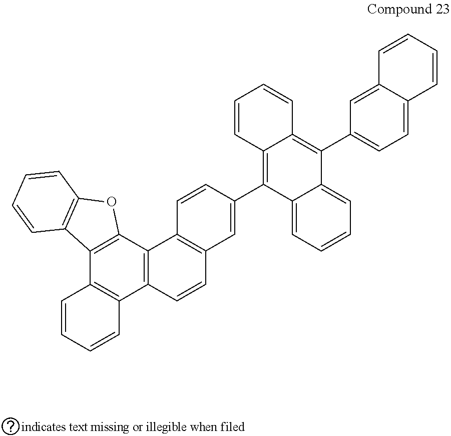 [SMILES: c1ccc2cc(-c3c4ccccc4c(-c4ccc5c(ccc6c7ccccc7c7c8ccccc8oc7c56)c4)c4ccccc34)ccc2c1]

where the specific synthesis method of Compound 12 is as follows:

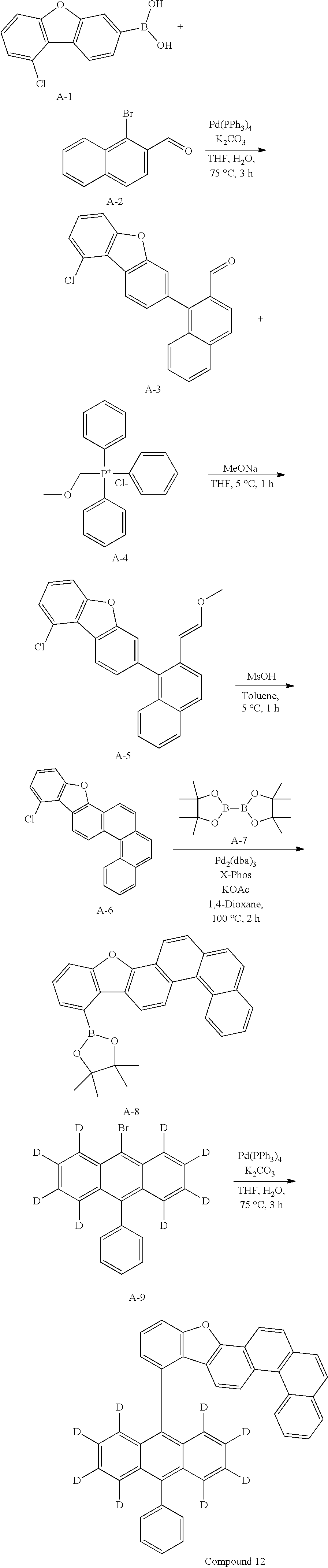

### Synthesis of Compound A-3

A-1 (20.00 g, 81.15 mmol), A-2 (19.08 g, 81.15 mmol), tetrakis(triphenylphosphine)palladium (1.88 g, 1.62 mmol), potassium carbonate (22.43 g, 162.3 mmol), tetrahydrofuran (400 mL) and deionized water (125 mL) were added to a 1000 mL three-necked round-bottom flask, and subjected to three vacuum-nitrogen purge cycles. A resulting reaction mixture was then heated to 70° C. and allowed to react for 4 h, and the reaction was monitored by thin layer chromatography (TLC, ethyl acetate:n-hexane=1:10 as developing agent) until the starting material A-1 was completely consumed.

The reaction mixture was cooled to 60° C. and concentrated under reduced pressure to remove the solvent, and ethyl acetate (600 mL) was added. A resulting organic layer was washed with deionized water three times (300 mL×3), and after phase separation, mixed with silica gel and dry-loaded onto a column for purification by silica gel column chromatography (200-300 mesh silica gel, ethyl acetate:n-hexane=1:15 as eluent). After elution, the eluate was concentrated under reduced pressure at 70° C. for 2 h to give a white solid A-3 (20.96 g, purity: 99.52%, yield: 72.4%), mass spectrum: 357.81 (M+H).

### Synthesis of Compound A-5

A-3 (19.00 g, 53.45 mmol), A-4 (21.9 g, 63.9 mmol) and tetrahydrofuran (280 mL) were added to a 1000 mL three-necked round-bottom flask, and subjected to three vacuum-nitrogen purge cycles. A resulting reaction mixture was then cooled to 5° C., and sodium methoxide (5.75 g, 106.50 mmol) was added in one portion. The reaction was maintained at 5° C. for 1 h and monitored by TLC (ethyl acetate:n-hexane=1:10 as developing agent) until the starting material A-3 was completely consumed. Deionized water (500 mL) was added, and the solvent was removed by concentration under reduced pressure. Ethyl acetate (750 mL) was added for extraction, and after phase separation, a resulting organic layer was concentrated under reduced pressure at 70° C. for 1 h to give a white solid A-5 (19.1 g, yield: 93.6%), mass spectrum: 385.86 (M+H). The obtained compound was directly used for the next reaction without purification.

### Synthesis of Compound A-6

A-5 (18.5 g, 48.07 mmol) and toluene (200 mL) were added to a 500 mL three-necked round-bottom flask, and subjected to three vacuum-nitrogen purge cycles. A resulting reaction mixture was then cooled to 5° C., and methanesulfonic acid (9.24 g, 96.14 mmol) was slowly added dropwise over 3 min. The reaction was maintained at 5° C. for 1 h and monitored by TLC (ethyl acetate:n-hexane=1:15 as developing agent) until the starting material A-5 was completely consumed. Methanol (220 mL) was added, and a large amount of white solid precipitated, which was crystallized once from toluene (230 mL) and methanol (200 mL), filtered with suction, and the filter cake was dried under vacuum at 80° C. for 1 h to give a white solid A-6 (11.65 g, purity: 99.42%, yield: 68.72%), mass spectrum: 353.82 (M+H).

### Synthesis of Compound A-8

A-6 (10.5 g, 29.76 mmol), A-7 (9.07 g, 35.71 mmol), tris(dibenzylideneacetone)dipalladium (1.12 g, 1.23 mmol), 2-dicyclohexylphosphino-2′,4′,6′-triisopropylbiphenyl (1.32 g, 2.46 mmol), potassium acetate (8.42 g, 61.32 mmol) and 1,4-dioxane (200 mL) were added to a 1000 mL three-necked round-bottom flask, and subjected to three vacuum-nitrogen purge cycles. A resulting reaction mixture was then heated to 100° C. and allowed to react for 2 h, and the reaction was monitored by TLC (ethyl acetate:n-hexane=1:10 as developing agent) until the starting material A-6 was completely consumed. The reaction mixture was cooled to 60° C., and concentrated under reduced pressure to remove the solvent, and ethyl acetate (300 mL) was added. A resulting organic layer was washed with deionized water three times (150 mL×3), and after phase separation, mixed with silica gel and dry-loaded onto a column for purification by silica gel column chromatography (200-300 mesh silica gel, ethyl acetate:n-hexane=1:15 as eluent). After elution, the eluate was concentrated under reduced pressure at 70° C. for 1 h gave a white solid A-8 (11.45 g, purity: 98.6%, yield: 86.55%), mass spectrum: 445.21 (M+H).

### Synthesis of Compound 12

A-8 (11.00 g, 24.76 mmol), A-9 (9.29 g, 27.23 mmol), tetrakis(triphenylphosphine)palladium (0.76 g, 0.88 mmol), potassium carbonate (6.13 g, 49.52 mmol), tetrahydrofuran (220 mL) and deionized water (60 mL) were added to a 1000 mL three-necked round-bottom flask, and subjected to three vacuum-nitrogen purge cycles. A resulting reaction mixture was then heated to 75° C. and allowed to react for 3 h, and the reaction was monitored by TLC (ethyl acetate:n-hexane=1:10 as developing agent) until the starting material A-8 was completely consumed. The reaction mixture was cooled to 60° C., and concentrated under reduced pressure to remove the solvent, and filtered with suction to give a yellow solid. Xylene (360 mL) was added, and the material was dissolved by heating to 120° C., and cooled to room temperature and filtered once. Column chromatography was performed on silica gel (25 g, 200-300 mesh), and the filter cake was rinsed with 150 mL of toluene until no product remained. The organic phases were combined, and then concentrated under reduced pressure at 70° C. for 2 h to give a yellow solid. It was crystallized twice from xylene and methanol and dried under vacuum at 100° C. for 8 h to give a yellow solid (crude compound 12, 11.47 g, purity: 99.94%, yield: 80.07%). The obtained 11.47 g of crude compound 12 was purified by sublimation to obtain sublimation-purified compound 12 (8.53 g, purity: 99.96%, yield: 74.36%), mass spectrum: 579.74 (M+H).

1H NMR (400 MHz, CDCl3) δ 8.27 (d, J=8.5 Hz, 1H), 7.95 (d, J=8.8 Hz, 1H), 7.91-7.86 (m, 3H), 7.81 (d, J=8.4 Hz, 1H), 7.71-7.64 (m, 2H), 7.64-7.51 (m, 7H), 7.50-7.43 (m, 1H), 7.39 (dd, J=7.8, 1.2 Hz, 1H), 7.33 (tt, J=7.5, 1.5 Hz, 1H).

where, the specific synthesis method of Compound 13 is as follows:

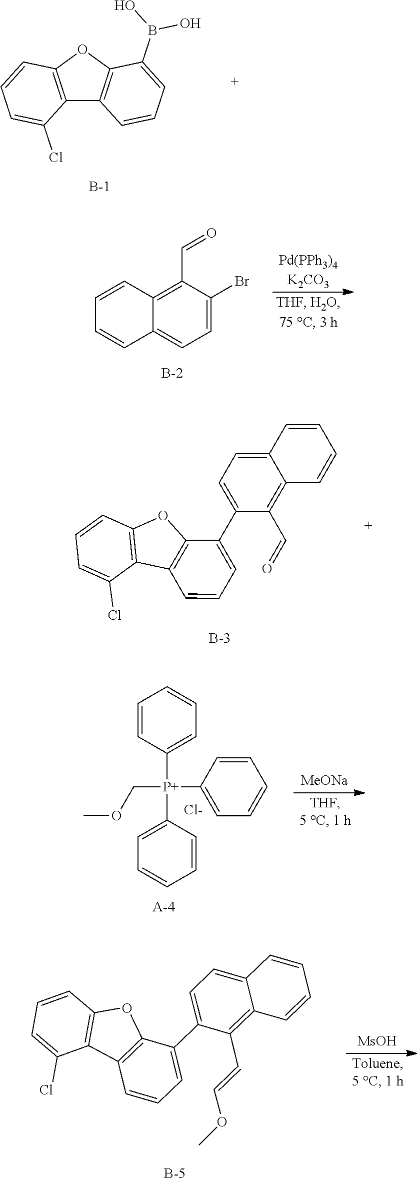

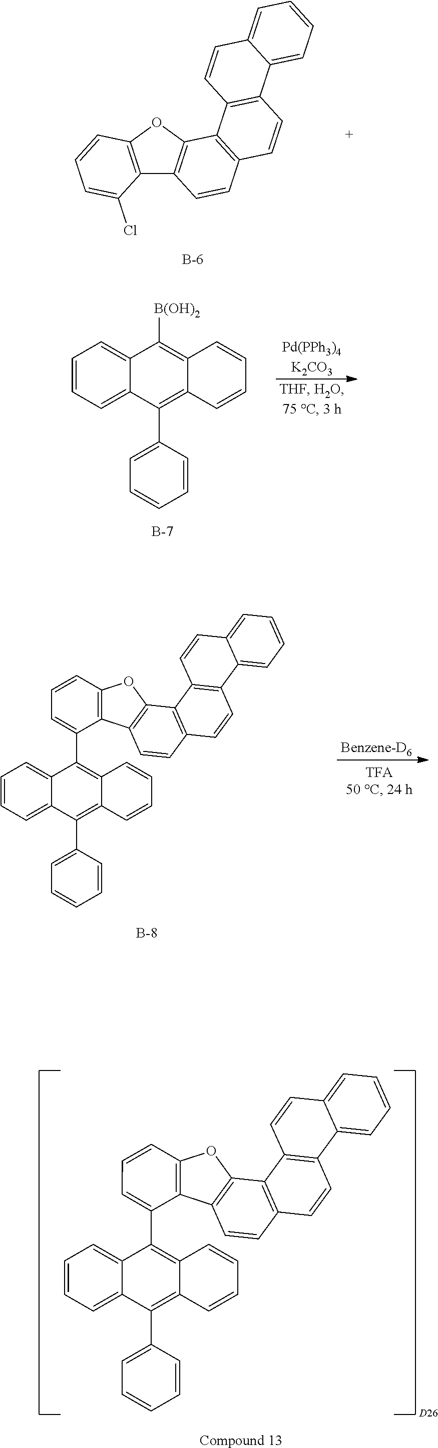

### Synthesis of Compound B-3

Compound B-3 was prepared by following the same synthetic and purification procedure as described for compound A-3, except that the corresponding starting materials were substituted accordingly. This afforded the target compound B-3 as a white solid (12.46 g, purity: 99.25%, yield: 71.52%), mass spectrum: 357.81 (M+H).

### Synthesis of Compound B-5

Compound B-5 was prepared by following the same synthetic and purification procedure as described for compound A-5, except that the corresponding starting materials were substituted accordingly. This afforded the target compound B-5 as a white solid (10.38 g, purity: 99.63%, yield: 75.26%), mass spectrum: 385.86 (M+H).

### Synthesis of Compound B-6

Compound B-6 was prepared by following the same synthetic and purification procedure as described for compound A-6, except that the corresponding starting materials were substituted accordingly. This afforded the target compound B-6 as a white solid (9.83 g, purity: 99.54%, yield: 68.44%), mass spectrum: 353.82 (M+H).

### Synthesis of Compound B-8

Compound B-8 was prepared by following the same synthetic and purification procedure as described for compound 12, except that the corresponding starting materials were substituted accordingly. This afforded the target compound B-8 as a white solid (8.53 g, purity: 99.93%, yield: 65.68%), mass spectrum: 571.69 (M+H).

### Synthesis of Compound 13

Compound B-8 (8.1 g, 14.19 mmol), deuterated benzene-D₆ (80 mL) and trifluoroacetic acid (1.39 g, 14.19 mmol) were added to a 250 mL single-necked round-bottom flask, and subjected to three vacuum-nitrogen purge cycles. The reaction mixture was then heated to 50° C. and stirred for 24 h. A resulting reaction mixture was cooled to room temperature and added with heavy water (30 mL) dropwise to quench the reaction, and stirred at room temperature for 0.5 h. Ethyl acetate (150 mL) was added, and a resulting organic layer was washed with deionized water three times (100 mL×3). The organic phases were combined, and then concentrated under reduced pressure at 65° C. for 1 h to give a white solid. The white solid was mixed with silica gel and dry-loaded onto a column for purification by silica gel column chromatography (200-300 mesh silica gel, n-hexane=100% as eluent). After elution, the eluates were concentrated under reduced pressure at 75° C. for 1 h to give a white solid (crude compound 13, 7.52 g, purity: 99.51%, deuteration content of 26: 98.57%, yield: 88.73%, where fraction bearing 26 deuterium atoms refers to the percentage of product molecules containing exactly 26 deuterium substitutions, as determined by the method described in Example 1 of Chinese Patent No. CN115266981A). The obtained 7.52 g of crude compound 13 was purified by sublimation to obtain sublimation-purified compound 13 (6.14 g, purity: 99.95%, yield: 81.64%), mass spectrum: 597.85 (M+H).

The preparation of other compounds followed the same synthetic procedure for compound 13, where B-6 and B-7 were employed to synthesize B-8, except that different starting materials were used, as shown in Table 1. The preparation of starting material B-6 can be carried out with reference to the steps disclosed in the examples of Chinese Patent Nos. CN117645592A and CN117924225A. For example, the starting material B-6 used for the synthesis of compound 3 can be prepared by following the synthetic route to compound CPD94 described in Chinese Patent No. CN117645592A, using the synthesis method of CPD94-5. The preparation methods of other compounds are not individually enumerated herein.

TABLE 1

| | | Starting material |
|---|---|---|
| | Starting material B-6 | B-7 |
| Compound 1 | 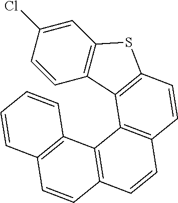 [SMILES: Clc1ccc2c(c1)sc1ccc3ccc4ccc5ccccc5c4c3c12] | 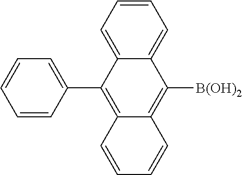 [SMILES: OB(O)c1c2ccccc2c(-c2ccccc2)c2ccccc12] |
| Compound 2 | 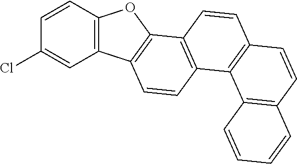 [SMILES: Clc1ccc2oc3c(ccc4c3ccc3ccc5ccccc5c34)c2c1] | 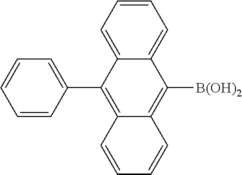 [SMILES: OB(O)c1c2ccccc2c(-c2ccccc2)c2ccccc12] |
| Compound 3 | 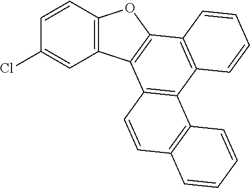 [SMILES: Clc1ccc2oc3c4ccccc4c4c5ccccc5ccc4c3c2c1] | 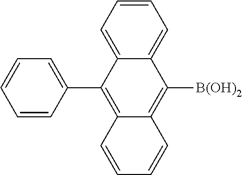 [SMILES: OB(O)c1c2ccccc2c(-c2ccccc2)c2ccccc12] |
| Compound 4 | 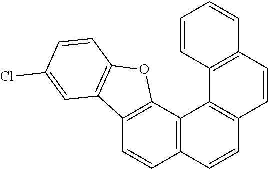 [SMILES: Clc1ccc2oc3c(ccc4ccc5ccc6ccccc6c5c43)c2c1] | 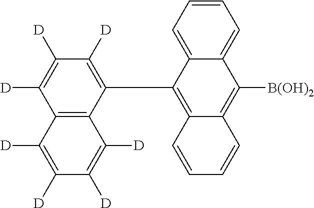 [SMILES: [2H]c1c([2H])c([2H])c2c(Cc3c4ccccc4c(B(O)O)c4ccccc34)c([2H])c([2H])c([2H])c2c1[2H]] |
| Compound 5 | 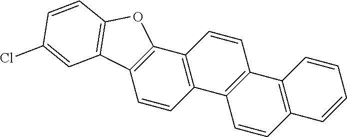 [SMILES: Clc1ccc2oc3c(ccc4c5ccc6ccccc6c5ccc43)c2c1] | 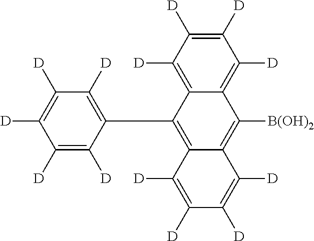 [SMILES: [2H]c1c([2H])c([2H])c(Cc2c3c([2H])c([2H])c([2H])c([2H])c3c(B(O)O)c3c([2H])c([2H])c([2H])c([2H])c23)c([2H])c1[2H]] |
| Compound 6 | 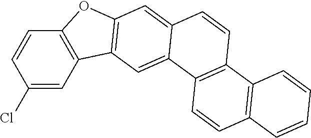 [SMILES: Clc1ccc2oc3cc4ccc5c6ccccc6ccc5c4cc3c2c1] | 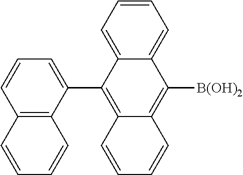 [SMILES: OB(O)c1c2ccccc2c(-c2cccc3ccccc23)c2ccccc12] |
| Compound 7 | 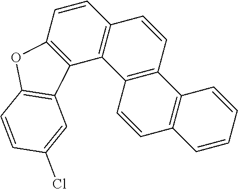 [SMILES: Clc1ccc2oc3ccc4ccc5c6ccccc6ccc5c4c3c2c1] | 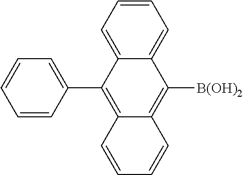 [SMILES: OB(O)c1c2ccccc2c(-c2ccccc2)c2ccccc12] |
| Compound 8 | 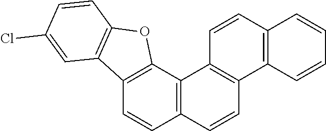 [SMILES: Clc1ccc2oc3c(ccc4ccc5c6ccccc6ccc5c43)c2c1] | 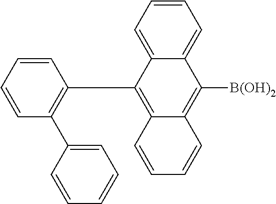 [SMILES: OB(O)c1c2ccccc2c(Cc2ccccc2-c2ccccc2)c2ccccc12] |
| Compound 9 | 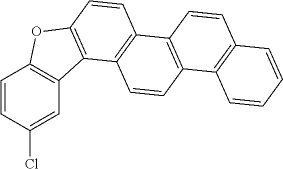 [SMILES: Clc1ccc2oc3ccc4c5ccc6ccccc6c5ccc4c3c2c1] | 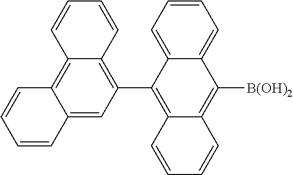 [SMILES: OB(O)c1c2ccccc2c(-c2cc3ccccc3c3ccccc23)c2ccccc12] |
| Compound 10 | 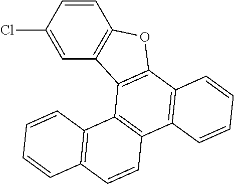 [SMILES: Clc1ccc2oc3c4ccccc4c4ccc5ccccc5c4c3c2c1] | 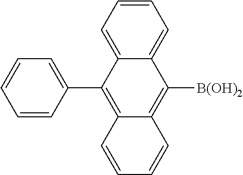 [SMILES: OB(O)c1c2ccccc2c(-c2ccccc2)c2ccccc12] |
| Compound 11 | 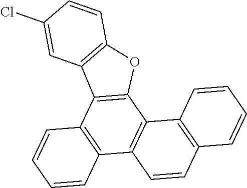 [SMILES: Clc1ccc2oc3c4c5ccccc5ccc4c4ccccc4c3c2c1] | 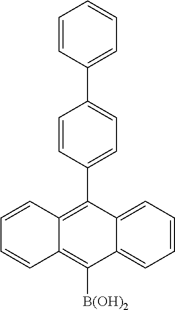 [SMILES: OB(O)c1c2ccccc2c(-c2ccc(-c3ccccc3)cc2)c2ccccc12] |
| Compound 14 | 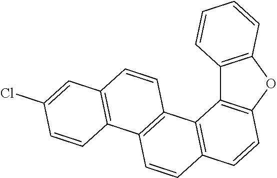 [SMILES: Clc1ccc2c(ccc3c2ccc2ccc4oc5ccccc5c4c23)c1] | 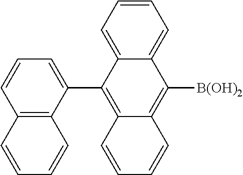 [SMILES: OB(O)c1c2ccccc2c(-c2cccc3ccccc23)c2ccccc12] |
| Compound 15 | 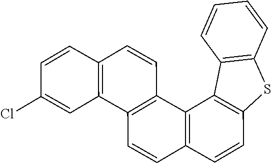 [SMILES: Clc1ccc2ccc3c(ccc4ccc5sc6ccccc6c5c43)c2c1] | 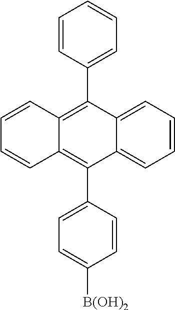 [SMILES: OB(O)c1ccc(-c2c3ccccc3c(-c3ccccc3)c3ccccc23)cc1] |
| Compound 16 | 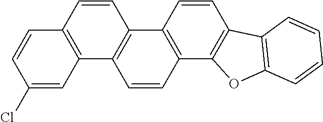 [SMILES: Clc1ccc2ccc3c(ccc4c3ccc3c5ccccc5oc34)c2c1] | 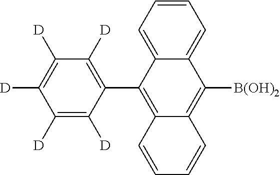 [SMILES: [2H]c1c([2H])c([2H])c(-c2c3ccccc3c(B(O)O)c3ccccc23)c([2H])c1[2H]] |
| Compound 17 | 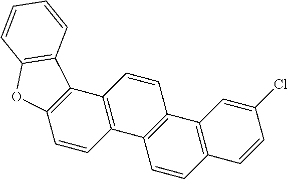 [SMILES: Clc1ccc2ccc3c(ccc4c3ccc3oc5ccccc5c34)c2c1] | 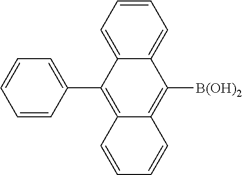 [SMILES: OB(O)c1c2ccccc2c(-c2ccccc2)c2ccccc12] |
| Compound 18 | 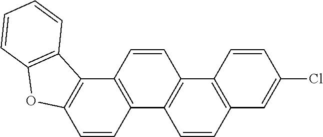 [SMILES: Clc1ccc2c(ccc3c2ccc2c3ccc3oc4ccccc4c32)c1] | 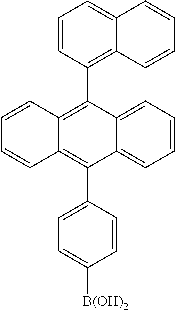 [SMILES: OB(O)c1ccc(-c2c3ccccc3c(-c3cccc4ccccc34)c3ccccc23)cc1] |
| Compound 19 | 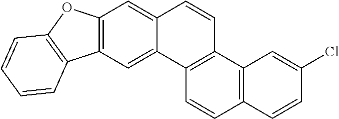 [SMILES: Clc1ccc2ccc3c4cc5c(cc4ccc3c2c1)oc1ccccc15] | 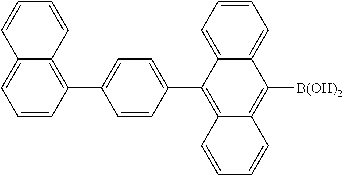 [SMILES: OB(O)c1c2ccccc2c(-c2ccc(-c3cccc4ccccc34)cc2)c2ccccc12] |
| Compound 20 | 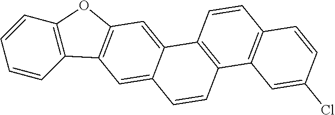 [SMILES: Clc1ccc2ccc3c4cc5oc6ccccc6c5cc4ccc3c2c1] | 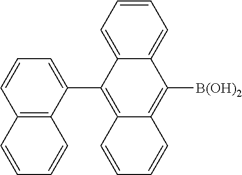 [SMILES: OB(O)c1c2ccccc2c(-c2cccc3ccccc23)c2ccccc12] |
| Compound 21 | 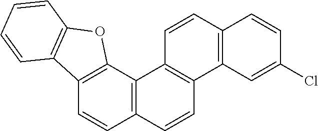 [SMILES: Clc1ccc2ccc3c(ccc4ccc5c6ccccc6oc5c43)c2c1] | 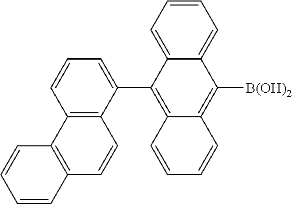 [SMILES: OB(O)c1c2ccccc2c(-c2cccc3c2ccc2ccccc23)c2ccccc12] |
| Compound 22 | 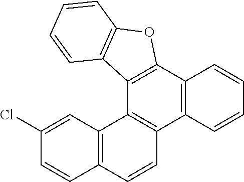 [SMILES: Clc1ccc2ccc3c4ccccc4c4oc5ccccc5c4c3c2c1] | 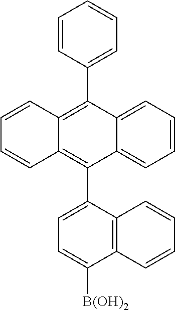 [SMILES: OB(O)c1ccc(-c2c3ccccc3c(-c3ccccc3)c3ccccc23)c2ccccc12] |
| Compound 23 | 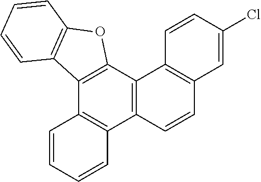 [SMILES: Clc1ccc2c(ccc3c4ccccc4c4c5ccccc5oc4c23)c1] | 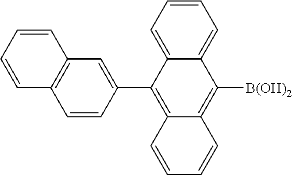 [SMILES: OB(O)c1c2ccccc2c(-c2ccc3ccccc3c2)c2ccccc12] |

| | | | MS |
|---|---|---|---|
| | Target compound | Yield | (M + H) |
| Compound 1 | 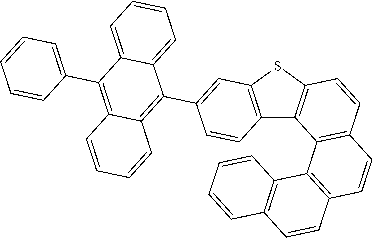 [SMILES: c1ccc(-c2c3ccccc3c(-c3ccc4c(c3)sc3ccc5ccc6ccc7ccccc7c6c5c34)c3ccccc23)cc1] | 71.86% | 587.75 |
| Compound 2 | 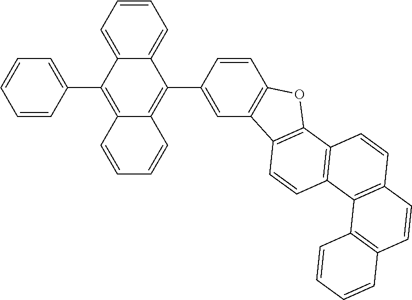 [SMILES: c1ccc(-c2c3ccccc3c(-c3ccc4oc5c(ccc6c5ccc5ccc7ccccc7c56)c4c3)c3ccccc23)cc1] | 77.32% | 571.69 |
| Compound 3 | 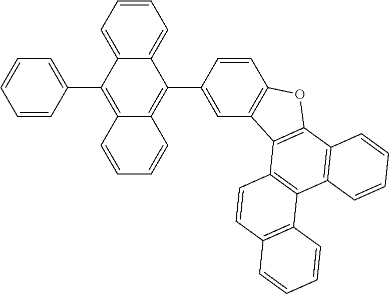 [SMILES: c1ccc(-c2c3ccccc3c(-c3ccc4oc5c6ccccc6c6c7ccccc7ccc6c5c4c3)c3ccccc23)cc1] | 80.53% | 571.69 |
| Compound 4 | 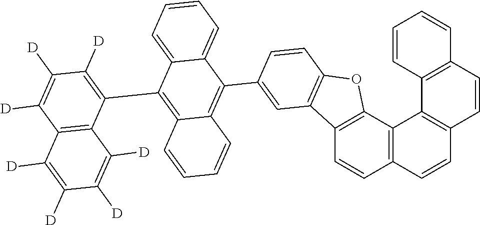 [SMILES: [2H]c1c([2H])c([2H])c2c(-c3c4ccccc4c(-c4ccc5oc6c(ccc7ccc8ccc9ccccc9c8c76)c5c4)c4ccccc34)c([2H])c([2H])c([2H])c2c1[2H]] | 78.5% | 628.79 |
| Compound 5 | 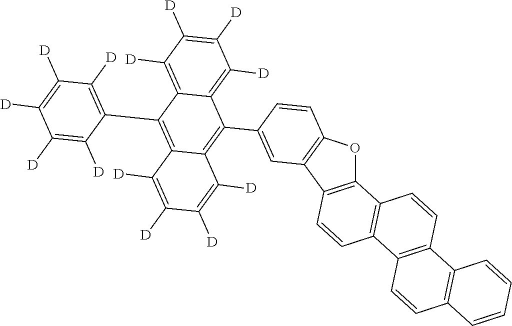 [SMILES: [2H]c1c([2H])c([2H])c(-c2c3c([2H])c([2H])c([2H])c([2H])c3c(-c3ccc4oc5c(ccc6c7ccc8ccccc8c7ccc65)c4c3)c3c([2H])c([2H])c([2H])c([2H])c23)c([2H])c1[2H]] | 73.26% | 584.77 |
| Compound 6 | 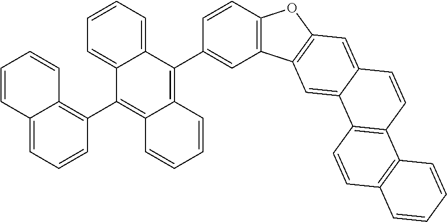 [SMILES: c1ccc2c(-c3c4ccccc4c(-c4ccc5oc6cc7ccc8c9ccccc9ccc8c7cc6c5c4)c4ccccc34)cccc2c1] | 83.65% | 621.75 |
| Compound 7 | 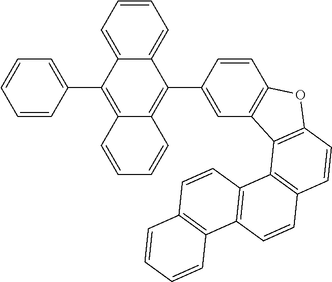 [SMILES: c1ccc(-c2c3ccccc3c(-c3ccc4oc5ccc6ccc7c8ccccc8ccc7c6c5c4c3)c3ccccc23)cc1] | 81.33% | 571.69 |
| Compound 8 | 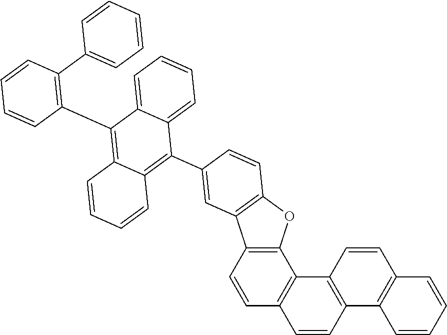 [SMILES: c1ccc(-c2ccccc2-c2c3ccccc3c(-c3ccc4oc5c(ccc6ccc7c8ccccc8ccc7c65)c4c3)c3ccccc23)cc1] | 79.45% | 647.79 |
| Compound 9 | 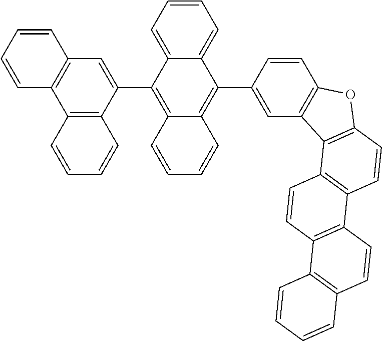 [SMILES: c1ccc2c(c1)cc(-c1c3ccccc3c(-c3ccc4oc5ccc6c7ccc8ccccc8c7ccc6c5c4c3)c3ccccc13)c1ccccc12] | 76.58% | 671.23 |
| Compound 10 | 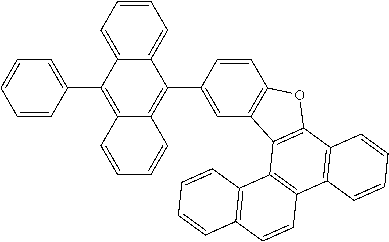 [SMILES: c1ccc(-c2c3ccccc3c(-c3ccc4oc5c6ccccc6c6ccc7ccccc7c6c5c4c3)c3ccccc23)cc1] | 82.39% | 571.69 |
| Compound 11 | 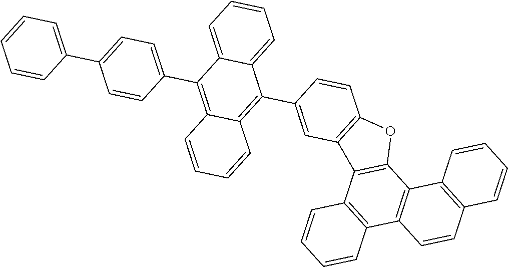 [SMILES: c1ccc(-c2ccc(-c3c4ccccc4c(-c4ccc5oc6c7c8ccccc8ccc7c7ccccc7c6c5c4)c4ccccc34)cc2)cc1] | 78.66% | 647.79 |
| Compound 14 | 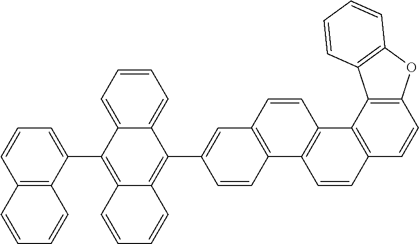 [SMILES: c1ccc2c(-c3c4ccccc4c(-c4ccc5c(ccc6c5ccc5ccc7oc8ccccc8c7c56)c4)c4ccccc34)cccc2c1] | 78.85% | 621.75 |
| Compound 15 | 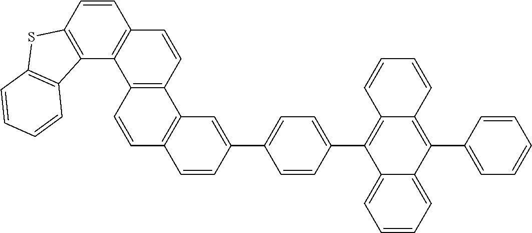 [SMILES: c1ccc(-c2c3ccccc3c(-c3ccc(-c4ccc5ccc6c(ccc7ccc8sc9ccccc9c8c76)c5c4)cc3)c3ccccc23)cc1] | 74.58% | 663.85 |
| Compound 16 | 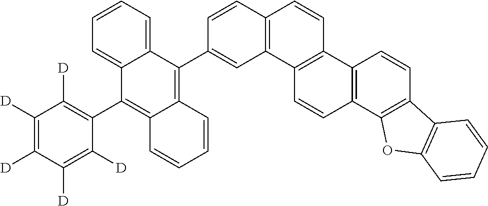 [SMILES: [2H]c1c([2H])c([2H])c(-c2c3ccccc3c(-c3ccc4ccc5c(ccc6c5ccc5c7ccccc7oc56)c4c3)c3ccccc23)c([2H])c1[2H]] | 79.74% | 576.72 |
| Compound 17 | 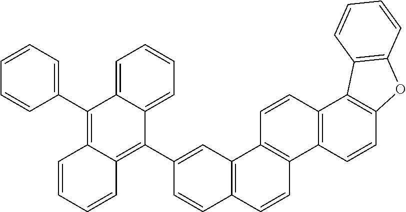 [SMILES: c1ccc(-c2c3ccccc3c(-c3ccc4ccc5c(ccc6c5ccc5oc7ccccc7c56)c4c3)c3ccccc23)cc1] | 70.43% | 571.69 |
| Compound 18 | 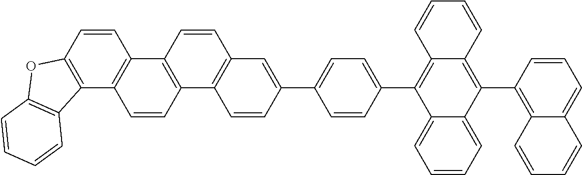 [SMILES: c1ccc2c(-c3c4ccccc4c(-c4ccc(-c5ccc6c(ccc7c6ccc6c7ccc7oc8ccccc8c76)c5)cc4)c4ccccc34)cccc2c1] | 72.56% | 697.85 |
| Compound 19 | 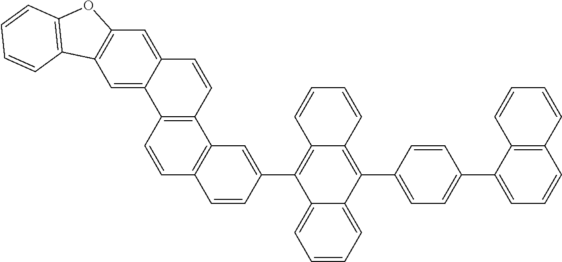 [SMILES: c1ccc2c(-c3ccc(-c4c5ccccc5c(-c5ccc6ccc7c8cc9c(cc8ccc7c6c5)oc5ccccc59)c5ccccc45)cc3)cccc2c1] | 70.51% | 697.85 |
| Compound 20 | 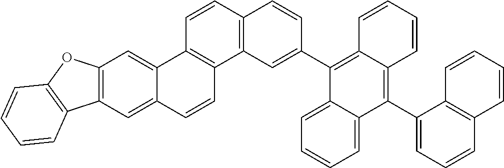 [SMILES: c1ccc2c(-c3c4ccccc4c(-c4ccc5ccc6c7cc8oc9ccccc9c8cc7ccc6c5c4)c4ccccc34)cccc2c1] | 75.64% | 621.75 |
| Compound 21 | 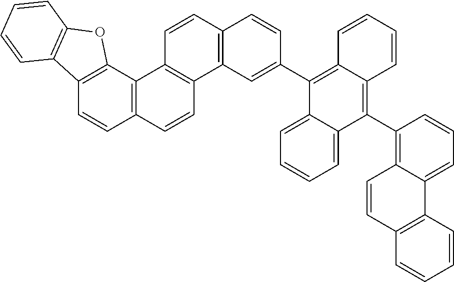 [SMILES: c1ccc2c(c1)ccc1c(-c3c4ccccc4c(-c4ccc5ccc6c(ccc7ccc8c9ccccc9oc8c76)c5c4)c4ccccc34)cccc12] | 77.83% | 671.23 |
| Compound 22 | 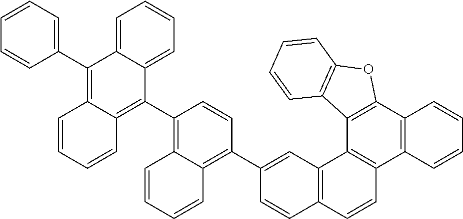 [SMILES: c1ccc(-c2c3ccccc3c(-c3ccc(-c4ccc5ccc6c7ccccc7c7oc8ccccc8c7c6c5c4)c4ccccc34)c3ccccc23)cc1] | 70.45% | 697.85 |
| Compound 23 | 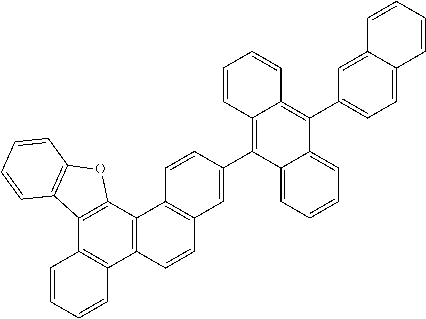 [SMILES: c1ccc2cc(-c3c4ccccc4c(-c4ccc5c(ccc6c7ccccc7c7c8ccccc8oc7c56)c4)c4ccccc34)ccc2c1] | 78.94% | 621.75 |

### Application Example: Manufacturing of Organic Electroluminescent Devices

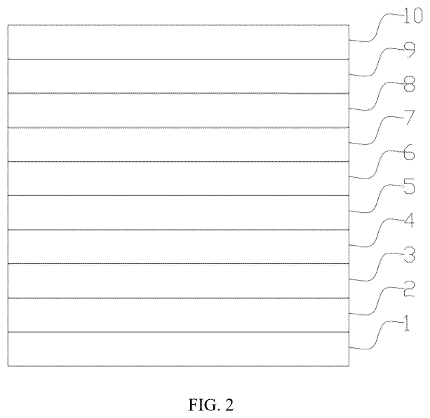

FIG. 2 is a schematic structural diagram of an organic electroluminescent device. The organic electroluminescent device includes a glass substrate 1, an anode 2, a hole injection layer 3, a first hole transport layer 4, a second hole transport layer 5, a light-emitting layer 6, a hole blocking layer 7, an electron transport layer 8, an electron injection layer 9, and a cathode 10, which are stacked in sequence.

Device manufacturing method:
a glass substrate 1 having an ITO transparent electrode (anode 2) on its surface was provided;
a thickness of the anode 2 was 100 nm;
the glass substrate 1 was ultrasonically cleaned in ethanol for 10 min, then oven-dried at 150° C., and treated with N₂ Plasma for 30 min;
compound HATCN was evaporated onto the surface of the anode 2 to form a hole injection layer 3 with a thickness of 5 nm;
compound HTM1 was evaporated onto the surface of the hole injection layer 3 to form a first hole transport layer 4 with a thickness of 60 nm;
compound HTM2 was evaporated onto the surface of the first hole transport layer 4 to form a second hole transport layer 5 with a thickness of 10 nm;
a host material and a blue light dopant material were co-evaporated onto the surface of the second hole transport layer 5 to form a light-emitting layer 6, where a weight ratio of the host material to the blue light dopant material was 97:3 (the host material types of each Example and Comparative Example were shown in Table 1), and a thickness of the light-emitting layer 6 was 25 nm;
a hole-blocking layer (HBL) material was evaporated onto the surface of the light-emitting layer to form a hole blocking layer 7 with a thickness of 5 nm;
an electron transport layer (ETL) was evaporated onto the surface of the hole blocking layer 7 to form an electron transport layer 8 with a thickness of 30 nm;
LiQ was evaporated onto the surface of the electron transport layer 8 to form an electron injection layer 9 with a thickness of 1 nm;
aluminum (Al) was evaporated onto the surface of the electron injection layer 9 to form a cathode 10 with a thickness of 100 nm, thereby yielding an organic electroluminescent device.

The structural formulas of HATCN, HTM1, HTM2, blue light dopant material, HBL, ETL, LiQ, and comparative compounds 1-6 are as follows:

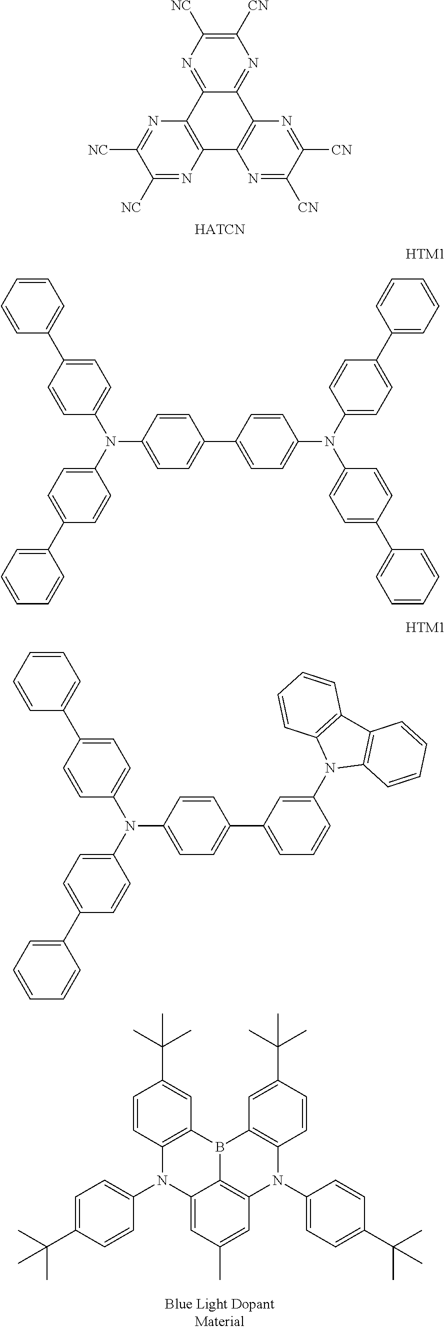

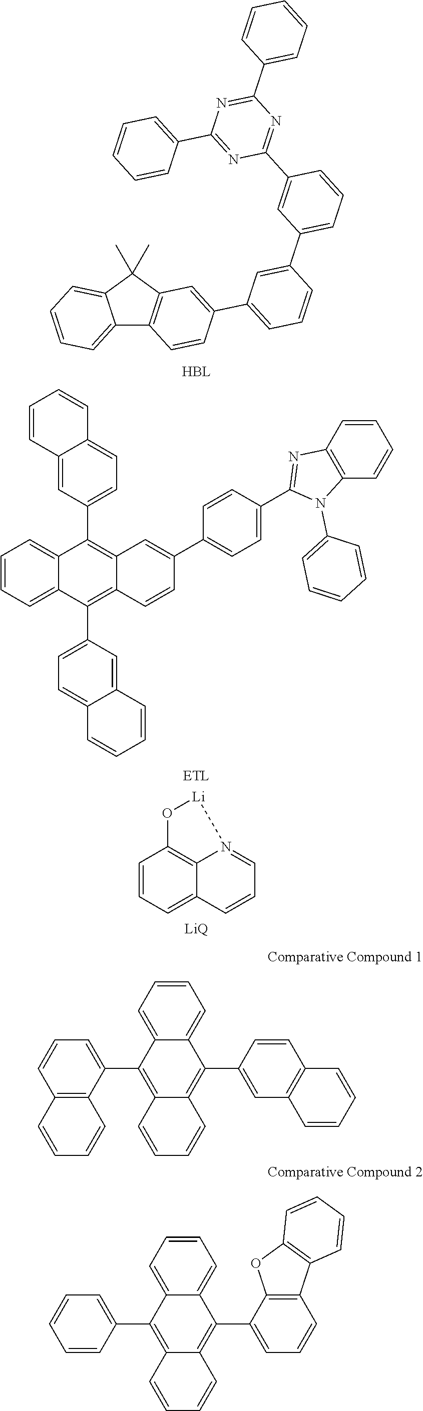

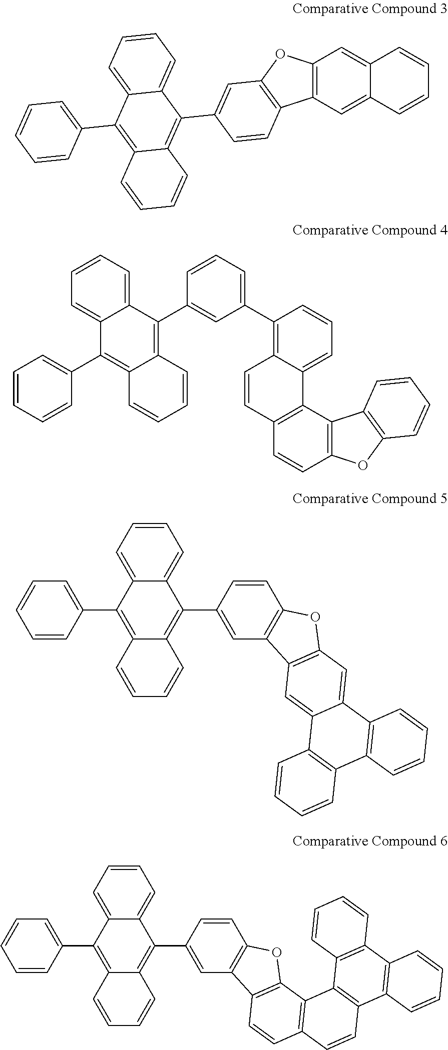 [SMILES: c1ccc(-c2c3ccccc3c(-c3ccc4c(c3)oc3cc5ccccc5cc34)c3ccccc23)cc1 ; c1ccc(-c2c3ccccc3c(-c3cccc(-c4cccc5c4ccc4ccc6oc7ccccc7c6c45)c3)c3ccccc23)cc1 ; c1ccc(-c2c3ccccc3c(-c3ccc4oc5cc6c7ccccc7c7ccccc7c6cc5c4c3)c3ccccc23)cc1 ; c1ccc(-c2c3ccccc3c(-c3ccc4oc5c(ccc6ccc7c8ccccc8c8ccccc8c7c65)c4c3)c3ccccc23)cc1]

### Evaluation: Device Performance Test

The device was driven at a constant current density using a constant-current source (Keithley 2400). The emission spectra were measured using a spectroradiometer (Konica Minolta CS 2000). The current-voltage-luminance (IVL) characteristics were recorded at a current density of 10 mA/cm², and the operational lifetime (LT₉₅) was evaluated at 50 mA/cm². The results are summarized in Table 2. All data are normalized relative to Comparative Example 1, which is set as a reference value of 100. For example, a driving voltage of 96 indicates that, under identical test conditions, the driving voltage of the device is 9600 of that of Comparative Example 1.

TABLE 2

| | Host | Driving | Current | |
|---|---|---|---|---|
| | material | voltage | efficiency | Lifetime |
| Example 1 | Compound 1 | 101 | 103 | 104 |
| Example 2 | Compound 2 | 99 | 102 | 103 |
| Example 3 | Compound 3 | 100 | 105 | 104 |
| Example 4 | Compound 4 | 97 | 101 | 113 |
| Example 5 | Compound 5 | 96 | 104 | 109 |
| Example 6 | Compound 6 | 98 | 104 | 106 |
| Example 7 | Compound 7 | 99 | 106 | 102 |
| Example 8 | Compound 8 | 98 | 102 | 104 |
| Example 9 | Compound 9 | 97 | 102 | 100 |
| Example 10 | Compound 10 | 100 | 104 | 106 |
| Example 11 | Compound 11 | 99 | 101 | 108 |
| Example 12 | Compound 12 | 98 | 105 | 122 |
| Example 13 | Compound 13 | 99 | 103 | 128 |
| Example 14 | Compound 14 | 97 | 104 | 107 |
| Example 15 | Compound 15 | 100 | 102 | 101 |
| Example 16 | Compound 16 | 98 | 103 | 115 |
| Example 17 | Compound 17 | 99 | 104 | 109 |
| Example 18 | Compound 18 | 96 | 105 | 102 |
| Example 19 | Compound 19 | 98 | 101 | 110 |
| Example 20 | Compound 20 | 98 | 103 | 103 |
| Example 21 | Compound 21 | 101 | 105 | 106 |
| Example 22 | Compound 22 | 100 | 104 | 104 |
| Example 23 | Compound 23 | 99 | 102 | 106 |
| Comparative | Comparative | 100 | 100 | 100 |
| Example 1 | Compound 1 | | | |
| Comparative | Comparative | 102 | 98 | 96 |
| Example 2 | Compound 2 | | | |
| Comparative | Comparative | 98 | 95 | 90 |
| Example 3 | Compound 3 | | | |
| Comparative | Comparative | 105 | 94 | 69 |
| Example 4 | Compound 4 | | | |
| Comparative | Comparative | 102 | 96 | 81 |
| Example 5 | Compound 5 | | | |
| Comparative | Comparative | 104 | 97 | 88 |
| Example 6 | Compound 6 | | | |

It can be seen from Table 2 that using the anthracene compounds in the examples of the present disclosure as blue light host materials resulted in significantly higher current efficiency than comparative compounds 1-6, and some compounds exhibited superior performance in terms of driving voltage or device lifetime.

In addition, the anthracene compounds have a low melting point and high optical and electrical stability, can be used in OLEDs as a host material, and have the potential of being applied to the AMOLED industry.

1. An anthracene compound, having a structure of formula (1): 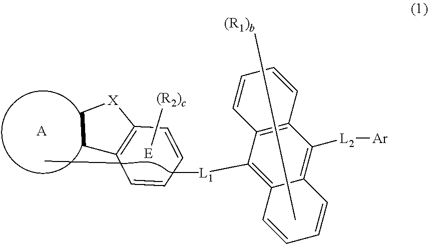
wherein ring A is selected from the following formula (2) or formula (3); 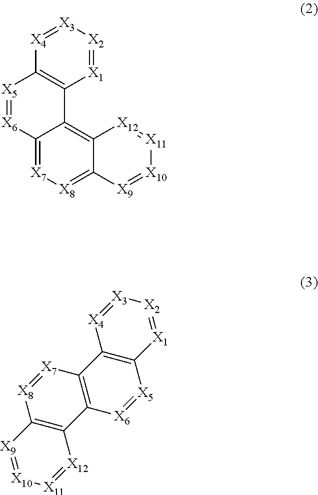
wherein X₁-X₁₂ are each independently selected from CR₀ or N; and two adjacent sites in X₁-X₄, X₅-X₆, X₇-X₈, X₉-X₁₂ are fused to a 5-membered ring containing X in formula (1);
R₀, R₁ and R₂ are each independently selected from hydrogen, deuterium, halogen, cyano, nitro, C₁-C₄₀ alkyl, C₁-C₄₀ heteroalkyl, C₂-C₄₀ alkenyl, C₂-C₄₀ alkynyl, C₃-C₄₀ cycloalkyl, C₃-C₄₀ heterocycloalkyl, C₆-C₆₀ aryl, C₃-C₆₀ heteroaryl, C₁-C₄₀ alkoxy, C₆-C₆₀ aryloxy, C₃-C₄₀ alkylsilyl, C₆-C₆₀ arylsilyl, C₁-C₄₀ alkylboranyl, C₆-C₆₀ arylboranyl, C₆-C₆₀ arylphosphino, or C₆-C₆₀ arylamino; and adjacent R₀ are capable of being connected into a fused ring;
b is an integer of 0-8; if b≥2, each R₁ is the same or different;
c is an integer of 0-4; if c≥2, each R₂ is the same or different;
X is selected from O or S;
L₁ and L₂ are each independently selected from single bond, substituted or unsubstituted C₆-C₆₀ arylene, or substituted or unsubstituted C₃-C₆₀ heteroarylene;
Ar is selected from substituted or unsubstituted C₆-C₃₆ aryl or substituted or unsubstituted C₂-C₃₆ heteroaryl;
each of substitutions in L₁, L₂ and Ar refers to being independently substituted with at least one of deuterium, halogen, cyano, isocyano, phosphino, C₁-C₆ alkyl, C₃-C₁₆ cycloalkyl, C₁-C₆ alkyl-substituted amino, C₁-C₆ hydrocarbyl-substituted or unsubstituted C₆-C₃₀ aryl, or C₁-C₆ hydrocarbyl-substituted or unsubstituted C₃-C₃₀ heteroaryl, wherein the number of substitutions ranges from monosubstitution to the maximum number of substitutions;
wherein a heteroatom in the heteroarylene, heteroaryl, heteroalkyl, or heterocycloalkyl is at least one selected from the group consisting of O, S, N, Se, Si, and Ge.

2. The anthracene compound according to claim 1, wherein the anthracene compound has a structure of formula (4): 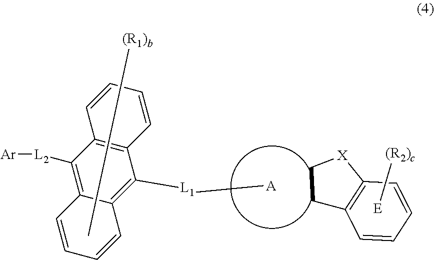
wherein a connection structure of ring A and L₁ moiety is: 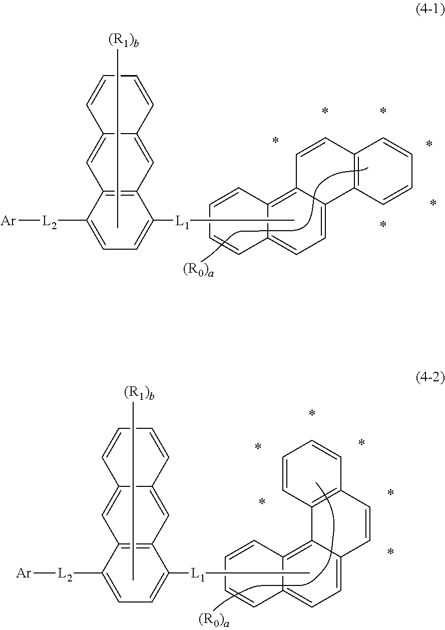
wherein * represents a site fused to a 5-membered ring containing X in formula (1);
a is an integer of 0-9; if a≥2, each R₀ is the same or different, and adjacent R₀ are capable of being connected into a fused ring.

3. The anthracene compound according to claim 1, wherein the anthracene compound has a structure of formula (6): 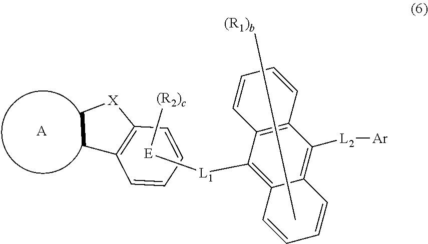
wherein c is an integer of 0-3; if c is 2 or 3, each R₂ is the same or different;
ring A is one of the structures of formula (A-1) to formula (A-8): 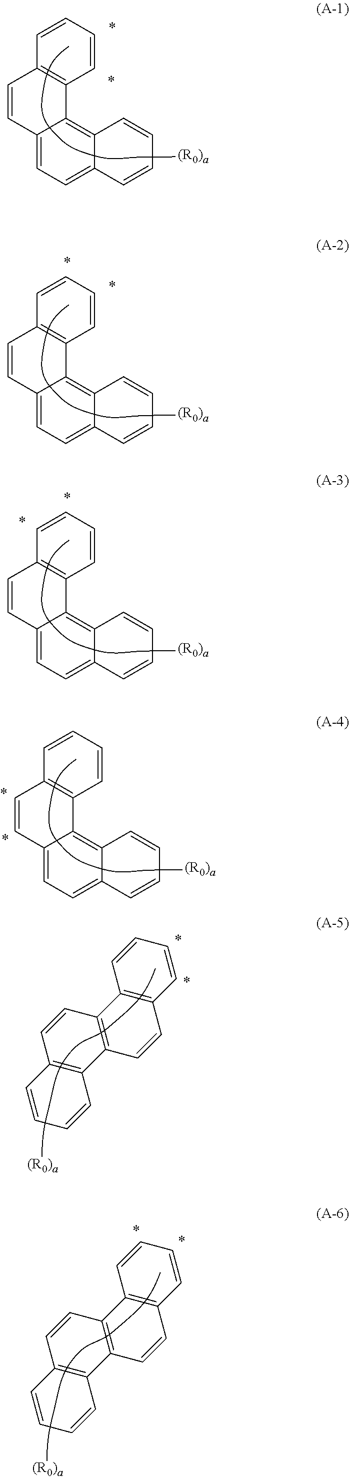 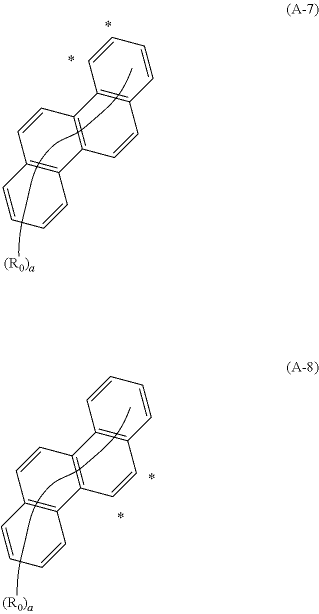
wherein * represents a site fused to a 5-membered ring containing X in formula (1);
a is an integer of 0-10; if a≥2, each R₀ is the same or different, and adjacent R₀ are capable of being connected into a fused ring.

4. The anthracene compound according to claim 1, wherein ring A is one of the structures of formula (A-91 to formula (A-161): 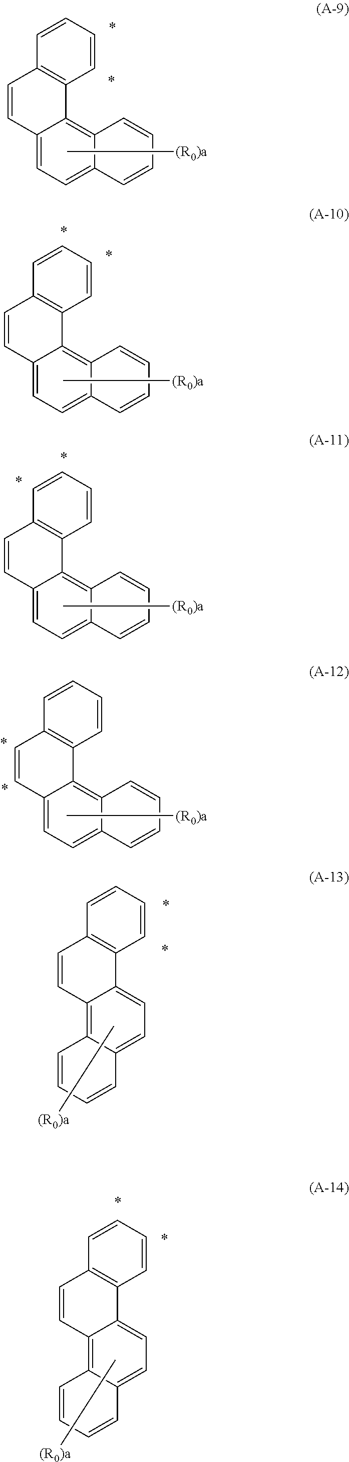 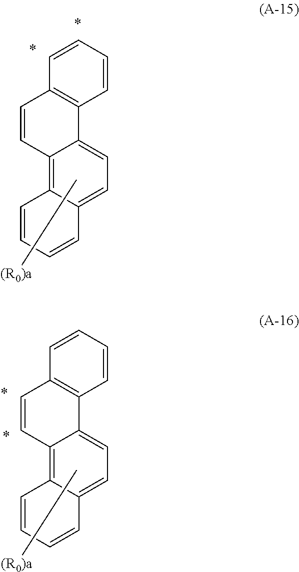
wherein * represents a site fused to a 5-membered ring containing X in formula (1);
a is an integer of 0-6; if a≥2, each R₀ is the same or different, and adjacent R₀ are capable of being connected into a fused ring.

5. The anthracene compound according to claim 1, wherein X₁-X₁₂ are all selected from CR₀.

6. The anthracene compound according to claim 1, wherein R₀, R₁ and R₂ are each independently selected from hydrogen, deuterium, halogen, cyano, C1-C30 alkyl, C1-C30 heteroalkyl, C3-C30 cycloalkyl, C3-C30 heterocycloalkyl, C6-C30 aryl, or C3-C30 heteroaryl; and adjacent R₀ are capable of being connected into a fused ring.

7. The anthracene compound according to claim 1, wherein L₁ and L₂ are each independently selected from single bond, substituted or unsubstituted C6-C30 arylene, or substituted or unsubstituted C3-C30 heteroarylene.

8. The anthracene compound according to claim 1, wherein Ar is selected from substituted or unsubstituted C6-C30 aryl, or substituted or unsubstituted C2-C30 heteroaryl.

9. The anthracene compound according to claim 1, wherein each of substitutions in L₁, L₂ and Ar refers to being independently substituted with at least one of deuterium, halogen, cyano, isocyano, phosphino, C1-C6 alkyl, C3-C16 cycloalkyl, C1-C6 alkyl-substituted amino, C1-C6 hydrocarbyl-substituted or unsubstituted C6-C18 aryl, or C1-C6 hydrocarbyl-substituted or unsubstituted C3-C18 heteroaryl, wherein the number of substitutions ranges from monosubstitution to the maximum number of substitutions.

10. The anthracene compound according to claim 6, wherein R₀, R₁ and R₂ are each independently selected from hydrogen, deuterium, halogen, cyano, C1-C10 alkyl, C1-C10 heteroalkyl, C3-C10 cycloalkyl, C3-C10 heterocycloalkyl, C6-C12 aryl, or C3-C12 heteroaryl.

11. The anthracene compound according to claim 7, wherein L₁ and L₂ are each independently selected from single bond, substituted or unsubstituted C6-C14 arylene, or substituted or unsubstituted C3-C12 heteroarylene.

12. The anthracene compound according to claim 9, wherein each of substitutions in L₁, L₂ and Ar refers to being independently substituted with at least one of deuterium, halogen, cyano, isocyano, C1-C6 alkyl, C3-C16 cycloalkyl, C1-C6 hydrocarbyl-substituted or unsubstituted C6-C12 aryl, or C1-C6 hydrocarbyl-substituted or unsubstituted C3-C12 heteroaryl, wherein the number of substitutions ranges from monosubstitution to the maximum number of substitutions.

13. The anthracene compound according to claim 1, wherein Ar is selected from oxaspirofluorenyl, substituted or unsubstituted phenyl, substituted or unsubstituted biphenyl, substituted or unsubstituted naphthyl, substituted or unsubstituted anthryl, substituted or unsubstituted dibenzofuranyl, substituted or unsubstituted dibenzothienyl, substituted or unsubstituted 9,9-dimethylfluorenyl, substituted or unsubstituted 9,9-diphenylfluorenyl, substituted or unsubstituted spirobifluorenyl, substituted or unsubstituted phenanthryl, substituted or unsubstituted pyrenyl, substituted or unsubstituted chrysenyl, substituted or unsubstituted carbazolyl, substituted or unsubstituted pyridyl, substituted or unsubstituted pyrimidinyl, substituted or unsubstituted fluoranthenyl, substituted or unsubstituted benzoxazolyl, substituted or unsubstituted benzimidazolyl, substituted or unsubstituted benzotriazolyl, substituted or unsubstituted benzophenanthryl, substituted or unsubstituted benzocyclohexyl, substituted or unsubstituted phenanthrooxazolyl, or substituted or unsubstituted benzocarbazolyl, or a combination of at least two thereof, substitutions are substitutions with at least one of deuterium, halogen, cyano, isocyano, C1-C6 alkyl, or C3-C16 cycloalkyl, wherein the number of substitutions ranges from monosubstitution to the maximum number of substitutions.

14. The anthracene compound according to claim 1, wherein the anthracene compound satisfies at least one of the following conditions:
(1) X₁-X₁₂ are all selected from CR₀, and R₀ is selected from hydrogen or deuterium;
(2) X is selected from 0;
wherein substitutions are each independently substitutions with deuterium or fluorine, wherein the number of substitutions ranges from monosubstitution to the maximum number of substitutions.

15. The anthracene compound according to claim 1, wherein R₁ is selected from hydrogen, deuterium, or substituted or unsubstituted phenyl;
wherein substitutions are each independently substitutions with deuterium or fluorine, wherein the number of substitutions ranges from monosubstitution to the maximum number of substitutions.

16. The anthracene compound according to claim 1, wherein R₂ is selected from hydrogen or deuterium;
wherein substitutions are each independently substitutions with deuterium or fluorine, wherein the number of substitutions ranges from monosubstitution to the maximum number of substitutions.

17. The anthracene compound according to claim 1, wherein L₁ and L₂ are each independently selected from single bond, a divalent group of dibenzofuran, a divalent group of dibenzothiophene, substituted or unsubstituted phenylene, substituted or unsubstituted biphenylene, substituted or unsubstituted naphthylene, substituted or unsubstituted phenanthrylene, or substituted or unsubstituted anthrylene;
wherein substitutions are each independently substitutions with deuterium or fluorine, wherein the number of substitutions ranges from monosubstitution to the maximum number of substitutions.

18. The anthracene compound according to claim 1, wherein Ar is selected from the group consisting of substituted or unsubstituted phenyl, substituted or unsubstituted biphenyl, substituted or unsubstituted naphthyl, substituted or unsubstituted phenanthryl, substituted or unsubstituted anthryl, dibenzofuranyl, and dibenzothienyl;
wherein substitutions are each independently substitutions with deuterium or fluorine, wherein the number of substitutions ranges from monosubstitution to the maximum number of substitutions.

19. An organic electroluminescent device, comprising the anthracene compound according to claim 1.

20. The organic electroluminescent device according to claim 19, comprising an anode and a cathode, wherein a light-emitting layer is disposed between the anode and the cathode; the light-emitting layer comprises the anthracene compound.
